(19) United States
(12) Patent Application Publication
(10) Pub. No.: US 2024/0268227 A1
(43) Pub. Date: Aug. 8, 2024

(54) CONDENSED POLYCYCLIC COMPOUND AND ORGANIC LIGHT-EMITTING DEVICE INCLUDING SAME

(71) Applicant: LT MATERIALS CO., LTD., Yongin-si, Gyeonggi-do (KR)

(72) Inventors: Gi-Back LEE, Yongin-si (KR); Jun-Tae MO, Yongin-si (KR); Dong-Jun KIM, Yongin-si (KR)

(73) Assignee: LT MATERIALS CO., LTD., Yongin-si, Gyeonggi-do (KR)

(21) Appl. No.: 18/288,675

(22) PCT Filed: Nov. 16, 2021
(86) PCT No.: PCT/KR2021/016791

§ 371 Date: Oct. 27, 2023

(30) Foreign Application Priority Data
May 10, 2021 (KR) 10-2021-0060133

(51) Int. Cl. H10K 85/60; C07D 307/77; C07D 307/91; C07D 333/50; C07D 405/04; C07D 405/10; C07D 409/12; C07D 493/04; C07D 495/04; C09K 11/06; H10K 50/11

(52) U.S. Cl. CPC H10K 85/6574; C07D 307/77; C07D 307/91; C07D 333/50; C07D 405/04; C07D 405/10; C07D 409/12; C07D 493/04; C07D 495/04; C09K 11/06; H10K 85/615; H10K 85/633; H10K 85/636; H10K 85/654; C09K 2211/1018; H10K 50/11; H10K 85/626; H10K 85/657; H10K 85/6576

(57) ABSTRACT

The present specification relates to a condensed polycyclic compound of Chemical Formula 1, and an organic light emitting device including same.

### TECHNICAL FIELD

The present specification relates to a condensed polycyclic compound, and an organic light emitting device including same.

The present specification claims priority to and the benefits of Korean Patent Application No. 10-2021-0060133, filed with the Korean Intellectual Property Office on May 10, 2021, the entire contents of which are incorporated herein by reference.

### BACKGROUND ART

An electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

### DISCLOSURE

### Technical Problem

The present specification is directed to providing a condensed polycyclic compound, and an organic light emitting device including same.

### Technical Solution

One embodiment of the present specification provides a condensed polycyclic compound of the following Chemical Formula 1.

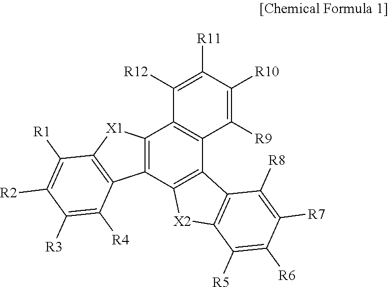

In Chemical Formula 1,

X1 and X2 are the same as or different from each other, and each independently CPR′; O; or S,

R and R′ are the same as or different from each other, and each independently hydrogen; deuterium; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; or a substituted or unsubstituted heteroaryl group having 2 to 60 carbon atoms,

R1 to R12 are the same as or different from each other, and each independently hydrogen; deuterium; a halogen group; a cyano group; —N(R21)m(R22)n; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; or a heteroaryl group having 2 to 60 carbon atoms substituted or unsubstituted and including a double bond of carbon and nitrogen,

m and n are each an integer of 1 to 5,

when m and n are each 2 or greater, substituents in the parentheses are the same as or different from each other,

at least one of R1 to R12 is -(L)q-(Z)r,

L is a direct bond; a substituted or unsubstituted arylene group having 6 to 60 carbon atoms; or a substituted or unsubstituted heteroarylene group having 2 to 60 carbon atoms,

Z is —N(R31)o(R32)p; or a heteroaryl group having 2 to 60 carbon atoms substituted or unsubstituted and including a double bond of carbon and nitrogen,

o, p, q and r are each an integer of 1 to 5,

when o, p, q and r are each 2 or greater, substituents in the parentheses are the same as or different from each other,

when two or more of R1 to R12 are -(L)q-(Z)r, Ls and Zs are each the same as or different from each other,

R21, R22, R31 and R32 are the same as or different from each other, and each independently hydrogen; deuterium; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; or a heteroaryl group having 2 to 60 carbon atoms substituted or unsubstituted and including O or S, or R21 and R22, and R31 and R32 each bond to form a substituted or unsubstituted aliphatic or aromatic hydrocarbon ring having 6 to 60 carbon atoms, or a substituted or unsubstituted aliphatic or aromatic heteroring having 2 to 60 carbon atoms, and

when any one of R21 and R22 or any one of R31 and R32 is a heteroaryl group having 2 to 60 carbon atoms substituted or unsubstituted and including O or S, the other one of R21 and R22 or the other one of R31 and R32 is a substituted or unsubstituted aryl group having 10 to 60 carbon atoms.

Another embodiment of the present specification provides an organic light emitting device including a first electrode; a second electrode; and one or more organic material layers provided between the first electrode and the second electrode, wherein one or more layers of the organic material layers include the condensed polycyclic compound of Chemical Formula 1.

### Advantageous Effects

A compound described in the present specification can be used as a material of an organic material layer of an organic light emitting device. The compound is capable of performing a role of a hole injection material, a hole transfer material, a light emitting material, an electron transfer material, an electron injection material, a charge generation material or the like in an organic light emitting device. Particularly, the compound can be used as a light emitting material of an organic light emitting device.

When the condensed polycyclic compound of Chemical Formula 1 is used as a light emitting material of an organic light emitting device, that is, included in a light emitting layer and used, an organic light emitting device having superior properties in terms of driving voltage and lifetime can be provided.

Specifically, the condensed polycyclic compound of Chemical Formula 1 has a core structure in which six rings are condensed. When adding an amine-based substituent thereto, the unshared electron pair of the amine improves a flow of holes, which strengthens hole properties, and when adding an azine-based substituent thereto, electron withdrawing properties are strengthened. Through this, an injection barrier can be improved and energy can be efficiently transferred from a light emitting region host to a dopant molecule through adjusting a band gap and a T₁ (energy level in triplet state) value.

In addition, thermal stability of the compound can be enhanced by increasing the glass transition temperature through the amine- and azine-based substituents, and since molecular stability is enhanced as well, a driving voltage of a device can be lowered, light emission efficiency can be enhanced, and lifetime properties of the device can be enhanced by thermal stability of the compound.

### DESCRIPTION OF DRAWINGS

FIG. 1 to FIG. 4 are diagrams each illustrating a lamination structure of an organic light emitting device according to one embodiment of the present specification.

### REFERENCE NUMERAL

100: Substrate
200: Anode
300: Organic Material Layer
301: Hole Injection Layer
302: Hole Transfer Layer
303: Electron Blocking Layer
304: Light Emitting Layer
305: Hole Blocking Layer
306: Electron Transfer Layer
307: Electron Injection Layer
400: Cathode

### MODE FOR DISCLOSURE

Hereinafter, the present specification will be described in more detail.

In the present specification, a description of a certain part “including” certain constituents means capable of further including other constituents, and does not exclude other constituents unless particularly stated on the contrary.

A term “substitution” means a hydrogen atom bonding to a carbon atom of a compound being changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of deuterium; a halogen group; a cyano group; a C1 to C60 alkyl group; a C2 to C60 alkenyl group; a C2 to C60 alkynyl group; a C3 to C60 cycloalkyl group; a C2 to C60 heterocycloalkyl group; a C6 to C60 aryl group; a C2 to C60 heteroaryl group; a silyl group; a phosphine oxide group; and an amine group, or being substituted with a substituent linking two or more substituents selected from among the substituents illustrated above, or being unsubstituted.

In the present specification, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present application, a “case of a substituent being not indicated in a chemical formula or compound structure” may mean that positions that may come as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present application, in a “case of a substituent being not indicated in a chemical formula or compound structure”, hydrogen and deuterium may be mixed in compounds when deuterium is not explicitly excluded such as a deuterium content being 0%, a hydrogen content being 100% or substituents being all hydrogen.

In one embodiment of the present application, deuterium is one of isotopes of hydrogen, is an element having deuteron formed with one proton and one neutron as a nucleus, and may be expressed as hydrogen-2, and the elemental symbol may also be written as D or ²H.

In one embodiment of the present application, an isotope means an atom with the same atomic number (Z) but with a different mass number (A), and may also be interpreted as an element with the same number of protons but with a different number of neutrons.

In one embodiment of the present application, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

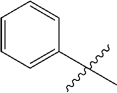 [SMILES: *c1ccccc1]

means that the total number of substituents that the phenyl group may have is 5 (T1 in the formula), and the number of deuterium among these is 1 (T2 in the formula). In other words, having a deuterium content of 20% in a phenyl group may be represented by the following structural formulae.

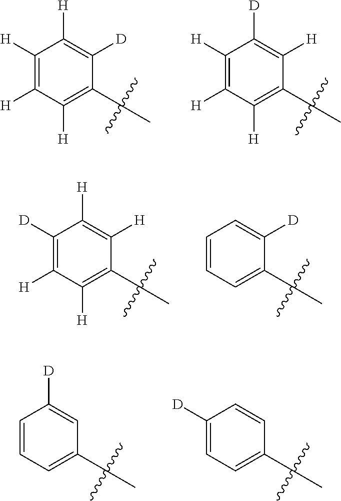 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present application, “a phenyl group having a deuterium content of 0%” may mean a phenyl group that does not include a deuterium atom, that is, a phenyl group that has 5 hydrogen atoms.

In the present specification, the alkyl group includes linear or branched, and may be further substituted with other substituents. The number of carbon atoms of the alkyl group may be from 1 to 60, specifically from 1 to 40 and more specifically from 1 to 20. Specific examples thereof may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group includes linear or branched, and may be further substituted with other substituents. The number of carbon atoms of the alkenyl group may be from 2 to 60, specifically from 2 to 40 and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes linear or branched, and may be further substituted with other substituents. The number of carbon atoms of the alkynyl group may be from 2 to 60, specifically from 2 to 40 and more specifically from 2 to 20.

In the present specification, the cycloalkyl group includes monocyclic or polycyclic, and may be further substituted with other substituents. Herein, the polycyclic means a group in which the cycloalkyl group is directly linked to or condensed with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes monocyclic or polycyclic, and may be further substituted with other substituents. Herein, the polycyclic means a group in which the heterocycloalkyl group is directly linked to or condensed with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes monocyclic or polycyclic, and may be further substituted with other substituents. Herein, the polycyclic means a group in which the aryl group is directly linked to or condensed with other cyclic groups. Herein, the other cyclic groups may be an aryl group, but may also be different types of cyclic groups such as a cycloalkyl group, a heterocycloalkyl group and a heteroaryl group. The aryl group includes a spiro group. The number of carbon atoms of the aryl group may be from 6 to 60, specifically from 6 to 40 and more specifically from 6 to 25. When the aryl group is dicyclic or higher, the number of carbon atoms may be from 8 to 60, from 8 to 40 or from 8 to 30. Specific examples of the aryl group may include a phenyl group, a biphenyl group, a ter-phenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a condensed ring group thereof, and the like, but are not limited thereto.

In the present specification, the fluorenyl group may be substituted, and adjacent substituents may bond to each other to form a ring.

When the fluorenyl group is substituted,

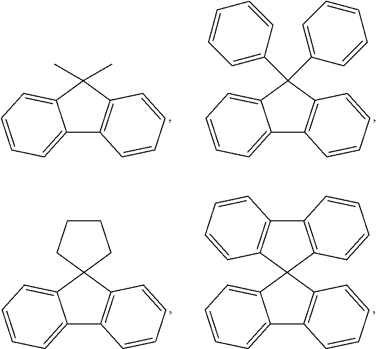

and the like may be included, however, the structure is not limited thereto.

In the present specification, the heteroaryl group includes O, S, SO₂, Se, N or Si as a heteroatom, includes monocyclic or polycyclic, and may be further substituted with other substituents. Herein, the polycyclic means a group in which the heteroaryl group is directly linked to or condensed with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. When the heteroaryl group is dicyclic or higher, the number of carbon atoms may be from 4 to 60, 4 to 40 or 4 to 25. Specific examples of the heteroaryl group may include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, 5,10-dihydrobenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a benzofuro[2,3-d]pyrimidyl group; a benzothieno[2,3-d]pyrimidyl group; a benzofuro[2,3-a]carbazolyl group, a benzothieno[2,3-a]carbazolyl group, a 1,3-dihydroindolo[2,3-a]carbazolyl group, a benzofuro[3,2-a]carbazolyl group, a benzothieno[3,2-a]carbazolyl group, a 1,3-dihydroindolo[3,2-a]carbazolyl group, a benzofuro[2,3-b]carbazolyl group, a benzothieno[2,3-b]carbazolyl group, a 1,3-dihydroindolo[2,3-b]carbazolyl group, a benzofuro[3,2-b]carbazolyl group, a benzothieno[3,2-b]carbazolyl group, a 1,3-dihydroindolo[3,2-b]carbazolyl group, a benzofuro[2,3-c]carbazolyl group, a benzothieno[2,3-c]carbazolyl group, a 1,3-dihydroindolo[2,3-c]carbazolyl group, a benzofuro[3,2-c]carbazolyl group, a benzothieno[3,2-c]carbazolyl group, a 1,3-dihydroindolo[3,2-c]carbazolyl group, a 1,3-dihydroindeno[2,1-b]carbazolyl group, a 5,11-dihydroindeno[1,2-b]carbazolyl group, a 5,12-dihydroindeno[1,2-c]carbazolyl group, a 5,8-dihydroindeno[2,1-c]carbazolyl group, a 7,12-dihydroindeno[1,2-a]carbazolyl group, a 11,12-dihydroindeno[2,1-a]carbazolyl group and the like, but are not limited thereto.

In the present specification, the silyl group is a substituent including Si and having the Si atom directly linked as a radical, and is represented by —Si(R101)(R102)(R103). R101 to R103 are the same as or different from each other, and may be each independently a substituent formed with at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group may include a trimethylsilyl group

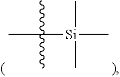 [SMILES: *[Si](C)(C)C]

a triethylsilyl group

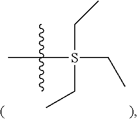

a t-butyldimethylsilyl group

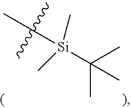

a vinyldimethylsilyl group

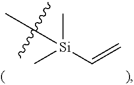

a propyldimethylsilyl group

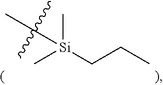

a triphenylsilyl group

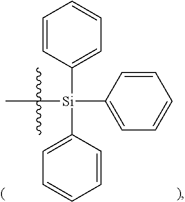

a diphenylsilyl group

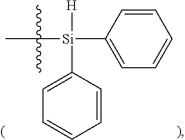

a phenylsilyl group

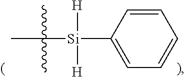

and the like, but are not limited thereto.

In the present specification, the phosphine oxide group is represented by —P(═O)(R104)(R105), and R104 and R105 are the same as or different from each other and may be each independently a substituent formed with at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and as the alkyl group and the aryl group, the examples described above may be used. Examples of the phosphine oxide group may include a dimethylphosphine oxide group, a diphenylphosphine oxide group, a dinaphthylphosphine oxide group and the like, but are not limited thereto.

In the present specification, the amine group is represented by —N(R106)(R107), and R106 and R107 are the same as or different from each other and may be each independently a substituent formed with at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the descriptions on the aryl group provided above may be applied to an arylene group except that the arylene group is a divalent group.

In the present specification, a phenylene group may be selected from among the following structures.

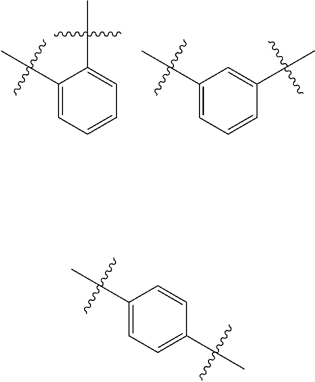 [SMILES: *c1ccccc1* ; *c1cccc(*)c1 ; *c1ccc(*)cc1]

In the present specification, the descriptions on the heteroaryl group provided above may be applied to a heteroarylene group except that the heteroarylene group is a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

In the present specification, the descriptions on the cycloalkyl group and the aryl group provided above may be applied to an aliphatic or aromatic hydrocarbon ring except for those that are not a monovalent group.

In the present specification, the descriptions on the heterocycloalkyl group and the heteroaryl group provided above may be applied to an aliphatic or aromatic heteroring except for those that are not a monovalent group.

In one embodiment of the present specification, X1 and X2 are the same as or different from each other, and each independently CRR′; O; or S.

In one embodiment of the present specification, at least one of X1 and X2 is O; or S.

In one embodiment of the present specification, X1 and X2 may be O.

In one embodiment of the present specification, X1 and X2 may be S.

In one embodiment of the present specification, X1 is O, and X2 may be S.

In one embodiment of the present specification, X1 is O, and X2 may be CRR′.

In one embodiment of the present specification, X1 is S, and X2 may be O.

In one embodiment of the present specification, X1 is S, and X2 may be CRR′.

In one embodiment of the present specification, X1 may be O; or S.

In one embodiment of the present specification, X2 may be O; S; or CRR′.

In one embodiment of the present specification, R and R′ are the same as or different from each other, and may be each independently a substituted or unsubstituted alkyl group having 1 to 60 carbon atoms.

In one embodiment of the present specification, R and R′ are the same as or different from each other, and may be each independently a substituted or unsubstituted alkyl group having 1 to 30 carbon atoms.

In one embodiment of the present specification, R and R′ are the same as or different from each other, and may be each independently a substituted or unsubstituted alkyl group having 1 to 10 carbon atoms.

In one embodiment of the present specification, R and R′ are the same as or different from each other, and may be each independently an alkyl group having 1 to 10 carbon atoms.

In one embodiment of the present specification, R and R′ are the same as each other, and may be an alkyl group having 1 to 10 carbon atoms.

In one embodiment of the present specification, R and R′ are the same as each other, and may be a methyl group.

In one embodiment of the present specification, R1 to R12 are the same as or different from each other, and each independently hydrogen; deuterium; a halogen group; a cyano group; —N(R21)m(R22)n; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; or a heteroaryl group having 2 to 60 carbon atoms substituted or unsubstituted and including a double bond of carbon and nitrogen, and at least one of R1 to R12 may be -(L)q-(Z)r.

In one embodiment of the present specification, R21 and R22 are the same as or different from each other, and each independently hydrogen; deuterium; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; or a heteroaryl group having 2 to 60 carbon atoms substituted or unsubstituted and including O or S, or R21 and R22 may each bond to form a substituted or unsubstituted aliphatic or aromatic hydrocarbon ring having 6 to 60 carbon atoms, or a substituted or unsubstituted aliphatic or aromatic heteroring having 2 to 60 carbon atoms.

In the present specification, —N(R21)m(R22)n may be represented by the following structural formula.

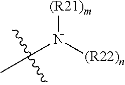

For example, when m and n are each 2, the structure may be represented as follows, R21′ and R22′ have the same definitions as R21 and R22, respectively, and R21 and R21′ are the same as or different from each other and R22 and R22′ are the same as or different from each other.

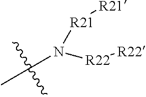

In one embodiment of the present specification, R21 and R22 may each bond to form a substituted or unsubstituted aliphatic or aromatic heteroring having 2 to 60 carbon atoms.

In one embodiment of the present specification, R21 and R22 may each bond to form an aliphatic or aromatic heteroring having 2 to 60 carbon atoms substituted or unsubstituted and including N.

In one embodiment of the present specification, R21 and R22 may each bond to form an aromatic heteroring having 2 to 60 carbon atoms substituted or unsubstituted and including N.

In one embodiment of the present specification, R21 and R22 may each bond to form a substituted or unsubstituted carbazole ring; or a substituted or unsubstituted benzocarbazole ring.

In the present specification, including a double bond of carbon and nitrogen means including a —C═N— bond in a substituent. For example, pyridine, pyrimidine, triazine, quinazoline, quinoxaline and the like include a double bond of carbon and nitrogen, and carbazole does not include a double bond of carbon and nitrogen.

In one embodiment of the present specification, one or two of R1 to R12 may be -(L)q-(Z)r.

In one embodiment of the present specification, any one of R1 to R12 may be -(L)q-(Z)r.

In one embodiment of the present specification, two of R1 to R12 may be -(L)q-(Z)r.

In one embodiment of the present specification, the rest of R1 to R12 that are not -(L)q-(Z)r may be hydrogen; deuterium; a substituted or unsubstituted aryl group having 6 to 60 carbon atoms; or a heteroaryl group having 2 to 60 carbon atoms substituted or unsubstituted and including a double bond of carbon and nitrogen.

In one embodiment of the present specification, the rest of R1 to R12 that are not -(L)q-(Z)r may be hydrogen; deuterium; a substituted or unsubstituted aryl group having 6 to 30 carbon atoms; or a heteroaryl group having 2 to 30 carbon atoms substituted or unsubstituted and including a double bond of carbon and nitrogen.

In one embodiment of the present specification, the rest of R1 to R12 that are not -(L)q-(Z)r may be hydrogen; deuterium; a substituted or unsubstituted aryl group having 6 to 15 carbon atoms; or a heteroaryl group having 2 to 15 carbon atoms substituted or unsubstituted and including a double bond of carbon and nitrogen.

In one embodiment of the present specification, the rest of R1 to R12 that are not -(L)q-(Z)r may be hydrogen; deuterium; a substituted or unsubstituted phenyl group; a substituted or unsubstituted pyrimidine group; a substituted or unsubstituted triazine group; a substituted or unsubstituted quinazoline group; a substituted or unsubstituted quinoxaline group; or a substituted or unsubstituted benzofuropyrimidine group.

In one embodiment of the present specification, the rest of R1 to R12 that are not -(L)q-(Z)r may be hydrogen; deuterium; or a substituted or unsubstituted aryl group having 6 to 60 carbon atoms.

In one embodiment of the present specification, the rest of R1 to R12 that are not -(L)q-(Z)r may be hydrogen; deuterium; or a substituted or unsubstituted aryl group having 6 to 30 carbon atoms.

In one embodiment of the present specification, the rest of R1 to R12 that are not -(L)q-(Z)r may be hydrogen; deuterium; or an aryl group having 6 to 30 carbon atoms.

In one embodiment of the present specification, the rest of R1 to R12 that are not -(L)q-(Z)r may be hydrogen; deuterium; or an aryl group having 6 to 15 carbon atoms.

In one embodiment of the present specification, the rest of R1 to R12 that are not -(L)q-(Z)r may be hydrogen; deuterium; or a phenyl group.

In one embodiment of the present specification, L is a direct bond; or a substituted or unsubstituted C6 to C60 arylene group.

In one embodiment of the present specification, L is a direct bond; or a substituted or unsubstituted C6 to C30 arylene group.

In one embodiment of the present specification, L is a direct bond; or a substituted or unsubstituted C6 to C15 arylene group.

In one embodiment of the present specification, L may be a direct bond; a substituted or unsubstituted phenylene group; a substituted or unsubstituted biphenylene group; or a substituted or unsubstituted naphthylene group.

In one embodiment of the present specification, L may be a direct bond; a phenylene group unsubstituted or substituted with deuterium; a biphenylene group unsubstituted or substituted with deuterium; or a naphthylene group unsubstituted or substituted with deuterium.

In one embodiment of the present specification, L may be a direct bond; a phenylene group; a biphenylene group; or a naphthylene group.

In one embodiment of the present specification, Z is —N(R31)o(R32)p; or a heteroaryl group having 2 to 60 carbon atoms substituted or unsubstituted and including a double bond of carbon and nitrogen.

In one embodiment of the present specification, Z is —N(R31)o(R32)p; or a heteroaryl group having 2 to 30 carbon atoms substituted or unsubstituted and including a double bond of carbon and nitrogen.

In one embodiment of the present specification, Z is —N(R31)o(R32)p; or a heteroaryl group having 2 to 15 carbon atoms substituted or unsubstituted and including a double bond of carbon and nitrogen.

In one embodiment of the present specification, Z may be —N(R31)o(R32)p; a substituted or unsubstituted pyrimidine group; a substituted or unsubstituted triazine group; a substituted or unsubstituted quinazoline group; a substituted or unsubstituted quinoxaline group; or a substituted or unsubstituted benzofuropyrimidine group.

In one embodiment of the present specification, Z may be —N(R31)o(R32)p; a pyrimidine group unsubstituted or substituted with an aryl group; a triazine group unsubstituted or substituted with an aryl group unsubstituted or substituted with deuterium or a carbazole group, or a heteroaryl group; a quinazoline group unsubstituted or substituted with an aryl group; a quinoxaline group unsubstituted or substituted with an aryl group; or a benzofuropyrimidine group unsubstituted or substituted with an aryl group.

In one embodiment of the present specification, being substituted with a carbazole group includes both cases of bonding to nitrogen of the carbazole and bonding to carbon of the carbazole.

In one embodiment of the present specification, when any one of R21 and R22 or any one of R31 and R32 is a heteroaryl group having 2 to 60 carbon atoms substituted or unsubstituted and including O or S, the other one of R21 and R22 or the other one of R31 and R32 is a substituted or unsubstituted aryl group having 10 to 60 carbon atoms.

In one embodiment of the present specification, when any one of R21 and R22 or any one of R31 and R32 is a heteroaryl group having 2 to 30 carbon atoms substituted or unsubstituted and including O or S, the other one of R21 and R22 or the other one of R31 and R32 is a substituted or unsubstituted aryl group having 10 to 30 carbon atoms.

In one embodiment of the present specification, when any one of R21 and R22 or any one of R31 and R32 is a substituted or unsubstituted dibenzofuran group; or a substituted or unsubstituted dibenzothiophene group, the other one of R21 and R22 or the other one of R31 and R32 is a substituted or unsubstituted aryl group having 10 to 20 carbon atoms.

In one embodiment of the present specification, R31 and R32 are the same as or different from each other, and each independently hydrogen; deuterium; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; or a heteroaryl group having 2 to 60 carbon atoms substituted or unsubstituted and including O or S, or R31 and R32 may each bond to form a substituted or unsubstituted aliphatic or aromatic hydrocarbon ring having 6 to 60 carbon atoms, or a substituted or unsubstituted aliphatic or aromatic heteroring having 2 to 60 carbon atoms.

In one embodiment of the present specification, R31 and R32 may bond to form a substituted or unsubstituted aliphatic or aromatic heteroring having 2 to 60 carbon atoms.

In one embodiment of the present specification, R31 and R32 may bond to form an aliphatic or aromatic heteroring having 2 to 60 carbon atoms substituted or unsubstituted and including N.

In one embodiment of the present specification, R31 and R32 may bond to form an aromatic heteroring having 2 to 60 carbon atoms substituted or unsubstituted and including N.

In one embodiment of the present specification, R31 and R32 may each bond to form a substituted or unsubstituted carbazole ring; or a substituted or unsubstituted benzocarbazole ring.

In one embodiment of the present specification, when Z is —N(R31)o(R32)p, the condensed polycyclic compound may satisfy any one of i) R31 and R32 are the same as or different from each other, and each independently a substituted or unsubstituted aryl group having 6 to 30 carbon atoms; ii) any one of R31 and R32 is a heteroaryl group having 2 to 30 carbon atoms substituted or unsubstituted and including O or S, and the other one of R31 and R32 is a substituted or unsubstituted aryl group having 10 to 30 carbon atoms; and iii) R31 and R32 bond to form an aliphatic or aromatic heteroring having 2 to 60 carbon atoms substituted or unsubstituted and including N.

In one embodiment of the present specification, Z may be represented by any one of the following Chemical Formulae Z-1 to Z-4.

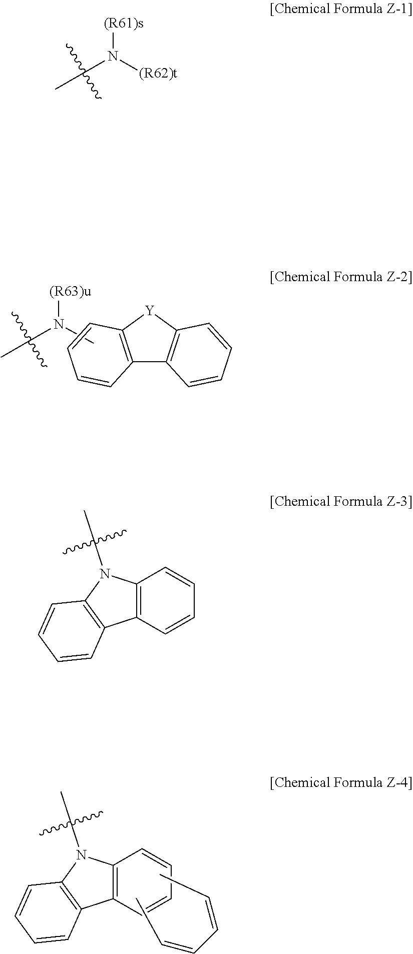

In Chemical Formulae Z-1 to Z-4,

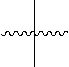

is a position bonding to L,

Y is O; or S,

R61 and R62 are the same as or different from each other, and each independently a substituted or unsubstituted aryl group having 6 to 60 carbon atoms,

R63 is a substituted or unsubstituted aryl group having 10 to 60 carbon atoms,

s, t and u are each an integer of 1 to 5, and

when s, t and u are each 2 or greater, substituents in the parentheses are the same as or different from each other.

In one embodiment of the present specification, R61 and R62 are the same as or different from each other, and may be each independently a substituted or unsubstituted aryl group having 6 to 30 carbon atoms.

In one embodiment of the present specification, R61 and R62 are the same as or different from each other, and may be each independently a substituted or unsubstituted aryl group having 6 to 20 carbon atoms.

In one embodiment of the present specification, R61 and R62 are the same as or different from each other, and may be each independently a substituted or unsubstituted aryl group having 6 to 15 carbon atoms.

In one embodiment of the present specification, R61 and R62 are the same as or different from each other, and may be each independently a substituted or unsubstituted phenyl group; a substituted or unsubstituted biphenyl group; a substituted or unsubstituted ter-phenyl group; a substituted or unsubstituted naphthyl group; or a substituted or unsubstituted fluorenyl group.

In one embodiment of the present specification, R61 and R62 are the same as or different from each other, and may be each independently a phenyl group unsubstituted or substituted with deuterium; a biphenyl group unsubstituted or substituted with deuterium; a ter-phenyl group unsubstituted or substituted with deuterium; a naphthyl group unsubstituted or substituted with deuterium; or a fluorenyl group unsubstituted or substituted with an alkyl group or an aryl group.

In one embodiment of the present specification, R61 and R62 are the same as or different from each other, and may be each independently a phenyl group unsubstituted or substituted with deuterium; a biphenyl group unsubstituted or substituted with deuterium; a ter-phenyl group unsubstituted or substituted with deuterium; a naphthyl group unsubstituted or substituted with deuterium; or a fluorenyl group unsubstituted or substituted with an alkyl group.

In one embodiment of the present specification, R63 may be a substituted or unsubstituted aryl group having 10 to 60 carbon atoms.

In one embodiment of the present specification, R63 may be a substituted or unsubstituted aryl group having 10 to 30 carbon atoms.

In one embodiment of the present specification, R63 may be a substituted or unsubstituted aryl group having 10 to 20 carbon atoms.

In one embodiment of the present specification, R63 may be a substituted or unsubstituted biphenyl group.

In one embodiment of the present specification, R63 may be a biphenyl group.

In one embodiment of the present specification, Z may be represented by the following Chemical Formula Z-5.

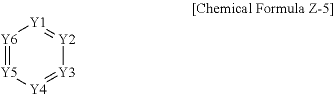

In Chemical Formula Z-5,

any one of Y1 to Y6 is C bonding to L,

the rest of Y1 to Y6 are N or C(R51), and at least two thereof are N,

R51 is hydrogen; deuterium; a substituted or unsubstituted aryl group having 6 to 60 carbon atoms; or a substituted or unsubstituted heteroaryl group having 2 to 60 carbon atoms,

adjacent group among Y1 to Y6 may bond to each other to form a substituted or unsubstituted aliphatic or aromatic hydrocarbon ring having 6 to 60 carbon atoms, or a substituted or unsubstituted aliphatic or aromatic heteroring having 2 to 60 carbon atoms, and

when two or more of Y1 to Y6 are C(R51), R51s are the same as or different from each other.

In one embodiment of the present specification, the rest of Y1 to Y6 not bonding to L may be N or C(R51), and two thereof may be N.

In one embodiment of the present specification, the rest of Y1 to Y6 not bonding to L may be N or C(R51), and three thereof may be N.

In one embodiment of the present specification, Y1 is C bonding to L, and two or more of Y2, Y4, Y5 and Y6 may be N.

In one embodiment of the present specification, R51 may be hydrogen; deuterium; a substituted or unsubstituted aryl group having 6 to 30 carbon atoms; or a substituted or unsubstituted heteroaryl group having 2 to 30 carbon atoms.

In one embodiment of the present specification, R51 may be hydrogen; deuterium; a substituted or unsubstituted phenyl group; a substituted or unsubstituted biphenyl group; a substituted or unsubstituted naphthyl group; a substituted or unsubstituted fluorenyl group; or a substituted or unsubstituted dibenzofuran group.

In one embodiment of the present specification, R51 may be hydrogen; deuterium; a phenyl group unsubstituted or substituted with deuterium; a biphenyl group unsubstituted or substituted with deuterium; a naphthyl group unsubstituted or substituted with deuterium; a fluorenyl group unsubstituted or substituted with an alkyl group; or a dibenzofuran group.

In one embodiment of the present specification, R51 may be hydrogen; deuterium; a substituted or unsubstituted phenyl group; a substituted or unsubstituted biphenyl group; a substituted or unsubstituted naphthyl group; or a substituted or unsubstituted dibenzofuran group.

In one embodiment of the present specification, R51 may be hydrogen; deuterium; a phenyl group unsubstituted or substituted with deuterium or a carbazole group; a biphenyl group unsubstituted or substituted with deuterium; a naphthyl group unsubstituted or substituted with deuterium; or a dibenzofuran group.

In one embodiment of the present specification, adjacent groups among Y1 to Y6 may bond to each other to form a substituted or unsubstituted ring.

In one embodiment of the present specification, Chemical Formula Z-5 may be selected from among the following structural formulae.

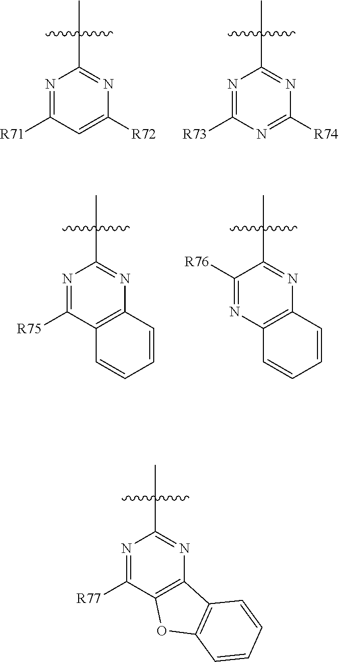

In the structural formulae,

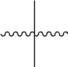

is a position bonding to L, and

R71 to R77 may be each independently a substituted or unsubstituted aryl group having 6 to 30 carbon atoms; or a substituted or unsubstituted heteroaryl group having 2 to 30 carbon atoms.

In one embodiment of the present specification, Chemical Formula 1 may be represented by any one of the following Chemical Formulae 1-1 to 1-4.

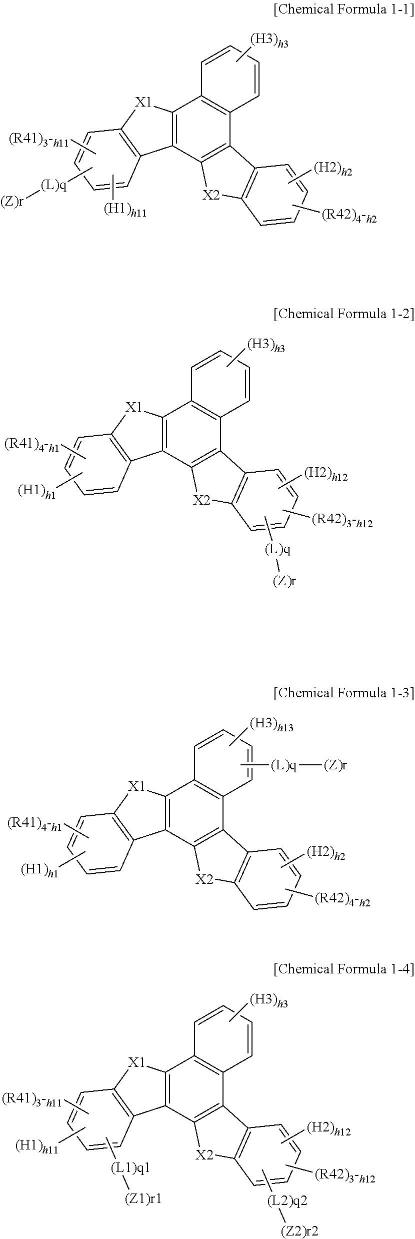

In Chemical Formulae 1-1 to 1-4,

X1, X2, L and Z have the same definitions as in Chemical Formula 1,

L1 and L2 each independently have the same definition as L in Chemical Formula 1,

Z1 and Z2 each independently have the same definition as Z in Chemical Formula 1,

q1 and q2 each independently have the same definition as q in Chemical Formula 1,

r1 and r2 each independently have the same definition as r in Chemical Formula 1,

H1 to H3 are each independently hydrogen; or deuterium,

R41 and R42 are each independently a substituted or unsubstituted aryl group having 6 to 60 carbon atoms,

h1 to h3 are each an integer of 0 to 4,

h11 to h13 are each an integer of 0 to 3,

when h1 to h3 are each 2 or greater or h11 to h13 are each 2 or greater, H1s to H3s are each the same as or different from each other, and

when h1 and h2 are each 2 or less or h11 and h12 are each 1 or less, R41s and R42s are each the same as or different from each other.

In one embodiment of the present specification, Chemical Formula 1 may have a deuterium content of 0% to 100%.

In one embodiment of the present specification, Chemical Formula 1 may have a deuterium content of 0%.

In one embodiment of the present specification, Chemical Formula 1 may have a deuterium content of greater than 0% and less than or equal to 100%.

In one embodiment of the present specification, when Chemical Formula 1 includes deuterium, the deuterium content may be from 10% to 100%.

In one embodiment of the present specification, when Chemical Formula 1 includes deuterium, the deuterium content may be from 20% to 100%.

In one embodiment of the present specification, Chemical Formula 1 may have a deuterium content of either 0%, or 10% to 100%.

In one embodiment of the present specification, Chemical Formula 1 may have a deuterium content of either 0%, or 20% to 100%.

In one embodiment of the present specification, Chemical Formula 1 may be represented by any one of the following compounds, but it not limited thereto.

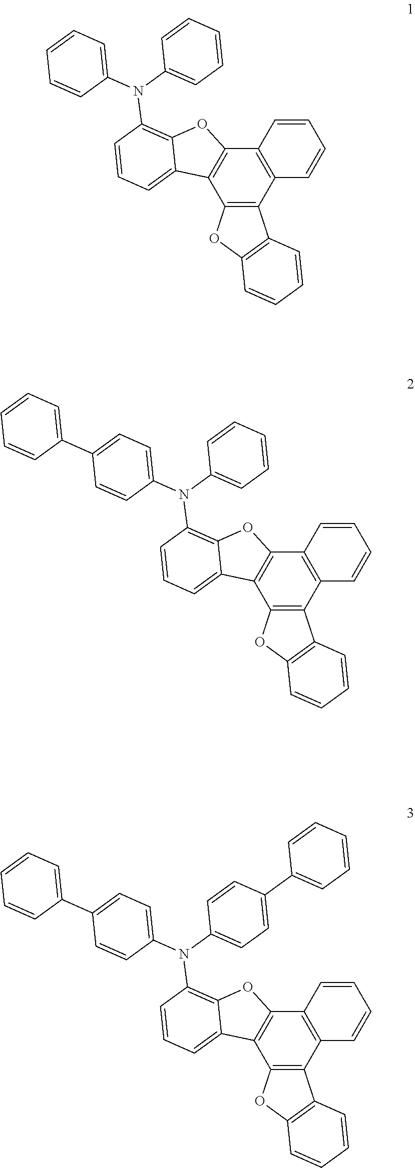 [SMILES: c1ccc(N(c2ccccc2)c2cccc3c2oc2c4ccccc4c4c5ccccc5oc4c32)cc1 ; c1ccc(-c2ccc(N(c3ccccc3)c3cccc4c3oc3c5ccccc5c5c6ccccc6oc5c43)cc2)cc1 ; c1ccc(-c2ccc(N(c3ccc(-c4ccccc4)cc3)c3cccc4c3oc3c5ccccc5c5c6ccccc6oc5c43)cc2)cc1]

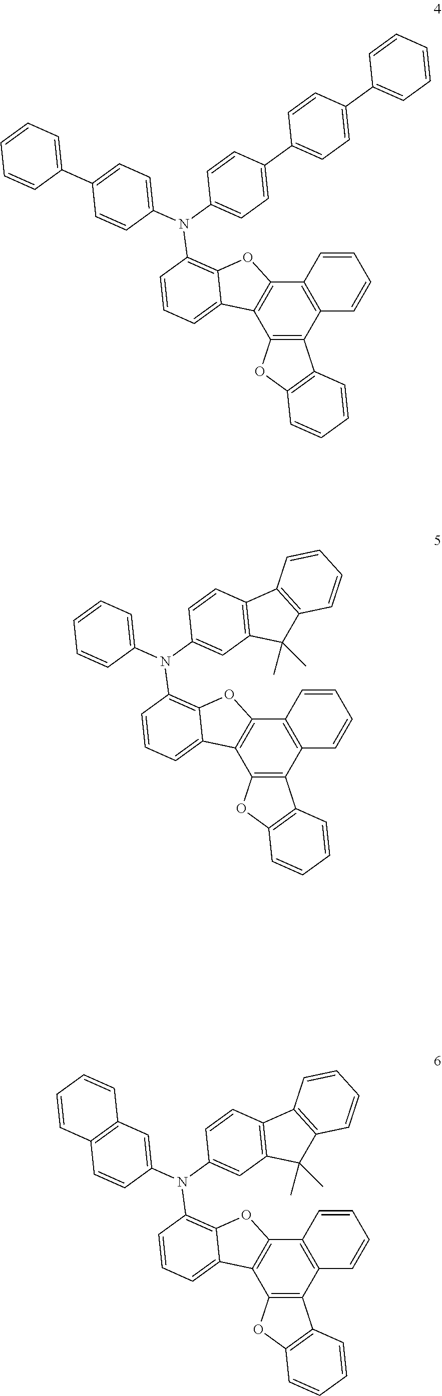 [SMILES: c1ccc(-c2ccc(-c3ccc(N(c4ccc(-c5ccccc5)cc4)c4cccc5c4oc4c6ccccc6c6c7ccccc7oc6c54)cc3)cc2)cc1 ; CC1(C)c2ccccc2-c2ccc(N(c3ccccc3)c3cccc4c3oc3c5ccccc5c5c6ccccc6oc5c43)cc21 ; CC1(C)c2ccccc2-c2ccc(N(c3ccc4ccccc4c3)c3cccc4c3oc3c5ccccc5c5c6ccccc6oc5c43)cc21]

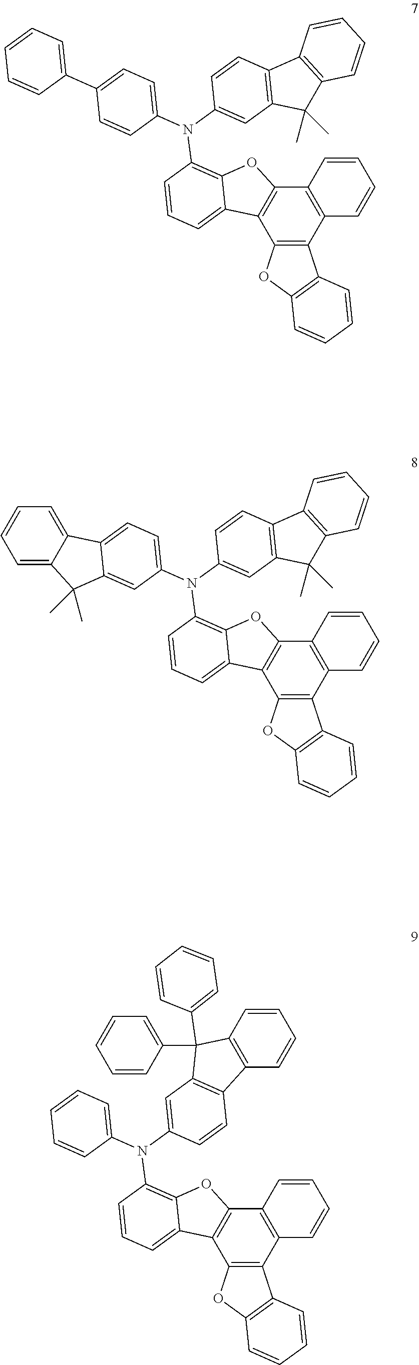 [SMILES: CC1(C)c2ccccc2-c2ccc(N(c3ccc(-c4ccccc4)cc3)c3cccc4c3oc3c5ccccc5c5c6ccccc6oc5c43)cc21 ; CC1(C)c2ccccc2-c2ccc(N(c3ccc4c(c3)C(C)(C)c3ccccc3-4)c3cccc4c3oc3c5ccccc5c5c6ccccc6oc5c43)cc21 ; c1ccc(N(c2ccc3c(c2)C(c2ccccc2)(c2ccccc2)c2ccccc2-3)c2cccc3c2oc2c4ccccc4c4c5ccccc5oc4c32)cc1]

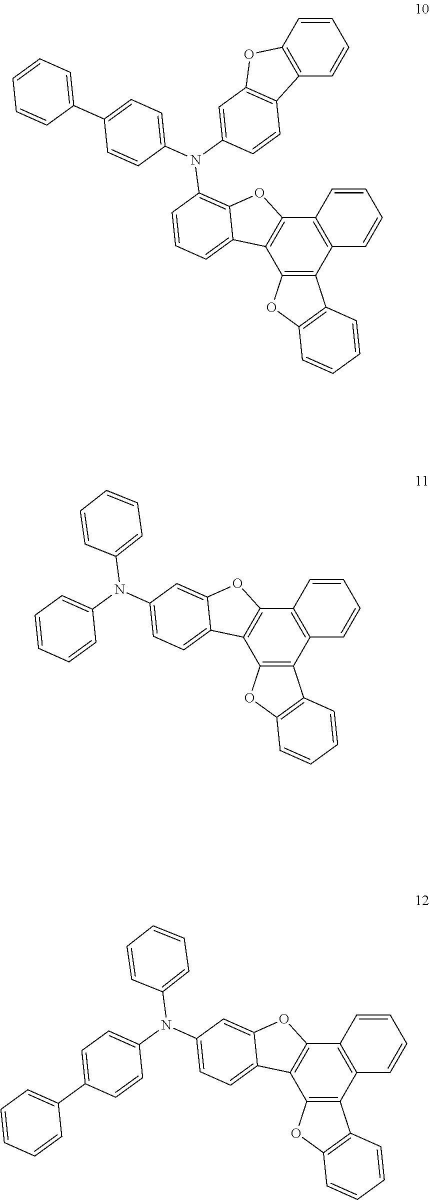 [SMILES: c1ccc(-c2ccc(N(c3ccc4c(c3)oc3ccccc34)c3cccc4c3oc3c5ccccc5c5c6ccccc6oc5c43)cc2)cc1 ; c1ccc(N(c2ccccc2)c2ccc3c(c2)oc2c4ccccc4c4c5ccccc5oc4c32)cc1 ; c1ccc(-c2ccc(N(c3ccccc3)c3ccc4c(c3)oc3c5ccccc5c5c6ccccc6oc5c43)cc2)cc1]

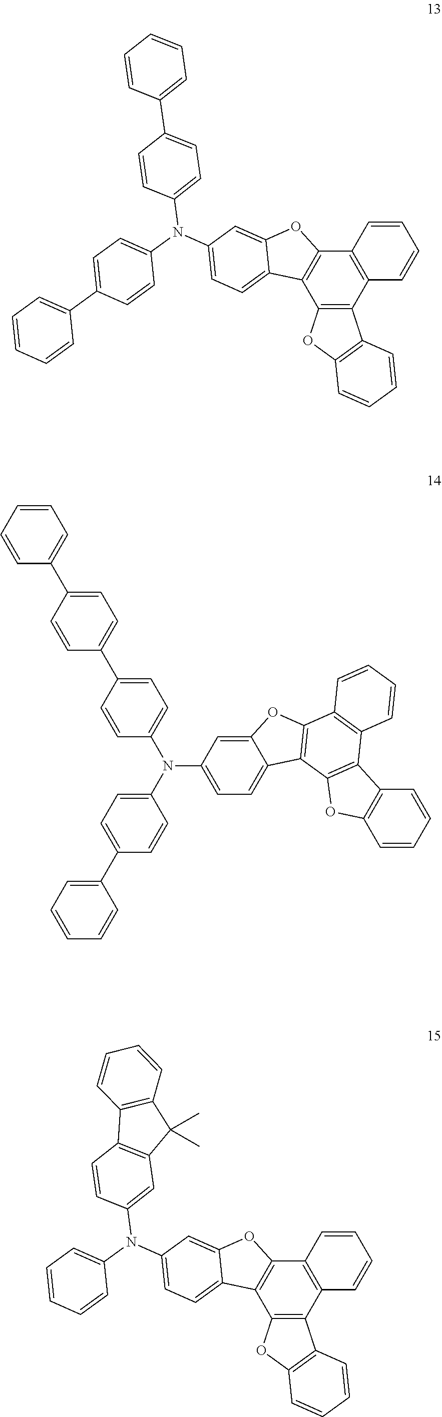 [SMILES: c1ccc(-c2ccc(N(c3ccc(-c4ccccc4)cc3)c3ccc4c(c3)oc3c5ccccc5c5c6ccccc6oc5c43)cc2)cc1 ; c1ccc(-c2ccc(-c3ccc(N(c4ccc(-c5ccccc5)cc4)c4ccc5c(c4)oc4c6ccccc6c6c7ccccc7oc6c54)cc3)cc2)cc1 ; CC1(C)c2ccccc2-c2ccc(N(c3ccccc3)c3ccc4c(c3)oc3c5ccccc5c5c6ccccc6oc5c43)cc21]

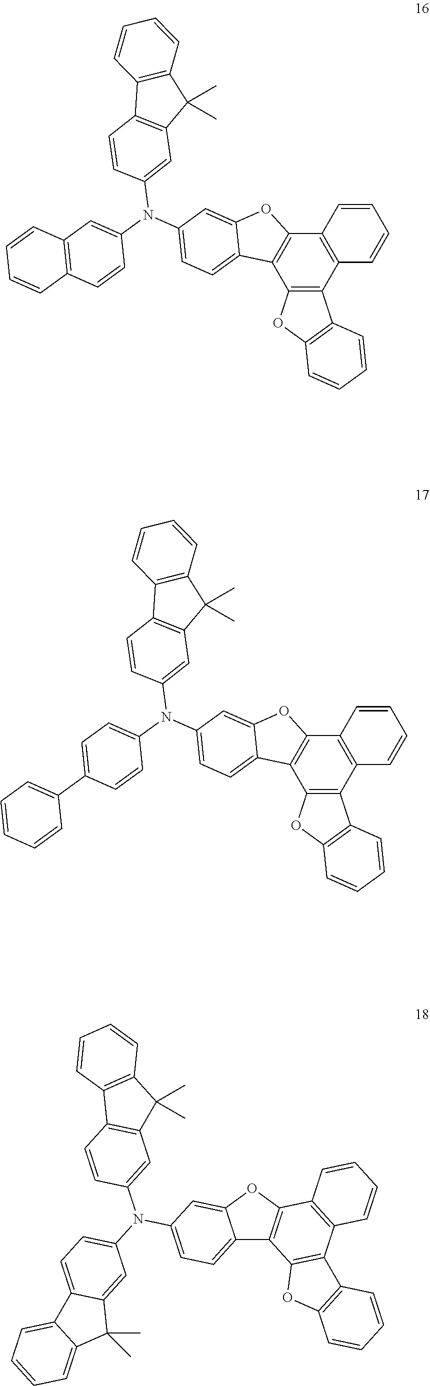 [SMILES: CC1(C)c2ccccc2-c2ccc(N(c3ccc4ccccc4c3)c3ccc4c(c3)oc3c5ccccc5c5c6ccccc6oc5c43)cc21 ; CC1(C)c2ccccc2-c2ccc(N(c3ccc(-c4ccccc4)cc3)c3ccc4c(c3)oc3c5ccccc5c5c6ccccc6oc5c43)cc21 ; CC1(C)c2ccccc2-c2ccc(N(c3ccc4c(c3)C(C)(C)c3ccccc3-4)c3ccc4c(c3)oc3c5ccccc5c5c6ccccc6oc5c43)cc21]

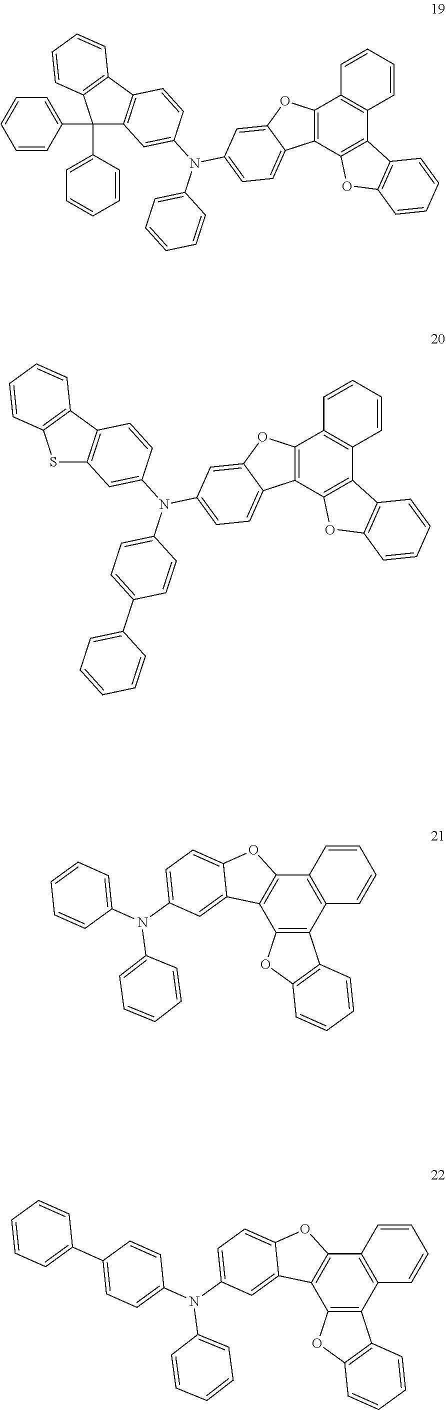 [SMILES: c1ccc(N(c2ccc3c(c2)C(c2ccccc2)(c2ccccc2)c2ccccc2-3)c2ccc3c(c2)oc2c4ccccc4c4c5ccccc5oc4c32)cc1 ; c1ccc(-c2ccc(N(c3ccc4c(c3)oc3c5ccccc5c5c6ccccc6oc5c43)c3ccc4c(c3)sc3ccccc34)cc2)cc1 ; c1ccc(N(c2ccccc2)c2ccc3oc4c5ccccc5c5c6ccccc6oc5c4c3c2)cc1 ; c1ccc(-c2ccc(N(c3ccccc3)c3ccc4oc5c6ccccc6c6c7ccccc7oc6c5c4c3)cc2)cc1]

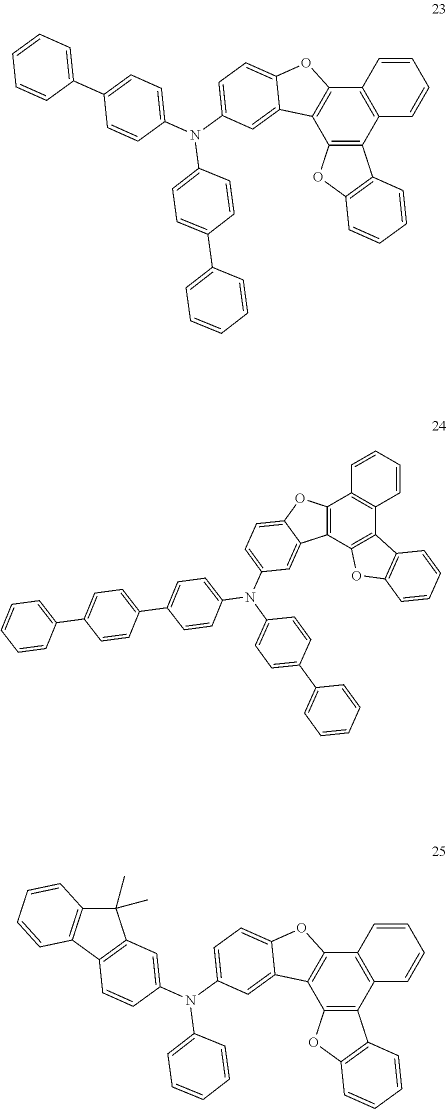 [SMILES: c1ccc(-c2ccc(N(c3ccc(-c4ccccc4)cc3)c3ccc4oc5c6ccccc6c6c7ccccc7oc6c5c4c3)cc2)cc1 ; c1ccc(-c2ccc(-c3ccc(N(c4ccc(-c5ccccc5)cc4)c4ccc5oc6c7ccccc7c7c8ccccc8oc7c6c5c4)cc3)cc2)cc1 ; CC1(C)c2ccccc2-c2ccc(N(c3ccccc3)c3ccc4oc5c6ccccc6c6c7ccccc7oc6c5c4c3)cc21]

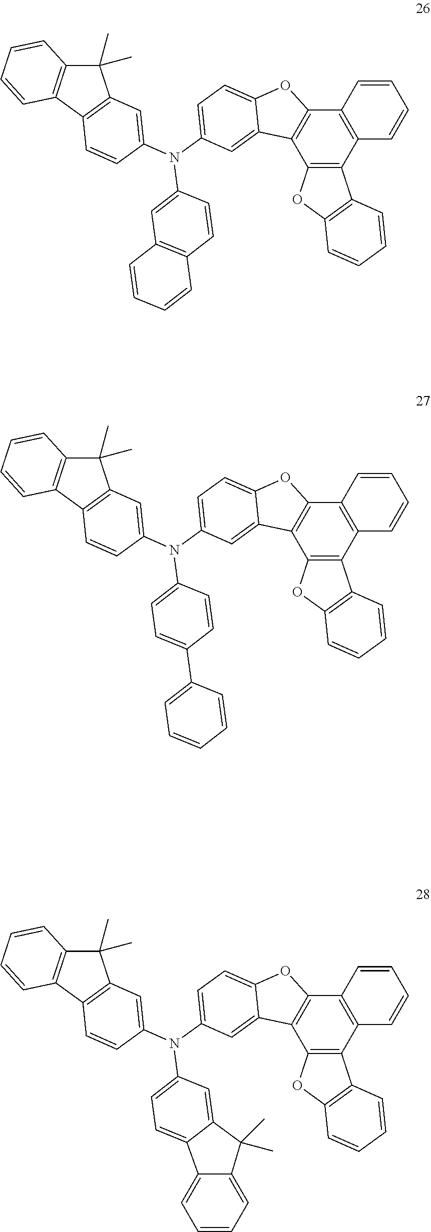 [SMILES: CC1(C)c2ccccc2-c2ccc(N(c3ccc4ccccc4c3)c3ccc4oc5c6ccccc6c6c7ccccc7oc6c5c4c3)cc21 ; CC1(C)c2ccccc2-c2ccc(N(c3ccc(-c4ccccc4)cc3)c3ccc4oc5c6ccccc6c6c7ccccc7oc6c5c4c3)cc21 ; CC1(C)c2ccccc2-c2ccc(N(c3ccc4c(c3)C(C)(C)c3ccccc3-4)c3ccc4oc5c6ccccc6c6c7ccccc7oc6c5c4c3)cc21]

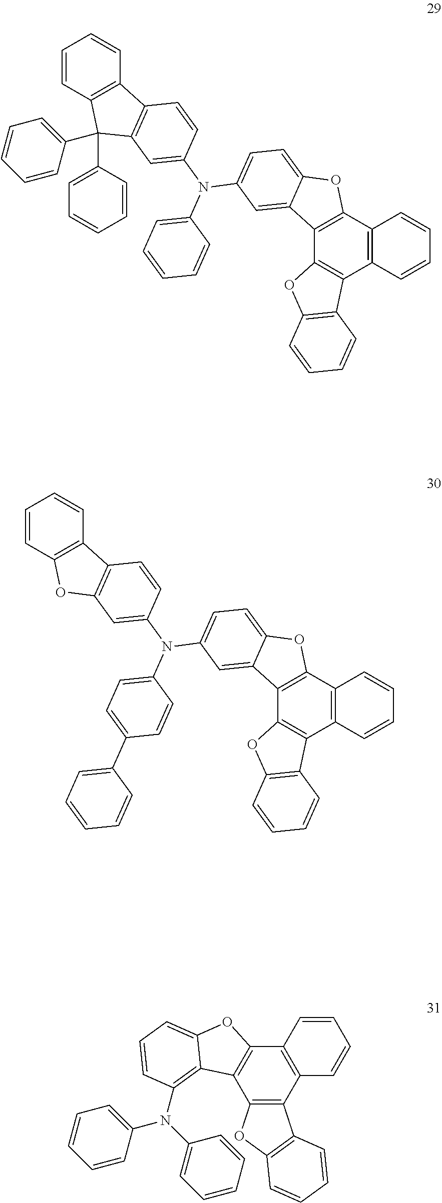 [SMILES: c1ccc(N(c2ccc3c(c2)C(c2ccccc2)(c2ccccc2)c2ccccc2-3)c2ccc3oc4c5ccccc5c5c6ccccc6oc5c4c3c2)cc1 ; c1ccc(-c2ccc(N(c3ccc4c(c3)oc3ccccc34)c3ccc4oc5c6ccccc6c6c7ccccc7oc6c5c4c3)cc2)cc1 ; c1ccc(N(c2ccccc2)c2cccc3oc4c5ccccc5c5c6ccccc6oc5c4c23)cc1]

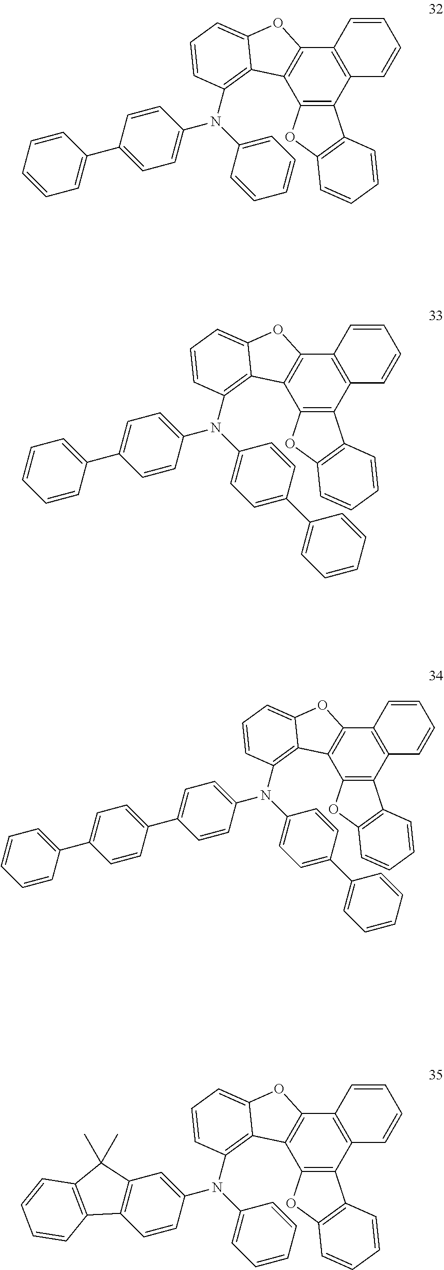 [SMILES: c1ccc(-c2ccc(N(c3ccccc3)c3cccc4oc5c6ccccc6c6c7ccccc7oc6c5c34)cc2)cc1 ; c1ccc(-c2ccc(N(c3ccc(-c4ccccc4)cc3)c3cccc4oc5c6ccccc6c6c7ccccc7oc6c5c34)cc2)cc1 ; c1ccc(-c2ccc(-c3ccc(N(c4ccc(-c5ccccc5)cc4)c4cccc5oc6c7ccccc7c7c8ccccc8oc7c6c45)cc3)cc2)cc1 ; CC1(C)c2ccccc2-c2ccc(N(c3ccccc3)c3cccc4oc5c6ccccc6c6c7ccccc7oc6c5c34)cc21]

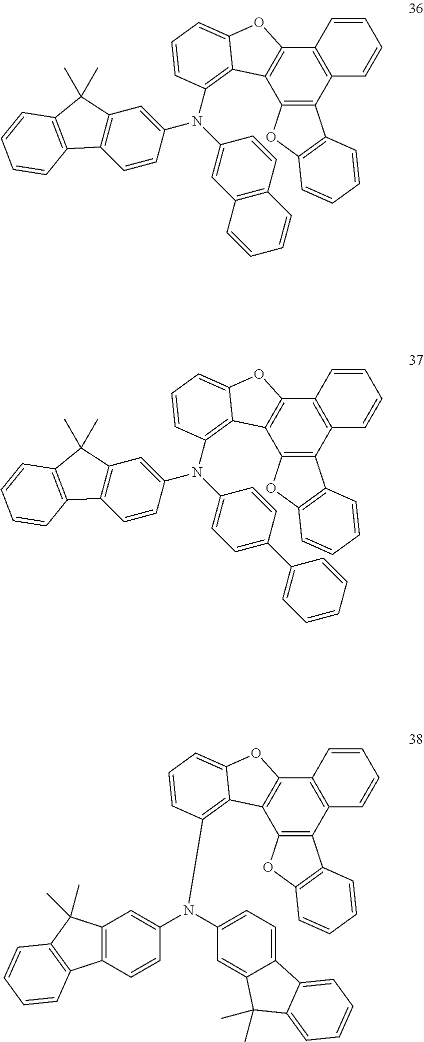 [SMILES: CC1(C)c2ccccc2-c2ccc(N(c3ccc4ccccc4c3)c3cccc4oc5c6ccccc6c6c7ccccc7oc6c5c34)cc21 ; CC1(C)c2ccccc2-c2ccc(N(c3ccc(-c4ccccc4)cc3)c3cccc4oc5c6ccccc6c6c7ccccc7oc6c5c34)cc21 ; CC1(C)c2ccccc2-c2ccc(N(c3ccc4c(c3)C(C)(C)c3ccccc3-4)c3cccc4oc5c6ccccc6c6c7ccccc7oc6c5c34)cc21]

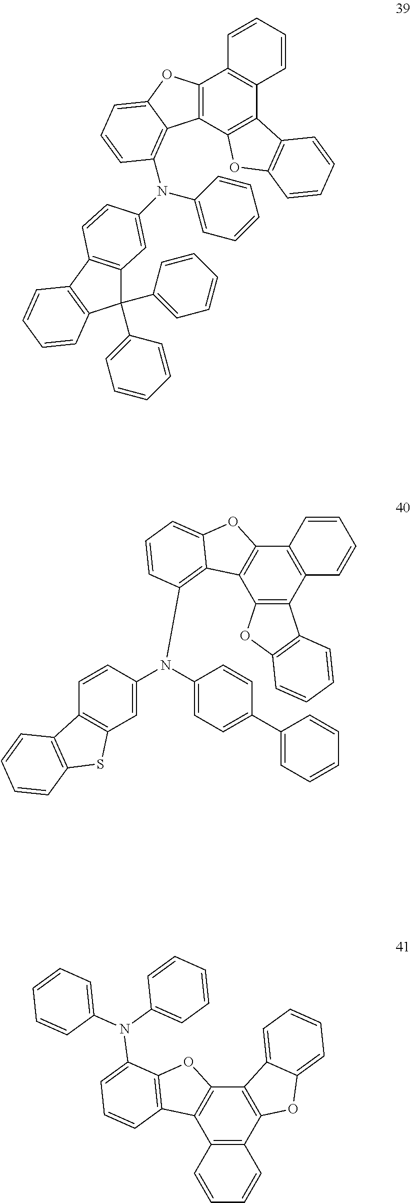 [SMILES: c1ccc(N(c2ccc3c(c2)C(c2ccccc2)(c2ccccc2)c2ccccc2-3)c2cccc3oc4c5ccccc5c5c6ccccc6oc5c4c23)cc1 ; c1ccc(-c2ccc(N(c3ccc4c(c3)sc3ccccc34)c3cccc4oc5c6ccccc6c6c7ccccc7oc6c5c34)cc2)cc1 ; c1ccc(N(c2ccccc2)c2cccc3c2oc2c3c3ccccc3c3oc4ccccc4c32)cc1]

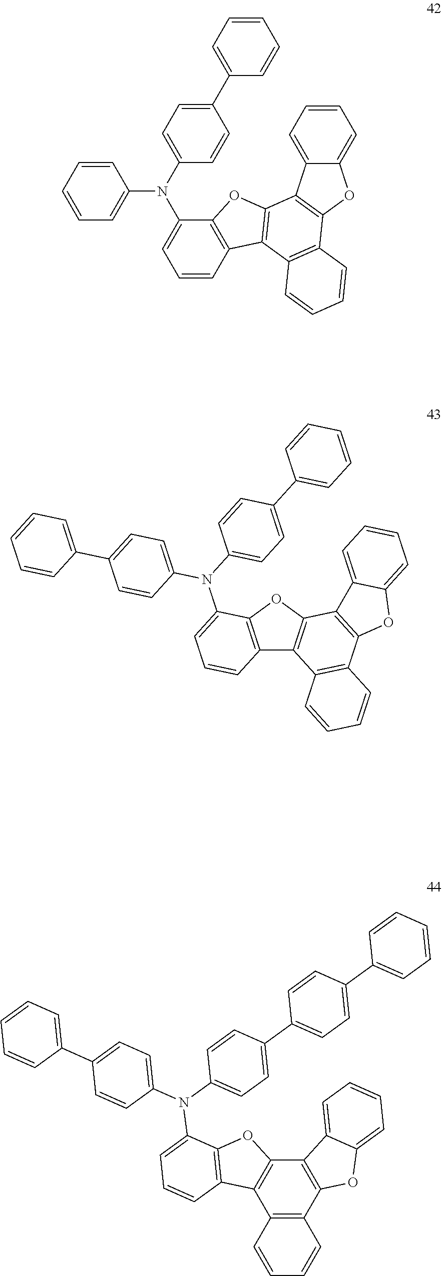 [SMILES: c1ccc(-c2ccc(N(c3ccccc3)c3cccc4c3oc3c4c4ccccc4c4oc5ccccc5c43)cc2)cc1 ; c1ccc(-c2ccc(N(c3ccc(-c4ccccc4)cc3)c3cccc4c3oc3c4c4ccccc4c4oc5ccccc5c43)cc2)cc1 ; c1ccc(-c2ccc(-c3ccc(N(c4ccc(-c5ccccc5)cc4)c4cccc5c4oc4c5c5ccccc5c5oc6ccccc6c54)cc3)cc2)cc1]

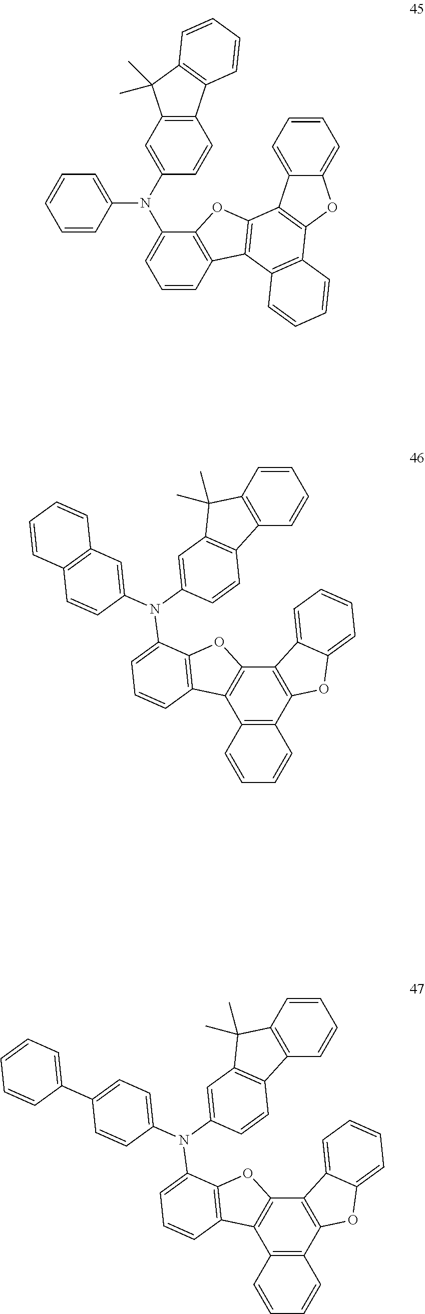 [SMILES: CC1(C)c2ccccc2-c2ccc(N(c3ccccc3)c3cccc4c3oc3c4c4ccccc4c4oc5ccccc5c43)cc21 ; CC1(C)c2ccccc2-c2ccc(N(c3ccc4ccccc4c3)c3cccc4c3oc3c4c4ccccc4c4oc5ccccc5c43)cc21 ; CC1(C)c2ccccc2-c2ccc(N(c3ccc(-c4ccccc4)cc3)c3cccc4c3oc3c4c4ccccc4c4oc5ccccc5c43)cc21]

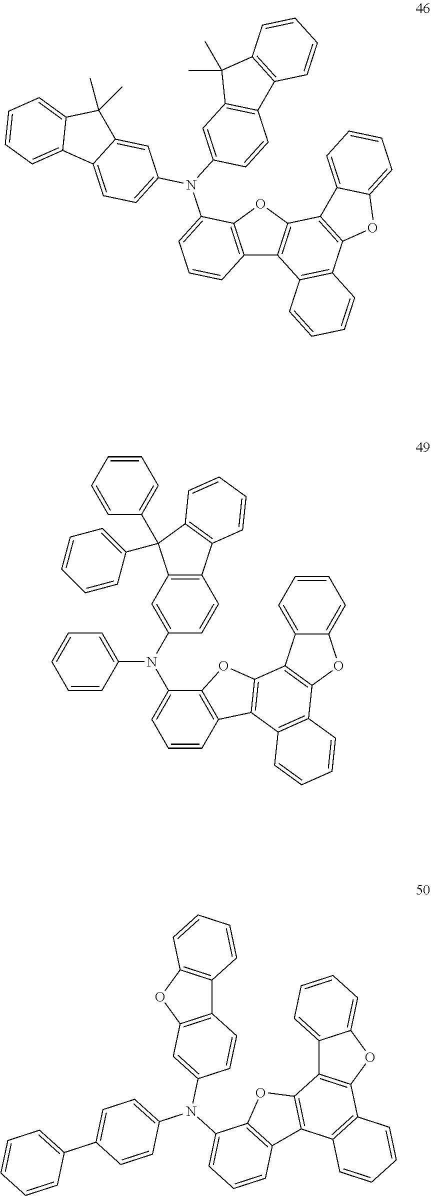 [SMILES: CC1(C)c2ccccc2-c2ccc(N(c3ccc4c(c3)C(C)(C)c3ccccc3-4)c3cccc4c3oc3c4c4ccccc4c4oc5ccccc5c43)cc21 ; c1ccc(N(c2ccc3c(c2)C(c2ccccc2)(c2ccccc2)c2ccccc2-3)c2cccc3c2oc2c3c3ccccc3c3oc4ccccc4c32)cc1 ; c1ccc(-c2ccc(N(c3ccc4c(c3)oc3ccccc34)c3cccc4c3oc3c4c4ccccc4c4oc5ccccc5c43)cc2)cc1]

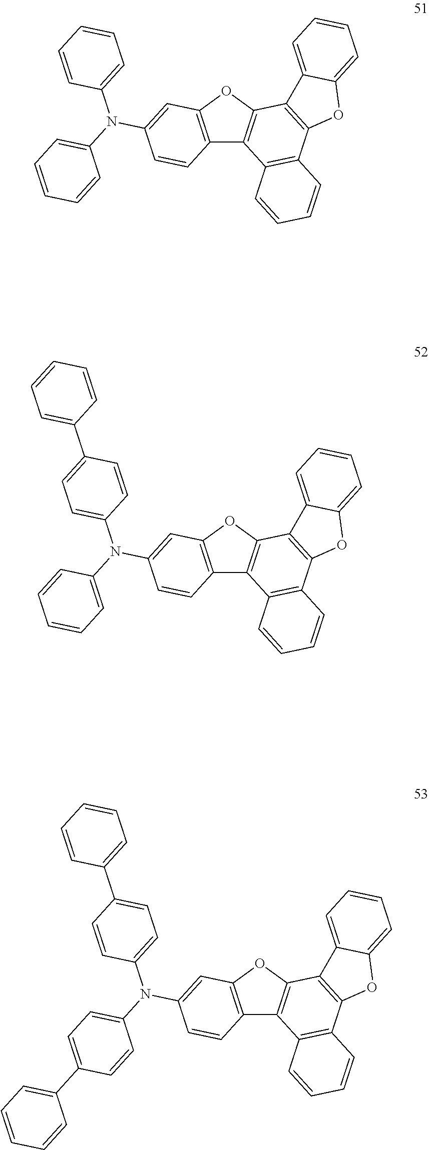 [SMILES: c1ccc(N(c2ccccc2)c2ccc3c(c2)oc2c3c3ccccc3c3oc4ccccc4c32)cc1 ; c1ccc(-c2ccc(N(c3ccccc3)c3ccc4c(c3)oc3c4c4ccccc4c4oc5ccccc5c43)cc2)cc1 ; c1ccc(-c2ccc(N(c3ccc(-c4ccccc4)cc3)c3ccc4c(c3)oc3c4c4ccccc4c4oc5ccccc5c43)cc2)cc1]

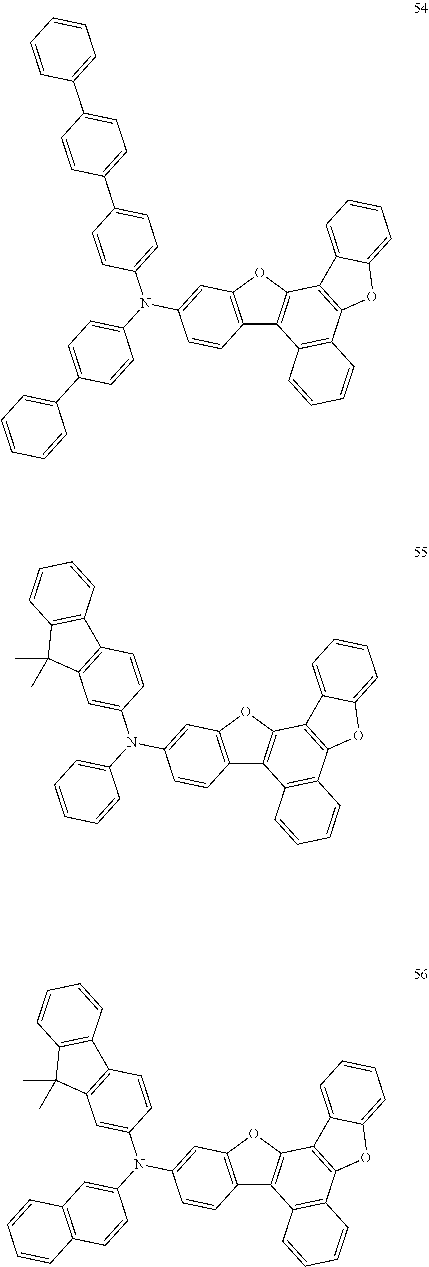 [SMILES: c1ccc(-c2ccc(-c3ccc(N(c4ccc(-c5ccccc5)cc4)c4ccc5c(c4)oc4c5c5ccccc5c5oc6ccccc6c54)cc3)cc2)cc1 ; CC1(C)c2ccccc2-c2ccc(N(c3ccccc3)c3ccc4c(c3)oc3c4c4ccccc4c4oc5ccccc5c43)cc21 ; CC1(C)c2ccccc2-c2ccc(N(c3ccc4ccccc4c3)c3ccc4c(c3)oc3c4c4ccccc4c4oc5ccccc5c43)cc21]

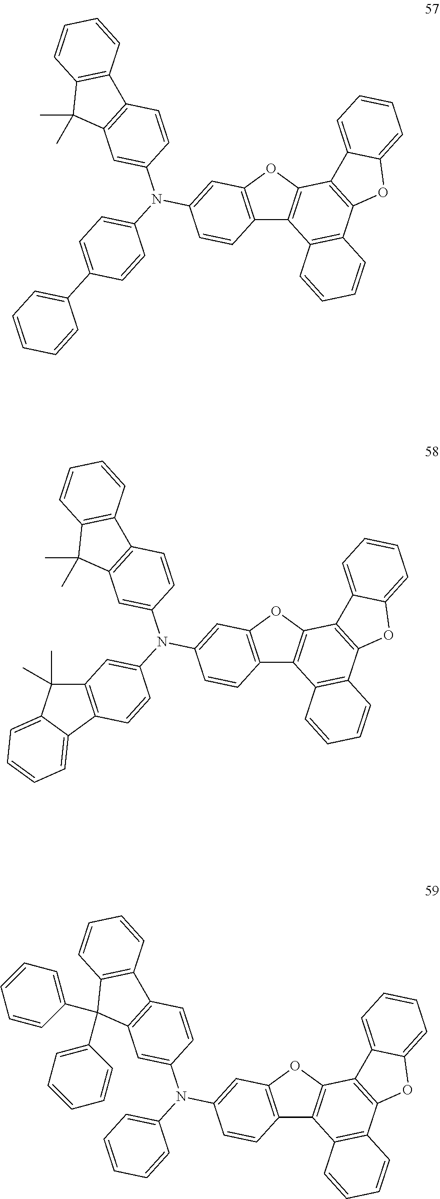 [SMILES: CC1(C)c2ccccc2-c2ccc(N(c3ccc(-c4ccccc4)cc3)c3ccc4c(c3)oc3c4c4ccccc4c4oc5ccccc5c43)cc21 ; CC1(C)c2ccccc2-c2ccc(N(c3ccc4c(c3)C(C)(C)c3ccccc3-4)c3ccc4c(c3)oc3c4c4ccccc4c4oc5ccccc5c43)cc21 ; c1ccc(N(c2ccc3c(c2)C(c2ccccc2)(c2ccccc2)c2ccccc2-3)c2ccc3c(c2)oc2c3c3ccccc3c3oc4ccccc4c32)cc1]

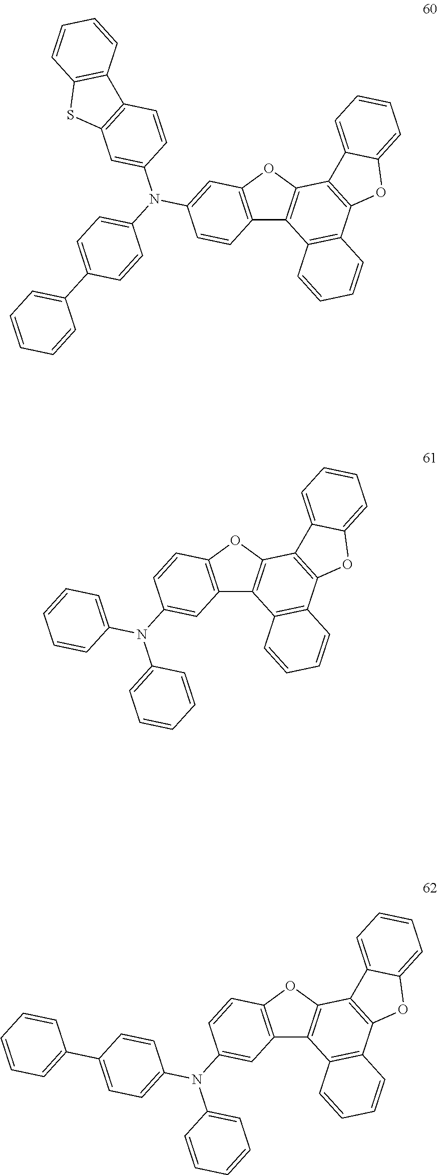 [SMILES: c1ccc(-c2ccc(N(c3ccc4c(c3)oc3c4c4ccccc4c4oc5ccccc5c43)c3ccc4c(c3)sc3ccccc34)cc2)cc1 ; c1ccc(N(c2ccccc2)c2ccc3oc4c(c3c2)c2ccccc2c2oc3ccccc3c24)cc1 ; c1ccc(-c2ccc(N(c3ccccc3)c3ccc4oc5c(c4c3)c3ccccc3c3oc4ccccc4c35)cc2)cc1]

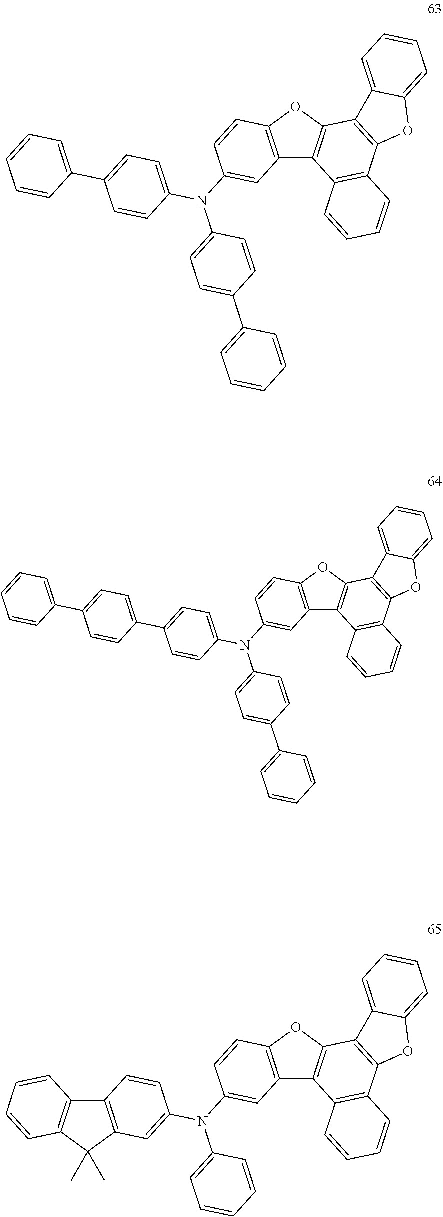 [SMILES: c1ccc(-c2ccc(N(c3ccc(-c4ccccc4)cc3)c3ccc4oc5c(c4c3)c3ccccc3c3oc4ccccc4c35)cc2)cc1 ; c1ccc(-c2ccc(-c3ccc(N(c4ccc(-c5ccccc5)cc4)c4ccc5oc6c(c5c4)c4ccccc4c4oc5ccccc5c46)cc3)cc2)cc1 ; CC1(C)c2ccccc2-c2ccc(N(c3ccccc3)c3ccc4oc5c(c4c3)c3ccccc3c3oc4ccccc4c35)cc21]

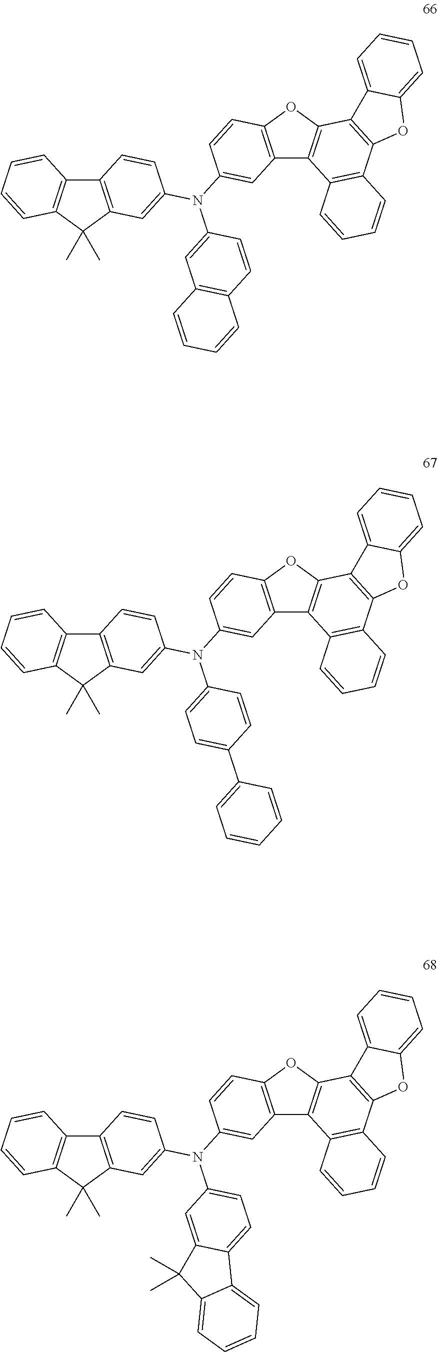 [SMILES: CC1(C)c2ccccc2-c2ccc(N(c3ccc4ccccc4c3)c3ccc4oc5c(c4c3)c3ccccc3c3oc4ccccc4c35)cc21 ; CC1(C)c2ccccc2-c2ccc(N(c3ccc(-c4ccccc4)cc3)c3ccc4oc5c(c4c3)c3ccccc3c3oc4ccccc4c35)cc21 ; CC1(C)c2ccccc2-c2ccc(N(c3ccc4c(c3)C(C)(C)c3ccccc3-4)c3ccc4oc5c(c4c3)c3ccccc3c3oc4ccccc4c35)cc21]

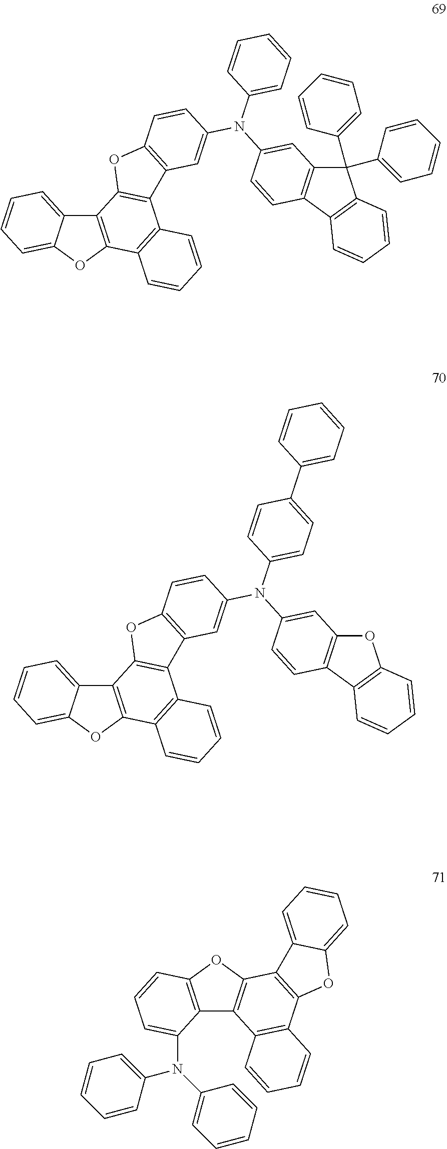 [SMILES: c1ccc(N(c2ccc3c(c2)C(c2ccccc2)(c2ccccc2)c2ccccc2-3)c2ccc3oc4c(c3c2)c2ccccc2c2oc3ccccc3c24)cc1 ; c1ccc(-c2ccc(N(c3ccc4c(c3)oc3ccccc34)c3ccc4oc5c(c4c3)c3ccccc3c3oc4ccccc4c35)cc2)cc1 ; c1ccc(N(c2ccccc2)c2cccc3oc4c5c6ccccc6oc5c5ccccc5c4c23)cc1]

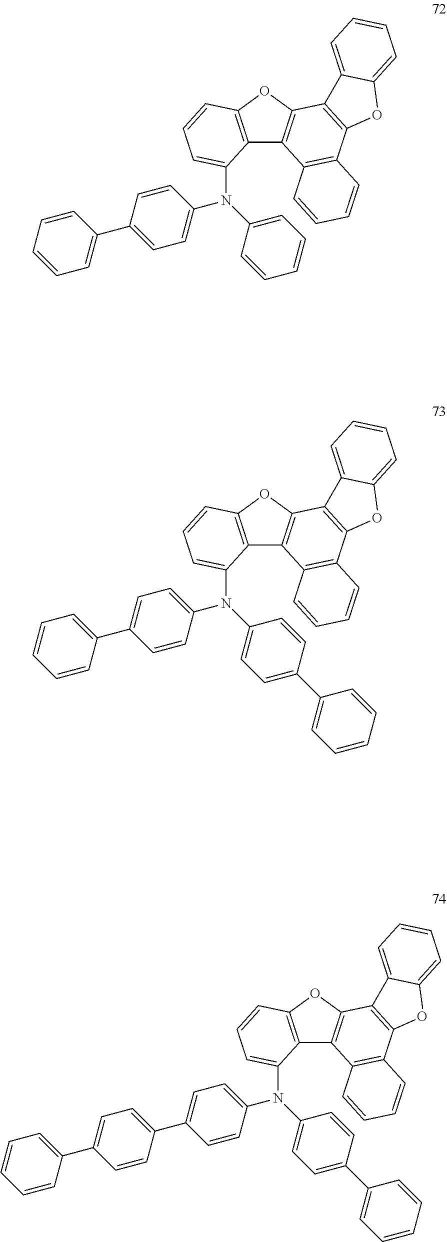 [SMILES: c1ccc(-c2ccc(N(c3ccccc3)c3cccc4oc5c6c7ccccc7oc6c6ccccc6c5c34)cc2)cc1 ; c1ccc(-c2ccc(N(c3ccc(-c4ccccc4)cc3)c3cccc4oc5c6c7ccccc7oc6c6ccccc6c5c34)cc2)cc1 ; c1ccc(-c2ccc(-c3ccc(N(c4ccc(-c5ccccc5)cc4)c4cccc5oc6c7c8ccccc8oc7c7ccccc7c6c45)cc3)cc2)cc1]

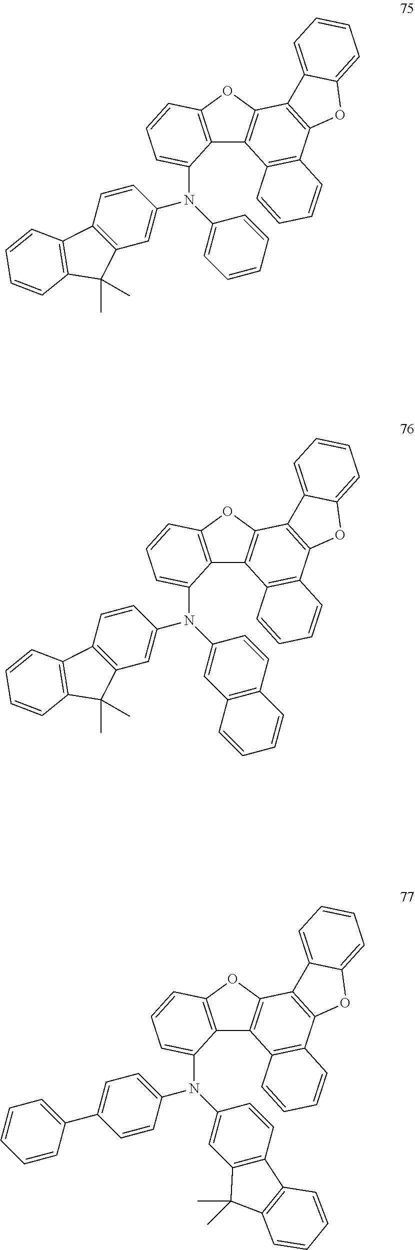 [SMILES: CC1(C)c2ccccc2-c2ccc(N(c3ccccc3)c3cccc4oc5c6c7ccccc7oc6c6ccccc6c5c34)cc21 ; CC1(C)c2ccccc2-c2ccc(N(c3ccc4ccccc4c3)c3cccc4oc5c6c7ccccc7oc6c6ccccc6c5c34)cc21 ; CC1(C)c2ccccc2-c2ccc(N(c3ccc(-c4ccccc4)cc3)c3cccc4oc5c6c7ccccc7oc6c6ccccc6c5c34)cc21]

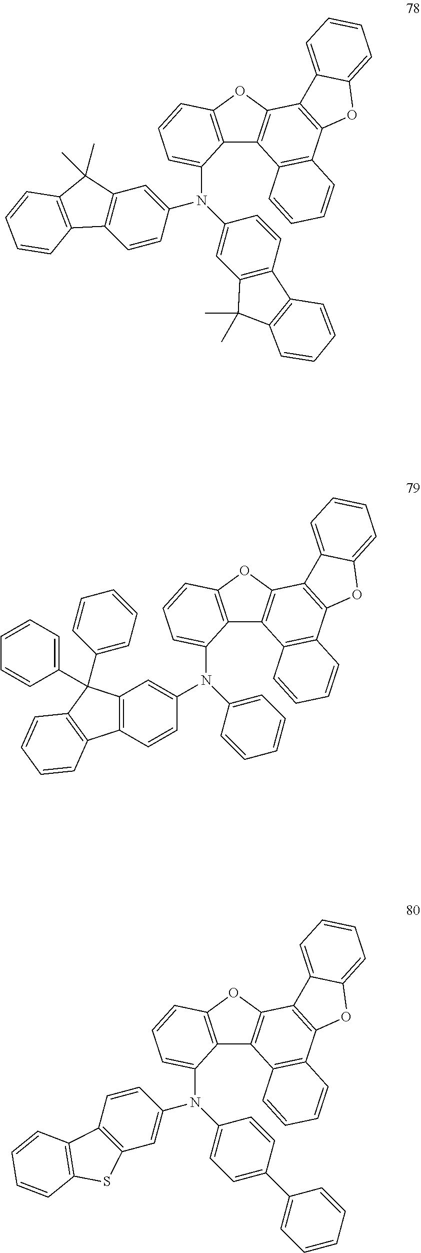 [SMILES: CC1(C)c2ccccc2-c2ccc(N(c3ccc4c(c3)C(C)(C)c3ccccc3-4)c3cccc4oc5c6c7ccccc7oc6c6ccccc6c5c34)cc21 ; c1ccc(N(c2ccc3c(c2)C(c2ccccc2)(c2ccccc2)c2ccccc2-3)c2cccc3oc4c5c6ccccc6oc5c5ccccc5c4c23)cc1 ; c1ccc(-c2ccc(N(c3ccc4c(c3)sc3ccccc34)c3cccc4oc5c6c7ccccc7oc6c6ccccc6c5c34)cc2)cc1]

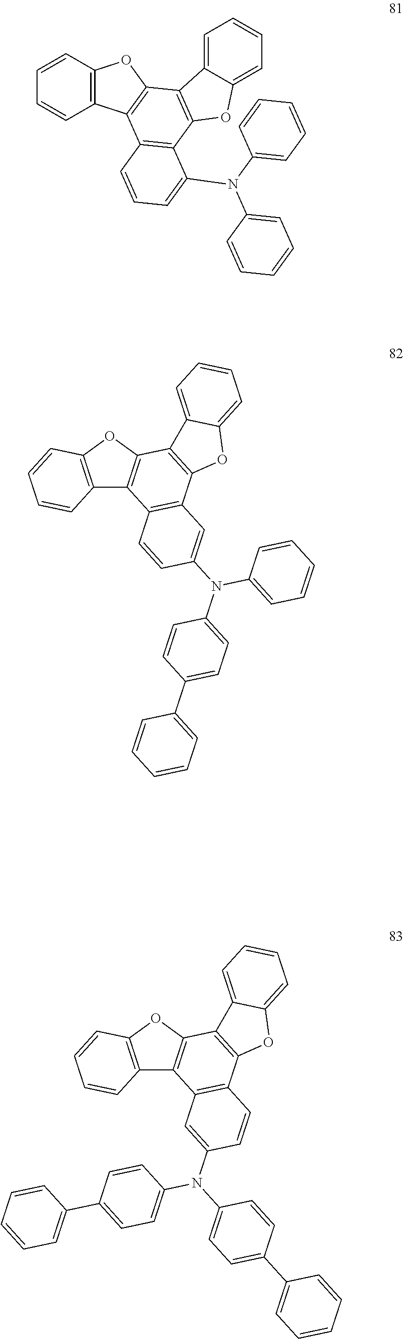 [SMILES: c1ccc(N(c2ccccc2)c2cccc3c2c2oc4ccccc4c2c2oc4ccccc4c32)cc1 ; c1ccc(-c2ccc(N(c3ccccc3)c3ccc4c(c3)c3oc5ccccc5c3c3oc5ccccc5c43)cc2)cc1 ; c1ccc(-c2ccc(N(c3ccc(-c4ccccc4)cc3)c3ccc4c(c3)c3c5ccccc5oc3c3c5ccccc5oc43)cc2)cc1]

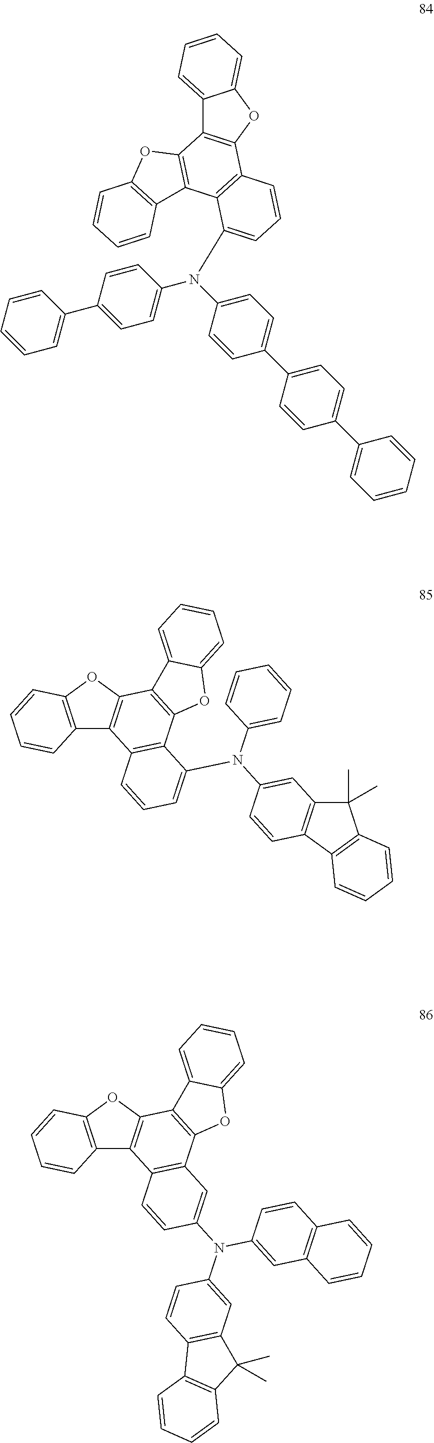 [SMILES: c1ccc(-c2ccc(-c3ccc(N(c4ccc(-c5ccccc5)cc4)c4cccc5c6oc7ccccc7c6c6oc7ccccc7c6c45)cc3)cc2)cc1 ; CC1(C)c2ccccc2-c2ccc(N(c3ccccc3)c3cccc4c3c3oc5ccccc5c3c3oc5ccccc5c43)cc21 ; CC1(C)c2ccccc2-c2ccc(N(c3ccc4ccccc4c3)c3ccc4c(c3)c3oc5ccccc5c3c3oc5ccccc5c43)cc21]

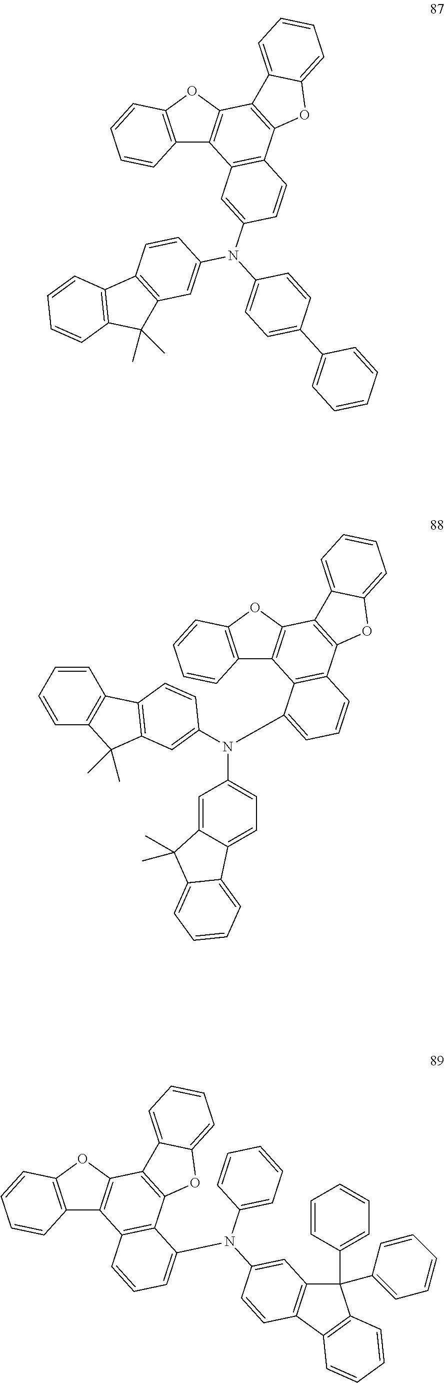 [SMILES: CC1(C)c2ccccc2-c2ccc(N(c3ccc(-c4ccccc4)cc3)c3ccc4c(c3)c3c5ccccc5oc3c3c5ccccc5oc43)cc21 ; CC1(C)c2ccccc2-c2ccc(N(c3ccc4c(c3)C(C)(C)c3ccccc3-4)c3cccc4c5oc6ccccc6c5c5oc6ccccc6c5c34)cc21 ; c1ccc(N(c2ccc3c(c2)C(c2ccccc2)(c2ccccc2)c2ccccc2-3)c2cccc3c2c2oc4ccccc4c2c2oc4ccccc4c32)cc1]

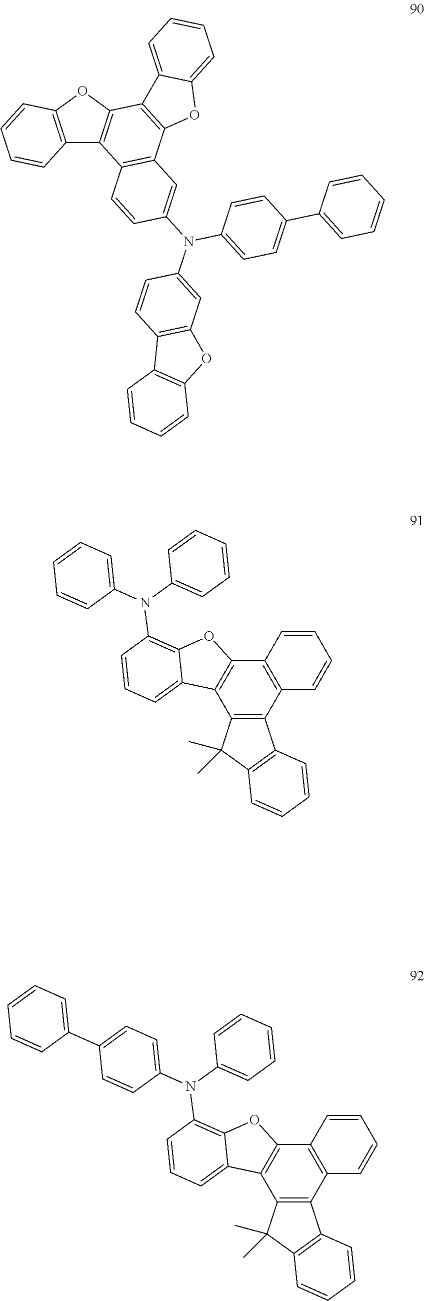 [SMILES: c1ccc(-c2ccc(N(c3ccc4c(c3)oc3ccccc34)c3ccc4c(c3)c3oc5ccccc5c3c3oc5ccccc5c43)cc2)cc1 ; CC1(C)c2ccccc2-c2c1c1c3cccc(N(c4ccccc4)c4ccccc4)c3oc1c1ccccc21 ; CC1(C)c2ccccc2-c2c1c1c3cccc(N(c4ccccc4)c4ccc(-c5ccccc5)cc4)c3oc1c1ccccc21]

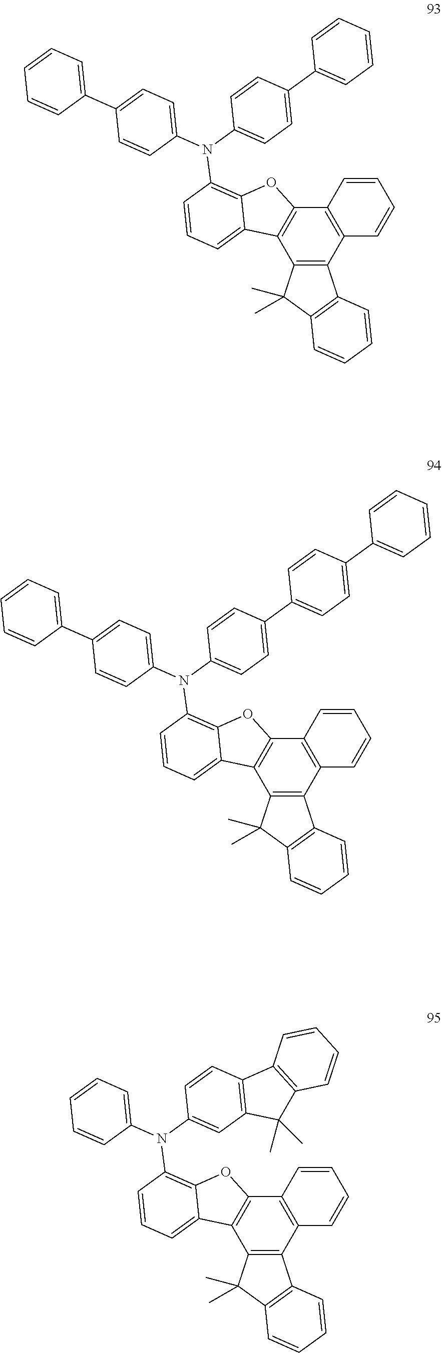 [SMILES: CC1(C)c2ccccc2-c2c1c1c3cccc(N(c4ccc(-c5ccccc5)cc4)c4ccc(-c5ccccc5)cc4)c3oc1c1ccccc21 ; CC1(C)c2ccccc2-c2c1c1c3cccc(N(c4ccc(-c5ccccc5)cc4)c4ccc(-c5ccc(-c6ccccc6)cc5)cc4)c3oc1c1ccccc21 ; CC1(C)c2ccccc2-c2ccc(N(c3ccccc3)c3cccc4c3oc3c5ccccc5c5c(c43)C(C)(C)c3ccccc3-5)cc21]

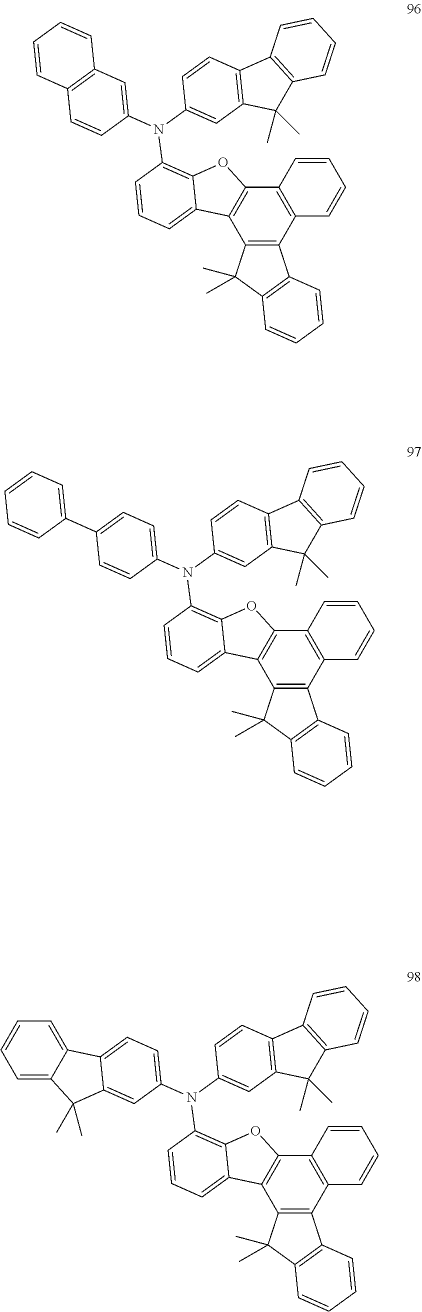 [SMILES: CC1(C)c2ccccc2-c2ccc(N(c3ccc4ccccc4c3)c3cccc4c3oc3c5ccccc5c5c(c43)C(C)(C)c3ccccc3-5)cc21 ; CC1(C)c2ccccc2-c2ccc(N(c3ccc(-c4ccccc4)cc3)c3cccc4c3oc3c5ccccc5c5c(c43)C(C)(C)c3ccccc3-5)cc21 ; CC1(C)c2ccccc2-c2ccc(N(c3ccc4c(c3)C(C)(C)c3ccccc3-4)c3cccc4c3oc3c5ccccc5c5c(c43)C(C)(C)c3ccccc3-5)cc21]

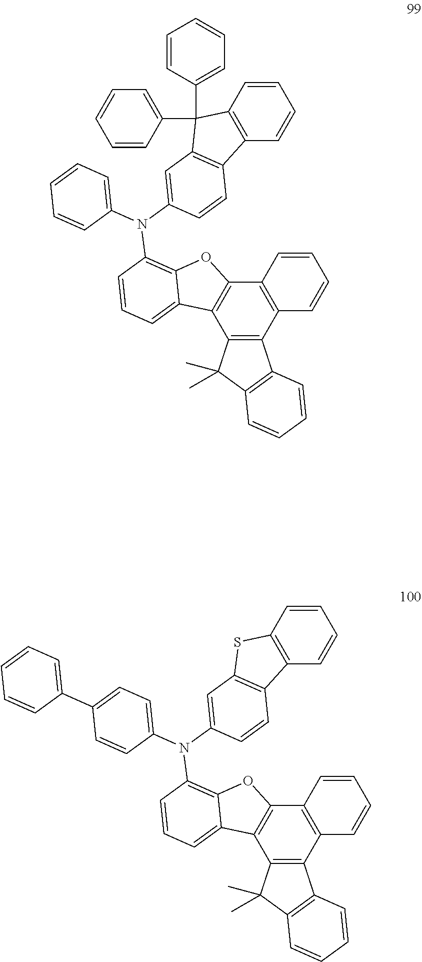 [SMILES: CC1(C)c2ccccc2-c2c1c1c3cccc(N(c4ccccc4)c4ccc5c(c4)C(c4ccccc4)(c4ccccc4)c4ccccc4-5)c3oc1c1ccccc21 ; CC1(C)c2ccccc2-c2c1c1c3cccc(N(c4ccc(-c5ccccc5)cc4)c4ccc5c(c4)sc4ccccc45)c3oc1c1ccccc21]

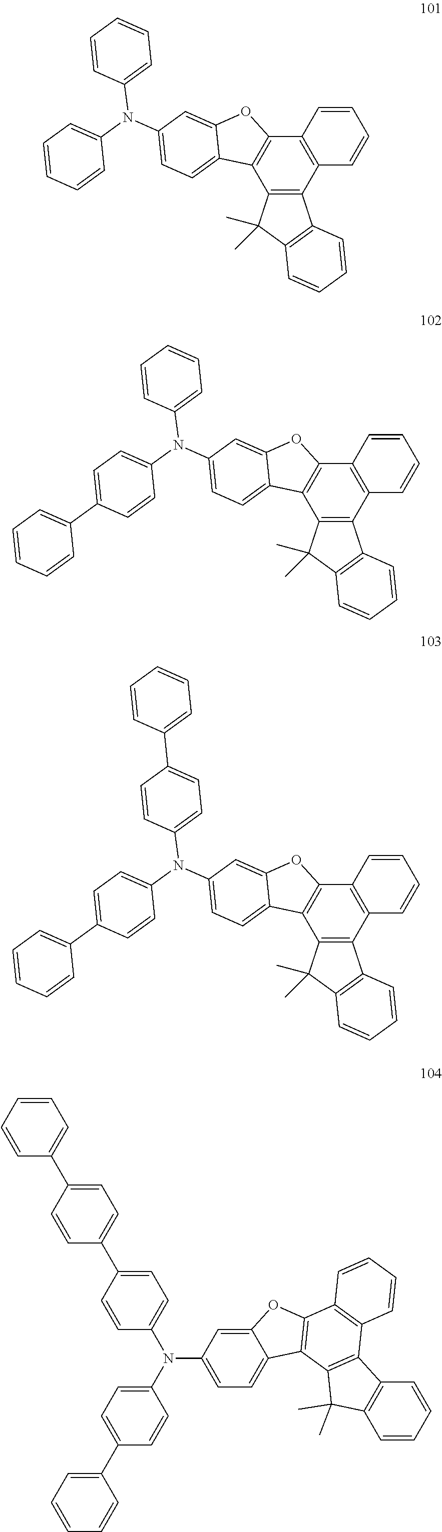 [SMILES: CC1(C)c2ccccc2-c2c1c1c3ccc(N(c4ccccc4)c4ccccc4)cc3oc1c1ccccc21 ; CC1(C)c2ccccc2-c2c1c1c3ccc(N(c4ccccc4)c4ccc(-c5ccccc5)cc4)cc3oc1c1ccccc21 ; CC1(C)c2ccccc2-c2c1c1c3ccc(N(c4ccc(-c5ccccc5)cc4)c4ccc(-c5ccccc5)cc4)cc3oc1c1ccccc21 ; CC1(C)c2ccccc2-c2c1c1c3ccc(N(c4ccc(-c5ccccc5)cc4)c4ccc(-c5ccc(-c6ccccc6)cc5)cc4)cc3oc1c1ccccc21]

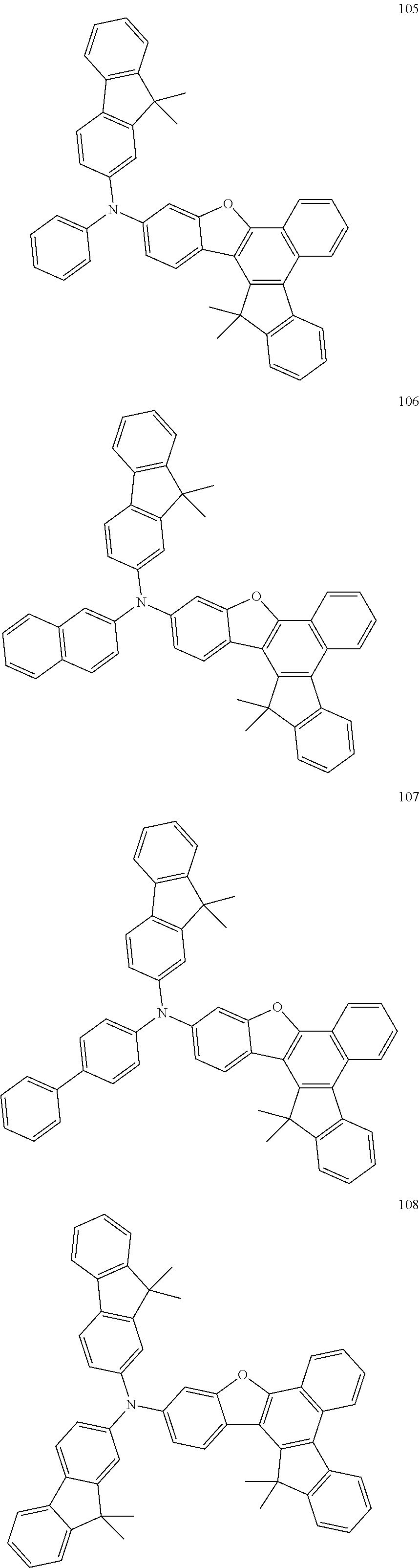 [SMILES: CC1(C)c2ccccc2-c2ccc(N(c3ccccc3)c3ccc4c(c3)oc3c5ccccc5c5c(c43)C(C)(C)c3ccccc3-5)cc21 ; CC1(C)c2ccccc2-c2ccc(N(c3ccc4ccccc4c3)c3ccc4c(c3)oc3c5ccccc5c5c(c43)C(C)(C)c3ccccc3-5)cc21 ; CC1(C)c2ccccc2-c2ccc(N(c3ccc(-c4ccccc4)cc3)c3ccc4c(c3)oc3c5ccccc5c5c(c43)C(C)(C)c3ccccc3-5)cc21 ; CC1(C)c2ccccc2-c2ccc(N(c3ccc4c(c3)C(C)(C)c3ccccc3-4)c3ccc4c(c3)oc3c5ccccc5c5c(c43)C(C)(C)c3ccccc3-5)cc21]

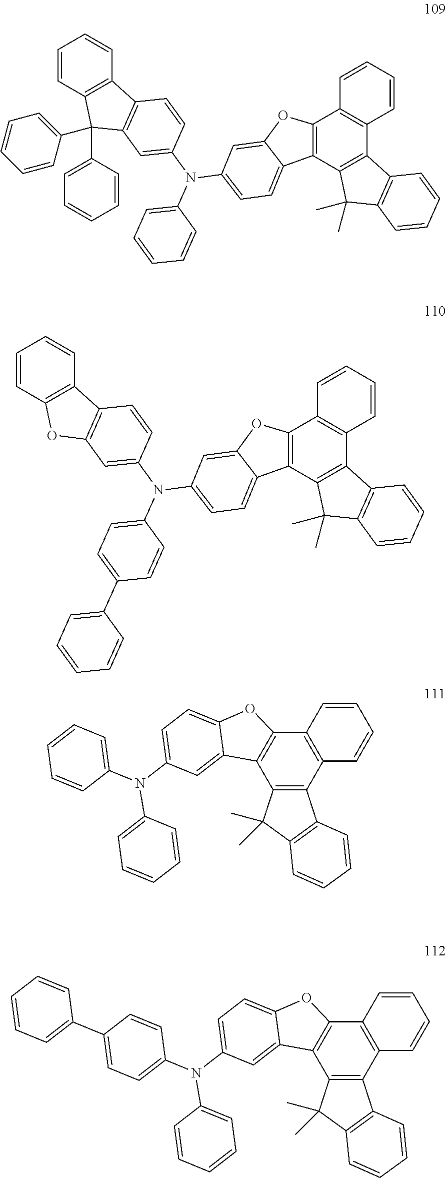 [SMILES: CC1(C)c2ccccc2-c2c1c1c3ccc(N(c4ccccc4)c4ccc5c(c4)C(c4ccccc4)(c4ccccc4)c4ccccc4-5)cc3oc1c1ccccc21 ; CC1(C)c2ccccc2-c2c1c1c3ccc(N(c4ccc(-c5ccccc5)cc4)c4ccc5c(c4)oc4ccccc45)cc3oc1c1ccccc21 ; CC1(C)c2ccccc2-c2c1c1c3cc(N(c4ccccc4)c4ccccc4)ccc3oc1c1ccccc21 ; CC1(C)c2ccccc2-c2c1c1c3cc(N(c4ccccc4)c4ccc(-c5ccccc5)cc4)ccc3oc1c1ccccc21]

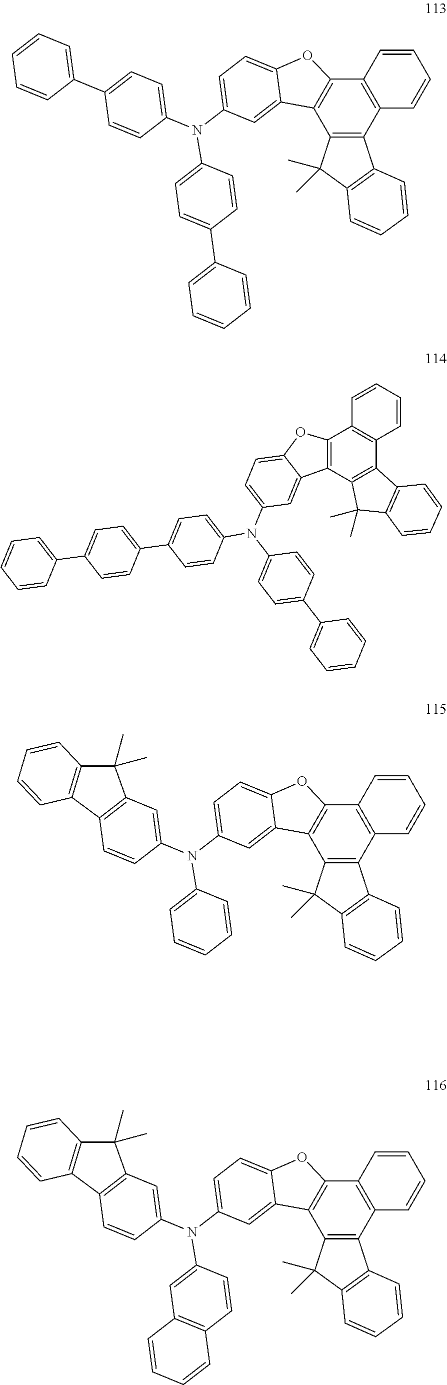 [SMILES: CC1(C)c2ccccc2-c2c1c1c3cc(N(c4ccc(-c5ccccc5)cc4)c4ccc(-c5ccccc5)cc4)ccc3oc1c1ccccc21 ; CC1(C)c2ccccc2-c2c1c1c3cc(N(c4ccc(-c5ccccc5)cc4)c4ccc(-c5ccc(-c6ccccc6)cc5)cc4)ccc3oc1c1ccccc21 ; CC1(C)c2ccccc2-c2ccc(N(c3ccccc3)c3ccc4oc5c6ccccc6c6c(c5c4c3)C(C)(C)c3ccccc3-6)cc21 ; CC1(C)c2ccccc2-c2ccc(N(c3ccc4ccccc4c3)c3ccc4oc5c6ccccc6c6c(c5c4c3)C(C)(C)c3ccccc3-6)cc21]

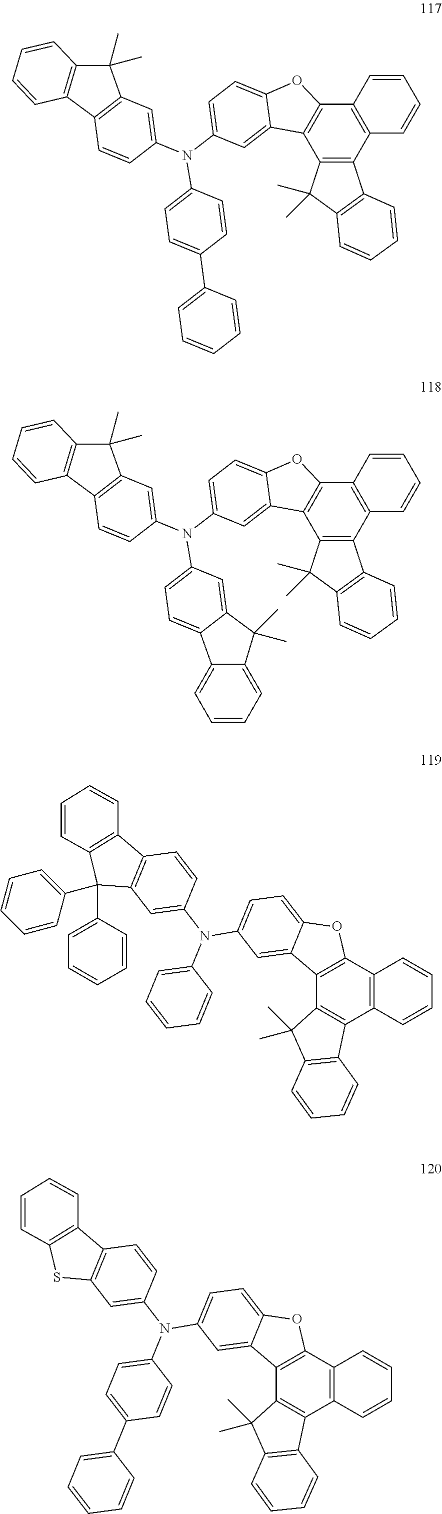 [SMILES: CC1(C)c2ccccc2-c2ccc(N(c3ccc(-c4ccccc4)cc3)c3ccc4oc5c6ccccc6c6c(c5c4c3)C(C)(C)c3ccccc3-6)cc21 ; CC1(C)c2ccccc2-c2ccc(N(c3ccc4c(c3)C(C)(C)c3ccccc3-4)c3ccc4oc5c6ccccc6c6c(c5c4c3)C(C)(C)c3ccccc3-6)cc21 ; CC1(C)c2ccccc2-c2c1c1c3cc(N(c4ccccc4)c4ccc5c(c4)C(c4ccccc4)(c4ccccc4)c4ccccc4-5)ccc3oc1c1ccccc21 ; CC1(C)c2ccccc2-c2c1c1c3cc(N(c4ccc(-c5ccccc5)cc4)c4ccc5c(c4)sc4ccccc45)ccc3oc1c1ccccc21]

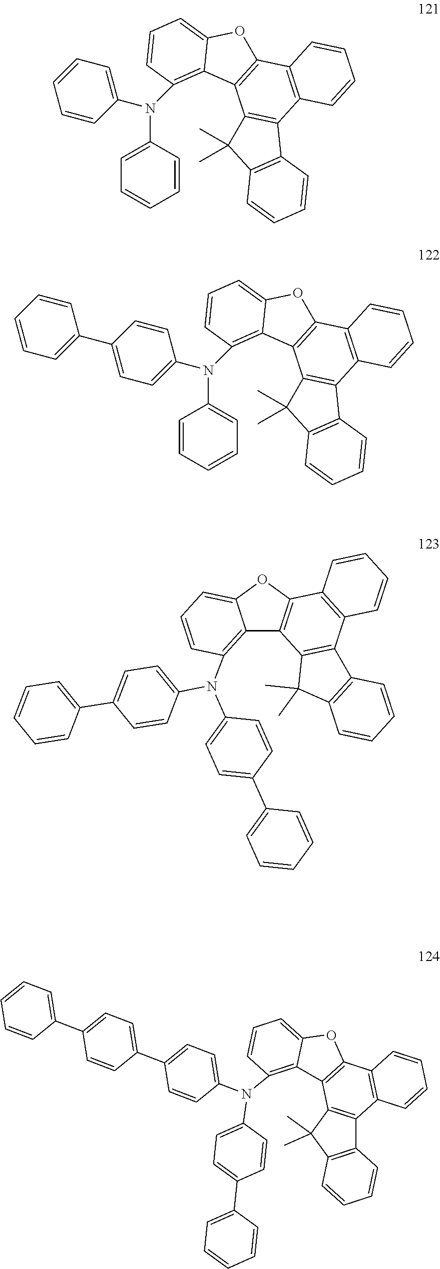 [SMILES: CC1(C)c2ccccc2-c2c1c1c(oc3cccc(N(c4ccccc4)c4ccccc4)c31)c1ccccc21 ; CC1(C)c2ccccc2-c2c1c1c(oc3cccc(N(c4ccccc4)c4ccc(-c5ccccc5)cc4)c31)c1ccccc21 ; CC1(C)c2ccccc2-c2c1c1c(oc3cccc(N(c4ccc(-c5ccccc5)cc4)c4ccc(-c5ccccc5)cc4)c31)c1ccccc21 ; CC1(C)c2ccccc2-c2c1c1c(oc3cccc(N(c4ccc(-c5ccccc5)cc4)c4ccc(-c5ccc(-c6ccccc6)cc5)cc4)c31)c1ccccc21]

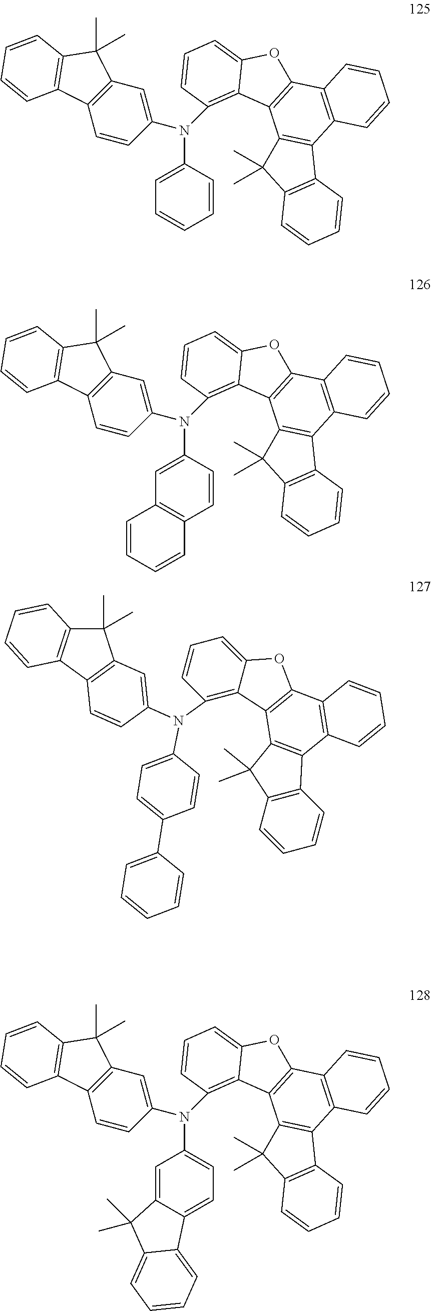

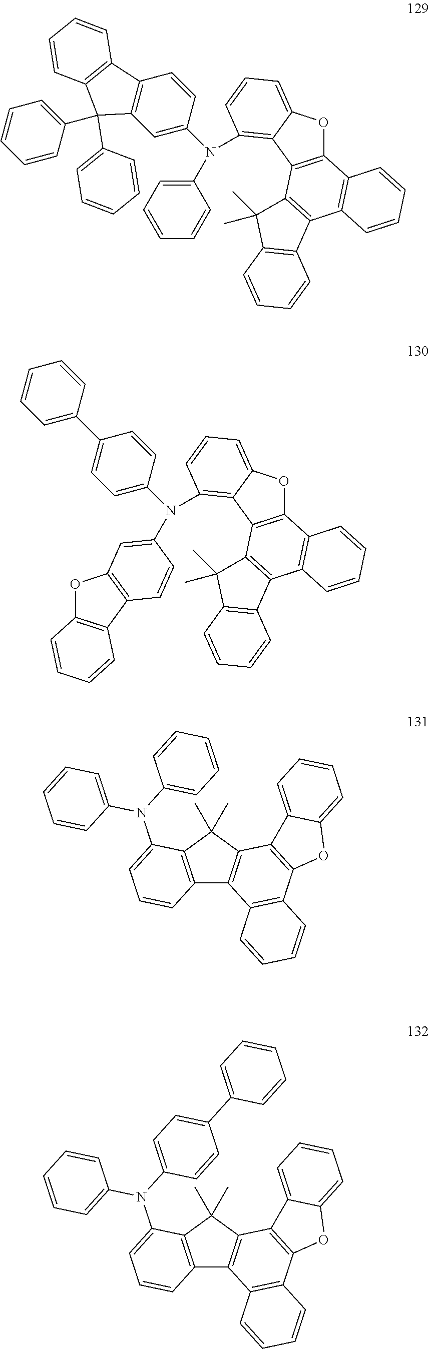 [SMILES: CC1(C)c2ccccc2-c2c1c1c(oc3cccc(N(c4ccccc4)c4ccc5c(c4)C(c4ccccc4)(c4ccccc4)c4ccccc4-5)c31)c1ccccc21 ; CC1(C)c2ccccc2-c2c1c1c(oc3cccc(N(c4ccc(-c5ccccc5)cc4)c4ccc5c(c4)oc4ccccc45)c31)c1ccccc21 ; CC1(C)c2c(cccc2N(c2ccccc2)c2ccccc2)-c2c1c1c3ccccc3oc1c1ccccc21 ; CC1(C)c2c(cccc2N(c2ccccc2)c2ccc(-c3ccccc3)cc2)-c2c1c1c3ccccc3oc1c1ccccc21]

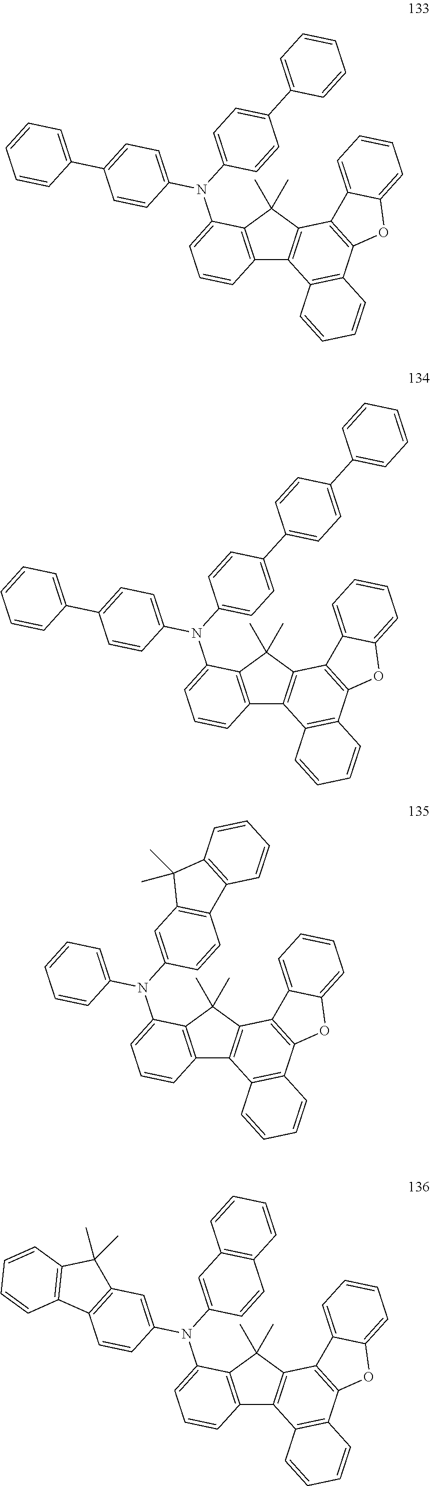 [SMILES: CC1(C)c2c(cccc2N(c2ccc(-c3ccccc3)cc2)c2ccc(-c3ccccc3)cc2)-c2c1c1c3ccccc3oc1c1ccccc21 ; CC1(C)c2c(cccc2N(c2ccc(-c3ccccc3)cc2)c2ccc(-c3ccc(-c4ccccc4)cc3)cc2)-c2c1c1c3ccccc3oc1c1ccccc21 ; CC1(C)c2ccccc2-c2ccc(N(c3ccccc3)c3cccc4c3C(C)(C)c3c-4c4ccccc4c4oc5ccccc5c34)cc21 ; CC1(C)c2ccccc2-c2ccc(N(c3ccc4ccccc4c3)c3cccc4c3C(C)(C)c3c-4c4ccccc4c4oc5ccccc5c34)cc21]

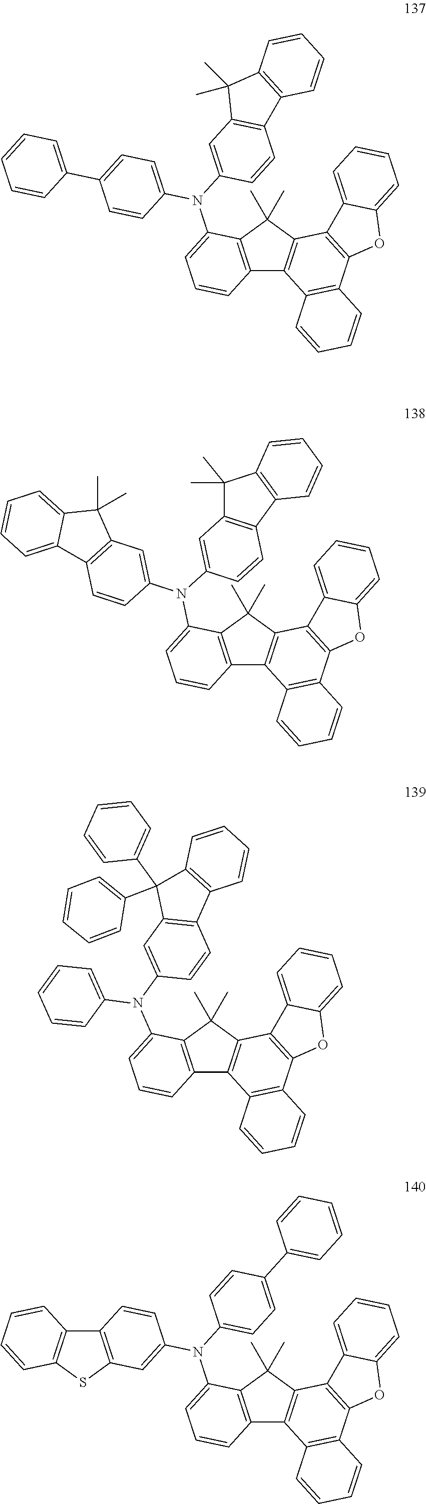 [SMILES: CC1(C)c2ccccc2-c2ccc(N(c3ccc(-c4ccccc4)cc3)c3cccc4c3C(C)(C)c3c-4c4ccccc4c4oc5ccccc5c34)cc21 ; CC1(C)c2ccccc2-c2ccc(N(c3ccc4c(c3)C(C)(C)c3ccccc3-4)c3cccc4c3C(C)(C)c3c-4c4ccccc4c4oc5ccccc5c34)cc21 ; CC1(C)c2c(cccc2N(c2ccccc2)c2ccc3c(c2)C(c2ccccc2)(c2ccccc2)c2ccccc2-3)-c2c1c1c3ccccc3oc1c1ccccc21 ; CC1(C)c2c(cccc2N(c2ccc(-c3ccccc3)cc2)c2ccc3c(c2)sc2ccccc23)-c2c1c1c3ccccc3oc1c1ccccc21]

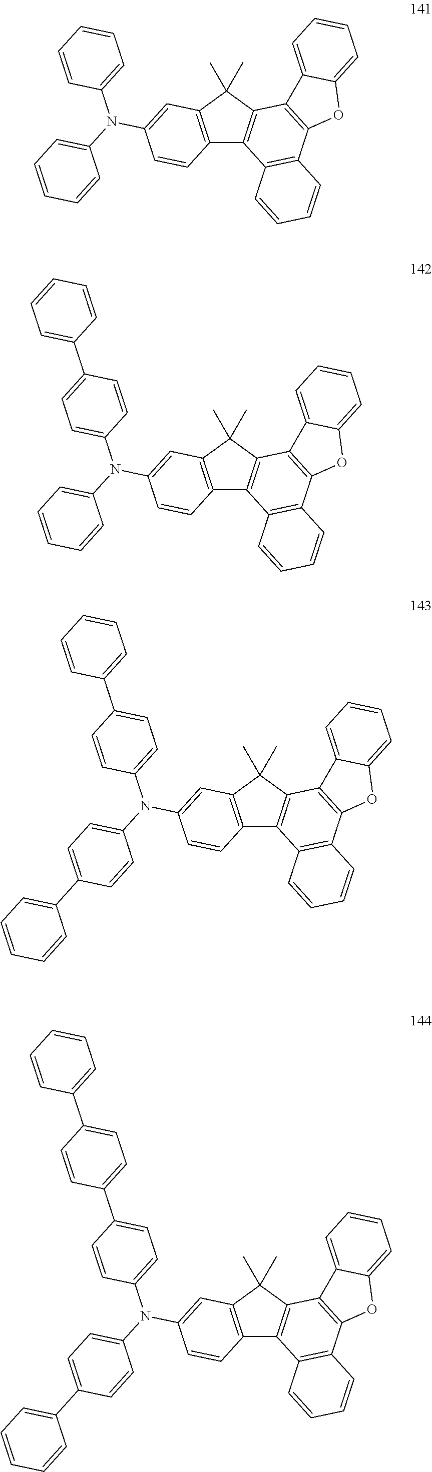 [SMILES: CC1(C)c2cc(N(c3ccccc3)c3ccccc3)ccc2-c2c1c1c3ccccc3oc1c1ccccc21 ; CC1(C)c2cc(N(c3ccccc3)c3ccc(-c4ccccc4)cc3)ccc2-c2c1c1c3ccccc3oc1c1ccccc21 ; CC1(C)c2cc(N(c3ccc(-c4ccccc4)cc3)c3ccc(-c4ccccc4)cc3)ccc2-c2c1c1c3ccccc3oc1c1ccccc21 ; CC1(C)c2cc(N(c3ccc(-c4ccccc4)cc3)c3ccc(-c4ccc(-c5ccccc5)cc4)cc3)ccc2-c2c1c1c3ccccc3oc1c1ccccc21]

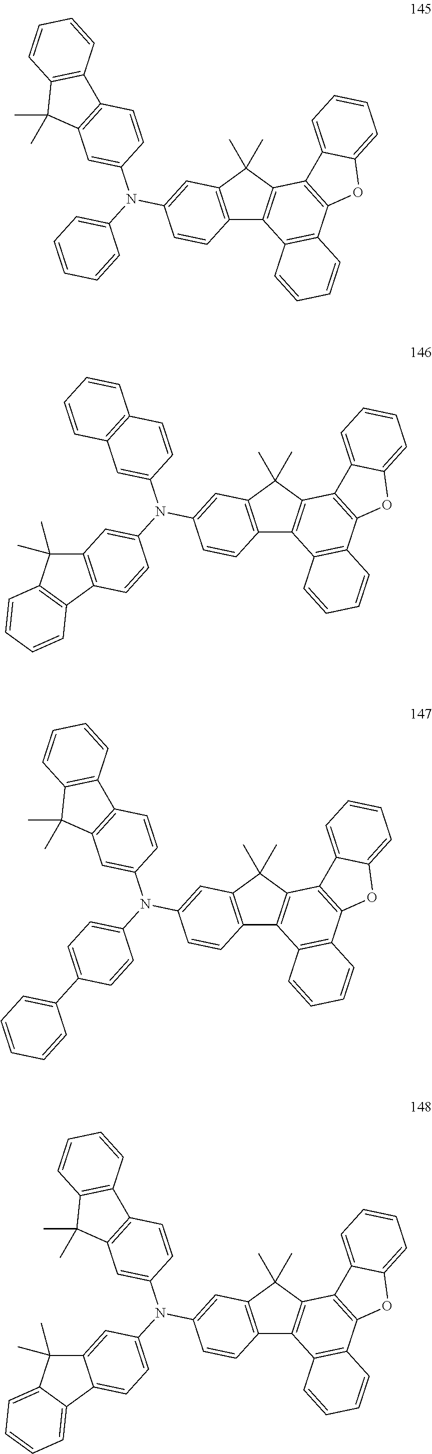 [SMILES: CC1(C)c2ccccc2-c2ccc(N(c3ccccc3)c3ccc4c(c3)C(C)(C)c3c-4c4ccccc4c4oc5ccccc5c34)cc21 ; CC1(C)c2ccccc2-c2ccc(N(c3ccc4c(c3)C(C)(C)c3c-4c4ccccc4c4oc5ccccc5c34)c3ccc4ccccc4c3)cc21 ; CC1(C)c2ccccc2-c2ccc(N(c3ccc(-c4ccccc4)cc3)c3ccc4c(c3)C(C)(C)c3c-4c4ccccc4c4oc5ccccc5c34)cc21 ; CC1(C)c2ccccc2-c2ccc(N(c3ccc4c(c3)C(C)(C)c3ccccc3-4)c3ccc4c(c3)C(C)(C)c3c-4c4ccccc4c4oc5ccccc5c34)cc21]

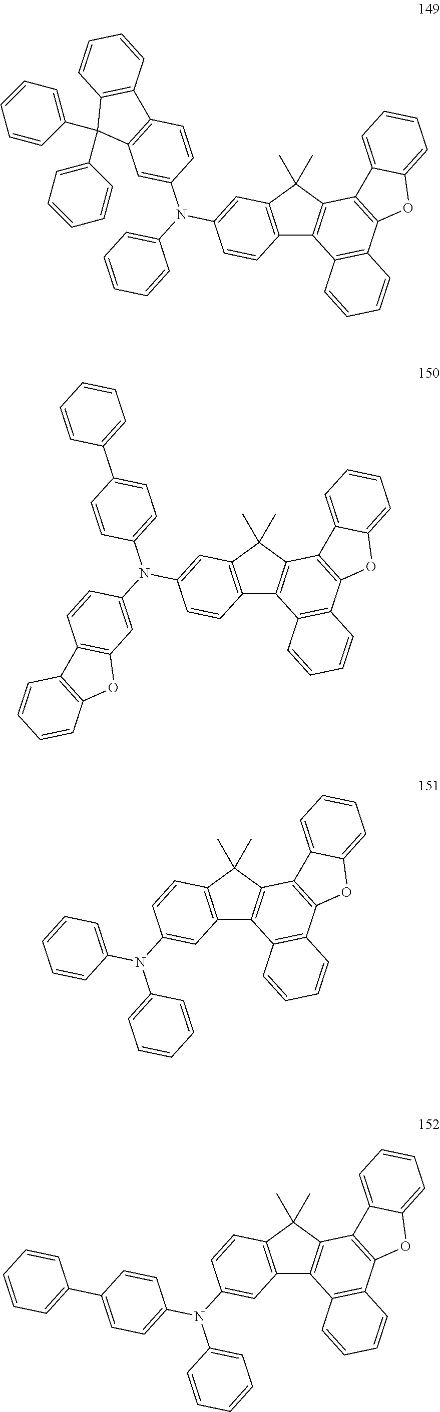 [SMILES: CC1(C)c2cc(N(c3ccccc3)c3ccc4c(c3)C(c3ccccc3)(c3ccccc3)c3ccccc3-4)ccc2-c2c1c1c3ccccc3oc1c1ccccc21 ; CC1(C)c2cc(N(c3ccc(-c4ccccc4)cc3)c3ccc4c(c3)oc3ccccc34)ccc2-c2c1c1c3ccccc3oc1c1ccccc21 ; CC1(C)c2ccc(N(c3ccccc3)c3ccccc3)cc2-c2c1c1c3ccccc3oc1c1ccccc21 ; CC1(C)c2ccc(N(c3ccccc3)c3ccc(-c4ccccc4)cc3)cc2-c2c1c1c3ccccc3oc1c1ccccc21]

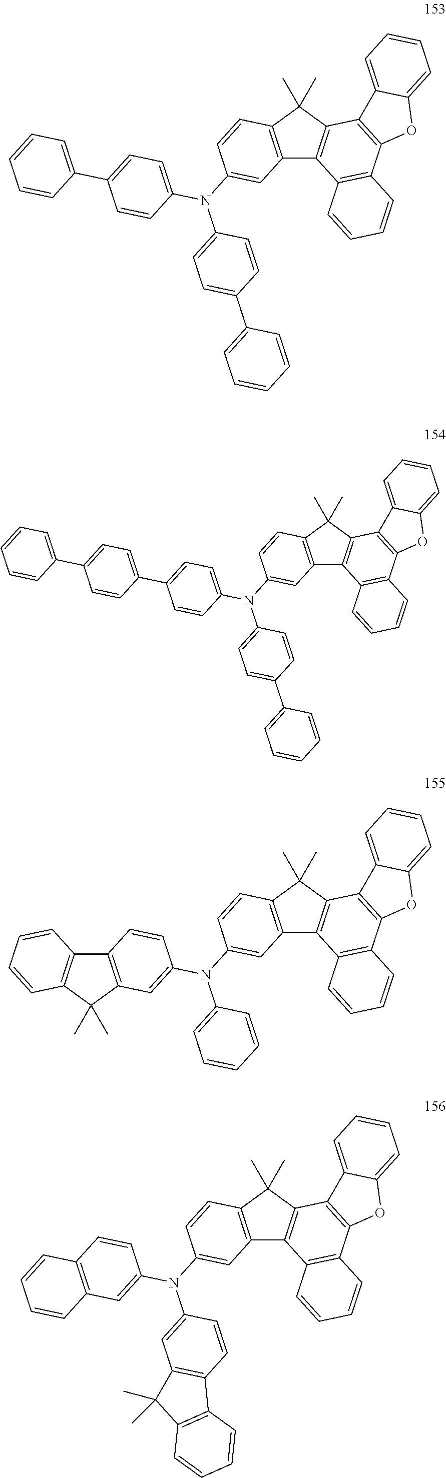 [SMILES: CC1(C)c2ccc(N(c3ccc(-c4ccccc4)cc3)c3ccc(-c4ccccc4)cc3)cc2-c2c1c1c3ccccc3oc1c1ccccc21 ; CC1(C)c2ccc(N(c3ccc(-c4ccccc4)cc3)c3ccc(-c4ccc(-c5ccccc5)cc4)cc3)cc2-c2c1c1c3ccccc3oc1c1ccccc21 ; CC1(C)c2ccccc2-c2ccc(N(c3ccccc3)c3ccc4c(c3)-c3c(c5c6ccccc6oc5c5ccccc35)C4(C)C)cc21 ; CC1(C)c2ccccc2-c2ccc(N(c3ccc4c(c3)-c3c(c5c6ccccc6oc5c5ccccc35)C4(C)C)c3ccc4ccccc4c3)cc21]

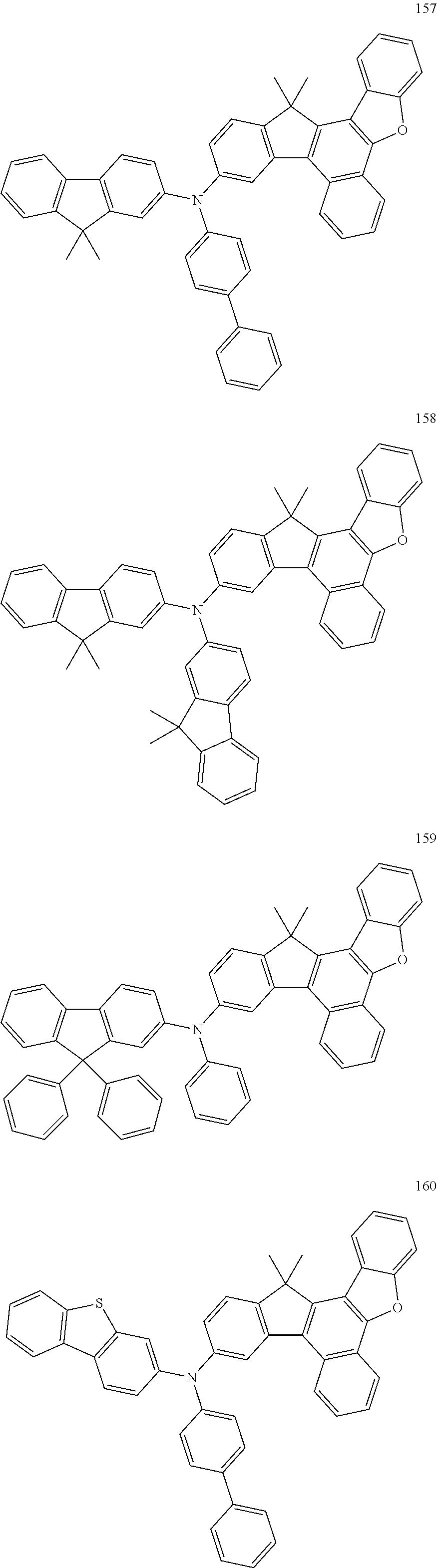 [SMILES: CC1(C)c2ccccc2-c2ccc(N(c3ccc(-c4ccccc4)cc3)c3ccc4c(c3)-c3c(c5c6ccccc6oc5c5ccccc35)C4(C)C)cc21 ; CC1(C)c2ccccc2-c2ccc(N(c3ccc4c(c3)-c3c(c5c6ccccc6oc5c5ccccc35)C4(C)C)c3ccc4c(c3)C(C)(C)c3ccccc3-4)cc21 ; CC1(C)c2ccc(N(c3ccccc3)c3ccc4c(c3)C(c3ccccc3)(c3ccccc3)c3ccccc3-4)cc2-c2c1c1c3ccccc3oc1c1ccccc21 ; CC1(C)c2ccc(N(c3ccc(-c4ccccc4)cc3)c3ccc4c(c3)sc3ccccc34)cc2-c2c1c1c3ccccc3oc1c1ccccc21]

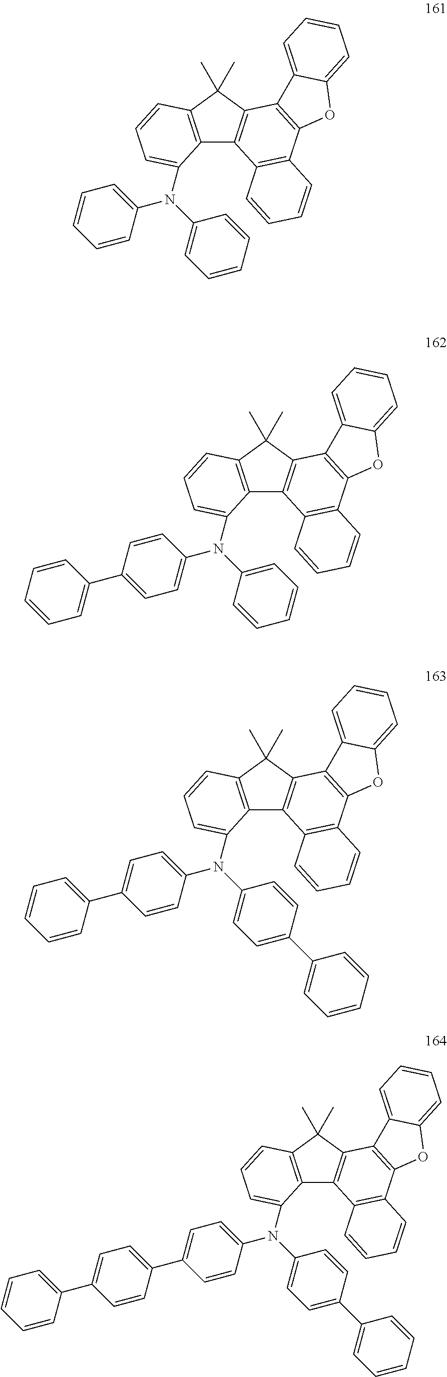 [SMILES: CC1(C)c2cccc(N(c3ccccc3)c3ccccc3)c2-c2c1c1c3ccccc3oc1c1ccccc21 ; CC1(C)c2cccc(N(c3ccccc3)c3ccc(-c4ccccc4)cc3)c2-c2c1c1c3ccccc3oc1c1ccccc21 ; CC1(C)c2cccc(N(c3ccc(-c4ccccc4)cc3)c3ccc(-c4ccccc4)cc3)c2-c2c1c1c3ccccc3oc1c1ccccc21 ; CC1(C)c2cccc(N(c3ccc(-c4ccccc4)cc3)c3ccc(-c4ccc(-c5ccccc5)cc4)cc3)c2-c2c1c1c3ccccc3oc1c1ccccc21]

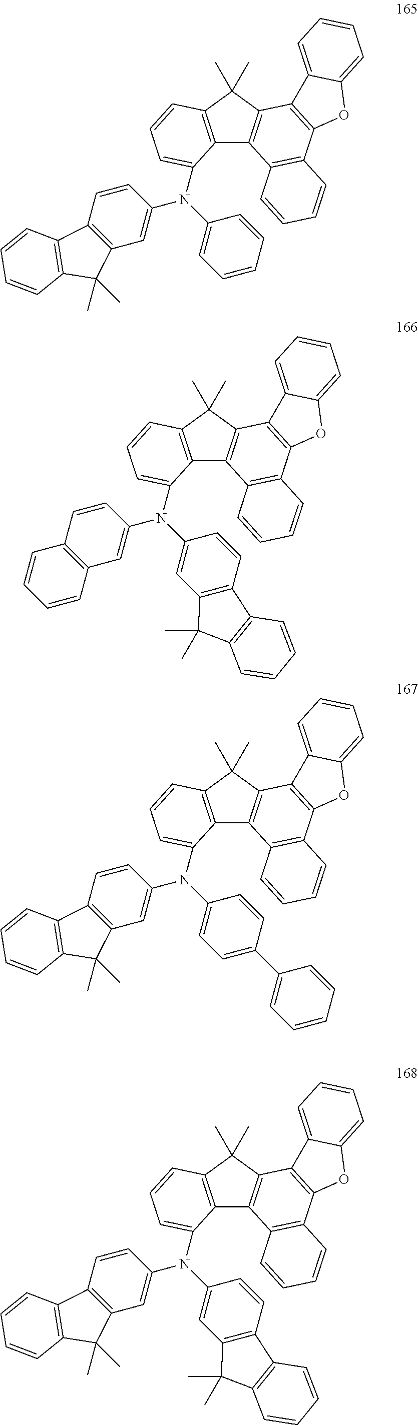 [SMILES: CC1(C)c2ccccc2-c2ccc(N(c3ccccc3)c3cccc4c3-c3c(c5c6ccccc6oc5c5ccccc35)C4(C)C)cc21 ; CC1(C)c2ccccc2-c2ccc(N(c3ccc4ccccc4c3)c3cccc4c3-c3c(c5c6ccccc6oc5c5ccccc35)C4(C)C)cc21 ; CC1(C)c2ccccc2-c2ccc(N(c3ccc(-c4ccccc4)cc3)c3cccc4c3-c3c(c5c6ccccc6oc5c5ccccc35)C4(C)C)cc21 ; CC1(C)c2ccccc2-c2ccc(N(c3ccc4c(c3)C(C)(C)c3ccccc3-4)c3cccc4c3-c3c(c5c6ccccc6oc5c5ccccc35)C4(C)C)cc21]

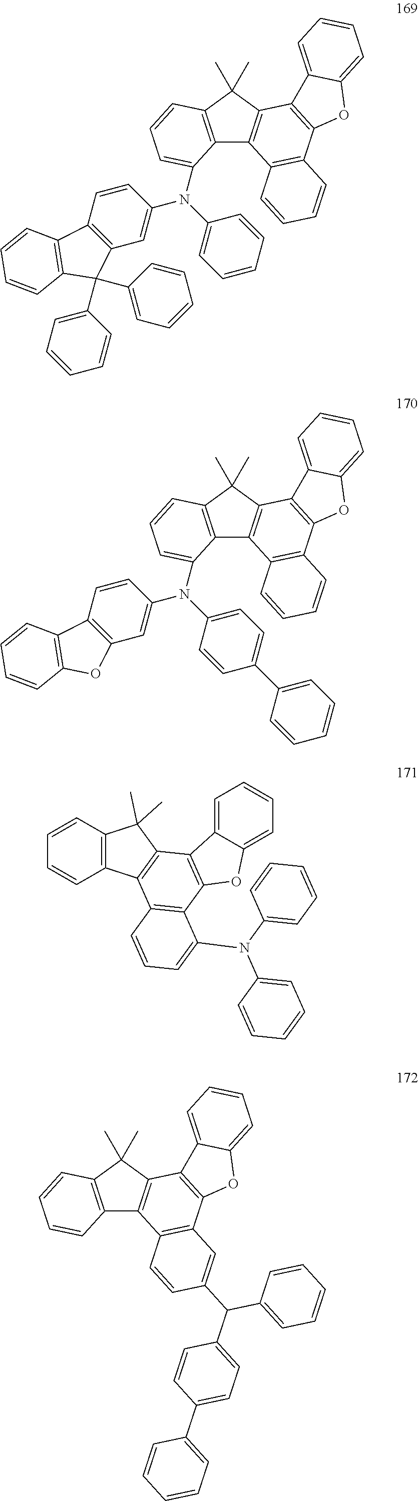 [SMILES: CC1(C)c2cccc(N(c3ccccc3)c3ccc4c(c3)C(c3ccccc3)(c3ccccc3)c3ccccc3-4)c2-c2c1c1c3ccccc3oc1c1ccccc21 ; CC1(C)c2cccc(N(c3ccc(-c4ccccc4)cc3)c3ccc4c(c3)oc3ccccc34)c2-c2c1c1c3ccccc3oc1c1ccccc21 ; CC1(C)c2ccccc2-c2c1c1c3ccccc3oc1c1c(N(c3ccccc3)c3ccccc3)cccc21 ; CC1(C)c2ccccc2-c2c1c1c3ccccc3oc1c1cc(C(c3ccccc3)c3ccc(-c4ccccc4)cc3)ccc21]

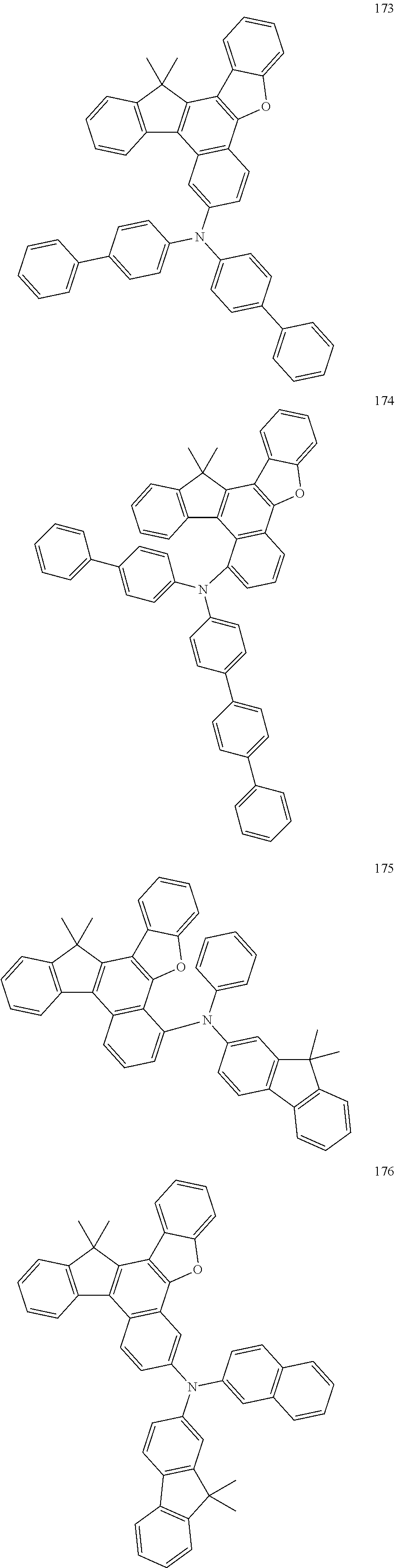

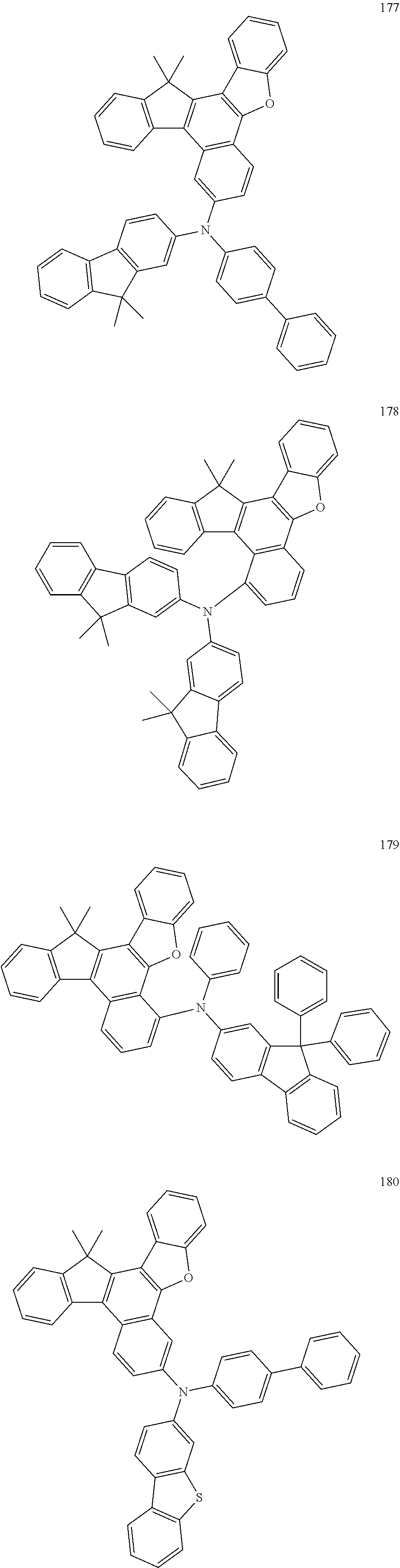 [SMILES: CC1(C)c2ccccc2-c2ccc(N(c3ccc(-c4ccccc4)cc3)c3ccc4c(c3)c3c(c5c6ccccc6oc45)C(C)(C)c4ccccc4-3)cc21 ; CC1(C)c2ccccc2-c2ccc(N(c3ccc4c(c3)C(C)(C)c3ccccc3-4)c3cccc4c3c3c(c5c6ccccc6oc45)C(C)(C)c4ccccc4-3)cc21 ; CC1(C)c2ccccc2-c2c1c1c3ccccc3oc1c1c(N(c3ccccc3)c3ccc4c(c3)C(c3ccccc3)(c3ccccc3)c3ccccc3-4)cccc21 ; CC1(C)c2ccccc2-c2c1c1c3ccccc3oc1c1cc(N(c3ccc(-c4ccccc4)cc3)c3ccc4c(c3)sc3ccccc34)ccc21]

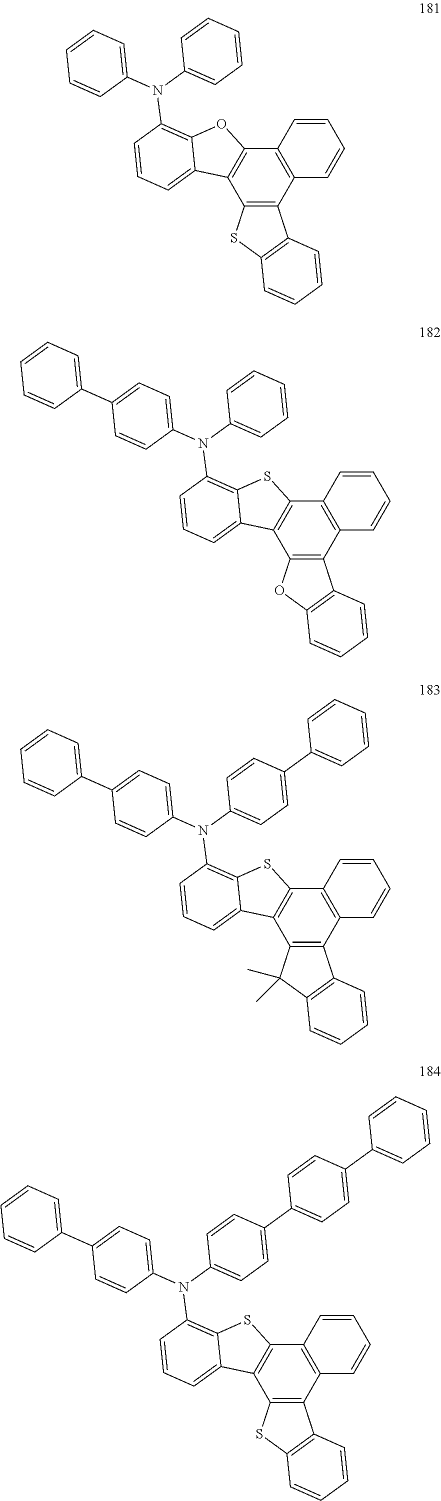

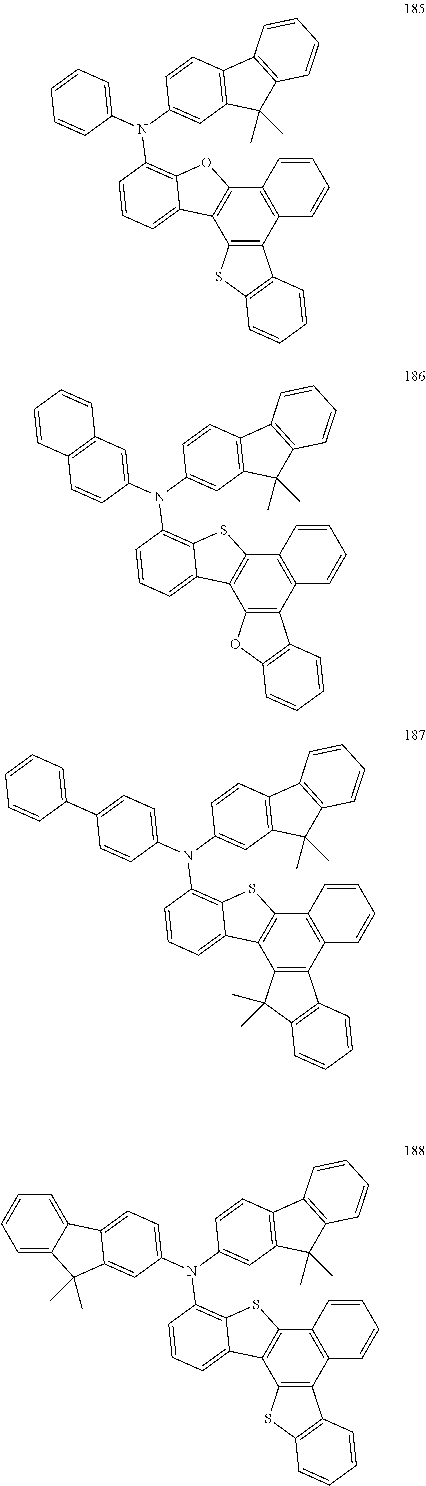

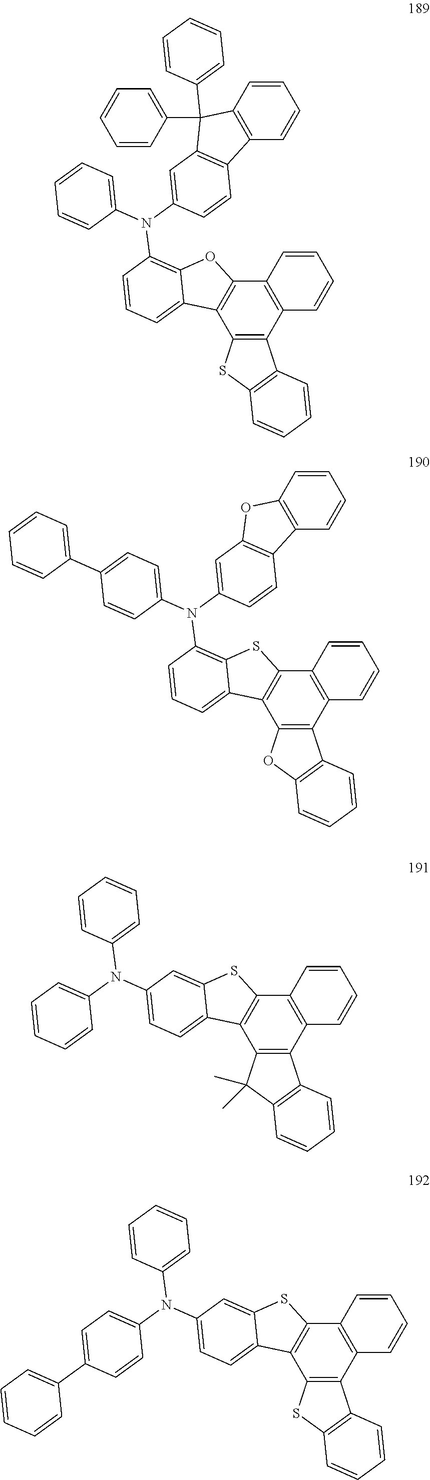

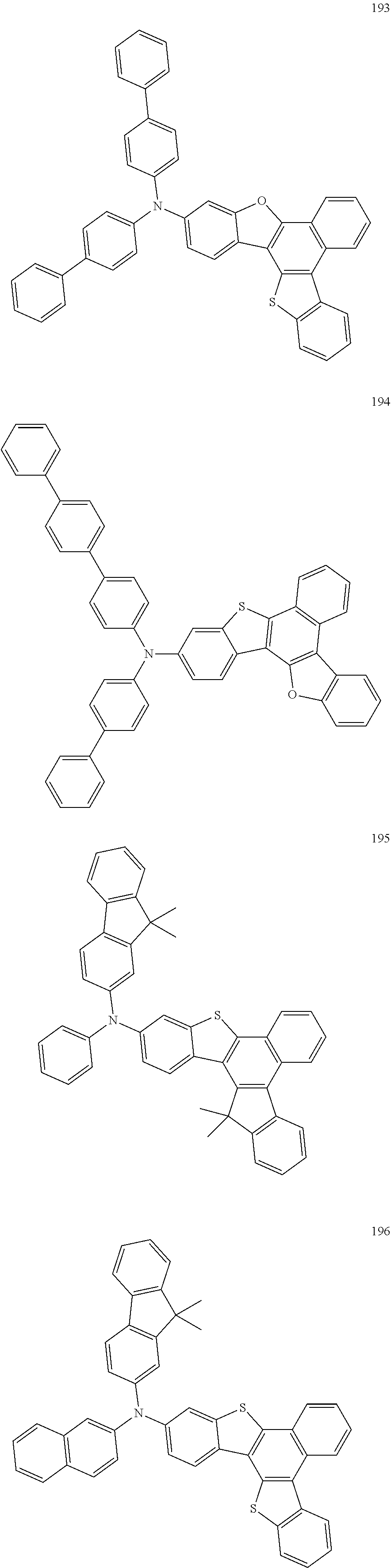 [SMILES: c1ccc(-c2ccc(N(c3ccc(-c4ccccc4)cc3)c3ccc4c(c3)oc3c5ccccc5c5c6ccccc6sc5c43)cc2)cc1 ; c1ccc(-c2ccc(-c3ccc(N(c4ccc(-c5ccccc5)cc4)c4ccc5c(c4)sc4c6ccccc6c6c7ccccc7oc6c54)cc3)cc2)cc1 ; CC1(C)c2ccccc2-c2ccc(N(c3ccccc3)c3ccc4c(c3)sc3c5ccccc5c5c(c43)C(C)(C)c3ccccc3-5)cc21 ; CC1(C)c2ccccc2-c2ccc(N(c3ccc4ccccc4c3)c3ccc4c(c3)sc3c5ccccc5c5c6ccccc6sc5c43)cc21]

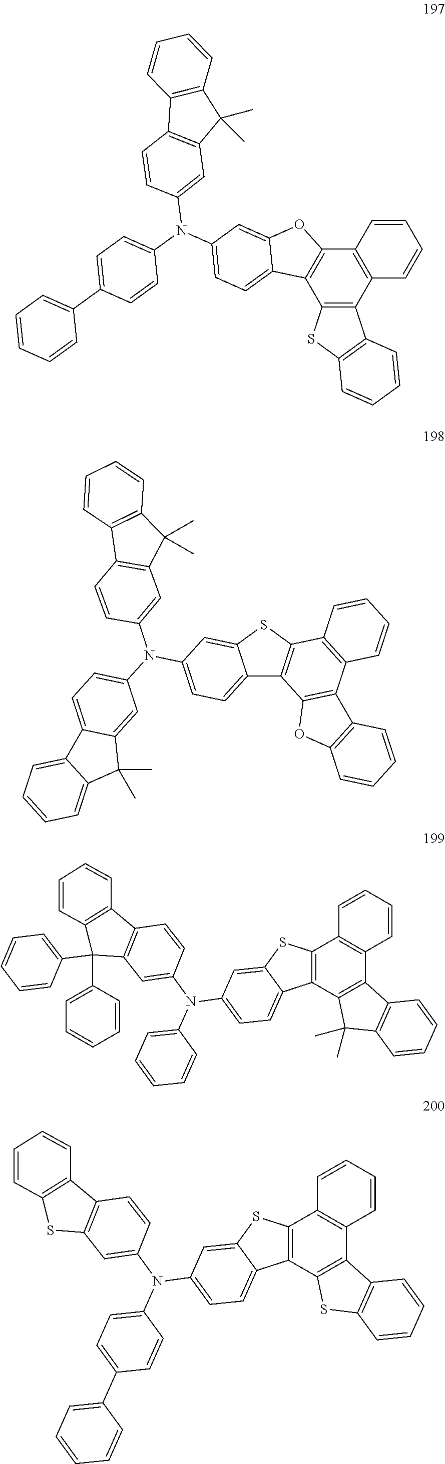 [SMILES: CC1(C)c2ccccc2-c2ccc(N(c3ccc(-c4ccccc4)cc3)c3ccc4c(c3)oc3c5ccccc5c5c6ccccc6sc5c43)cc21 ; CC1(C)c2ccccc2-c2ccc(N(c3ccc4c(c3)C(C)(C)c3ccccc3-4)c3ccc4c(c3)sc3c5ccccc5c5c6ccccc6oc5c43)cc21 ; CC1(C)c2ccccc2-c2c1c1c3ccc(N(c4ccccc4)c4ccc5c(c4)C(c4ccccc4)(c4ccccc4)c4ccccc4-5)cc3sc1c1ccccc21 ; c1ccc(-c2ccc(N(c3ccc4c(c3)sc3ccccc34)c3ccc4c(c3)sc3c5ccccc5c5c6ccccc6sc5c43)cc2)cc1]

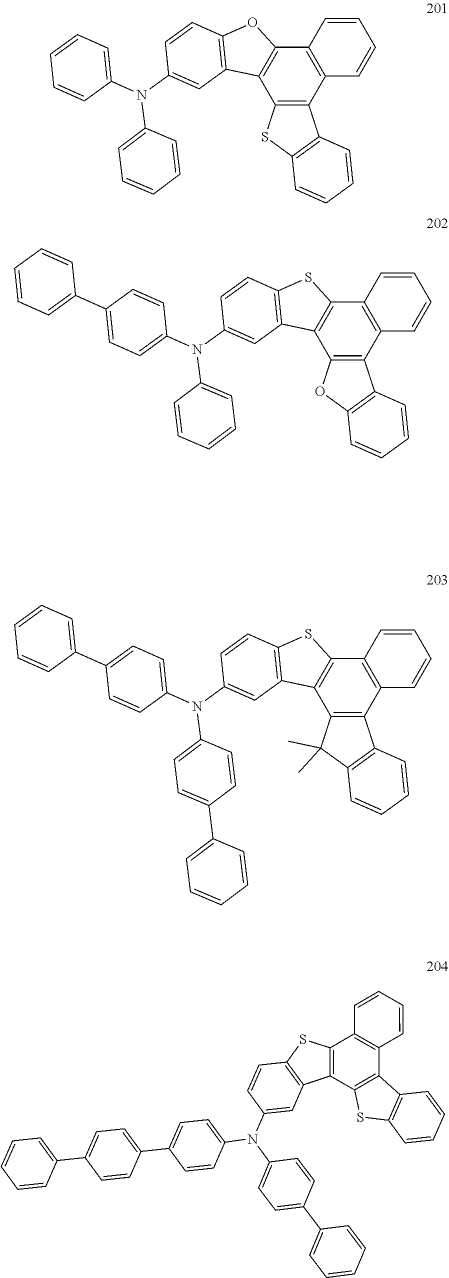 [SMILES: c1ccc(N(c2ccccc2)c2ccc3oc4c5ccccc5c5c6ccccc6sc5c4c3c2)cc1 ; c1ccc(-c2ccc(N(c3ccccc3)c3ccc4sc5c6ccccc6c6c7ccccc7oc6c5c4c3)cc2)cc1 ; CC1(C)c2ccccc2-c2c1c1c3cc(N(c4ccc(-c5ccccc5)cc4)c4ccc(-c5ccccc5)cc4)ccc3sc1c1ccccc21 ; c1ccc(-c2ccc(-c3ccc(N(c4ccc(-c5ccccc5)cc4)c4ccc5sc6c7ccccc7c7c8ccccc8sc7c6c5c4)cc3)cc2)cc1]

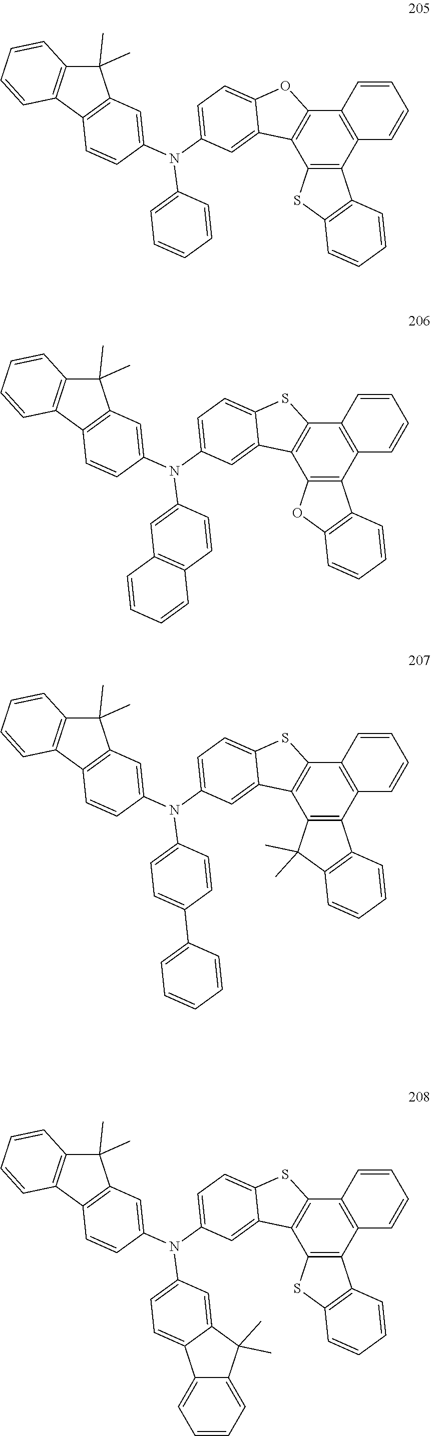 [SMILES: CC1(C)c2ccccc2-c2ccc(N(c3ccccc3)c3ccc4oc5c6ccccc6c6c7ccccc7sc6c5c4c3)cc21 ; CC1(C)c2ccccc2-c2ccc(N(c3ccc4ccccc4c3)c3ccc4sc5c6ccccc6c6c7ccccc7oc6c5c4c3)cc21 ; CC1(C)c2ccccc2-c2ccc(N(c3ccc(-c4ccccc4)cc3)c3ccc4sc5c6ccccc6c6c(c5c4c3)C(C)(C)c3ccccc3-6)cc21 ; CC1(C)c2ccccc2-c2ccc(N(c3ccc4c(c3)C(C)(C)c3ccccc3-4)c3ccc4sc5c6ccccc6c6c7ccccc7sc6c5c4c3)cc21]

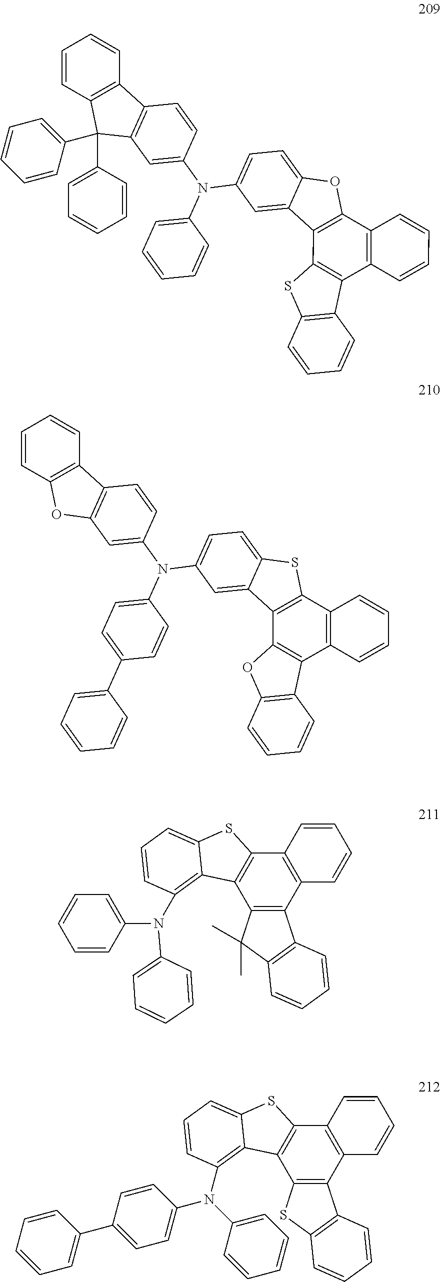 [SMILES: c1ccc(N(c2ccc3c(c2)C(c2ccccc2)(c2ccccc2)c2ccccc2-3)c2ccc3oc4c5ccccc5c5c6ccccc6sc5c4c3c2)cc1 ; c1ccc(-c2ccc(N(c3ccc4c(c3)oc3ccccc34)c3ccc4sc5c6ccccc6c6c7ccccc7oc6c5c4c3)cc2)cc1 ; CC1(C)c2ccccc2-c2c1c1c(sc3cccc(N(c4ccccc4)c4ccccc4)c31)c1ccccc21 ; c1ccc(-c2ccc(N(c3ccccc3)c3cccc4sc5c6ccccc6c6c7ccccc7sc6c5c34)cc2)cc1]

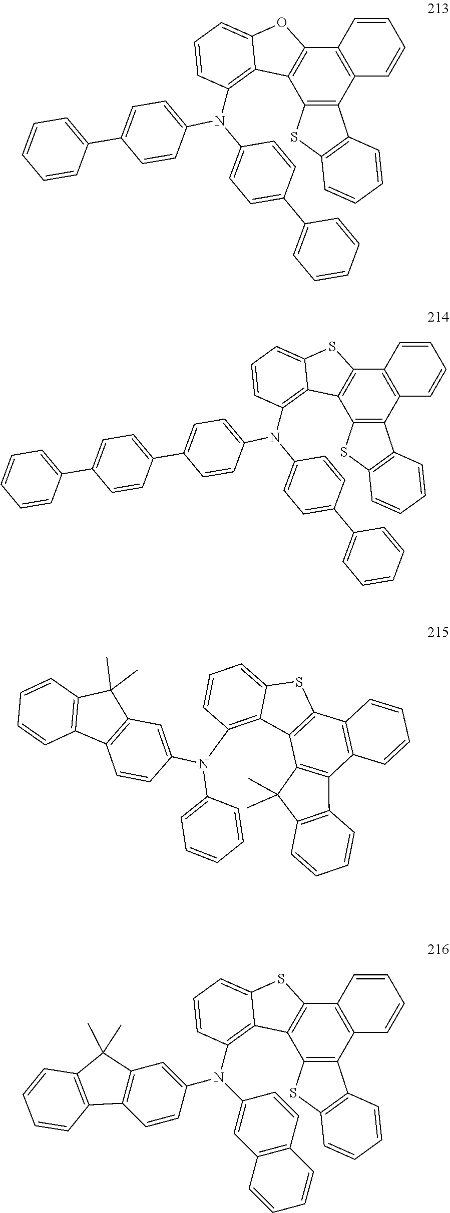 [SMILES: c1ccc(-c2ccc(N(c3ccc(-c4ccccc4)cc3)c3cccc4oc5c6ccccc6c6c7ccccc7sc6c5c34)cc2)cc1 ; c1ccc(-c2ccc(-c3ccc(N(c4ccc(-c5ccccc5)cc4)c4cccc5sc6c7ccccc7c7c8ccccc8sc7c6c45)cc3)cc2)cc1 ; CC1(C)c2ccccc2-c2ccc(N(c3ccccc3)c3cccc4sc5c6ccccc6c6c(c5c34)C(C)(C)c3ccccc3-6)cc21 ; CC1(C)c2ccccc2-c2ccc(N(c3ccc4ccccc4c3)c3cccc4sc5c6ccccc6c6c7ccccc7sc6c5c34)cc21]

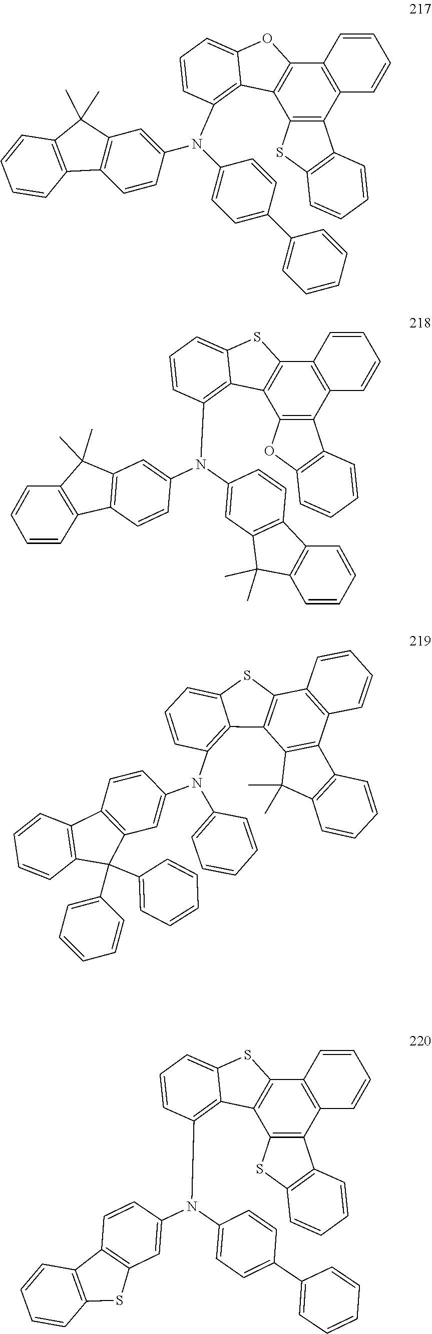

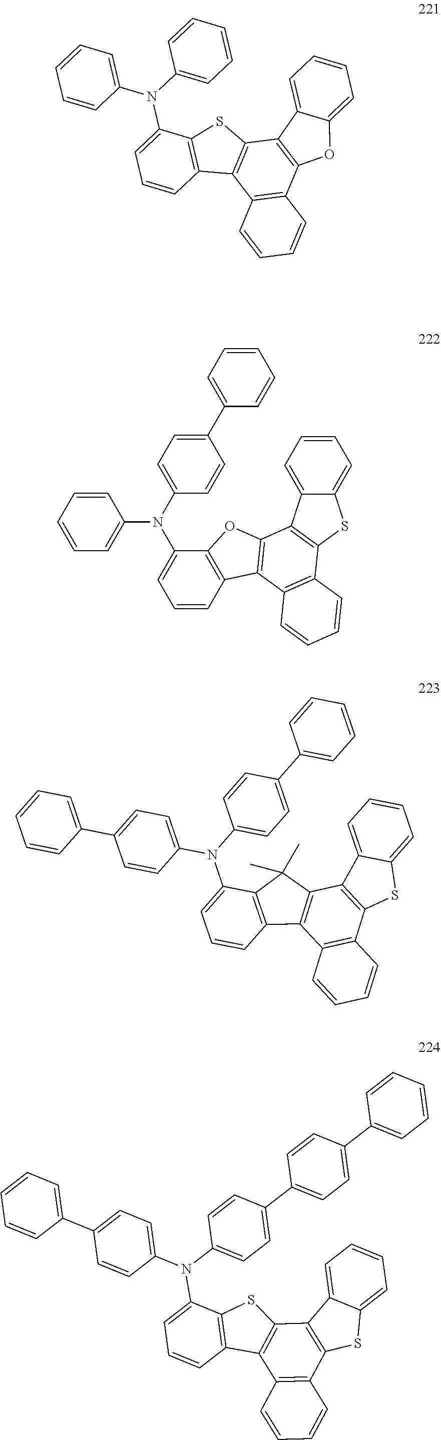 [SMILES: c1ccc(N(c2ccccc2)c2cccc3c2sc2c3c3ccccc3c3oc4ccccc4c32)cc1 ; c1ccc(-c2ccc(N(c3ccccc3)c3cccc4c3oc3c4c4ccccc4c4sc5ccccc5c34)cc2)cc1 ; CC1(C)c2c(cccc2N(c2ccc(-c3ccccc3)cc2)c2ccc(-c3ccccc3)cc2)-c2c1c1c3ccccc3sc1c1ccccc21 ; c1ccc(-c2ccc(-c3ccc(N(c4ccc(-c5ccccc5)cc4)c4cccc5c4sc4c5c5ccccc5c5sc6ccccc6c54)cc3)cc2)cc1]

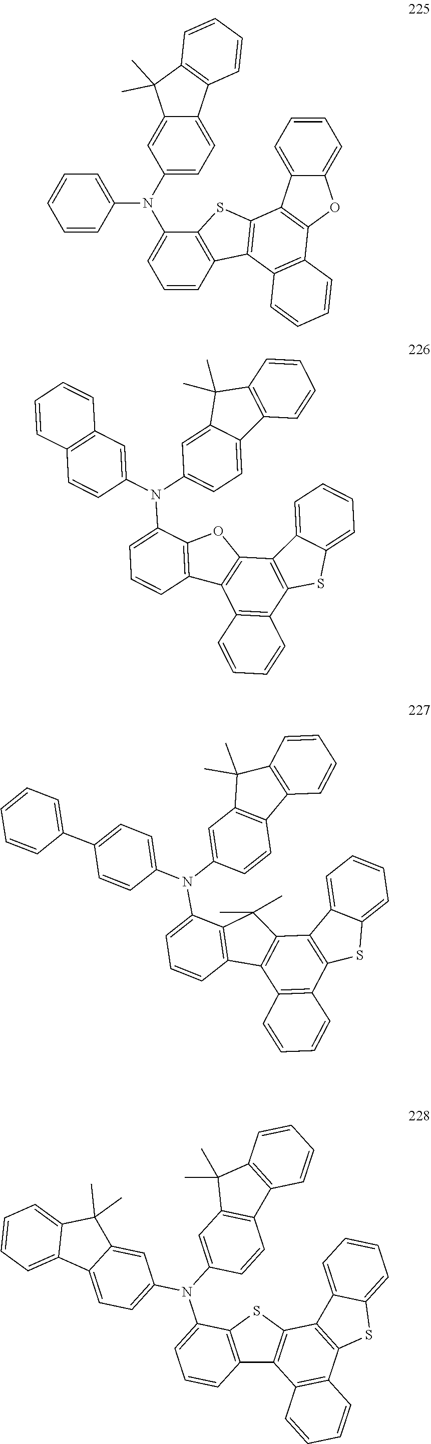

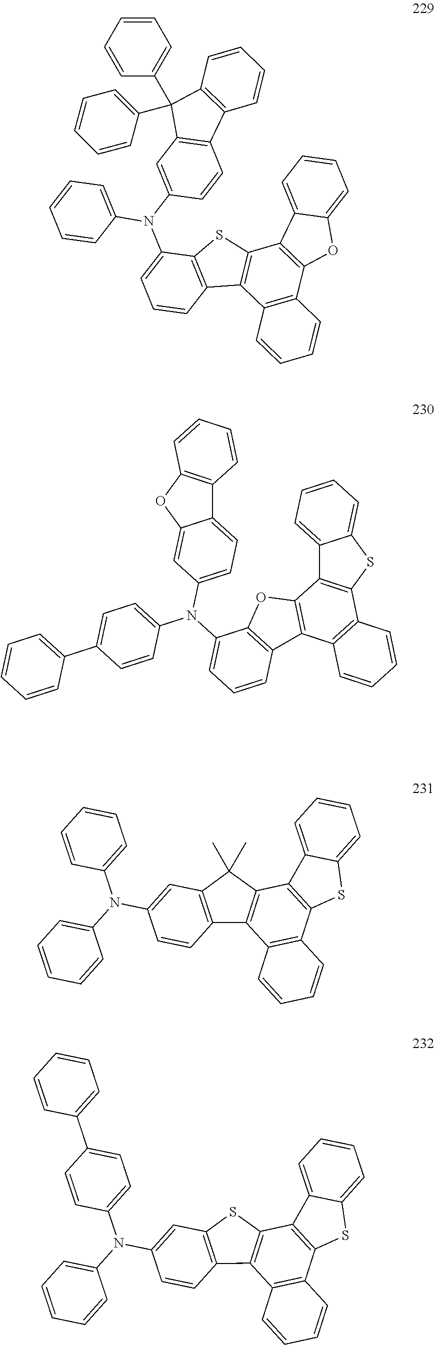 [SMILES: c1ccc(N(c2ccc3c(c2)C(c2ccccc2)(c2ccccc2)c2ccccc2-3)c2cccc3c2sc2c3c3ccccc3c3oc4ccccc4c32)cc1 ; c1ccc(-c2ccc(N(c3ccc4c(c3)oc3ccccc34)c3cccc4c3oc3c4c4ccccc4c4sc5ccccc5c34)cc2)cc1 ; CC1(C)c2cc(N(c3ccccc3)c3ccccc3)ccc2-c2c1c1c3ccccc3sc1c1ccccc21 ; c1ccc(-c2ccc(N(c3ccccc3)c3ccc4c(c3)sc3c4c4ccccc4c4sc5ccccc5c43)cc2)cc1]

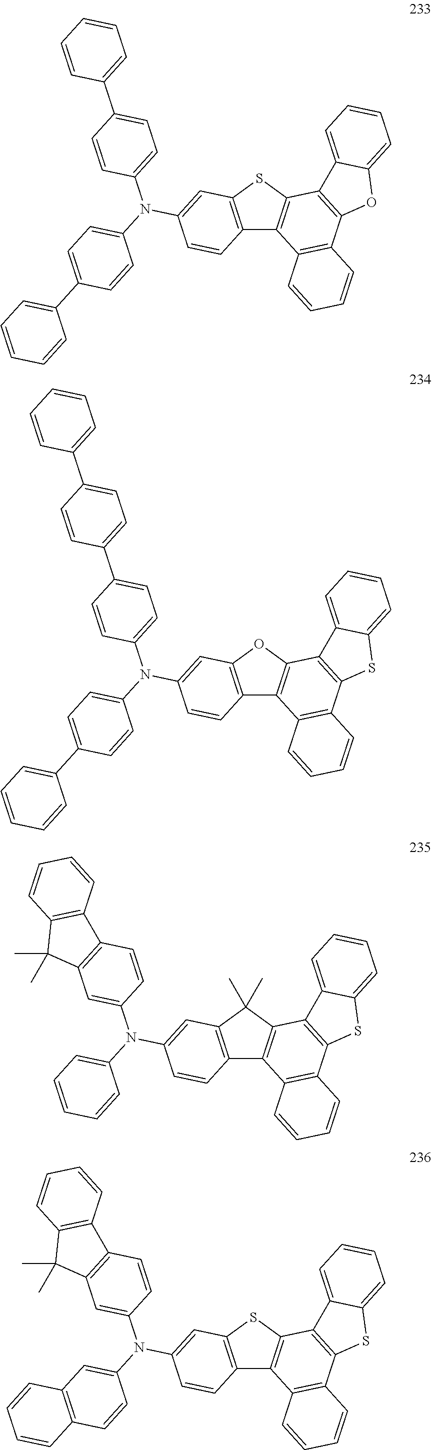

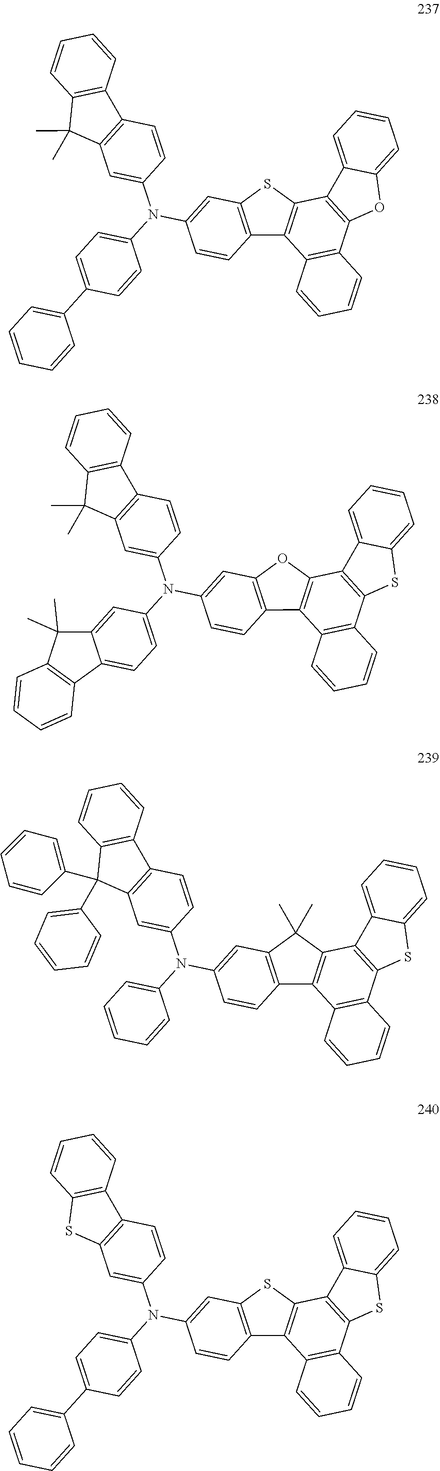 [SMILES: CC1(C)c2ccccc2-c2ccc(N(c3ccc(-c4ccccc4)cc3)c3ccc4c(c3)sc3c4c4ccccc4c4oc5ccccc5c43)cc21 ; CC1(C)c2ccccc2-c2ccc(N(c3ccc4c(c3)C(C)(C)c3ccccc3-4)c3ccc4c(c3)oc3c4c4ccccc4c4sc5ccccc5c34)cc21 ; CC1(C)c2cc(N(c3ccccc3)c3ccc4c(c3)C(c3ccccc3)(c3ccccc3)c3ccccc3-4)ccc2-c2c1c1c3ccccc3sc1c1ccccc21 ; c1ccc(-c2ccc(N(c3ccc4c(c3)sc3ccccc34)c3ccc4c(c3)sc3c4c4ccccc4c4sc5ccccc5c43)cc2)cc1]

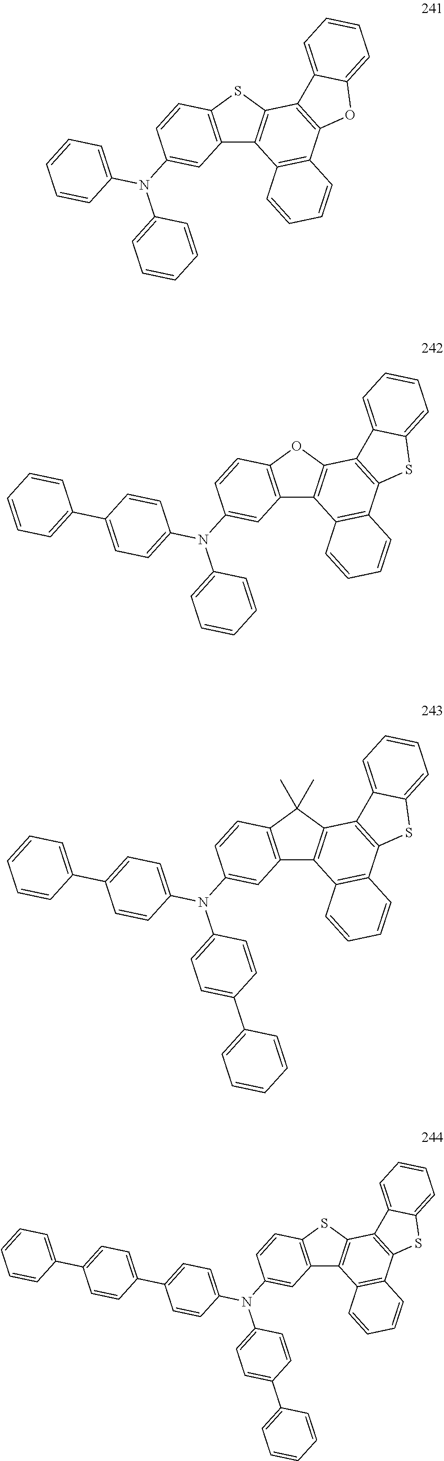 [SMILES: c1ccc(N(c2ccccc2)c2ccc3sc4c(c3c2)c2ccccc2c2oc3ccccc3c24)cc1 ; c1ccc(-c2ccc(N(c3ccccc3)c3ccc4oc5c(c4c3)c3ccccc3c3sc4ccccc4c53)cc2)cc1 ; CC1(C)c2ccc(N(c3ccc(-c4ccccc4)cc3)c3ccc(-c4ccccc4)cc3)cc2-c2c1c1c3ccccc3sc1c1ccccc21 ; c1ccc(-c2ccc(-c3ccc(N(c4ccc(-c5ccccc5)cc4)c4ccc5sc6c(c5c4)c4ccccc4c4sc5ccccc5c46)cc3)cc2)cc1]

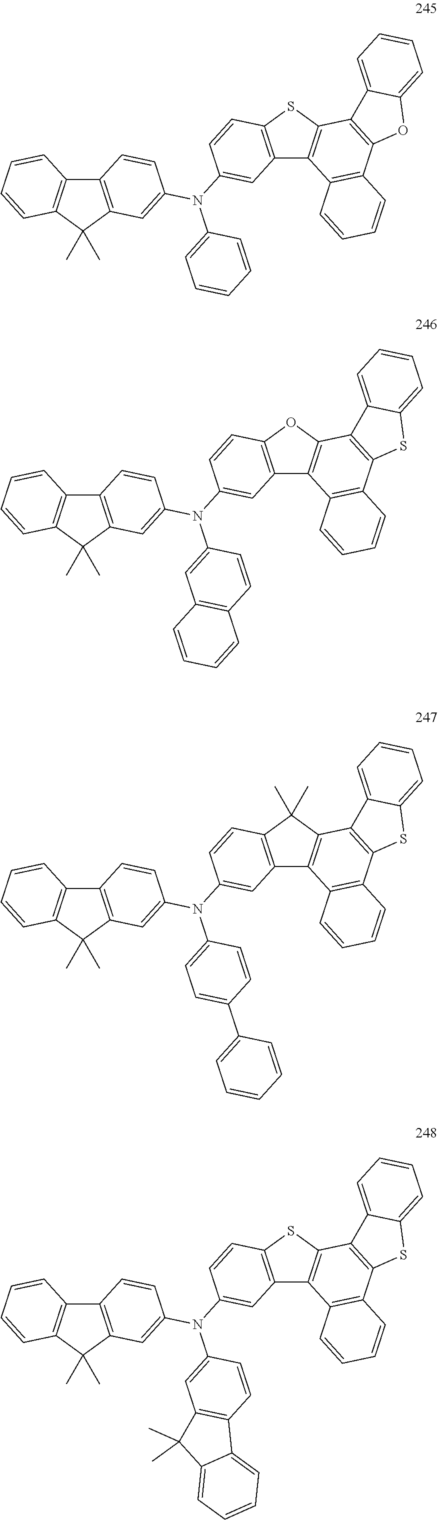 [SMILES: CC1(C)c2ccccc2-c2ccc(N(c3ccccc3)c3ccc4sc5c(c4c3)c3ccccc3c3oc4ccccc4c35)cc21 ; CC1(C)c2ccccc2-c2ccc(N(c3ccc4ccccc4c3)c3ccc4oc5c(c4c3)c3ccccc3c3sc4ccccc4c53)cc21 ; CC1(C)c2ccccc2-c2ccc(N(c3ccc(-c4ccccc4)cc3)c3ccc4c(c3)-c3c(c5c6ccccc6sc5c5ccccc35)C4(C)C)cc21 ; CC1(C)c2ccccc2-c2ccc(N(c3ccc4c(c3)C(C)(C)c3ccccc3-4)c3ccc4sc5c(c4c3)c3ccccc3c3sc4ccccc4c35)cc21]

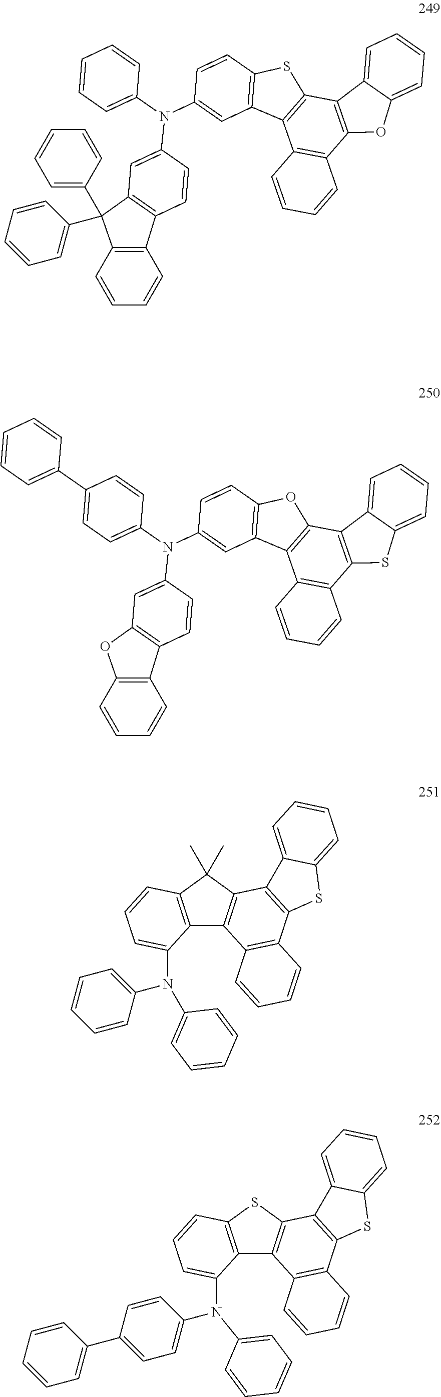 [SMILES: c1ccc(N(c2ccc3c(c2)C(c2ccccc2)(c2ccccc2)c2ccccc2-3)c2ccc3sc4c(c3c2)c2ccccc2c2oc3ccccc3c24)cc1 ; c1ccc(-c2ccc(N(c3ccc4c(c3)oc3ccccc34)c3ccc4oc5c(c4c3)c3ccccc3c3sc4ccccc4c53)cc2)cc1 ; CC1(C)c2cccc(N(c3ccccc3)c3ccccc3)c2-c2c1c1c3ccccc3sc1c1ccccc21 ; c1ccc(-c2ccc(N(c3ccccc3)c3cccc4sc5c6c7ccccc7sc6c6ccccc6c5c34)cc2)cc1]

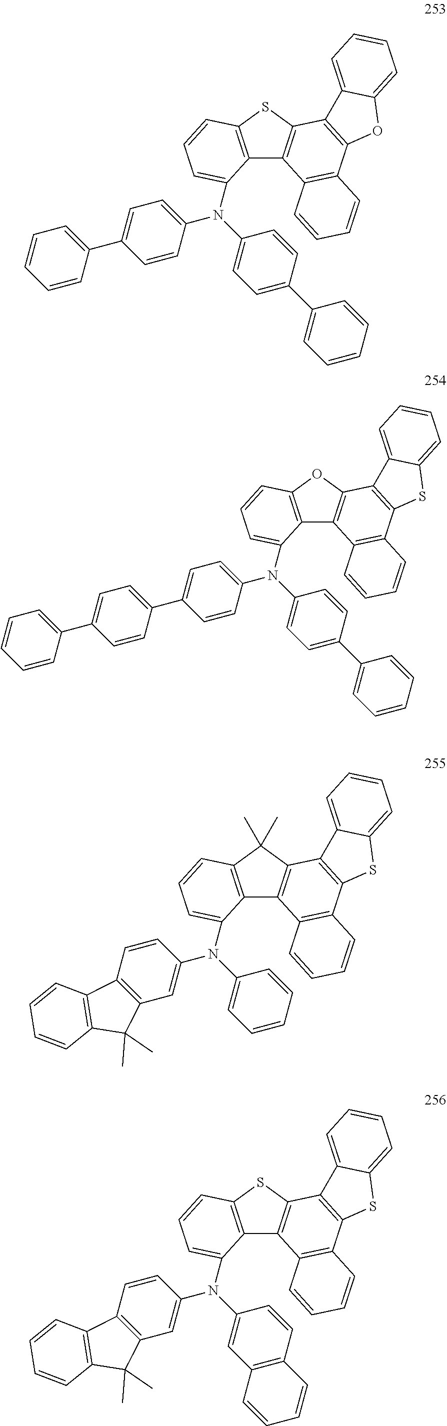 [SMILES: c1ccc(-c2ccc(N(c3ccc(-c4ccccc4)cc3)c3cccc4sc5c6c7ccccc7oc6c6ccccc6c5c34)cc2)cc1 ; c1ccc(-c2ccc(-c3ccc(N(c4ccc(-c5ccccc5)cc4)c4cccc5oc6c7c8ccccc8sc7c7ccccc7c6c45)cc3)cc2)cc1 ; CC1(C)c2ccccc2-c2ccc(N(c3ccccc3)c3cccc4c3-c3c(c5c6ccccc6sc5c5ccccc35)C4(C)C)cc21 ; CC1(C)c2ccccc2-c2ccc(N(c3ccc4ccccc4c3)c3cccc4sc5c6c7ccccc7sc6c6ccccc6c5c34)cc21]

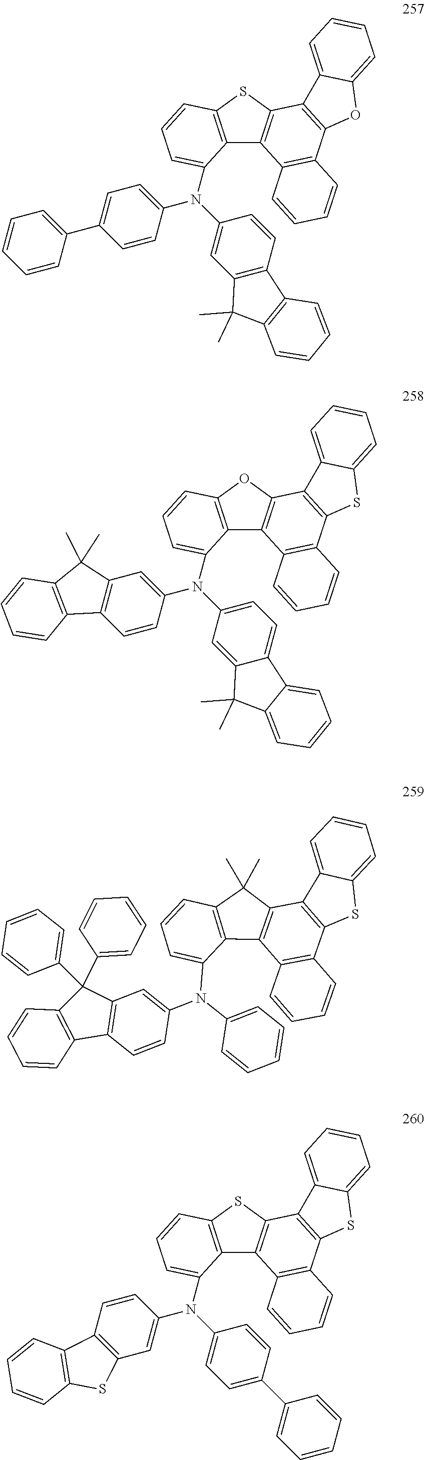 [SMILES: CC1(C)c2ccccc2-c2ccc(N(c3ccc(-c4ccccc4)cc3)c3cccc4sc5c6c7ccccc7oc6c6ccccc6c5c34)cc21 ; CC1(C)c2ccccc2-c2ccc(N(c3ccc4c(c3)C(C)(C)c3ccccc3-4)c3cccc4oc5c6c7ccccc7sc6c6ccccc6c5c34)cc21 ; CC1(C)c2cccc(N(c3ccccc3)c3ccc4c(c3)C(c3ccccc3)(c3ccccc3)c3ccccc3-4)c2-c2c1c1c3ccccc3sc1c1ccccc21 ; c1ccc(-c2ccc(N(c3ccc4c(c3)sc3ccccc34)c3cccc4sc5c6c7ccccc7sc6c6ccccc6c5c34)cc2)cc1]

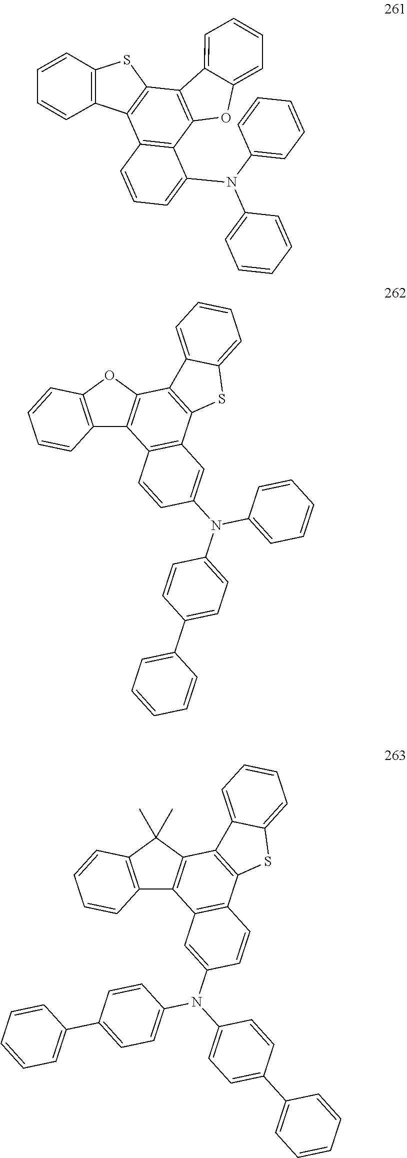

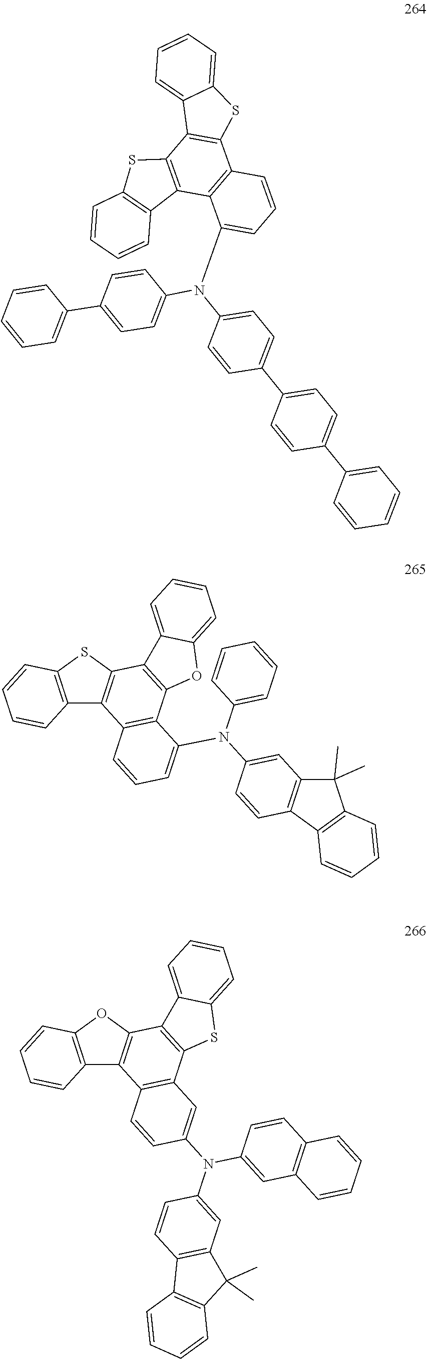 [SMILES: c1ccc(-c2ccc(-c3ccc(N(c4ccc(-c5ccccc5)cc4)c4cccc5c6sc7ccccc7c6c6sc7ccccc7c6c45)cc3)cc2)cc1 ; CC1(C)c2ccccc2-c2ccc(N(c3ccccc3)c3cccc4c3c3oc5ccccc5c3c3sc5ccccc5c43)cc21 ; CC1(C)c2ccccc2-c2ccc(N(c3ccc4ccccc4c3)c3ccc4c(c3)c3sc5ccccc5c3c3oc5ccccc5c43)cc21]

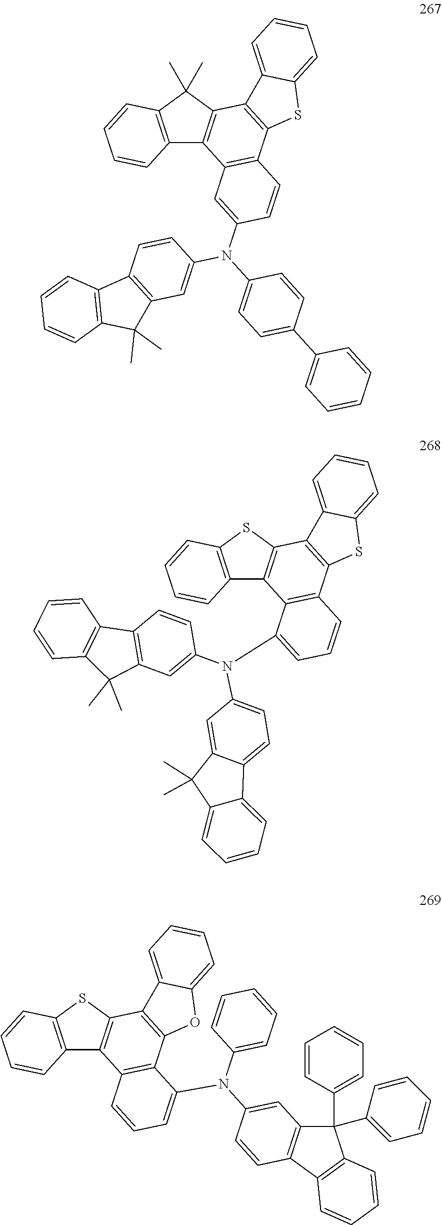

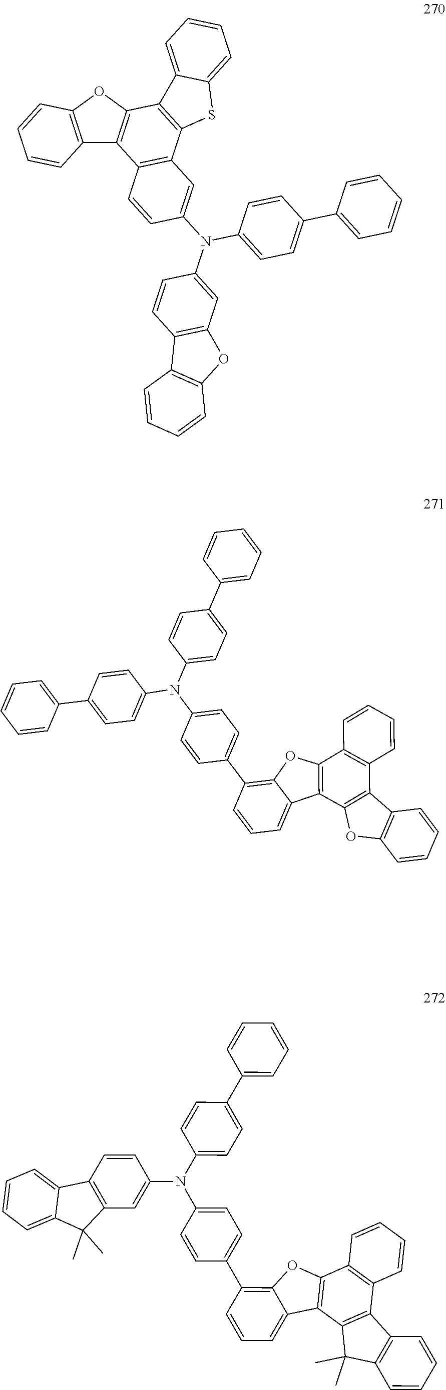 [SMILES: c1ccc(-c2ccc(N(c3ccc4c(c3)oc3ccccc34)c3ccc4c(c3)c3sc5ccccc5c3c3oc5ccccc5c43)cc2)cc1 ; c1ccc(-c2ccc(N(c3ccc(-c4ccccc4)cc3)c3ccc(-c4cccc5c4oc4c6ccccc6c6c7ccccc7oc6c54)cc3)cc2)cc1 ; CC1(C)c2ccccc2-c2ccc(N(c3ccc(-c4ccccc4)cc3)c3ccc(-c4cccc5c4oc4c6ccccc6c6c(c54)C(C)(C)c4ccccc4-6)cc3)cc21]

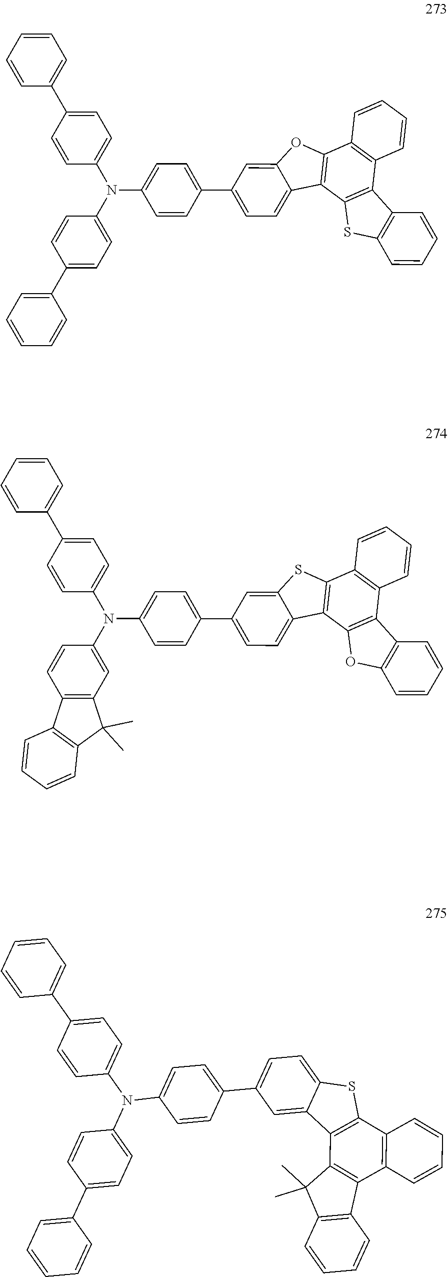 [SMILES: c1ccc(-c2ccc(N(c3ccc(-c4ccccc4)cc3)c3ccc(-c4ccc5c(c4)oc4c6ccccc6c6c7ccccc7sc6c54)cc3)cc2)cc1 ; CC1(C)c2ccccc2-c2ccc(N(c3ccc(-c4ccccc4)cc3)c3ccc(-c4ccc5c(c4)sc4c6ccccc6c6c7ccccc7oc6c54)cc3)cc21 ; CC1(C)c2ccccc2-c2c1c1c3cc(-c4ccc(N(c5ccc(-c6ccccc6)cc5)c5ccc(-c6ccccc6)cc5)cc4)ccc3sc1c1ccccc21]

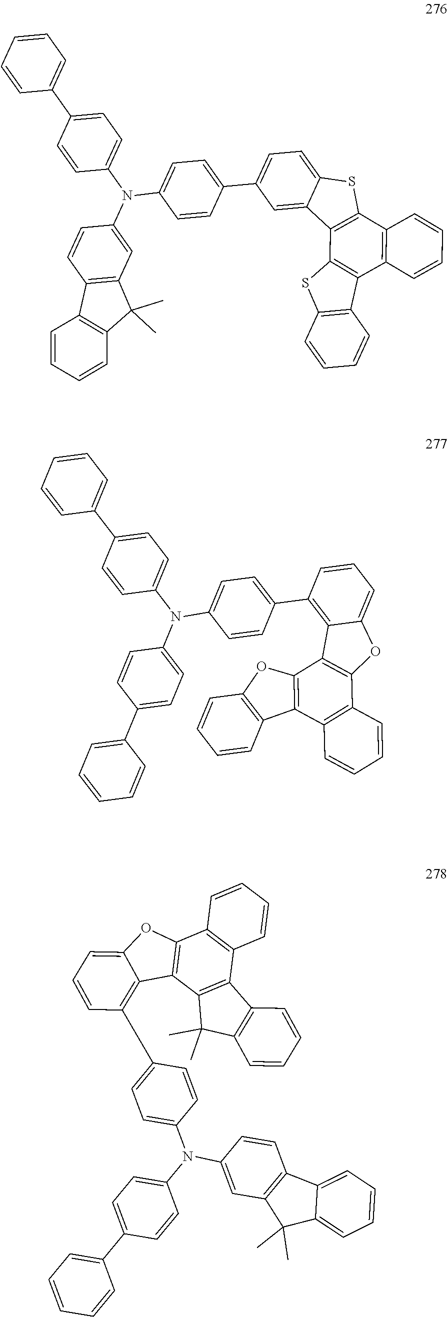 [SMILES: CC1(C)c2ccccc2-c2ccc(N(c3ccc(-c4ccccc4)cc3)c3ccc(-c4ccc5sc6c7ccccc7c7c8ccccc8sc7c6c5c4)cc3)cc21 ; c1ccc(-c2ccc(N(c3ccc(-c4ccccc4)cc3)c3ccc(-c4cccc5oc6c7ccccc7c7c8ccccc8oc7c6c45)cc3)cc2)cc1 ; CC1(C)c2ccccc2-c2ccc(N(c3ccc(-c4ccccc4)cc3)c3ccc(-c4cccc5oc6c7ccccc7c7c(c6c45)C(C)(C)c4ccccc4-7)cc3)cc21]

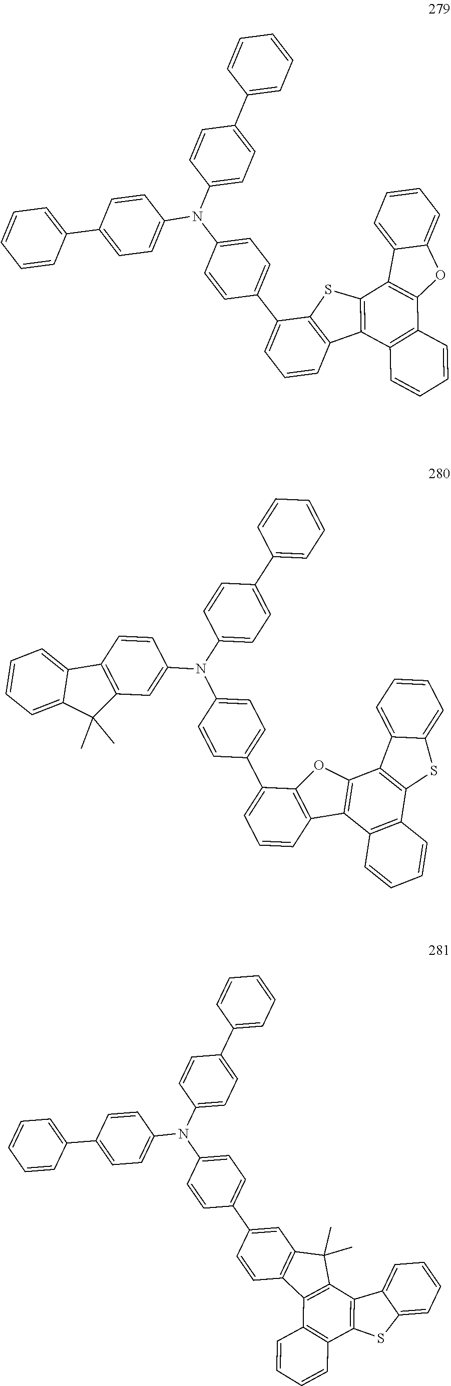 [SMILES: c1ccc(-c2ccc(N(c3ccc(-c4ccccc4)cc3)c3ccc(-c4cccc5c4sc4c5c5ccccc5c5oc6ccccc6c54)cc3)cc2)cc1 ; CC1(C)c2ccccc2-c2ccc(N(c3ccc(-c4ccccc4)cc3)c3ccc(-c4cccc5c4oc4c5c5ccccc5c5sc6ccccc6c45)cc3)cc21 ; CC1(C)c2cc(-c3ccc(N(c4ccc(-c5ccccc5)cc4)c4ccc(-c5ccccc5)cc4)cc3)ccc2-c2c1c1c3ccccc3sc1c1ccccc21]

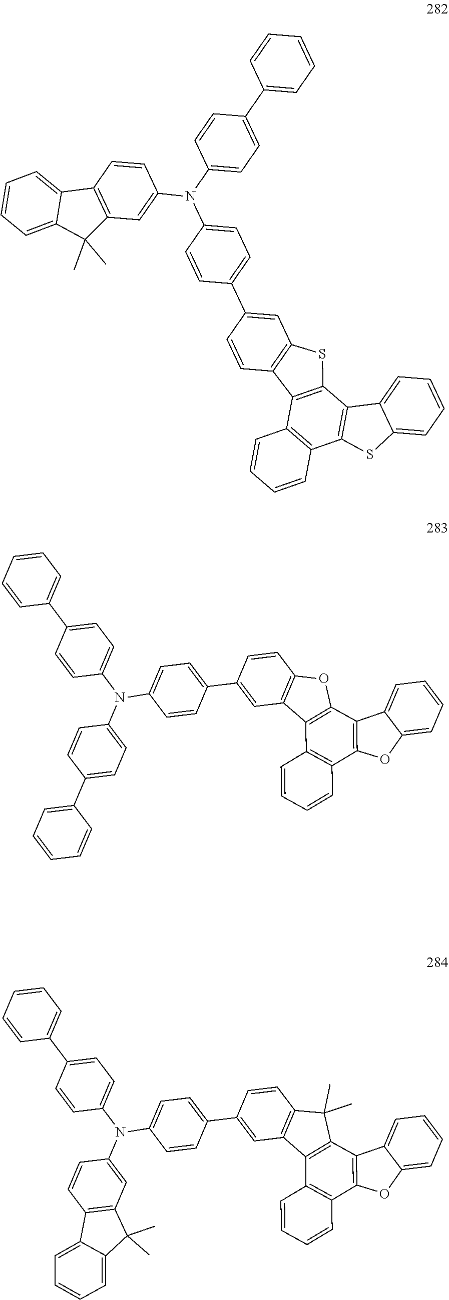 [SMILES: CC1(C)c2ccccc2-c2ccc(N(c3ccc(-c4ccccc4)cc3)c3ccc(-c4ccc5c(c4)sc4c5c5ccccc5c5sc6ccccc6c54)cc3)cc21 ; c1ccc(-c2ccc(N(c3ccc(-c4ccccc4)cc3)c3ccc(-c4ccc5oc6c(c5c4)c4ccccc4c4oc5ccccc5c46)cc3)cc2)cc1 ; CC1(C)c2ccccc2-c2ccc(N(c3ccc(-c4ccccc4)cc3)c3ccc(-c4ccc5c(c4)-c4c(c6c7ccccc7oc6c6ccccc46)C5(C)C)cc3)cc21]

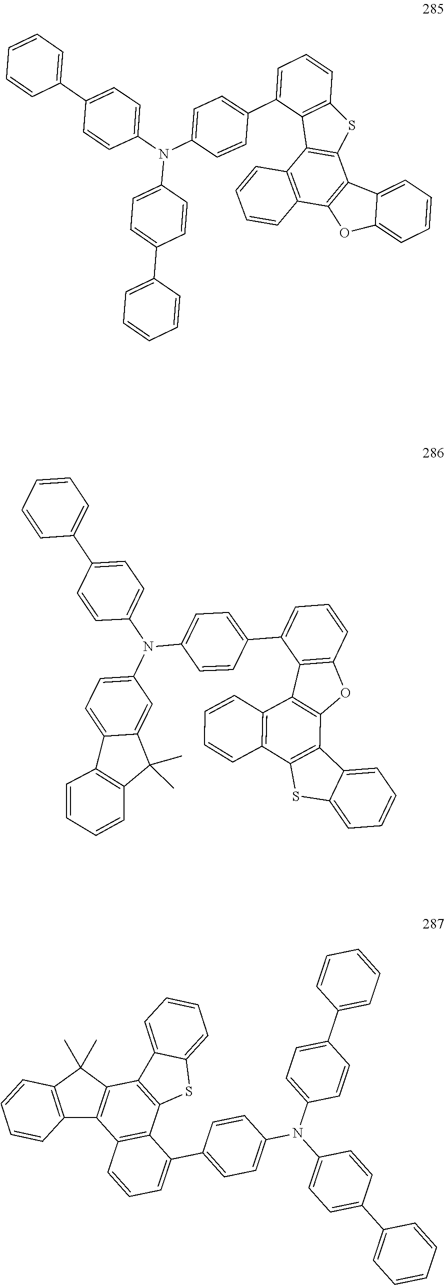 [SMILES: c1ccc(-c2ccc(N(c3ccc(-c4ccccc4)cc3)c3ccc(-c4cccc5sc6c7c8ccccc8oc7c7ccccc7c6c45)cc3)cc2)cc1 ; CC1(C)c2ccccc2-c2ccc(N(c3ccc(-c4ccccc4)cc3)c3ccc(-c4cccc5oc6c7c8ccccc8sc7c7ccccc7c6c45)cc3)cc21 ; CC1(C)c2ccccc2-c2c1c1c3ccccc3sc1c1c(-c3ccc(N(c4ccc(-c5ccccc5)cc4)c4ccc(-c5ccccc5)cc4)cc3)cccc21]

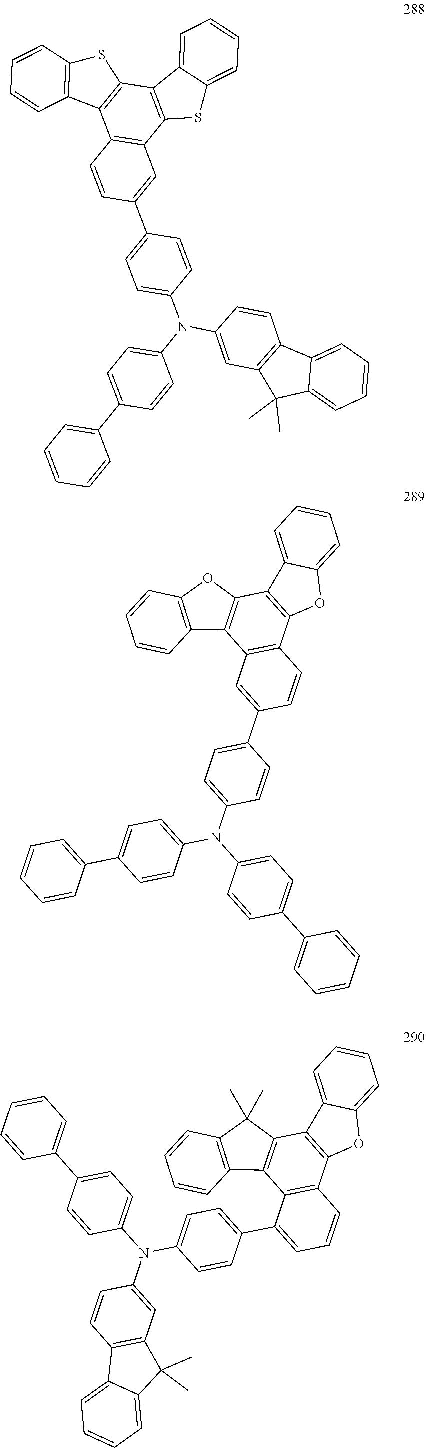 [SMILES: CC1(C)c2ccccc2-c2ccc(N(c3ccc(-c4ccccc4)cc3)c3ccc(-c4ccc5c(c4)c4sc6ccccc6c4c4sc6ccccc6c54)cc3)cc21 ; c1ccc(-c2ccc(N(c3ccc(-c4ccccc4)cc3)c3ccc(-c4ccc5c(c4)c4c6ccccc6oc4c4c6ccccc6oc54)cc3)cc2)cc1 ; CC1(C)c2ccccc2-c2ccc(N(c3ccc(-c4ccccc4)cc3)c3ccc(-c4cccc5c4c4c(c6c7ccccc7oc56)C(C)(C)c5ccccc5-4)cc3)cc21]

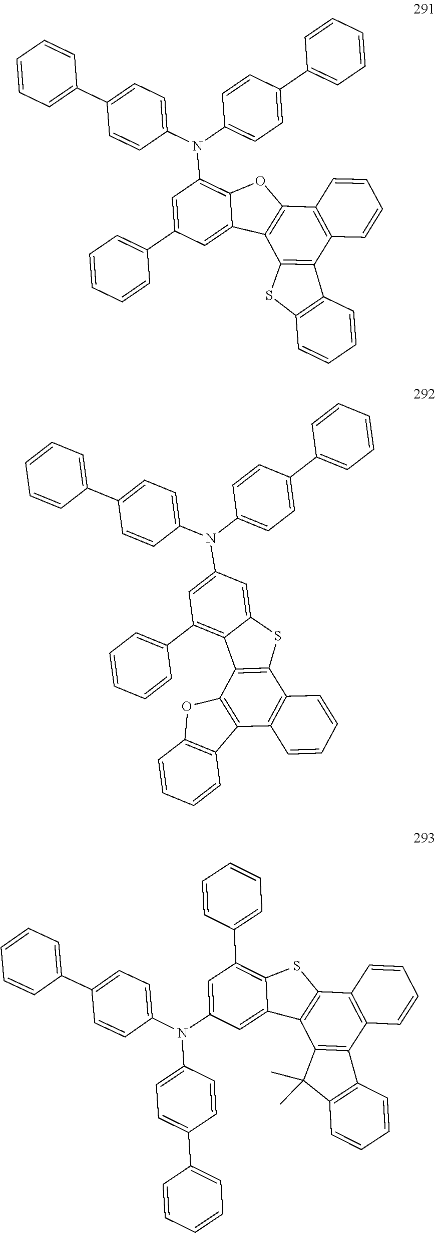

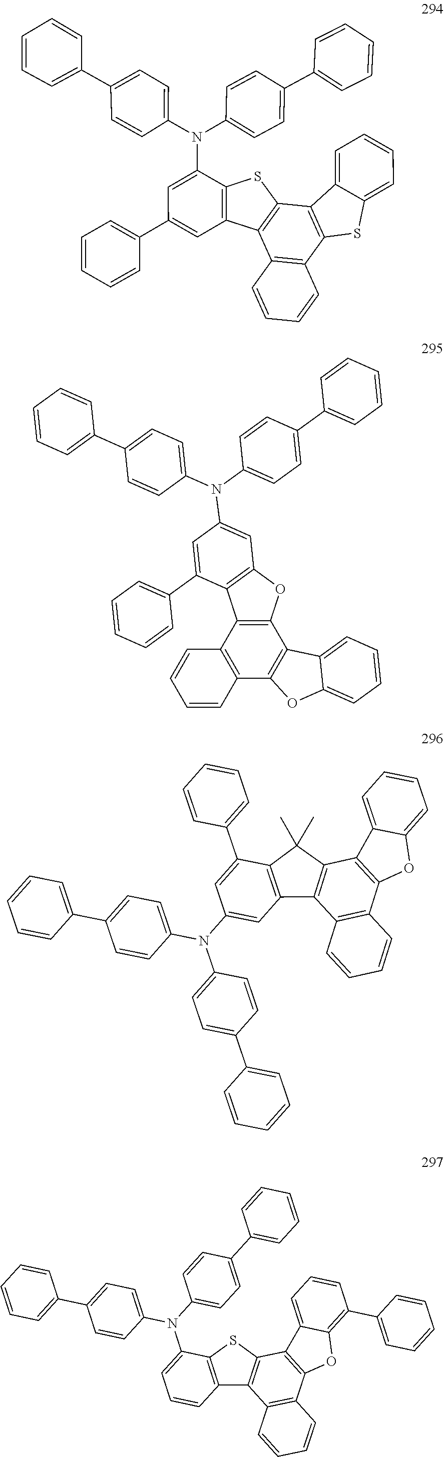 [SMILES: c1ccc(-c2ccc(N(c3ccc(-c4ccccc4)cc3)c3cc(-c4ccccc4)cc4c3sc3c4c4ccccc4c4sc5ccccc5c43)cc2)cc1 ; c1ccc(-c2ccc(N(c3ccc(-c4ccccc4)cc3)c3cc(-c4ccccc4)c4c(c3)oc3c5c6ccccc6oc5c5ccccc5c34)cc2)cc1 ; CC1(C)c2c(-c3ccccc3)cc(N(c3ccc(-c4ccccc4)cc3)c3ccc(-c4ccccc4)cc3)cc2-c2c1c1c3ccccc3oc1c1ccccc21 ; c1ccc(-c2ccc(N(c3ccc(-c4ccccc4)cc3)c3cccc4c3sc3c4c4ccccc4c4oc5c(-c6ccccc6)cccc5c43)cc2)cc1]

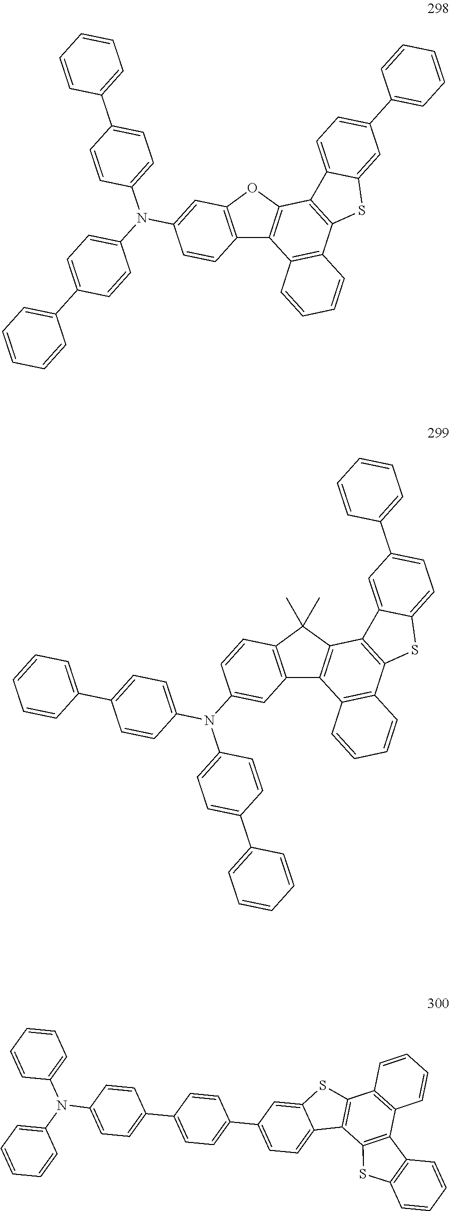 [SMILES: c1ccc(-c2ccc(N(c3ccc(-c4ccccc4)cc3)c3ccc4c(c3)oc3c4c4ccccc4c4sc5cc(-c6ccccc6)ccc5c34)cc2)cc1 ; CC1(C)c2ccc(N(c3ccc(-c4ccccc4)cc3)c3ccc(-c4ccccc4)cc3)cc2-c2c1c1c3cc(-c4ccccc4)ccc3sc1c1ccccc21 ; c1ccc(N(c2ccccc2)c2ccc(-c3ccc(-c4ccc5c(c4)sc4c6ccccc6c6c7ccccc7sc6c54)cc3)cc2)cc1]

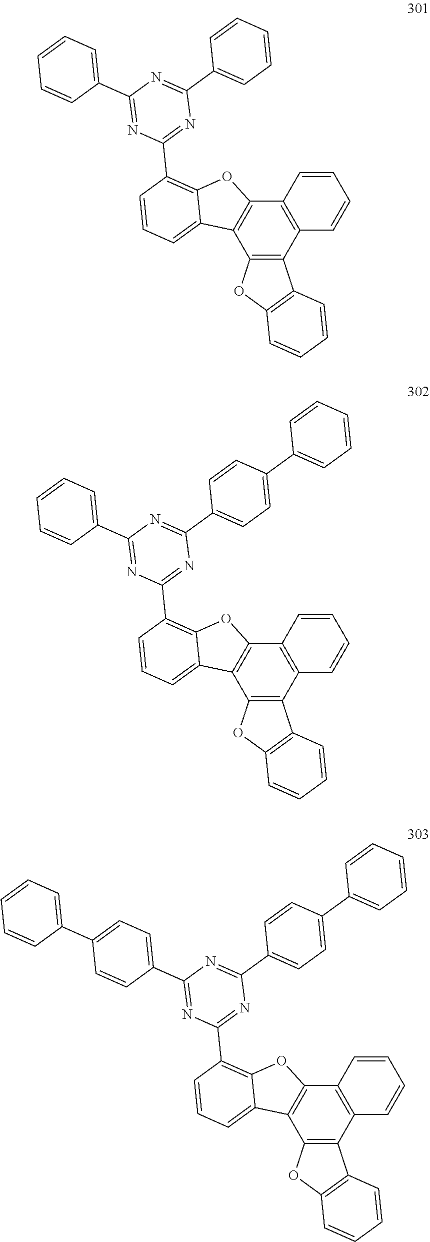 [SMILES: c1ccc(-c2nc(-c3ccccc3)nc(-c3cccc4c3oc3c5ccccc5c5c6ccccc6oc5c43)n2)cc1 ; c1ccc(-c2ccc(-c3nc(-c4ccccc4)nc(-c4cccc5c4oc4c6ccccc6c6c7ccccc7oc6c54)n3)cc2)cc1 ; c1ccc(-c2ccc(-c3nc(-c4ccc(-c5ccccc5)cc4)nc(-c4cccc5c4oc4c6ccccc6c6c7ccccc7oc6c54)n3)cc2)cc1]

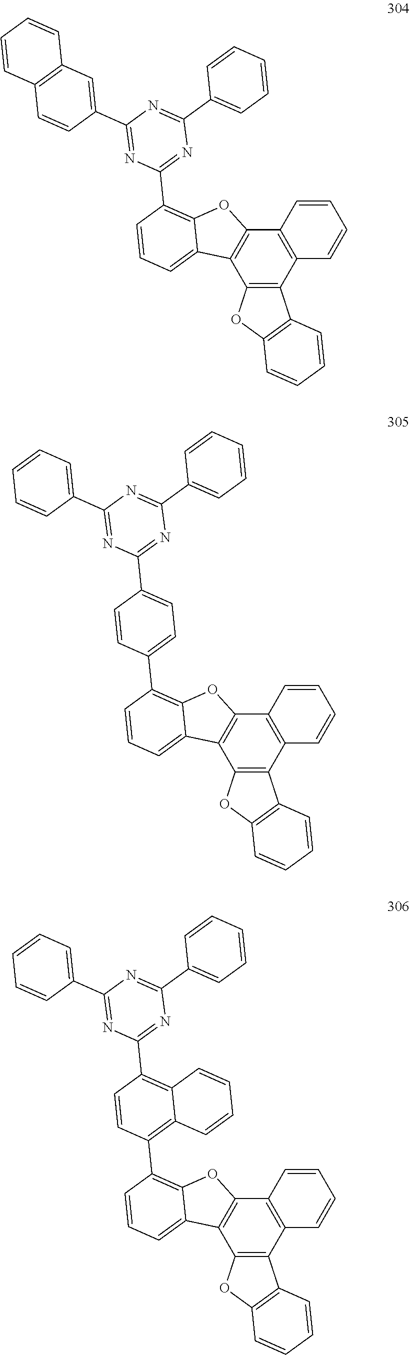 [SMILES: c1ccc(-c2nc(-c3ccc4ccccc4c3)nc(-c3cccc4c3oc3c5ccccc5c5c6ccccc6oc5c43)n2)cc1 ; c1ccc(-c2nc(-c3ccccc3)nc(-c3ccc(-c4cccc5c4oc4c6ccccc6c6c7ccccc7oc6c54)cc3)n2)cc1 ; c1ccc(-c2nc(-c3ccccc3)nc(-c3ccc(-c4cccc5c4oc4c6ccccc6c6c7ccccc7oc6c54)c4ccccc34)n2)cc1]

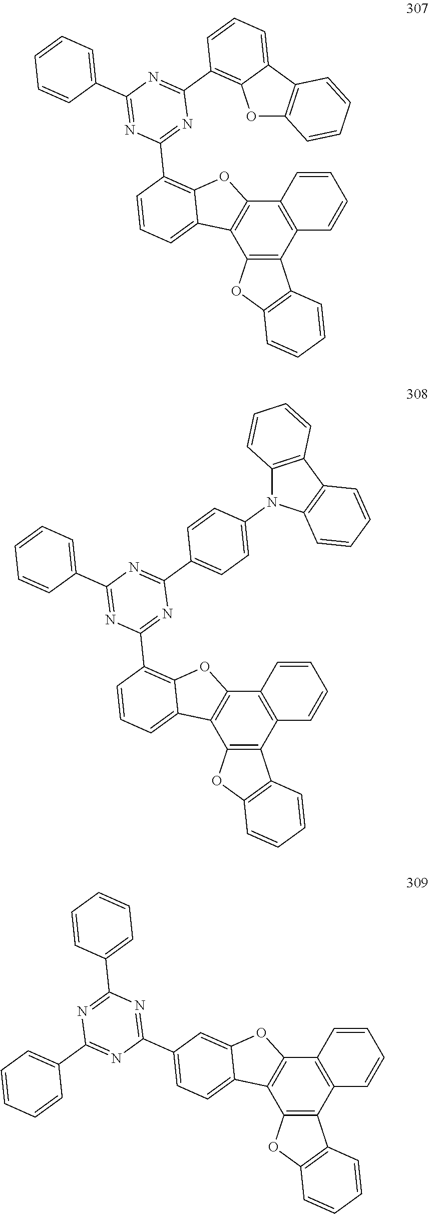 [SMILES: c1ccc(-c2nc(-c3cccc4c3oc3ccccc34)nc(-c3cccc4c3oc3c5ccccc5c5c6ccccc6oc5c43)n2)cc1 ; c1ccc(-c2nc(-c3ccc(-n4c5ccccc5c5ccccc54)cc3)nc(-c3cccc4c3oc3c5ccccc5c5c6ccccc6oc5c43)n2)cc1 ; c1ccc(-c2nc(-c3ccccc3)nc(-c3ccc4c(c3)oc3c5ccccc5c5c6ccccc6oc5c43)n2)cc1]

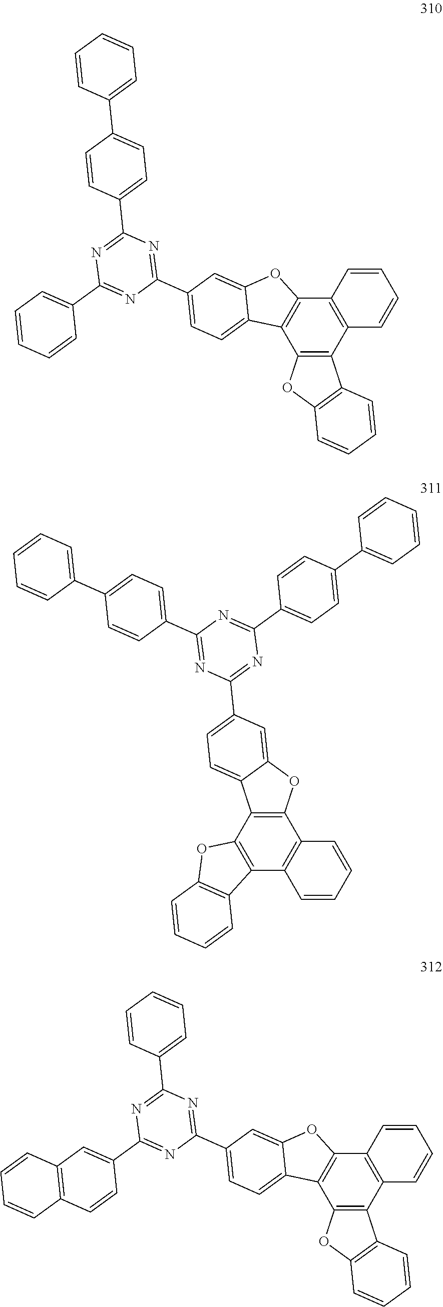 [SMILES: c1ccc(-c2ccc(-c3nc(-c4ccccc4)nc(-c4ccc5c(c4)oc4c6ccccc6c6c7ccccc7oc6c54)n3)cc2)cc1 ; c1ccc(-c2ccc(-c3nc(-c4ccc(-c5ccccc5)cc4)nc(-c4ccc5c(c4)oc4c6ccccc6c6c7ccccc7oc6c54)n3)cc2)cc1 ; c1ccc(-c2nc(-c3ccc4ccccc4c3)nc(-c3ccc4c(c3)oc3c5ccccc5c5c6ccccc6oc5c43)n2)cc1]

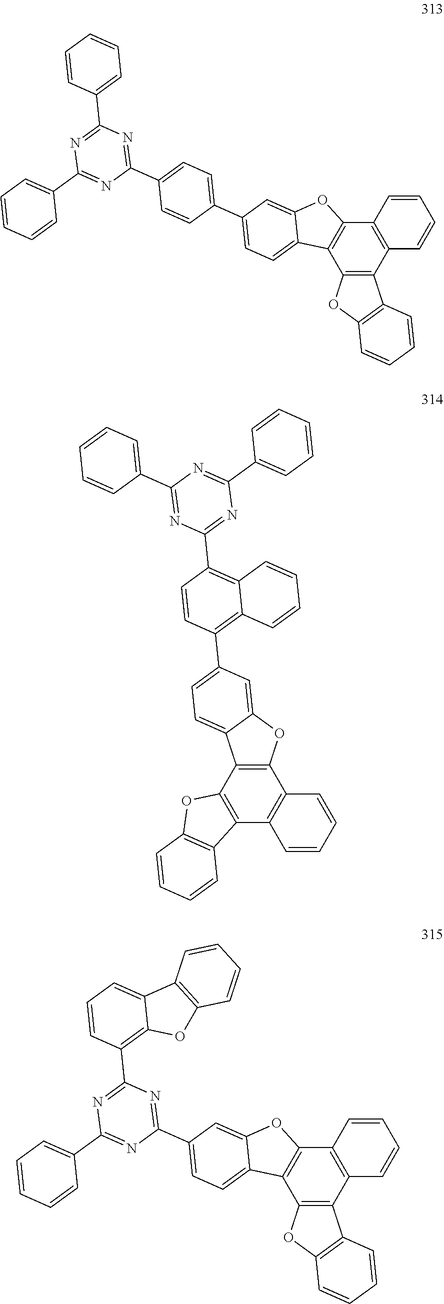 [SMILES: c1ccc(-c2nc(-c3ccccc3)nc(-c3ccc(-c4ccc5c(c4)oc4c6ccccc6c6c7ccccc7oc6c54)cc3)n2)cc1 ; c1ccc(-c2nc(-c3ccccc3)nc(-c3ccc(-c4ccc5c(c4)oc4c6ccccc6c6c7ccccc7oc6c54)c4ccccc34)n2)cc1 ; c1ccc(-c2nc(-c3ccc4c(c3)oc3c5ccccc5c5c6ccccc6oc5c43)nc(-c3cccc4c3oc3ccccc34)n2)cc1]

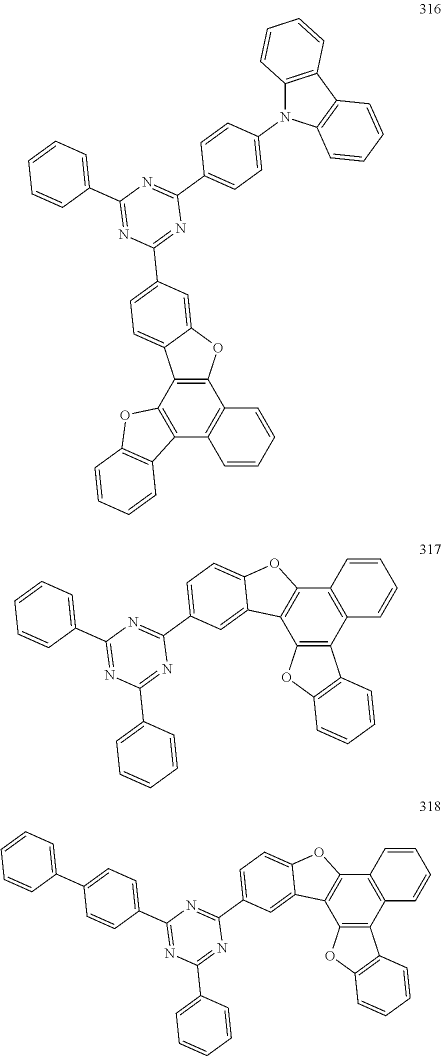 [SMILES: c1ccc(-c2nc(-c3ccc(-n4c5ccccc5c5ccccc54)cc3)nc(-c3ccc4c(c3)oc3c5ccccc5c5c6ccccc6oc5c43)n2)cc1 ; c1ccc(-c2nc(-c3ccccc3)nc(-c3ccc4oc5c6ccccc6c6c7ccccc7oc6c5c4c3)n2)cc1 ; c1ccc(-c2ccc(-c3nc(-c4ccccc4)nc(-c4ccc5oc6c7ccccc7c7c8ccccc8oc7c6c5c4)n3)cc2)cc1]

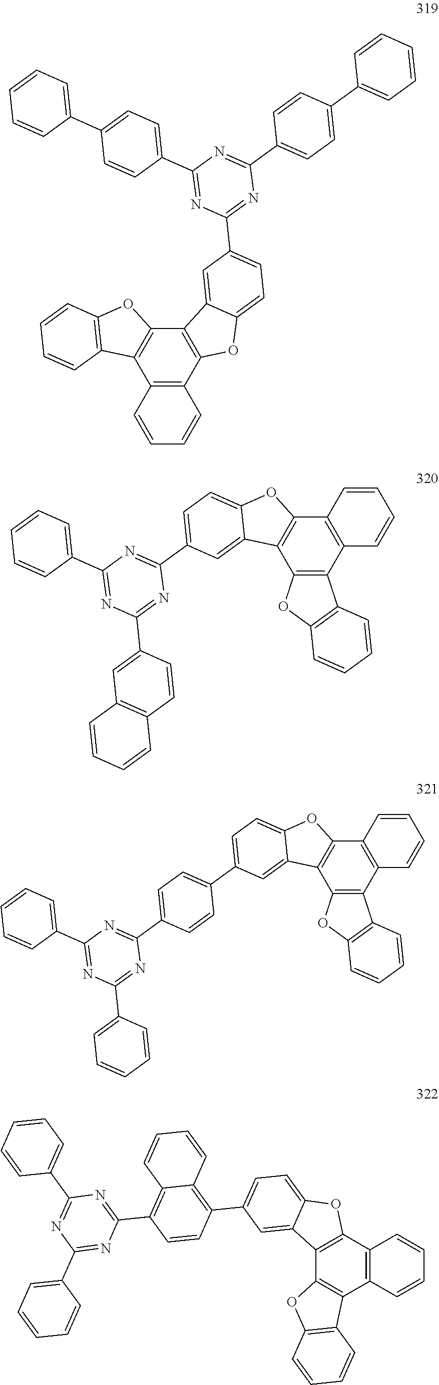 [SMILES: c1ccc(-c2ccc(-c3nc(-c4ccc(-c5ccccc5)cc4)nc(-c4ccc5oc6c7ccccc7c7c8ccccc8oc7c6c5c4)n3)cc2)cc1 ; c1ccc(-c2nc(-c3ccc4ccccc4c3)nc(-c3ccc4oc5c6ccccc6c6c7ccccc7oc6c5c4c3)n2)cc1 ; c1ccc(-c2nc(-c3ccccc3)nc(-c3ccc(-c4ccc5oc6c7ccccc7c7c8ccccc8oc7c6c5c4)cc3)n2)cc1 ; c1ccc(-c2nc(-c3ccccc3)nc(-c3ccc(-c4ccc5oc6c7ccccc7c7c8ccccc8oc7c6c5c4)c4ccccc34)n2)cc1]

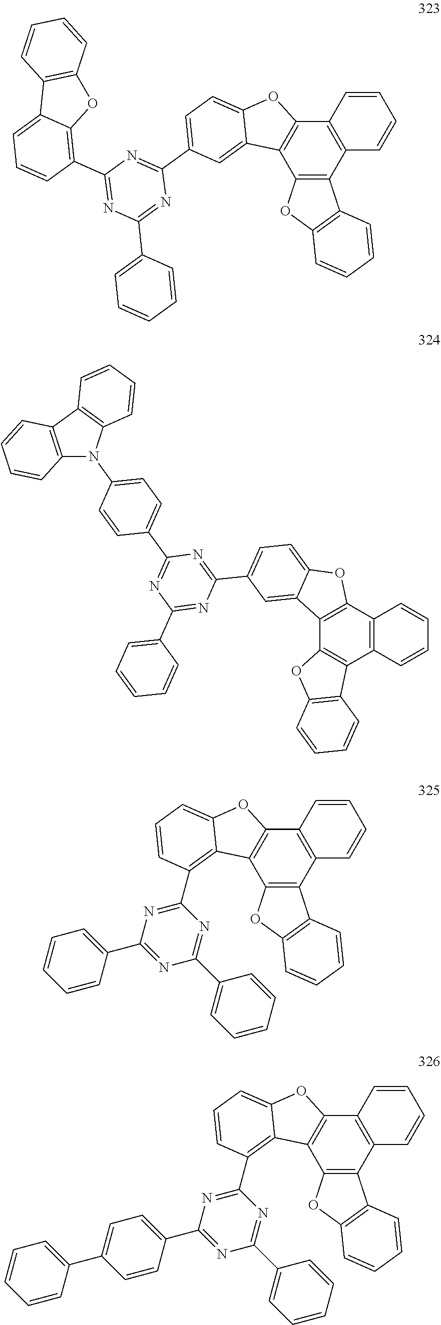 [SMILES: c1ccc(-c2nc(-c3ccc4oc5c6ccccc6c6c7ccccc7oc6c5c4c3)nc(-c3cccc4c3oc3ccccc34)n2)cc1 ; c1ccc(-c2nc(-c3ccc(-n4c5ccccc5c5ccccc54)cc3)nc(-c3ccc4oc5c6ccccc6c6c7ccccc7oc6c5c4c3)n2)cc1 ; c1ccc(-c2nc(-c3ccccc3)nc(-c3cccc4oc5c6ccccc6c6c7ccccc7oc6c5c34)n2)cc1 ; c1ccc(-c2ccc(-c3nc(-c4ccccc4)nc(-c4cccc5oc6c7ccccc7c7c8ccccc8oc7c6c45)n3)cc2)cc1]

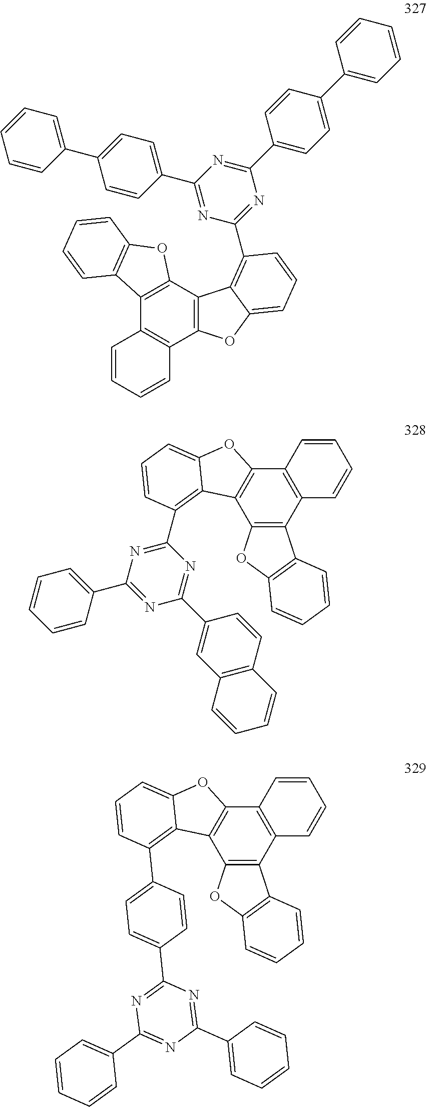 [SMILES: c1ccc(-c2ccc(-c3nc(-c4ccc(-c5ccccc5)cc4)nc(-c4cccc5oc6c7ccccc7c7c8ccccc8oc7c6c45)n3)cc2)cc1 ; c1ccc(-c2nc(-c3ccc4ccccc4c3)nc(-c3cccc4oc5c6ccccc6c6c7ccccc7oc6c5c34)n2)cc1 ; c1ccc(-c2nc(-c3ccccc3)nc(-c3ccc(-c4cccc5oc6c7ccccc7c7c8ccccc8oc7c6c45)cc3)n2)cc1]

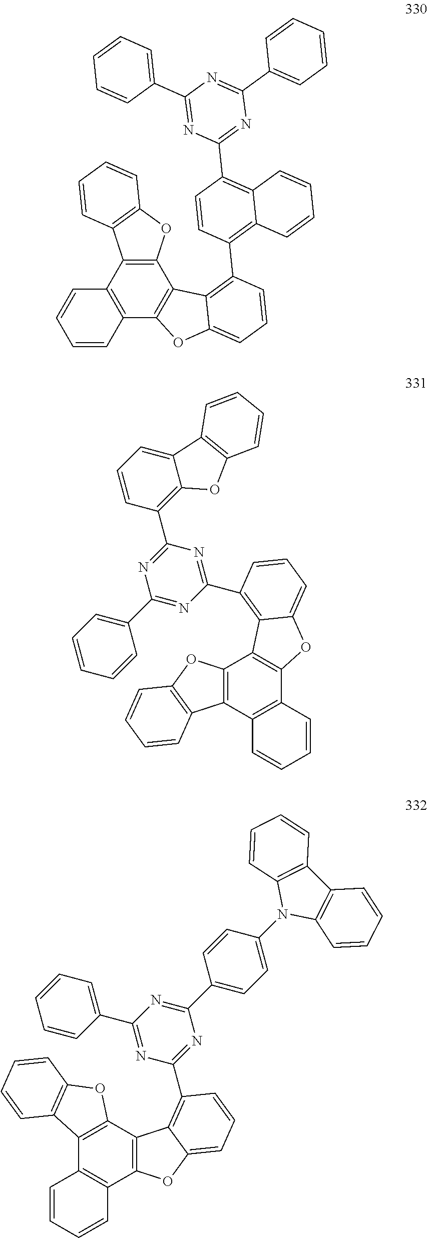

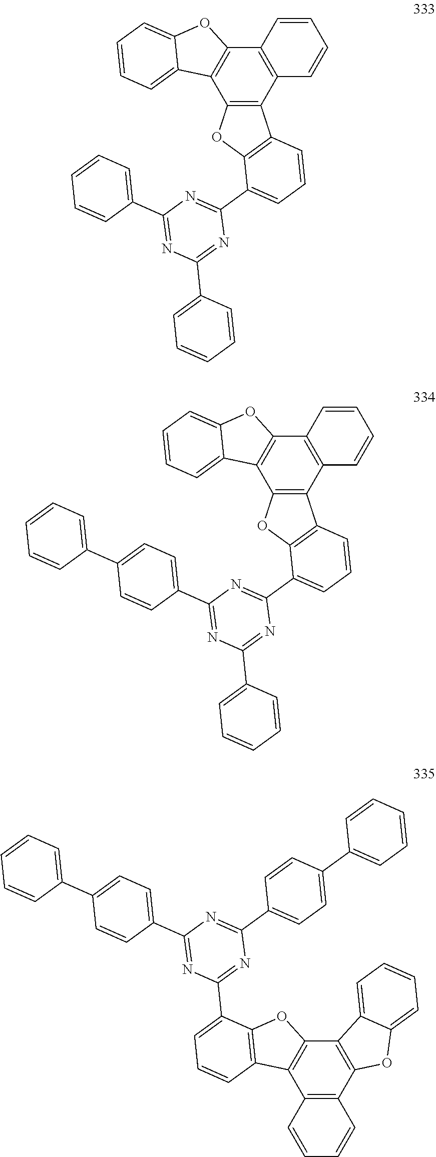 [SMILES: c1ccc(-c2nc(-c3ccccc3)nc(-c3cccc4c3oc3c4c4ccccc4c4oc5ccccc5c43)n2)cc1 ; c1ccc(-c2ccc(-c3nc(-c4ccccc4)nc(-c4cccc5c4oc4c5c5ccccc5c5oc6ccccc6c54)n3)cc2)cc1 ; c1ccc(-c2ccc(-c3nc(-c4ccc(-c5ccccc5)cc4)nc(-c4cccc5c4oc4c5c5ccccc5c5oc6ccccc6c54)n3)cc2)cc1]

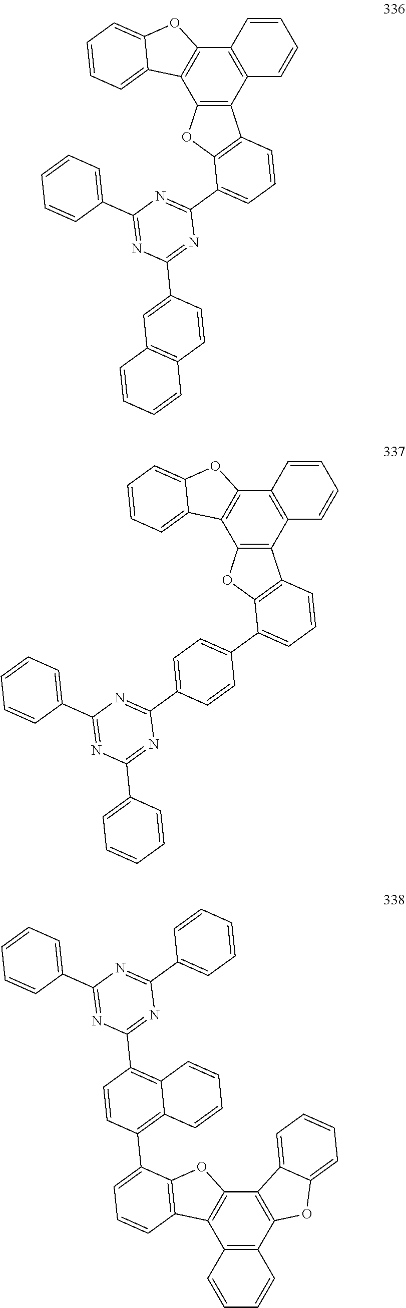 [SMILES: c1ccc(-c2nc(-c3ccc4ccccc4c3)nc(-c3cccc4c3oc3c4c4ccccc4c4oc5ccccc5c43)n2)cc1 ; c1ccc(-c2nc(-c3ccccc3)nc(-c3ccc(-c4cccc5c4oc4c5c5ccccc5c5oc6ccccc6c54)cc3)n2)cc1 ; c1ccc(-c2nc(-c3ccccc3)nc(-c3ccc(-c4cccc5c4oc4c5c5ccccc5c5oc6ccccc6c54)c4ccccc34)n2)cc1]

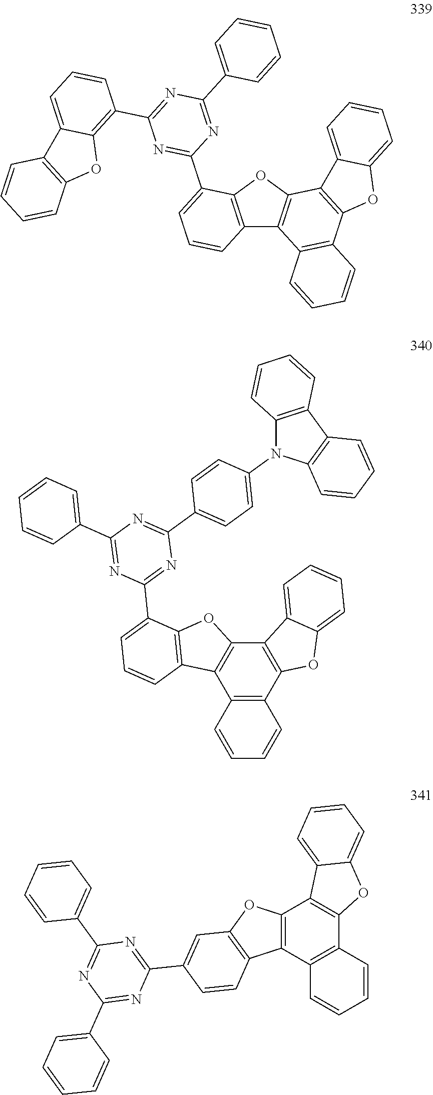 [SMILES: c1ccc(-c2nc(-c3cccc4c3oc3ccccc34)nc(-c3cccc4c3oc3c4c4ccccc4c4oc5ccccc5c43)n2)cc1 ; c1ccc(-c2nc(-c3ccc(-n4c5ccccc5c5ccccc54)cc3)nc(-c3cccc4c3oc3c4c4ccccc4c4oc5ccccc5c43)n2)cc1 ; c1ccc(-c2nc(-c3ccccc3)nc(-c3ccc4c(c3)oc3c4c4ccccc4c4oc5ccccc5c43)n2)cc1]

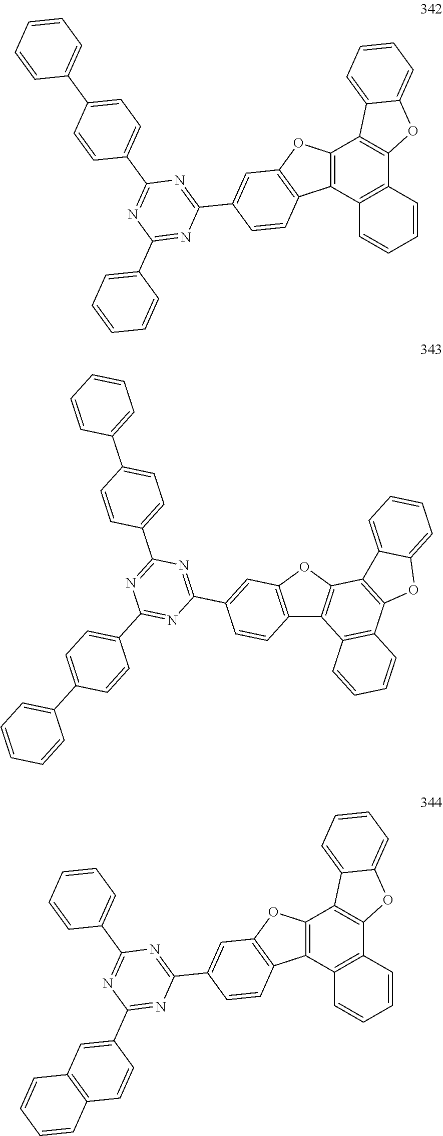

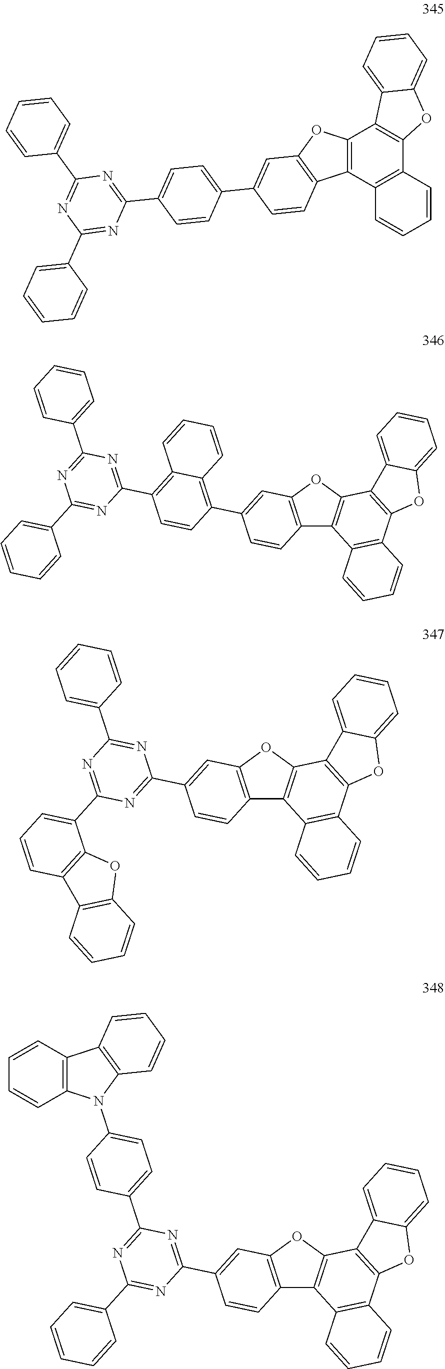

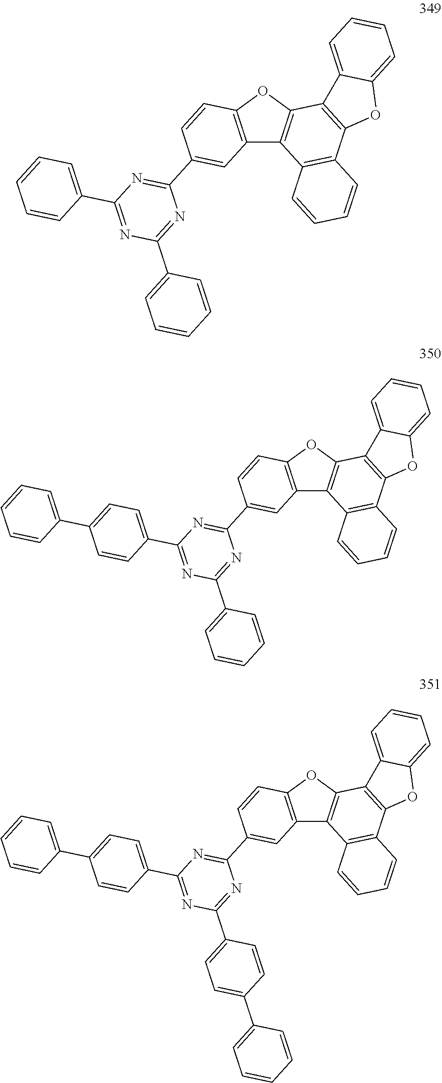 [SMILES: c1ccc(-c2nc(-c3ccccc3)nc(-c3ccc4oc5c(c4c3)c3ccccc3c3oc4ccccc4c35)n2)cc1 ; c1ccc(-c2ccc(-c3nc(-c4ccccc4)nc(-c4ccc5oc6c(c5c4)c4ccccc4c4oc5ccccc5c46)n3)cc2)cc1 ; c1ccc(-c2ccc(-c3nc(-c4ccc(-c5ccccc5)cc4)nc(-c4ccc5oc6c(c5c4)c4ccccc4c4oc5ccccc5c46)n3)cc2)cc1]

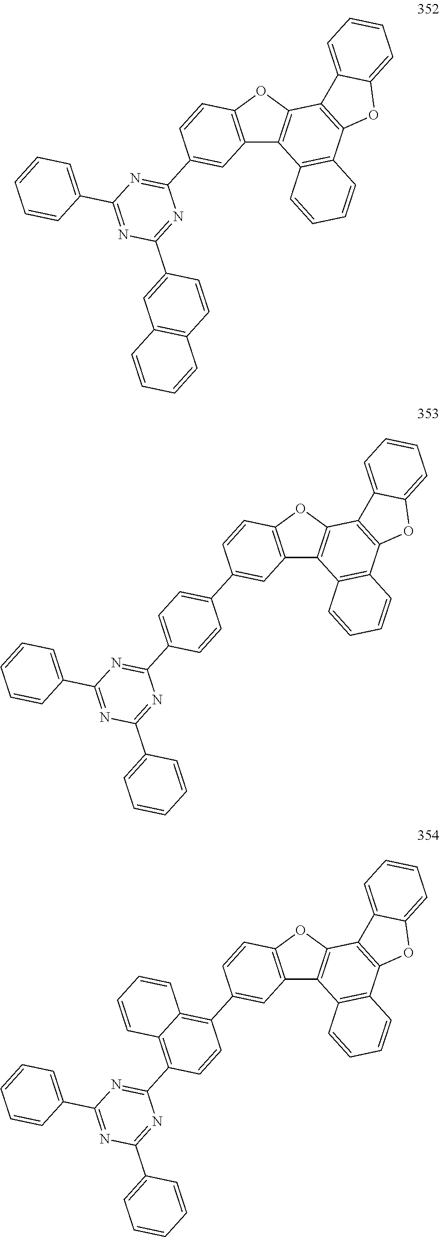 [SMILES: c1ccc(-c2nc(-c3ccc4ccccc4c3)nc(-c3ccc4oc5c(c4c3)c3ccccc3c3oc4ccccc4c35)n2)cc1 ; c1ccc(-c2nc(-c3ccccc3)nc(-c3ccc(-c4ccc5oc6c(c5c4)c4ccccc4c4oc5ccccc5c46)cc3)n2)cc1 ; c1ccc(-c2nc(-c3ccccc3)nc(-c3ccc(-c4ccc5oc6c(c5c4)c4ccccc4c4oc5ccccc5c46)c4ccccc34)n2)cc1]

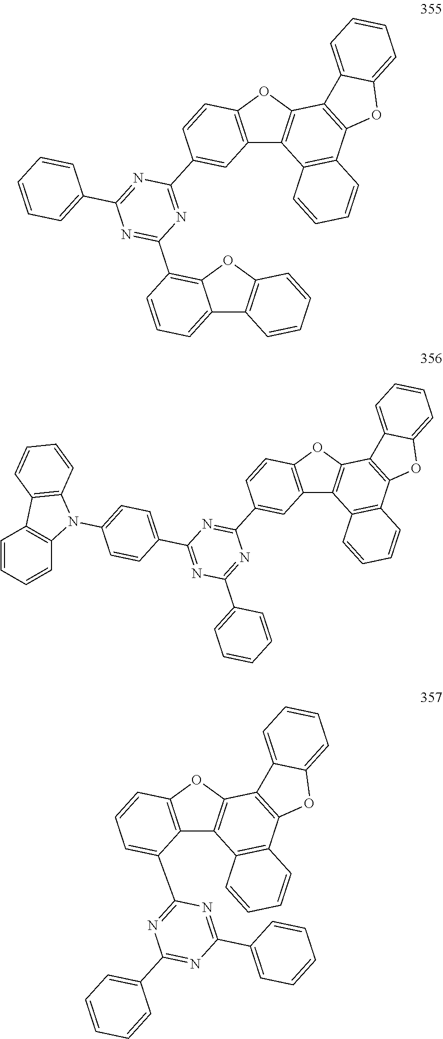 [SMILES: c1ccc(-c2nc(-c3ccc4oc5c(c4c3)c3ccccc3c3oc4ccccc4c35)nc(-c3cccc4c3oc3ccccc34)n2)cc1 ; c1ccc(-c2nc(-c3ccc(-n4c5ccccc5c5ccccc54)cc3)nc(-c3ccc4oc5c(c4c3)c3ccccc3c3oc4ccccc4c35)n2)cc1 ; c1ccc(-c2nc(-c3ccccc3)nc(-c3cccc4oc5c6c7ccccc7oc6c6ccccc6c5c34)n2)cc1]

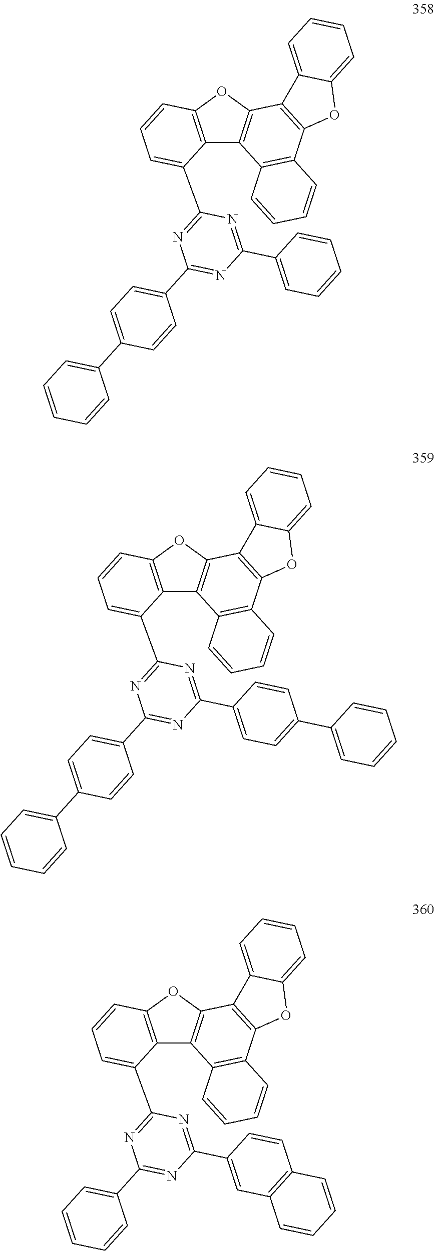 [SMILES: c1ccc(-c2ccc(-c3nc(-c4ccccc4)nc(-c4cccc5oc6c7c8ccccc8oc7c7ccccc7c6c45)n3)cc2)cc1 ; c1ccc(-c2ccc(-c3nc(-c4ccc(-c5ccccc5)cc4)nc(-c4cccc5oc6c7c8ccccc8oc7c7ccccc7c6c45)n3)cc2)cc1 ; c1ccc(-c2nc(-c3ccc4ccccc4c3)nc(-c3cccc4oc5c6c7ccccc7oc6c6ccccc6c5c34)n2)cc1]

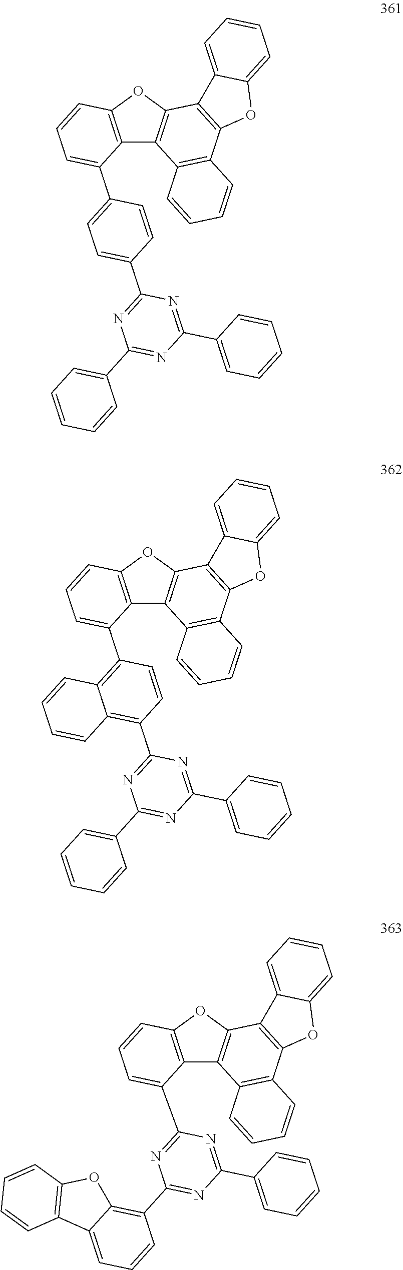 [SMILES: c1ccc(-c2nc(-c3ccccc3)nc(-c3ccc(-c4cccc5oc6c7c8ccccc8oc7c7ccccc7c6c45)cc3)n2)cc1 ; c1ccc(-c2nc(-c3ccccc3)nc(-c3ccc(-c4cccc5oc6c7c8ccccc8oc7c7ccccc7c6c45)c4ccccc34)n2)cc1 ; c1ccc(-c2nc(-c3cccc4c3oc3ccccc34)nc(-c3cccc4oc5c6c7ccccc7oc6c6ccccc6c5c34)n2)cc1]

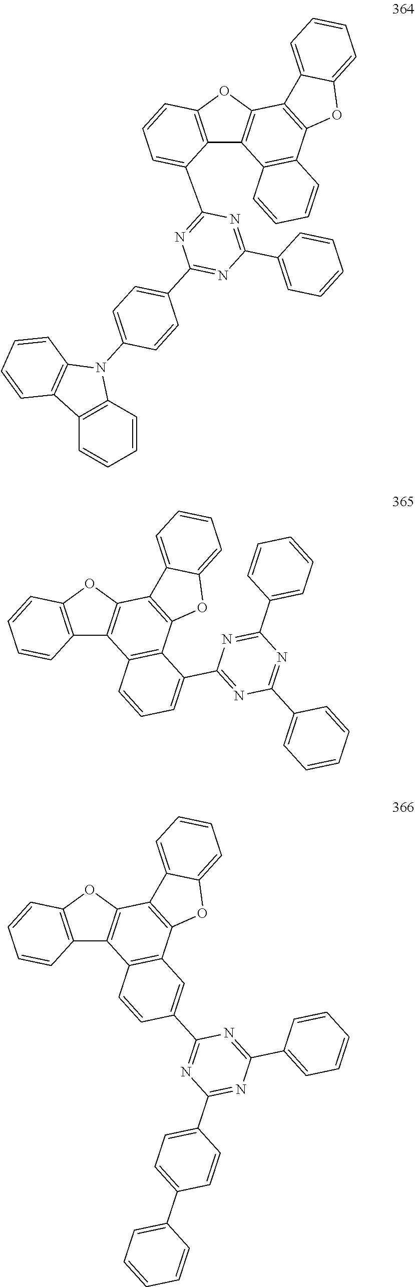 [SMILES: c1ccc(-c2nc(-c3ccc(-n4c5ccccc5c5ccccc54)cc3)nc(-c3cccc4oc5c6c7ccccc7oc6c6ccccc6c5c34)n2)cc1 ; c1ccc(-c2nc(-c3ccccc3)nc(-c3cccc4c3c3oc5ccccc5c3c3oc5ccccc5c43)n2)cc1 ; c1ccc(-c2ccc(-c3nc(-c4ccccc4)nc(-c4ccc5c(c4)c4oc6ccccc6c4c4oc6ccccc6c54)n3)cc2)cc1]

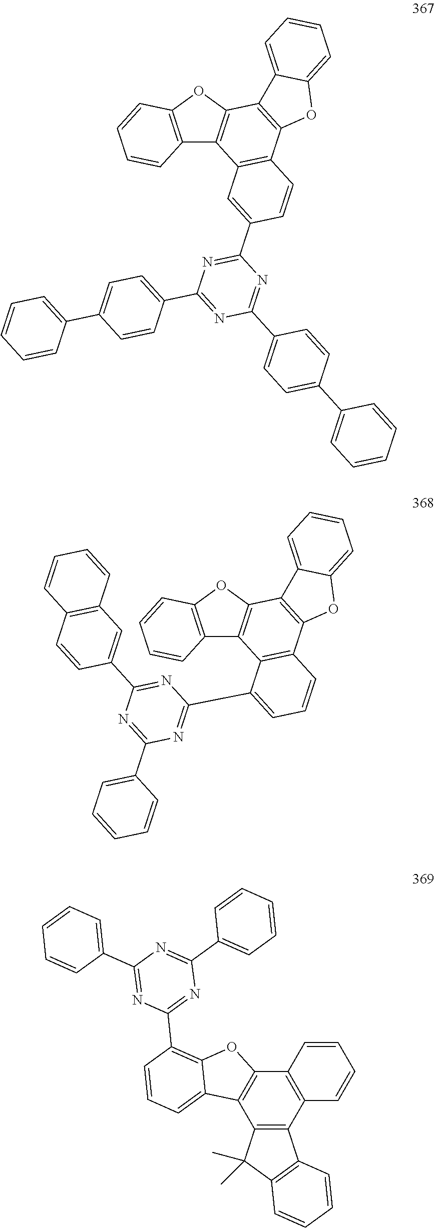 [SMILES: c1ccc(-c2ccc(-c3nc(-c4ccc(-c5ccccc5)cc4)nc(-c4ccc5c(c4)c4c6ccccc6oc4c4c6ccccc6oc54)n3)cc2)cc1 ; c1ccc(-c2nc(-c3ccc4ccccc4c3)nc(-c3cccc4c5oc6ccccc6c5c5oc6ccccc6c5c34)n2)cc1 ; CC1(C)c2ccccc2-c2c1c1c3cccc(-c4nc(-c5ccccc5)nc(-c5ccccc5)n4)c3oc1c1ccccc21]

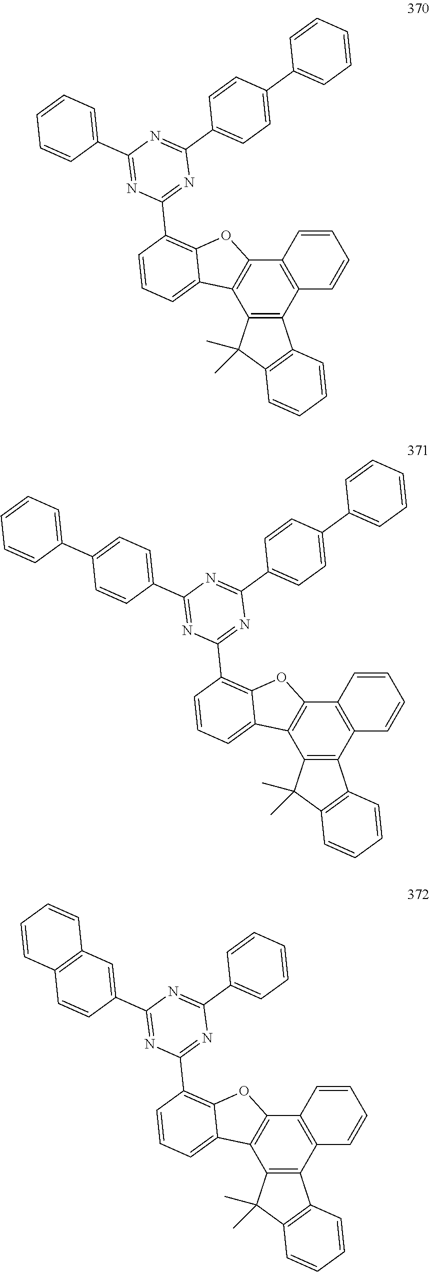 [SMILES: CC1(C)c2ccccc2-c2c1c1c3cccc(-c4nc(-c5ccccc5)nc(-c5ccc(-c6ccccc6)cc5)n4)c3oc1c1ccccc21 ; CC1(C)c2ccccc2-c2c1c1c3cccc(-c4nc(-c5ccc(-c6ccccc6)cc5)nc(-c5ccc(-c6ccccc6)cc5)n4)c3oc1c1ccccc21 ; CC1(C)c2ccccc2-c2c1c1c3cccc(-c4nc(-c5ccccc5)nc(-c5ccc6ccccc6c5)n4)c3oc1c1ccccc21]

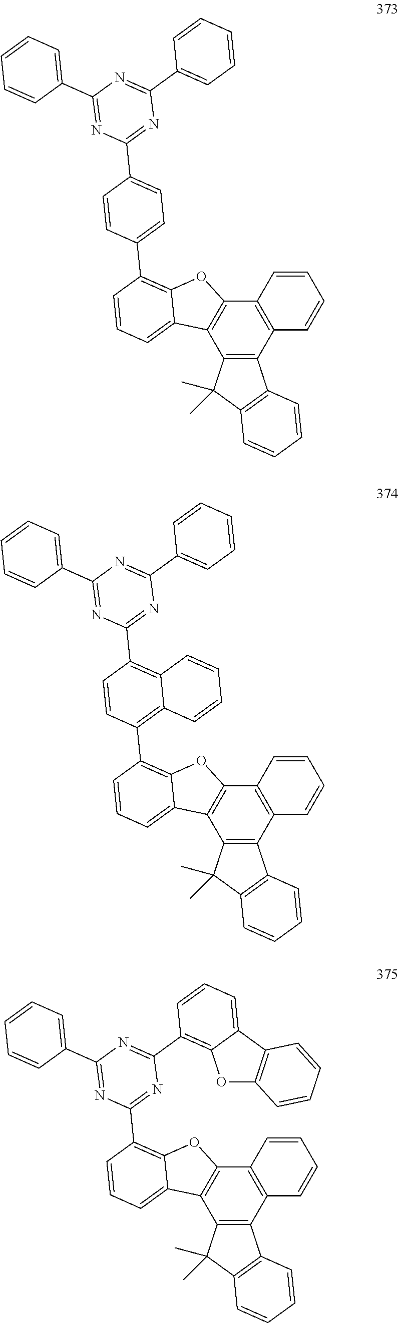 [SMILES: CC1(C)c2ccccc2-c2c1c1c3cccc(-c4ccc(-c5nc(-c6ccccc6)nc(-c6ccccc6)n5)cc4)c3oc1c1ccccc21 ; CC1(C)c2ccccc2-c2c1c1c3cccc(-c4ccc(-c5nc(-c6ccccc6)nc(-c6ccccc6)n5)c5ccccc45)c3oc1c1ccccc21 ; CC1(C)c2ccccc2-c2c1c1c3cccc(-c4nc(-c5ccccc5)nc(-c5cccc6c5oc5ccccc56)n4)c3oc1c1ccccc21]

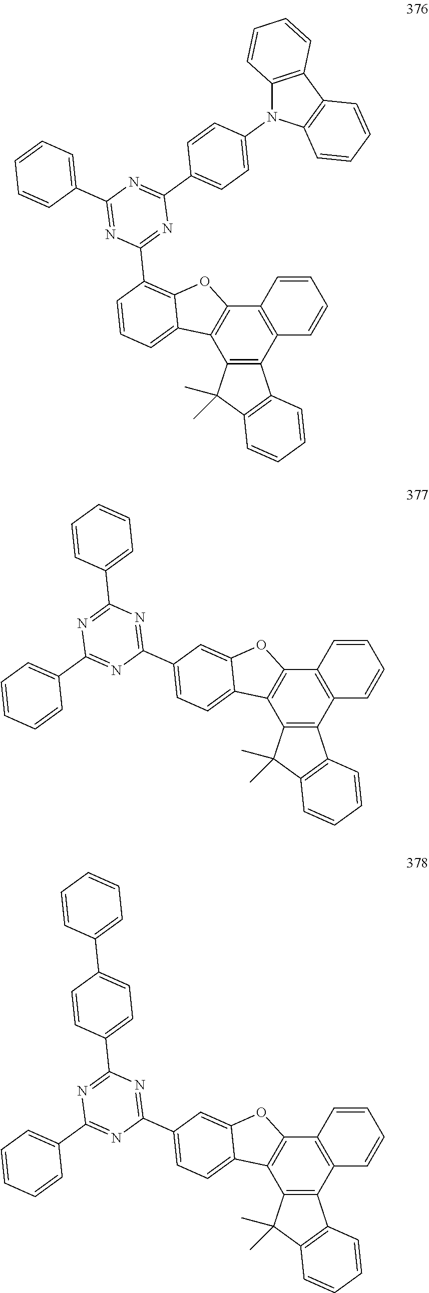 [SMILES: CC1(C)c2ccccc2-c2c1c1c3cccc(-c4nc(-c5ccccc5)nc(-c5ccc(-n6c7ccccc7c7ccccc76)cc5)n4)c3oc1c1ccccc21 ; CC1(C)c2ccccc2-c2c1c1c3ccc(-c4nc(-c5ccccc5)nc(-c5ccccc5)n4)cc3oc1c1ccccc21 ; CC1(C)c2ccccc2-c2c1c1c3ccc(-c4nc(-c5ccccc5)nc(-c5ccc(-c6ccccc6)cc5)n4)cc3oc1c1ccccc21]

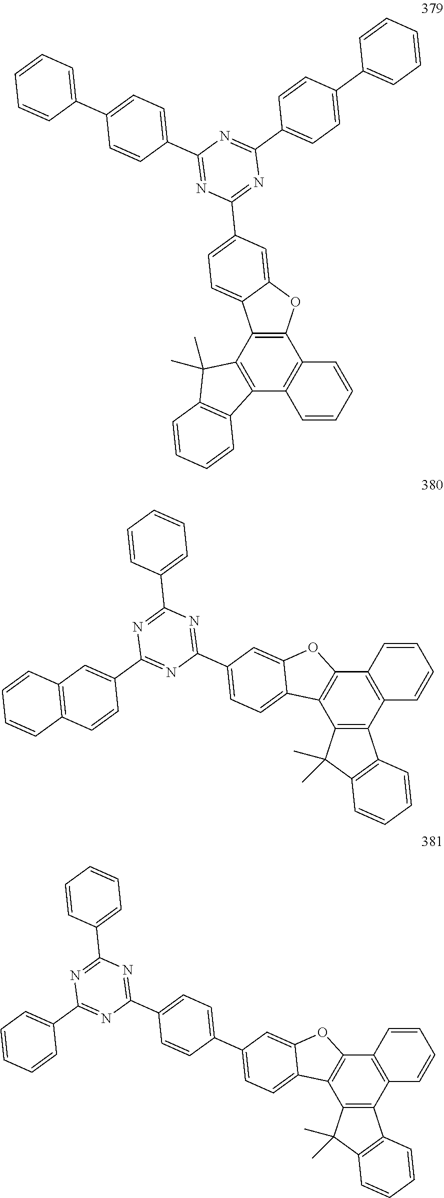 [SMILES: CC1(C)c2ccccc2-c2c1c1c3ccc(-c4nc(-c5ccc(-c6ccccc6)cc5)nc(-c5ccc(-c6ccccc6)cc5)n4)cc3oc1c1ccccc21 ; CC1(C)c2ccccc2-c2c1c1c3ccc(-c4nc(-c5ccccc5)nc(-c5ccc6ccccc6c5)n4)cc3oc1c1ccccc21 ; CC1(C)c2ccccc2-c2c1c1c3ccc(-c4ccc(-c5nc(-c6ccccc6)nc(-c6ccccc6)n5)cc4)cc3oc1c1ccccc21]

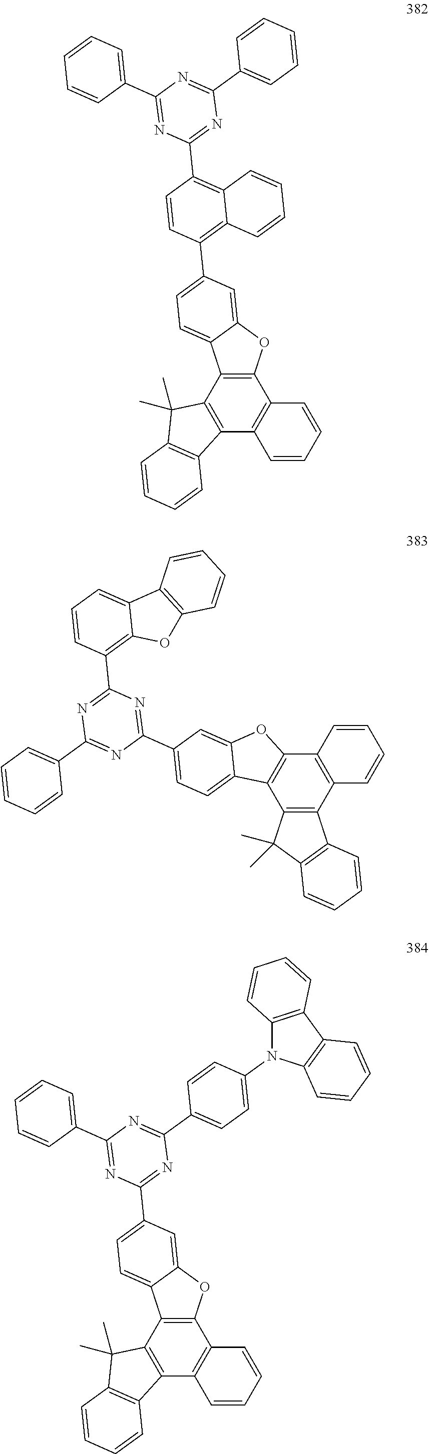 [SMILES: CC1(C)c2ccccc2-c2c1c1c3ccc(-c4ccc(-c5nc(-c6ccccc6)nc(-c6ccccc6)n5)c5ccccc45)cc3oc1c1ccccc21 ; CC1(C)c2ccccc2-c2c1c1c3ccc(-c4nc(-c5ccccc5)nc(-c5cccc6c5oc5ccccc56)n4)cc3oc1c1ccccc21 ; CC1(C)c2ccccc2-c2c1c1c3ccc(-c4nc(-c5ccccc5)nc(-c5ccc(-n6c7ccccc7c7ccccc76)cc5)n4)cc3oc1c1ccccc21]

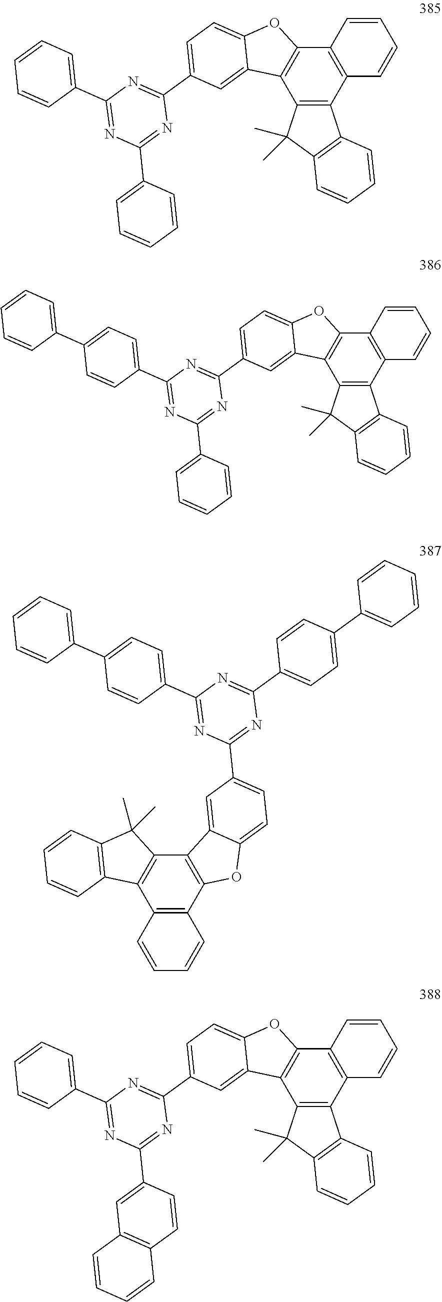 [SMILES: CC1(C)c2ccccc2-c2c1c1c3cc(-c4nc(-c5ccccc5)nc(-c5ccccc5)n4)ccc3oc1c1ccccc21 ; CC1(C)c2ccccc2-c2c1c1c3cc(-c4nc(-c5ccccc5)nc(-c5ccc(-c6ccccc6)cc5)n4)ccc3oc1c1ccccc21 ; CC1(C)c2ccccc2-c2c1c1c3cc(-c4nc(-c5ccc(-c6ccccc6)cc5)nc(-c5ccc(-c6ccccc6)cc5)n4)ccc3oc1c1ccccc21 ; CC1(C)c2ccccc2-c2c1c1c3cc(-c4nc(-c5ccccc5)nc(-c5ccc6ccccc6c5)n4)ccc3oc1c1ccccc21]

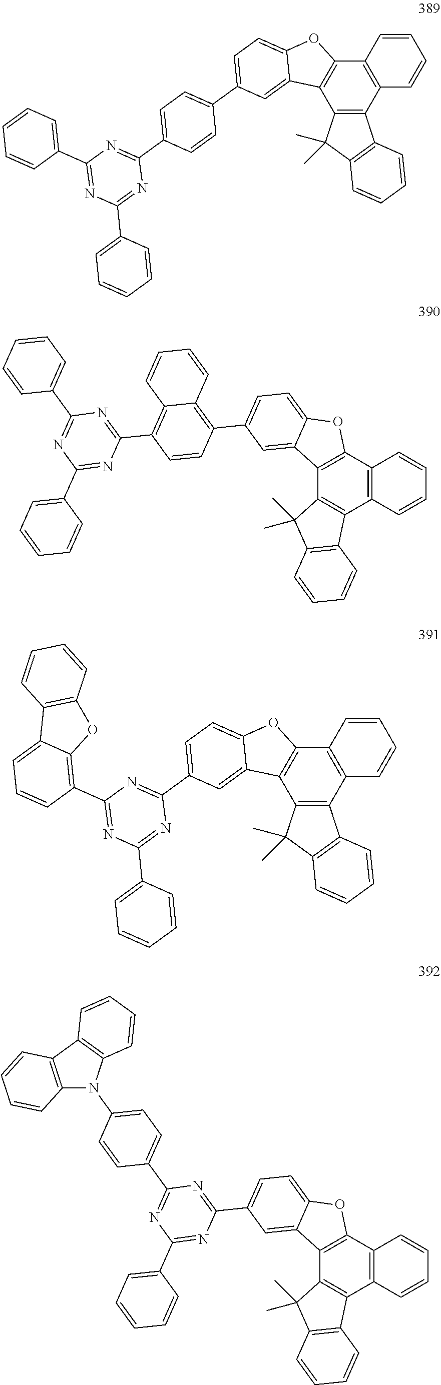 [SMILES: CC1(C)c2ccccc2-c2c1c1c3cc(-c4ccc(-c5nc(-c6ccccc6)nc(-c6ccccc6)n5)cc4)ccc3oc1c1ccccc21 ; CC1(C)c2ccccc2-c2c1c1c3cc(-c4ccc(-c5nc(-c6ccccc6)nc(-c6ccccc6)n5)c5ccccc45)ccc3oc1c1ccccc21 ; CC1(C)c2ccccc2-c2c1c1c3cc(-c4nc(-c5ccccc5)nc(-c5cccc6c5oc5ccccc56)n4)ccc3oc1c1ccccc21 ; CC1(C)c2ccccc2-c2c1c1c3cc(-c4nc(-c5ccccc5)nc(-c5ccc(-n6c7ccccc7c7ccccc76)cc5)n4)ccc3oc1c1ccccc21]

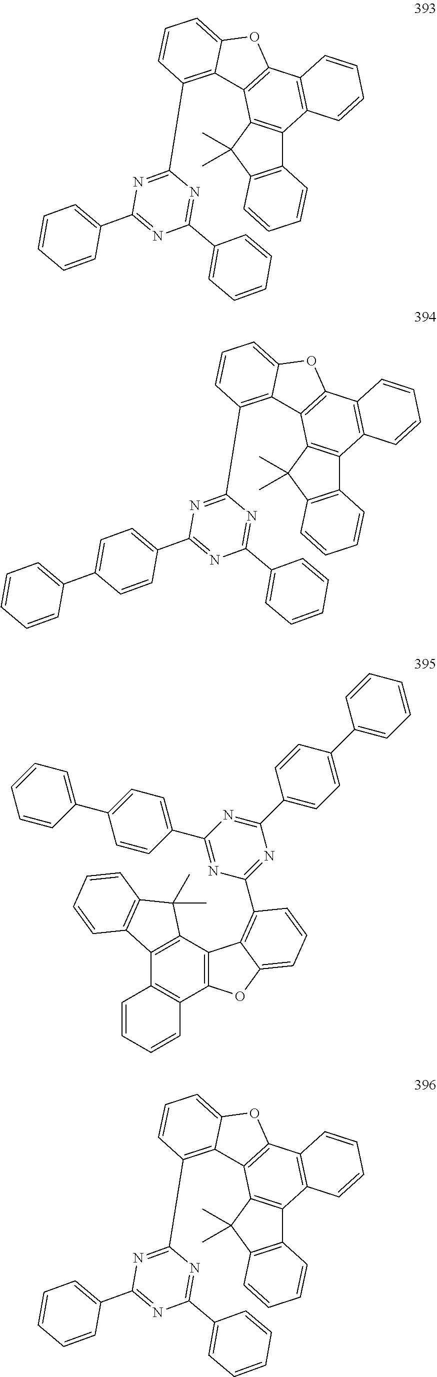

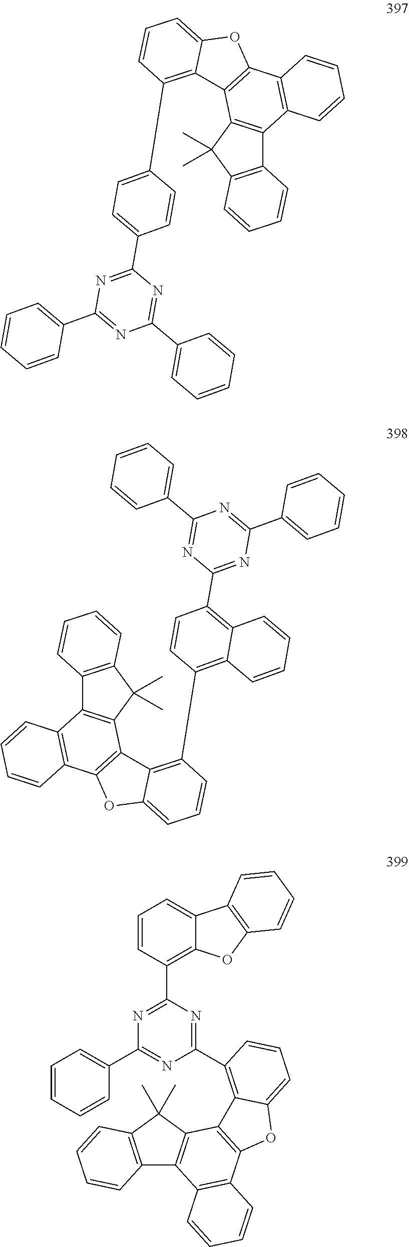 [SMILES: CC1(C)c2ccccc2-c2c1c1c(oc3cccc(-c4ccc(-c5nc(-c6ccccc6)nc(-c6ccccc6)n5)cc4)c31)c1ccccc21 ; CC1(C)c2ccccc2-c2c1c1c(oc3cccc(-c4ccc(-c5nc(-c6ccccc6)nc(-c6ccccc6)n5)c5ccccc45)c31)c1ccccc21 ; CC1(C)c2ccccc2-c2c1c1c(oc3cccc(-c4nc(-c5ccccc5)nc(-c5cccc6c5oc5ccccc56)n4)c31)c1ccccc21]

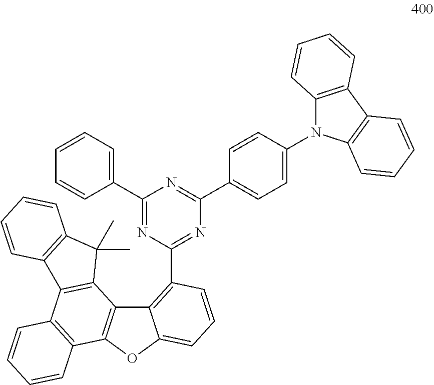 [SMILES: CC1(C)c2ccccc2-c2c1c1c(oc3cccc(-c4nc(-c5ccccc5)nc(-c5ccc(-n6c7ccccc7c7ccccc76)cc5)n4)c31)c1ccccc21]

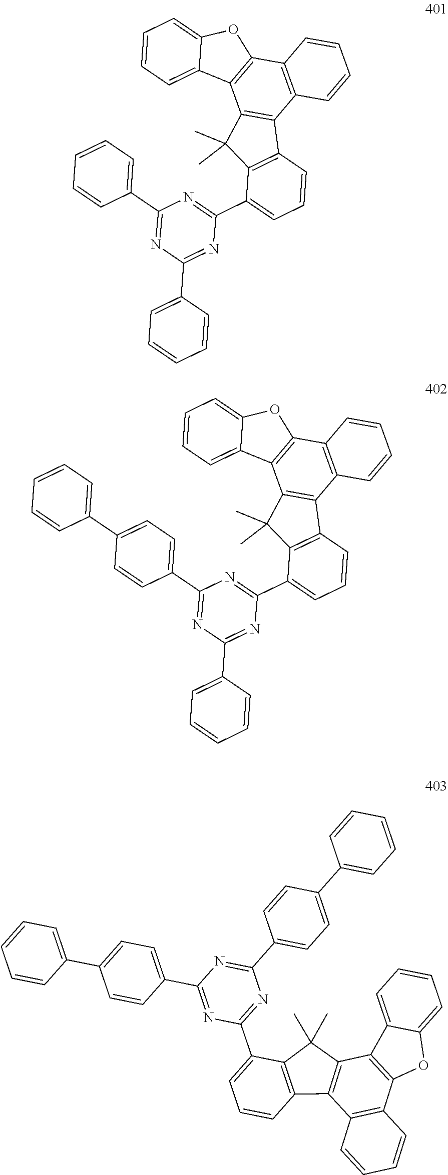 [SMILES: CC1(C)c2c(-c3nc(-c4ccccc4)nc(-c4ccccc4)n3)cccc2-c2c1c1c3ccccc3oc1c1ccccc21 ; CC1(C)c2c(-c3nc(-c4ccccc4)nc(-c4ccc(-c5ccccc5)cc4)n3)cccc2-c2c1c1c3ccccc3oc1c1ccccc21 ; CC1(C)c2c(-c3nc(-c4ccc(-c5ccccc5)cc4)nc(-c4ccc(-c5ccccc5)cc4)n3)cccc2-c2c1c1c3ccccc3oc1c1ccccc21]

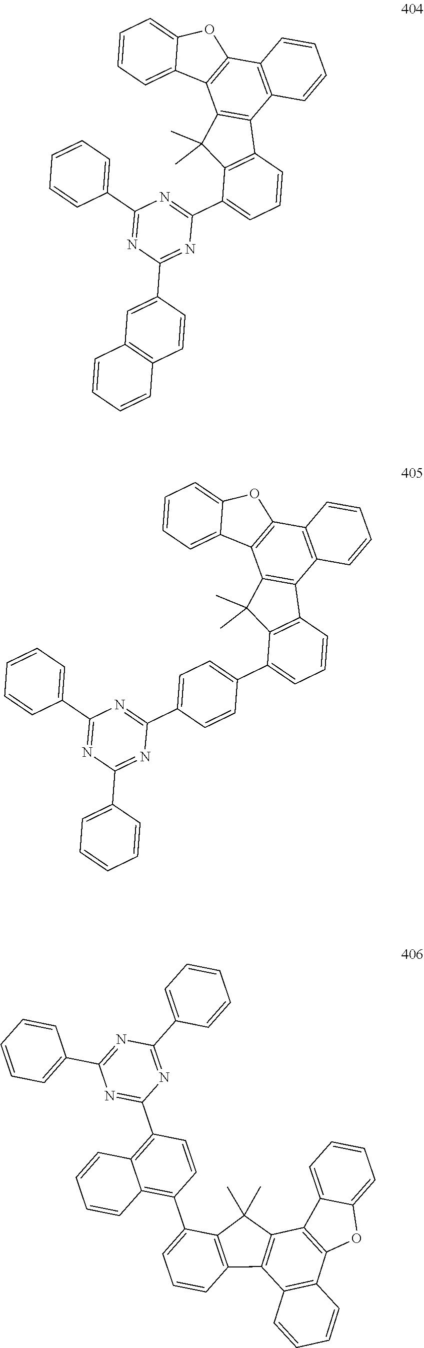 [SMILES: CC1(C)c2c(-c3nc(-c4ccccc4)nc(-c4ccc5ccccc5c4)n3)cccc2-c2c1c1c3ccccc3oc1c1ccccc21 ; CC1(C)c2c(-c3ccc(-c4nc(-c5ccccc5)nc(-c5ccccc5)n4)cc3)cccc2-c2c1c1c3ccccc3oc1c1ccccc21 ; CC1(C)c2c(-c3ccc(-c4nc(-c5ccccc5)nc(-c5ccccc5)n4)c4ccccc34)cccc2-c2c1c1c3ccccc3oc1c1ccccc21]

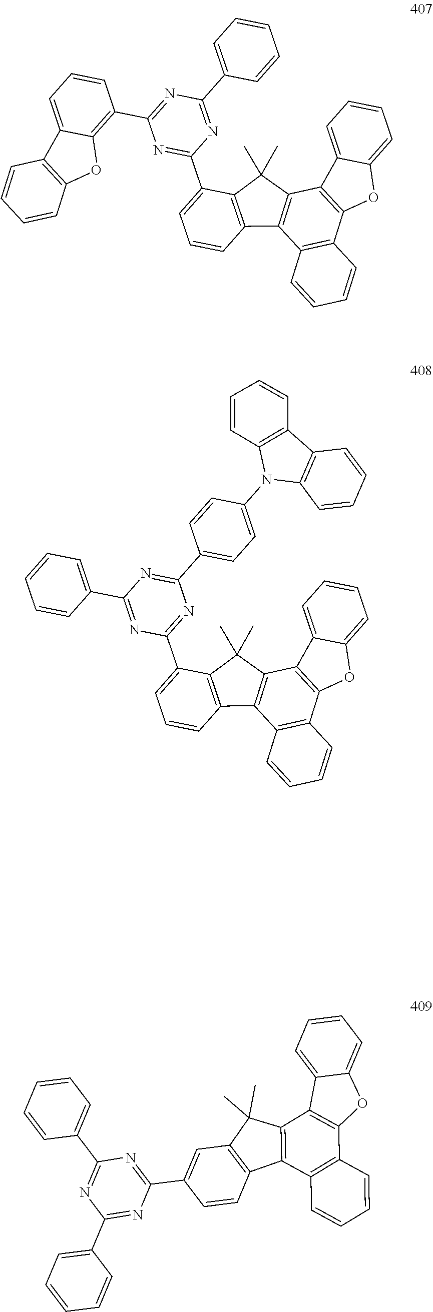 [SMILES: CC1(C)c2c(-c3nc(-c4ccccc4)nc(-c4cccc5c4oc4ccccc45)n3)cccc2-c2c1c1c3ccccc3oc1c1ccccc21 ; CC1(C)c2c(-c3nc(-c4ccccc4)nc(-c4ccc(-n5c6ccccc6c6ccccc65)cc4)n3)cccc2-c2c1c1c3ccccc3oc1c1ccccc21 ; CC1(C)c2cc(-c3nc(-c4ccccc4)nc(-c4ccccc4)n3)ccc2-c2c1c1c3ccccc3oc1c1ccccc21]

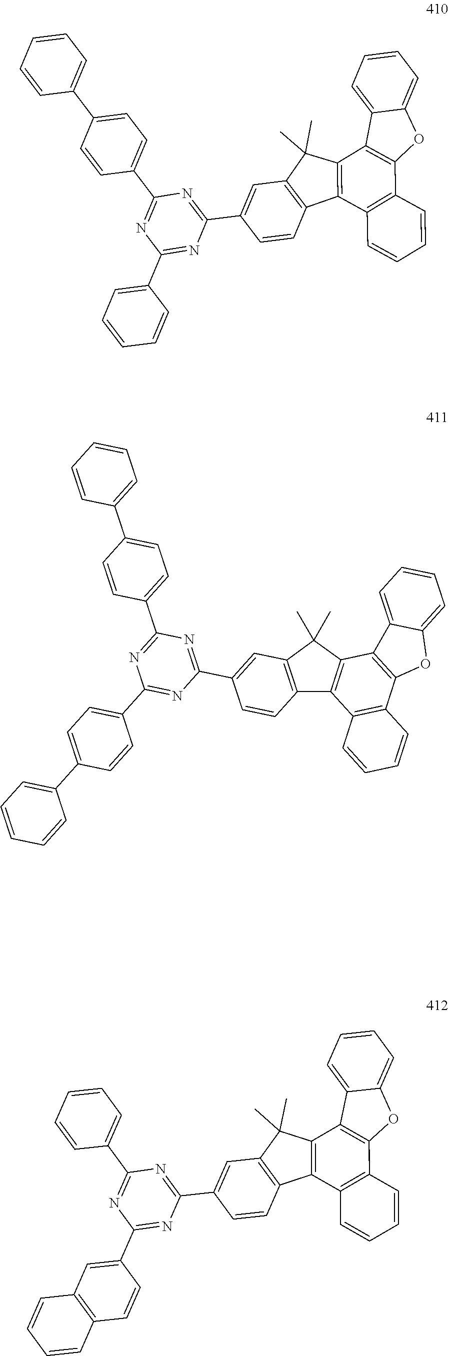 [SMILES: CC1(C)c2cc(-c3nc(-c4ccccc4)nc(-c4ccc(-c5ccccc5)cc4)n3)ccc2-c2c1c1c3ccccc3oc1c1ccccc21 ; CC1(C)c2cc(-c3nc(-c4ccc(-c5ccccc5)cc4)nc(-c4ccc(-c5ccccc5)cc4)n3)ccc2-c2c1c1c3ccccc3oc1c1ccccc21 ; CC1(C)c2cc(-c3nc(-c4ccccc4)nc(-c4ccc5ccccc5c4)n3)ccc2-c2c1c1c3ccccc3oc1c1ccccc21]

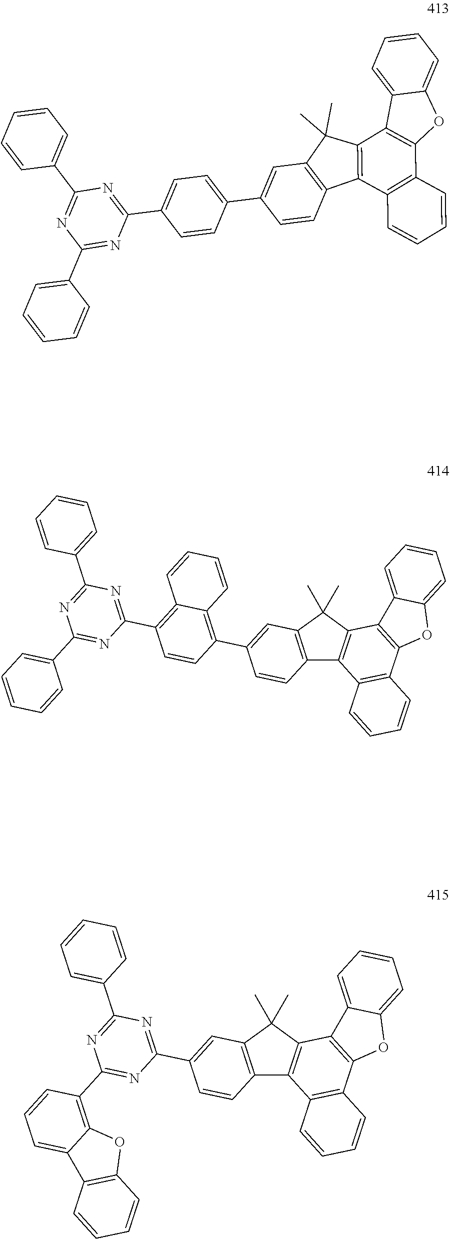 [SMILES: CC1(C)c2cc(-c3ccc(-c4nc(-c5ccccc5)nc(-c5ccccc5)n4)cc3)ccc2-c2c1c1c3ccccc3oc1c1ccccc21 ; CC1(C)c2cc(-c3ccc(-c4nc(-c5ccccc5)nc(-c5ccccc5)n4)c4ccccc34)ccc2-c2c1c1c3ccccc3oc1c1ccccc21 ; CC1(C)c2cc(-c3nc(-c4ccccc4)nc(-c4cccc5c4oc4ccccc45)n3)ccc2-c2c1c1c3ccccc3oc1c1ccccc21]

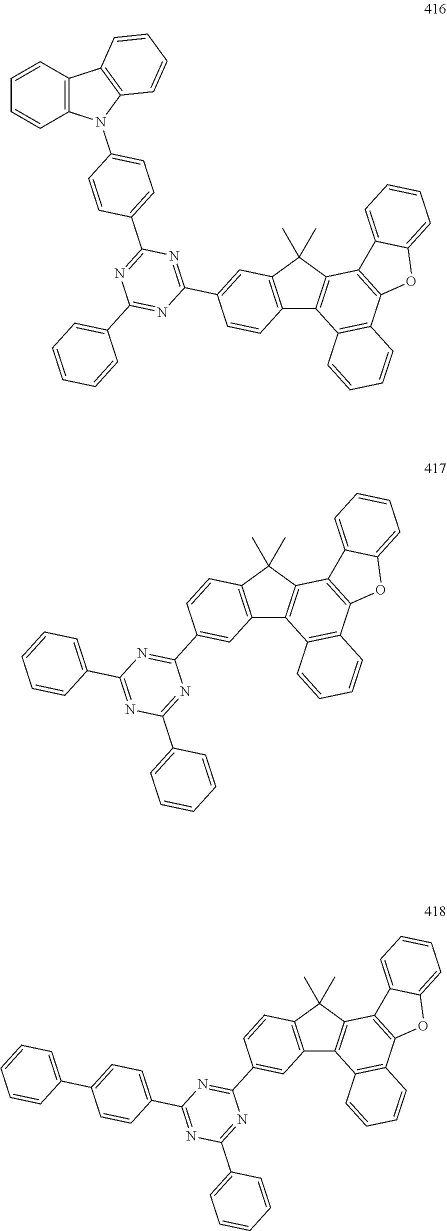 [SMILES: CC1(C)c2cc(-c3nc(-c4ccccc4)nc(-c4ccc(-n5c6ccccc6c6ccccc65)cc4)n3)ccc2-c2c1c1c3ccccc3oc1c1ccccc21 ; CC1(C)c2ccc(-c3nc(-c4ccccc4)nc(-c4ccccc4)n3)cc2-c2c1c1c3ccccc3oc1c1ccccc21 ; CC1(C)c2ccc(-c3nc(-c4ccccc4)nc(-c4ccc(-c5ccccc5)cc4)n3)cc2-c2c1c1c3ccccc3oc1c1ccccc21]

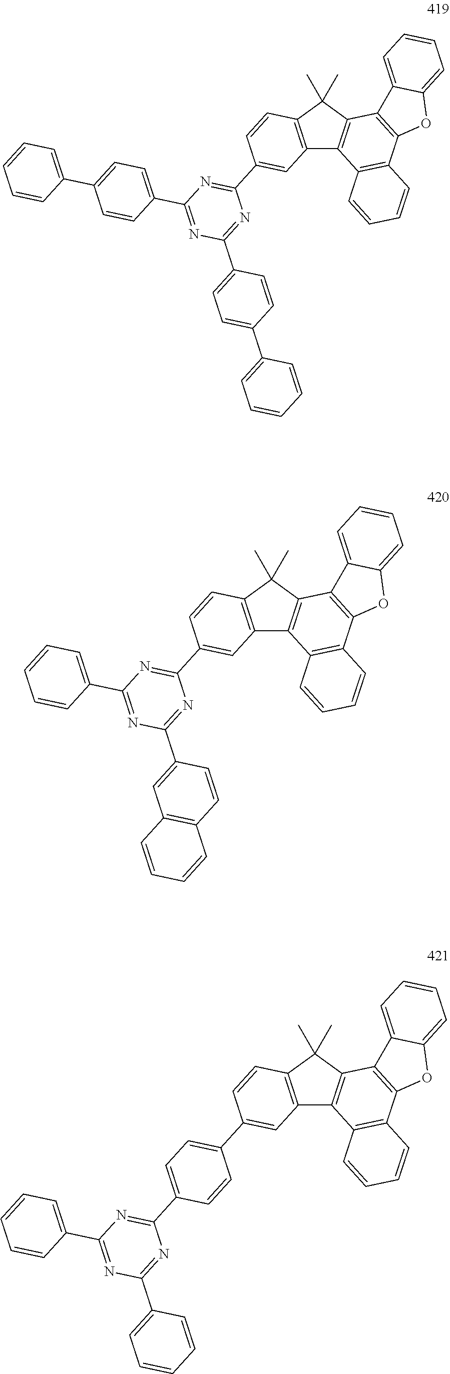 [SMILES: CC1(C)c2ccc(-c3nc(-c4ccc(-c5ccccc5)cc4)nc(-c4ccc(-c5ccccc5)cc4)n3)cc2-c2c1c1c3ccccc3oc1c1ccccc21 ; CC1(C)c2ccc(-c3nc(-c4ccccc4)nc(-c4ccc5ccccc5c4)n3)cc2-c2c1c1c3ccccc3oc1c1ccccc21 ; CC1(C)c2ccc(-c3ccc(-c4nc(-c5ccccc5)nc(-c5ccccc5)n4)cc3)cc2-c2c1c1c3ccccc3oc1c1ccccc21]

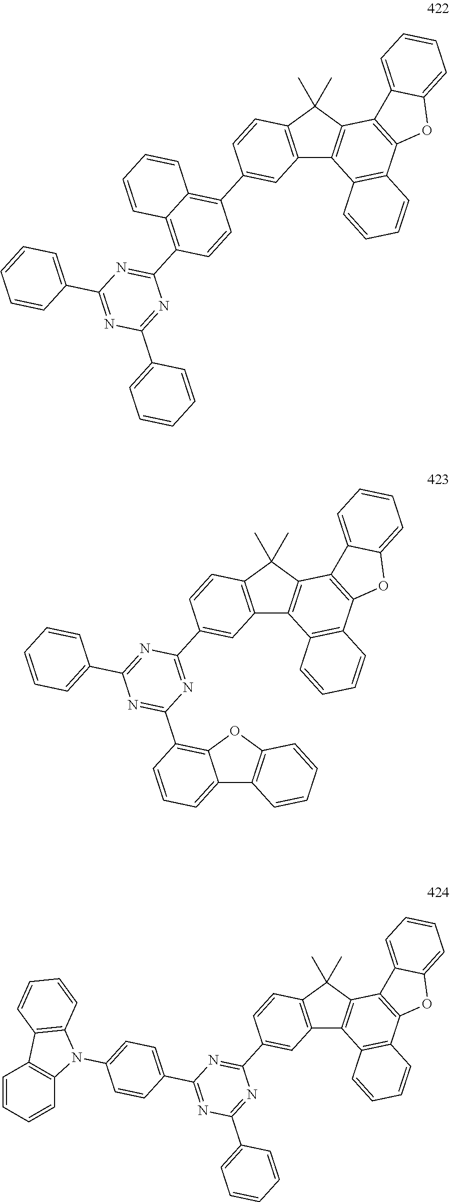 [SMILES: CC1(C)c2ccc(-c3ccc(-c4nc(-c5ccccc5)nc(-c5ccccc5)n4)c4ccccc34)cc2-c2c1c1c3ccccc3oc1c1ccccc21 ; CC1(C)c2ccc(-c3nc(-c4ccccc4)nc(-c4cccc5c4oc4ccccc45)n3)cc2-c2c1c1c3ccccc3oc1c1ccccc21 ; CC1(C)c2ccc(-c3nc(-c4ccccc4)nc(-c4ccc(-n5c6ccccc6c6ccccc65)cc4)n3)cc2-c2c1c1c3ccccc3oc1c1ccccc21]

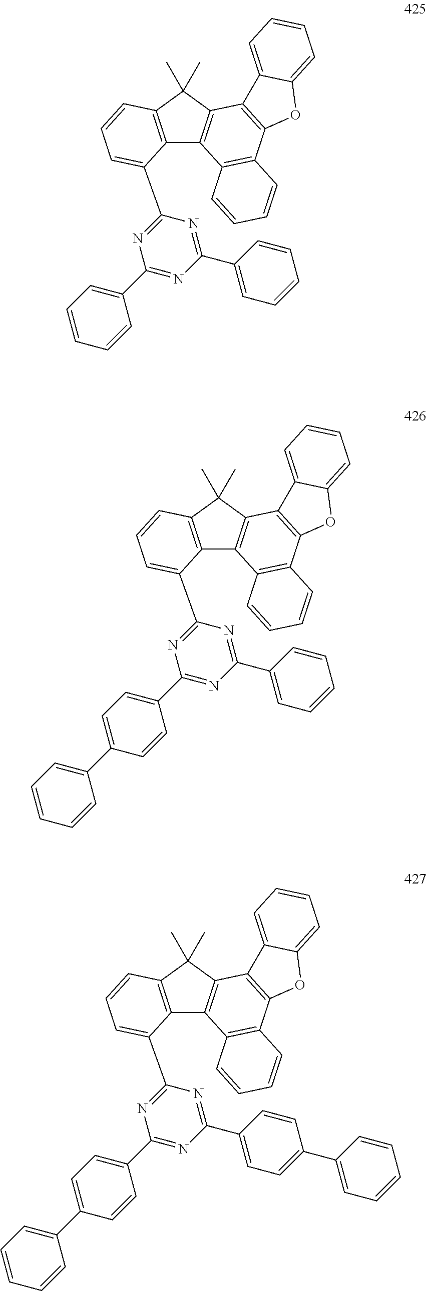 [SMILES: CC1(C)c2cccc(-c3nc(-c4ccccc4)nc(-c4ccccc4)n3)c2-c2c1c1c3ccccc3oc1c1ccccc21 ; CC1(C)c2cccc(-c3nc(-c4ccccc4)nc(-c4ccc(-c5ccccc5)cc4)n3)c2-c2c1c1c3ccccc3oc1c1ccccc21 ; CC1(C)c2cccc(-c3nc(-c4ccc(-c5ccccc5)cc4)nc(-c4ccc(-c5ccccc5)cc4)n3)c2-c2c1c1c3ccccc3oc1c1ccccc21]

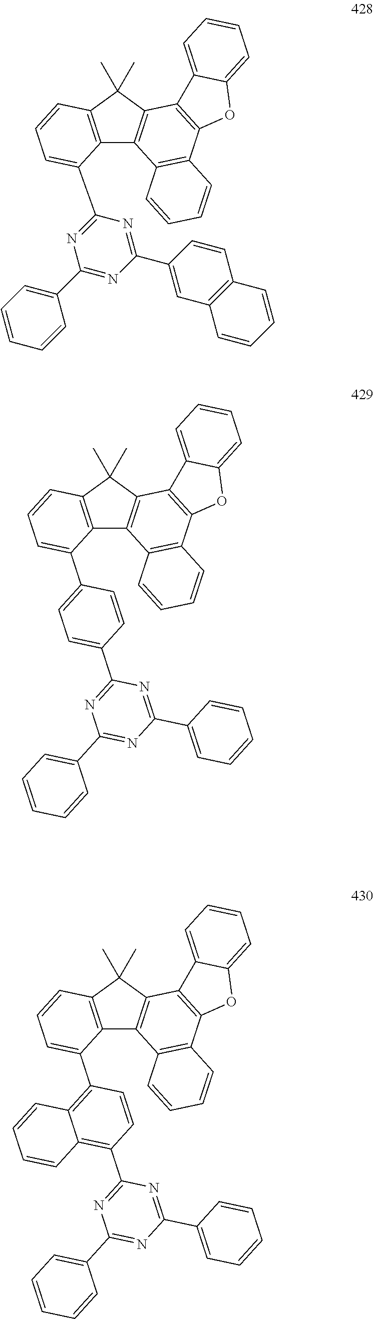 [SMILES: CC1(C)c2cccc(-c3nc(-c4ccccc4)nc(-c4ccc5ccccc5c4)n3)c2-c2c1c1c3ccccc3oc1c1ccccc21 ; CC1(C)c2cccc(-c3ccc(-c4nc(-c5ccccc5)nc(-c5ccccc5)n4)cc3)c2-c2c1c1c3ccccc3oc1c1ccccc21 ; CC1(C)c2cccc(-c3ccc(-c4nc(-c5ccccc5)nc(-c5ccccc5)n4)c4ccccc34)c2-c2c1c1c3ccccc3oc1c1ccccc21]

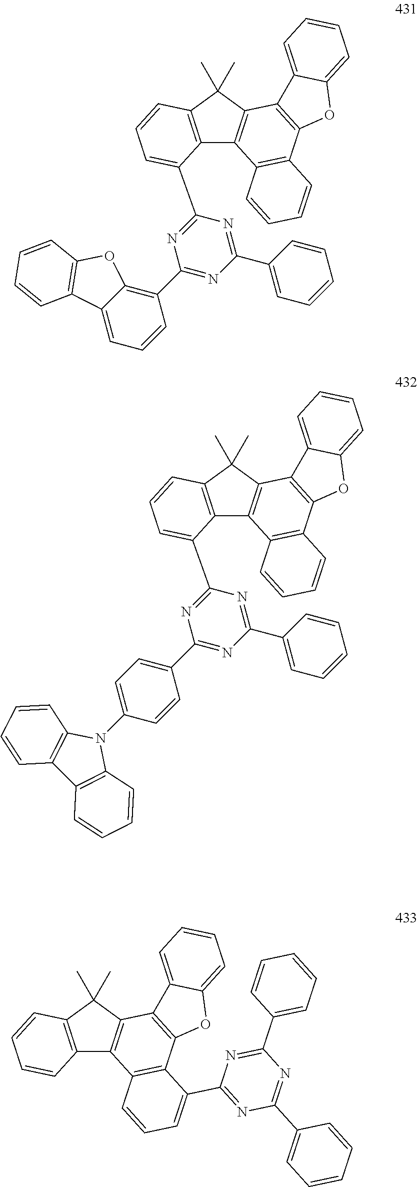 [SMILES: CC1(C)c2cccc(-c3nc(-c4ccccc4)nc(-c4cccc5c4oc4ccccc45)n3)c2-c2c1c1c3ccccc3oc1c1ccccc21 ; CC1(C)c2cccc(-c3nc(-c4ccccc4)nc(-c4ccc(-n5c6ccccc6c6ccccc65)cc4)n3)c2-c2c1c1c3ccccc3oc1c1ccccc21 ; CC1(C)c2ccccc2-c2c1c1c3ccccc3oc1c1c(-c3nc(-c4ccccc4)nc(-c4ccccc4)n3)cccc21]

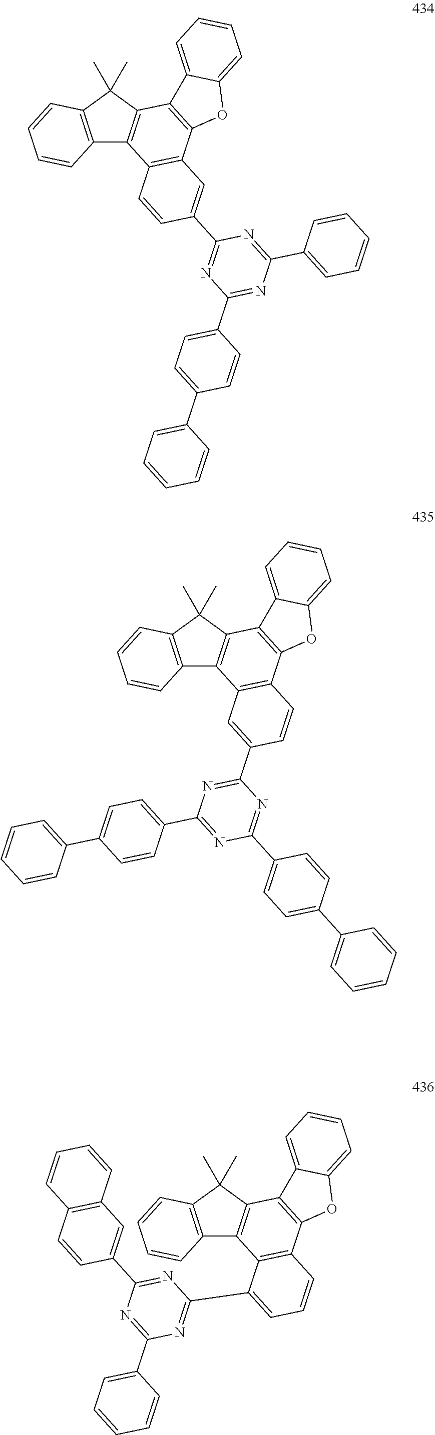 [SMILES: CC1(C)c2ccccc2-c2c1c1c3ccccc3oc1c1cc(-c3nc(-c4ccccc4)nc(-c4ccc(-c5ccccc5)cc4)n3)ccc21 ; CC1(C)c2ccccc2-c2c1c1c3ccccc3oc1c1ccc(-c3nc(-c4ccc(-c5ccccc5)cc4)nc(-c4ccc(-c5ccccc5)cc4)n3)cc21 ; CC1(C)c2ccccc2-c2c1c1c3ccccc3oc1c1cccc(-c3nc(-c4ccccc4)nc(-c4ccc5ccccc5c4)n3)c21]

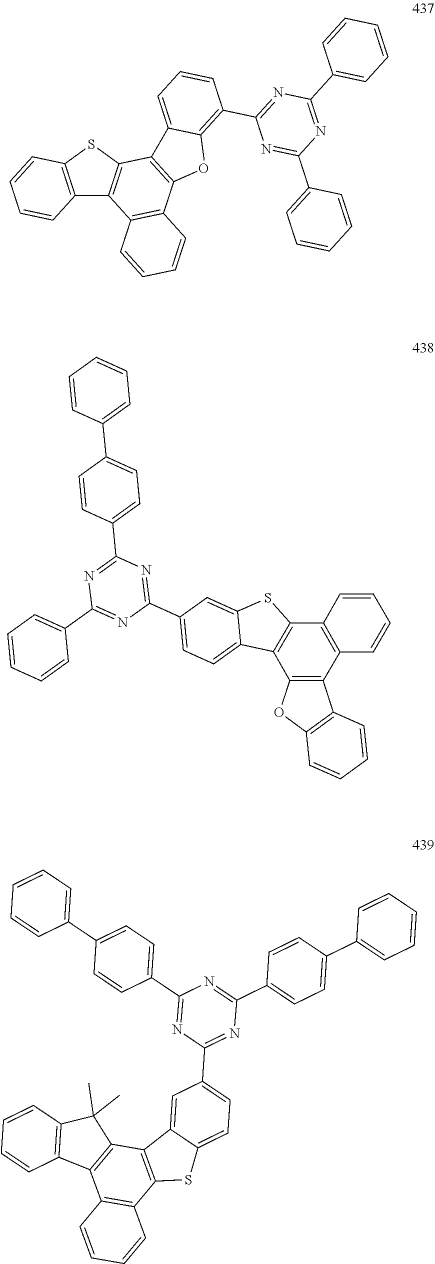 [SMILES: c1ccc(-c2nc(-c3ccccc3)nc(-c3cccc4c3oc3c5ccccc5c5c6ccccc6sc5c43)n2)cc1 ; c1ccc(-c2ccc(-c3nc(-c4ccccc4)nc(-c4ccc5c(c4)sc4c6ccccc6c6c7ccccc7oc6c54)n3)cc2)cc1 ; CC1(C)c2ccccc2-c2c1c1c3cc(-c4nc(-c5ccc(-c6ccccc6)cc5)nc(-c5ccc(-c6ccccc6)cc5)n4)ccc3sc1c1ccccc21]

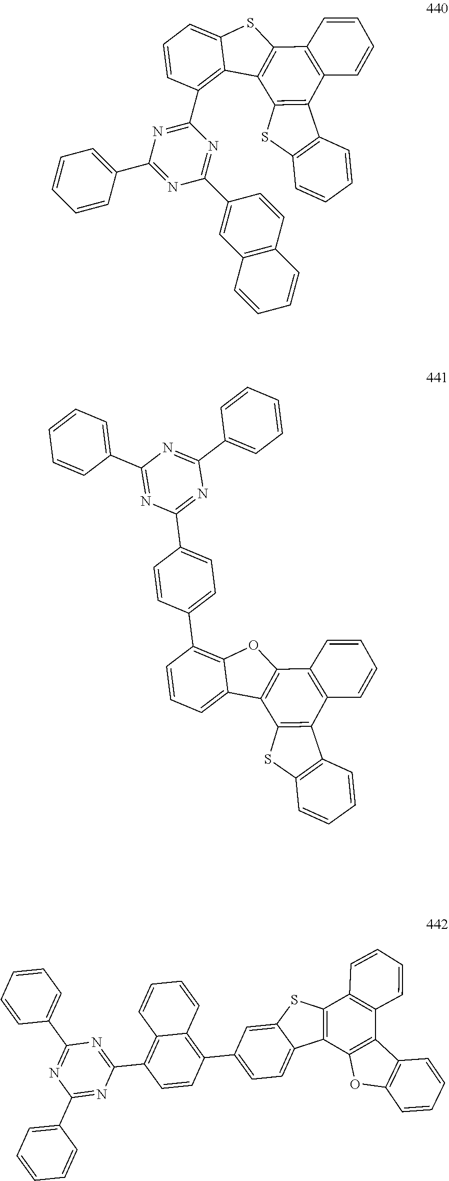 [SMILES: c1ccc(-c2nc(-c3ccc4ccccc4c3)nc(-c3cccc4sc5c6ccccc6c6c7ccccc7sc6c5c34)n2)cc1 ; c1ccc(-c2nc(-c3ccccc3)nc(-c3ccc(-c4cccc5c4oc4c6ccccc6c6c7ccccc7sc6c54)cc3)n2)cc1 ; c1ccc(-c2nc(-c3ccccc3)nc(-c3ccc(-c4ccc5c(c4)sc4c6ccccc6c6c7ccccc7oc6c54)c4ccccc34)n2)cc1]

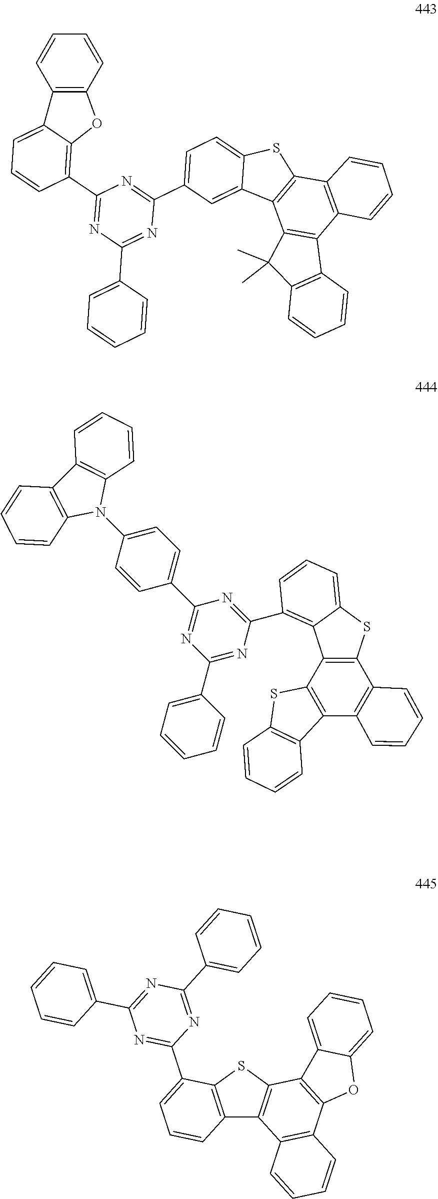 [SMILES: CC1(C)c2ccccc2-c2c1c1c3cc(-c4nc(-c5ccccc5)nc(-c5cccc6c5oc5ccccc56)n4)ccc3sc1c1ccccc21 ; c1ccc(-c2nc(-c3ccc(-n4c5ccccc5c5ccccc54)cc3)nc(-c3cccc4sc5c6ccccc6c6c7ccccc7sc6c5c34)n2)cc1 ; c1ccc(-c2nc(-c3ccccc3)nc(-c3cccc4c3sc3c4c4ccccc4c4oc5ccccc5c43)n2)cc1]

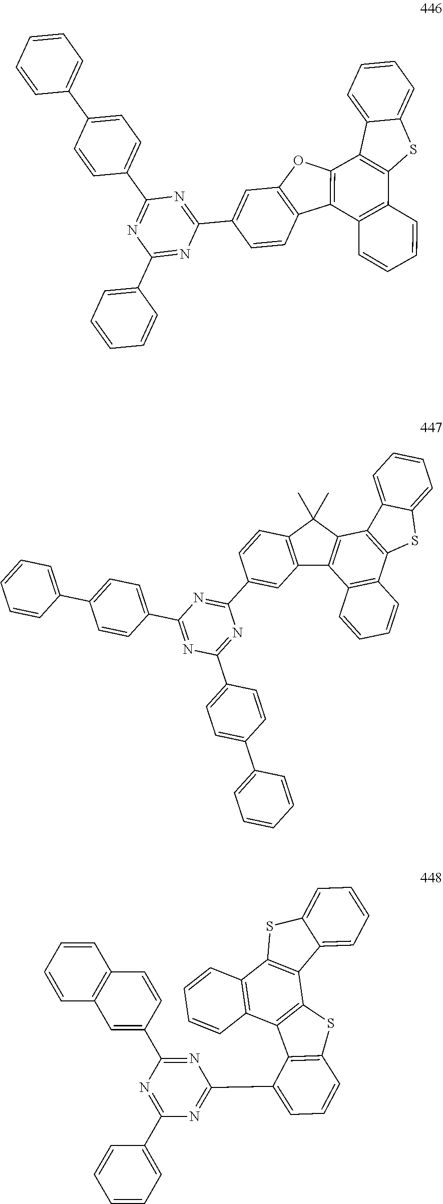 [SMILES: c1ccc(-c2ccc(-c3nc(-c4ccccc4)nc(-c4ccc5c(c4)oc4c5c5ccccc5c5sc6ccccc6c45)n3)cc2)cc1 ; CC1(C)c2ccc(-c3nc(-c4ccc(-c5ccccc5)cc4)nc(-c4ccc(-c5ccccc5)cc4)n3)cc2-c2c1c1c3ccccc3sc1c1ccccc21 ; c1ccc(-c2nc(-c3ccc4ccccc4c3)nc(-c3cccc4sc5c6c7ccccc7sc6c6ccccc6c5c34)n2)cc1]

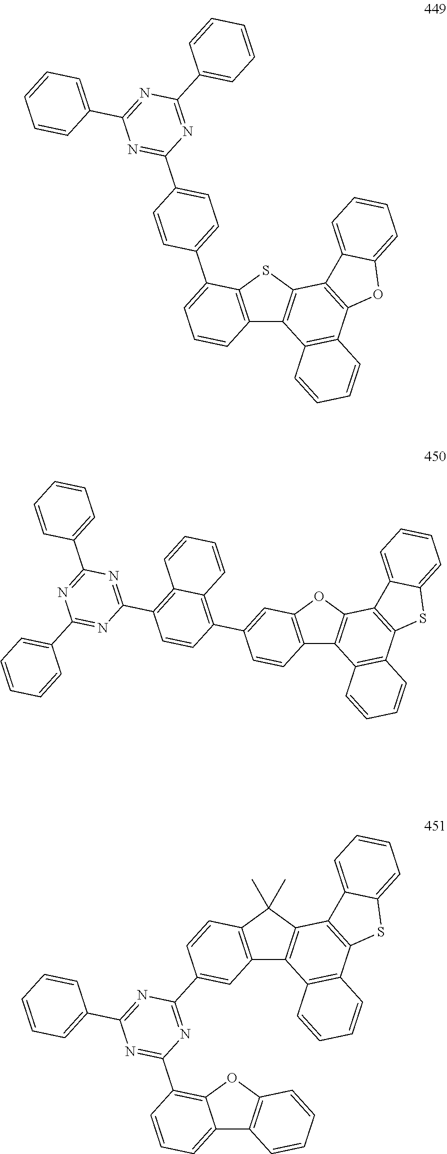 [SMILES: c1ccc(-c2nc(-c3ccccc3)nc(-c3ccc(-c4cccc5c4sc4c5c5ccccc5c5oc6ccccc6c54)cc3)n2)cc1 ; c1ccc(-c2nc(-c3ccccc3)nc(-c3ccc(-c4ccc5c(c4)oc4c5c5ccccc5c5sc6ccccc6c45)c4ccccc34)n2)cc1 ; CC1(C)c2ccc(-c3nc(-c4ccccc4)nc(-c4cccc5c4oc4ccccc45)n3)cc2-c2c1c1c3ccccc3sc1c1ccccc21]

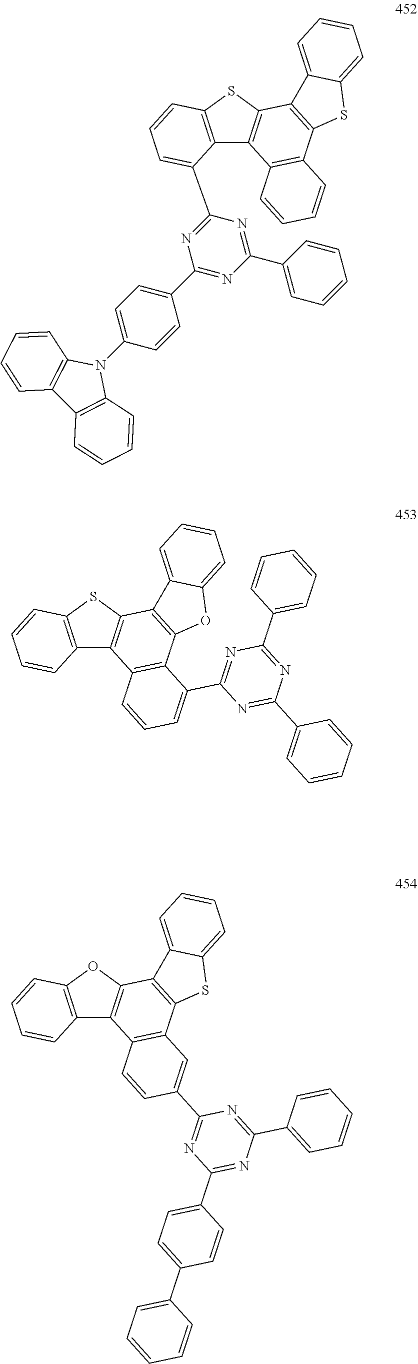 [SMILES: c1ccc(-c2nc(-c3ccc(-n4c5ccccc5c5ccccc54)cc3)nc(-c3cccc4sc5c6c7ccccc7sc6c6ccccc6c5c34)n2)cc1 ; c1ccc(-c2nc(-c3ccccc3)nc(-c3cccc4c3c3oc5ccccc5c3c3sc5ccccc5c43)n2)cc1 ; c1ccc(-c2ccc(-c3nc(-c4ccccc4)nc(-c4ccc5c(c4)c4sc6ccccc6c4c4oc6ccccc6c54)n3)cc2)cc1]

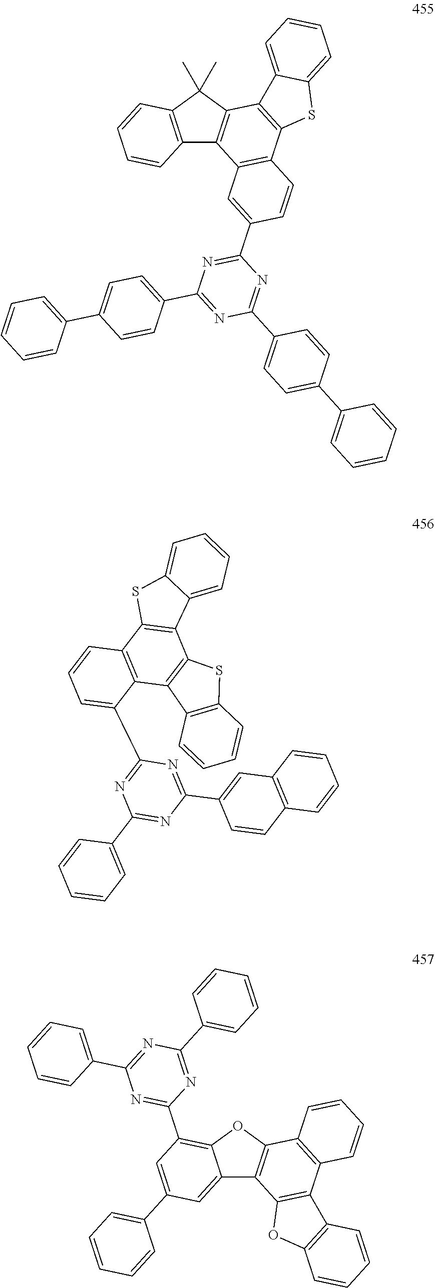 [SMILES: CC1(C)c2ccccc2-c2c1c1c3ccccc3sc1c1ccc(-c3nc(-c4ccc(-c5ccccc5)cc4)nc(-c4ccc(-c5ccccc5)cc4)n3)cc21 ; c1ccc(-c2nc(-c3ccc4ccccc4c3)nc(-c3cccc4c5sc6ccccc6c5c5sc6ccccc6c5c34)n2)cc1 ; c1ccc(-c2cc(-c3nc(-c4ccccc4)nc(-c4ccccc4)n3)c3oc4c5ccccc5c5c6ccccc6oc5c4c3c2)cc1]

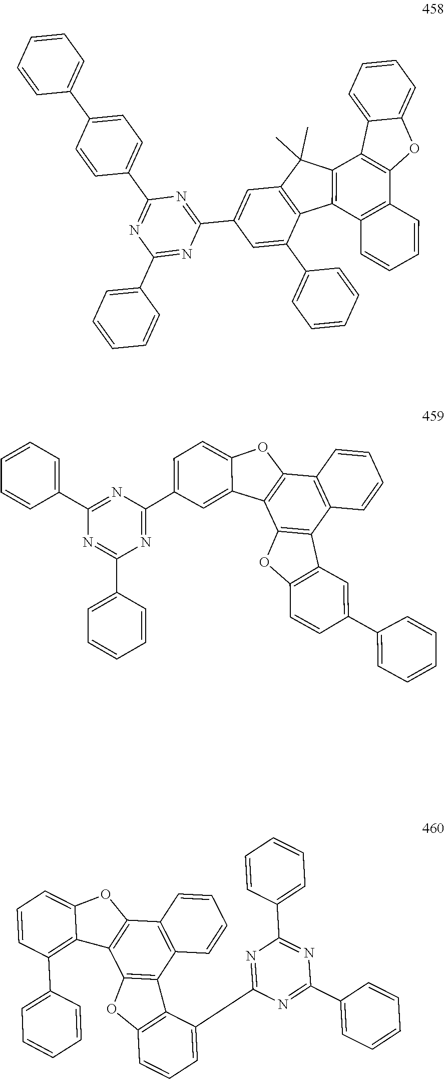 [SMILES: CC1(C)c2cc(-c3nc(-c4ccccc4)nc(-c4ccc(-c5ccccc5)cc4)n3)cc(-c3ccccc3)c2-c2c1c1c3ccccc3oc1c1ccccc21 ; c1ccc(-c2ccc3oc4c(c3c2)c2ccccc2c2oc3ccc(-c5nc(-c6ccccc6)nc(-c6ccccc6)n5)cc3c24)cc1 ; c1ccc(-c2nc(-c3ccccc3)nc(-c3cccc4oc5c(c6ccccc6c6oc7cccc(-c8ccccc8)c7c65)c34)n2)cc1]

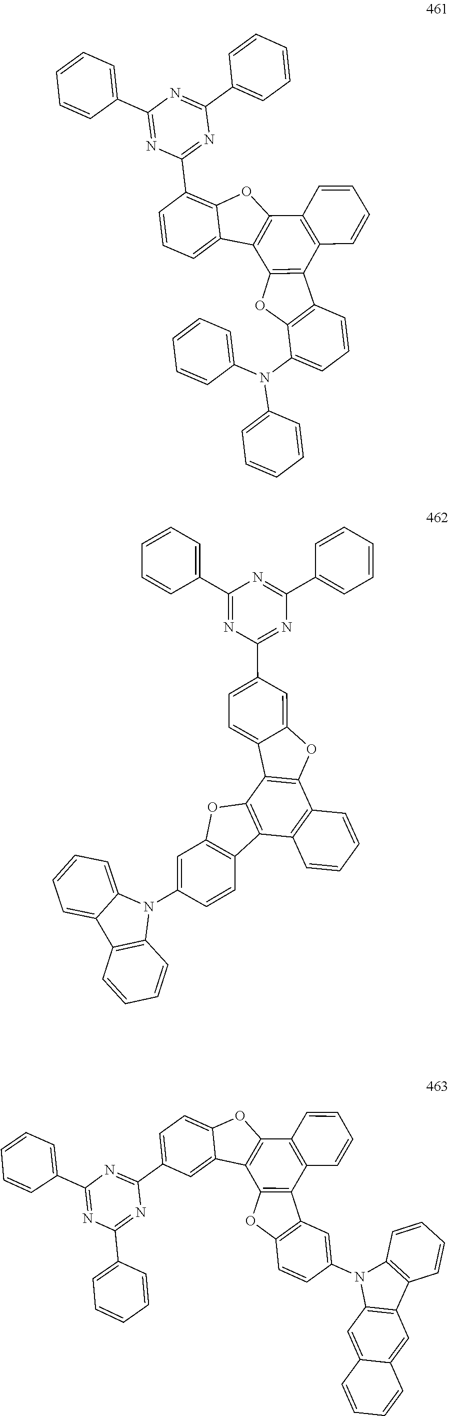 [SMILES: c1ccc(-c2nc(-c3ccccc3)nc(-c3cccc4c3oc3c5ccccc5c5c6cccc(N(c7ccccc7)c7ccccc7)c6oc5c43)n2)cc1 ; c1ccc(-c2nc(-c3ccccc3)nc(-c3ccc4c(c3)oc3c5ccccc5c5c6ccc(-n7c8ccccc8c8ccccc87)cc6oc5c43)n2)cc1 ; c1ccc(-c2nc(-c3ccccc3)nc(-c3ccc4oc5c6ccccc6c6c7cc(-n8c9ccccc9c9cc%10ccccc%10cc98)ccc7oc6c5c4c3)n2)cc1]

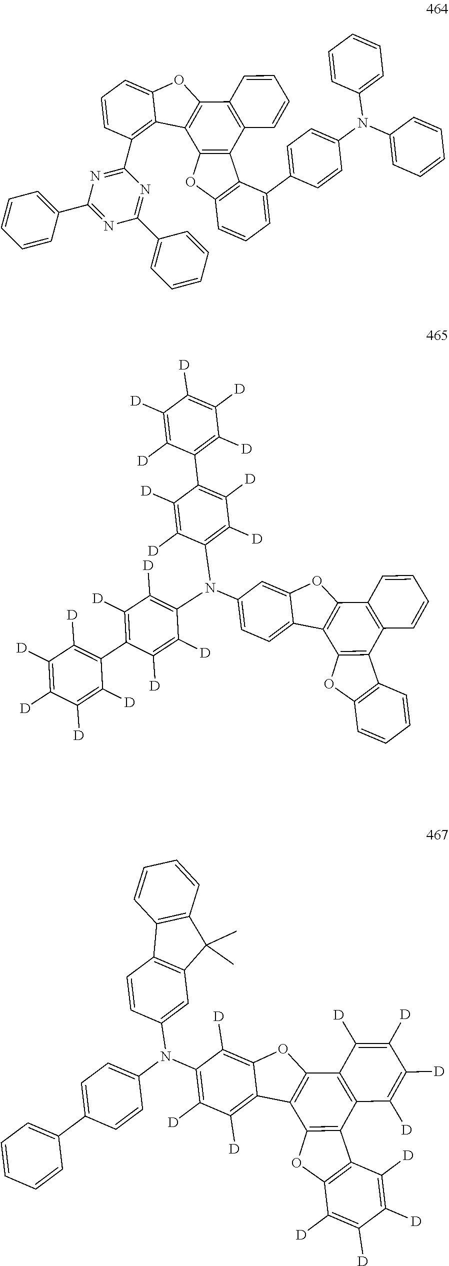 [SMILES: c1ccc(-c2nc(-c3ccccc3)nc(-c3cccc4oc5c6ccccc6c6c(oc7cccc(-c8ccc(N(c9ccccc9)c9ccccc9)cc8)c76)c5c34)n2)cc1 ; [2H]c1c([2H])c([2H])c(-c2c([2H])c([2H])c(N(c3ccc4c(c3)oc3c5ccccc5c5c6ccccc6oc5c43)c3c([2H])c([2H])c(-c4c([2H])c([2H])c([2H])c([2H])c4[2H])c([2H])c3[2H])c([2H])c2[2H])c([2H])c1[2H] ; [2H]c1c([2H])c([2H])c2c(oc3c4c(oc5c([2H])c(N(c6ccc(-c7ccccc7)cc6)c6ccc7c(c6)C(C)(C)c6ccccc6-7)c([2H])c([2H])c54)c4c([2H])c([2H])c([2H])c([2H])c4c32)c1[2H]]

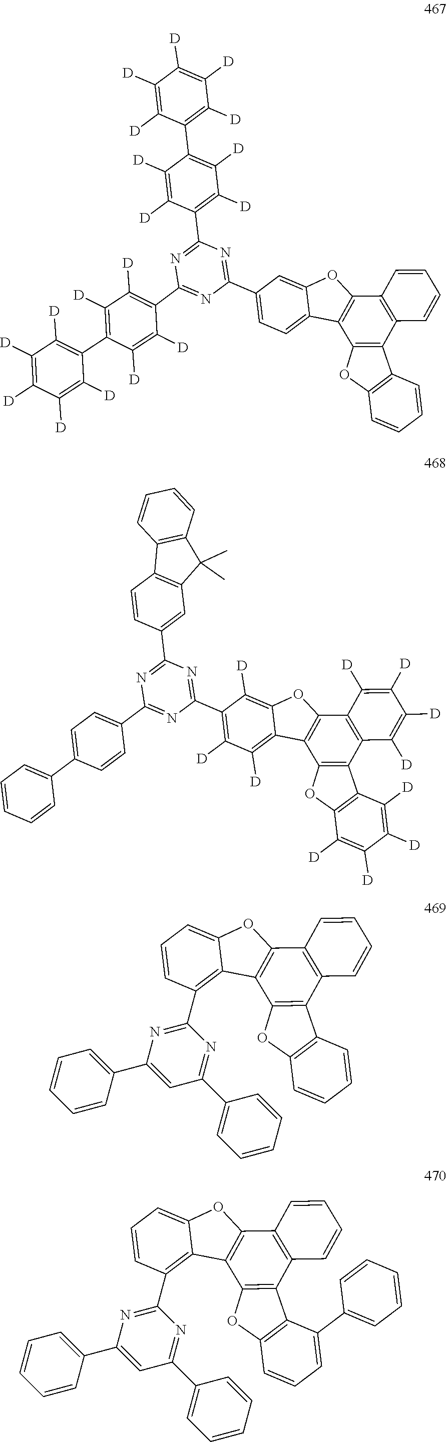

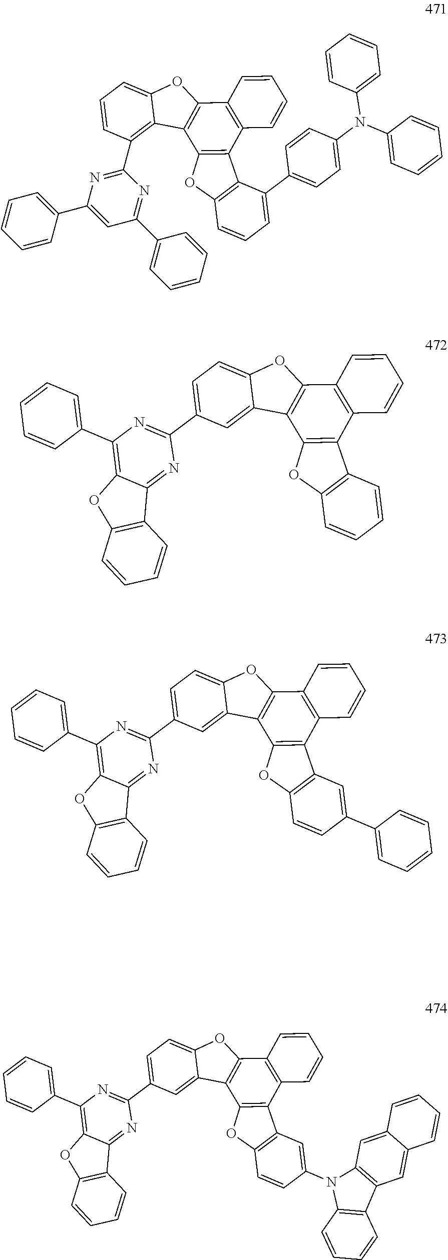 [SMILES: c1ccc(-c2cc(-c3ccccc3)nc(-c3cccc4oc5c6ccccc6c6c(oc7cccc(-c8ccc(N(c9ccccc9)c9ccccc9)cc8)c76)c5c34)n2)cc1 ; c1ccc(-c2nc(-c3ccc4oc5c6ccccc6c6c7ccccc7oc6c5c4c3)nc3c2oc2ccccc23)cc1 ; c1ccc(-c2ccc3oc4c(c3c2)c2ccccc2c2oc3ccc(-c5nc(-c6ccccc6)c6oc7ccccc7c6n5)cc3c24)cc1 ; c1ccc(-c2nc(-c3ccc4oc5c6ccccc6c6c7cc(-n8c9ccccc9c9cc%10ccccc%10cc98)ccc7oc6c5c4c3)nc3c2oc2ccccc23)cc1]

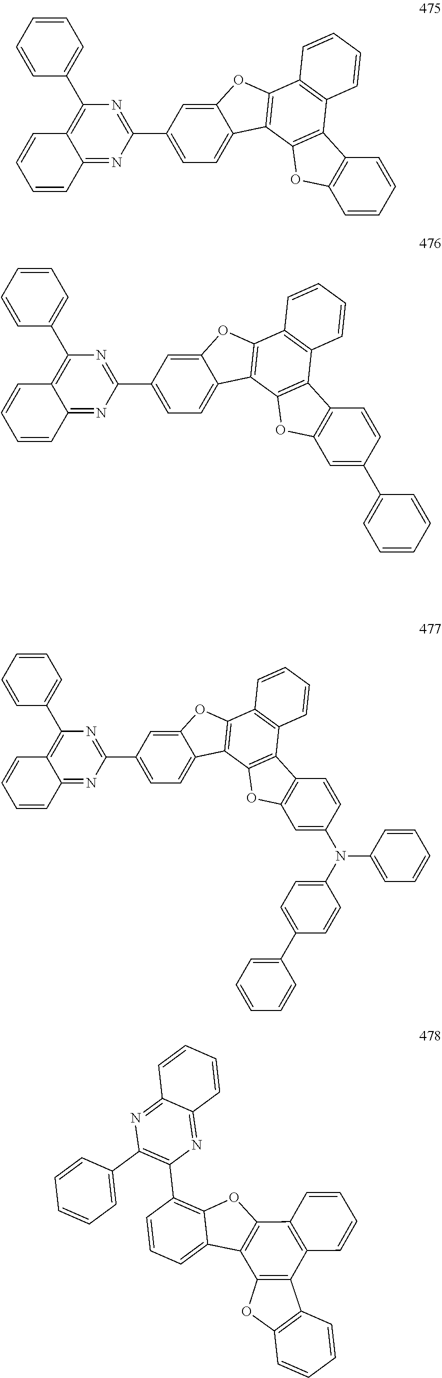 [SMILES: c1ccc(-c2nc(-c3ccc4c(c3)oc3c5ccccc5c5c6ccccc6oc5c43)nc3ccccc23)cc1 ; c1ccc(-c2ccc3c(c2)oc2c3c3ccccc3c3oc4cc(-c5nc(-c6ccccc6)c6ccccc6n5)ccc4c32)cc1 ; c1ccc(-c2ccc(N(c3ccccc3)c3ccc4c(c3)oc3c4c4ccccc4c4oc5cc(-c6nc(-c7ccccc7)c7ccccc7n6)ccc5c43)cc2)cc1 ; c1ccc(-c2nc3ccccc3nc2-c2cccc3c2oc2c4ccccc4c4c5ccccc5oc4c32)cc1]

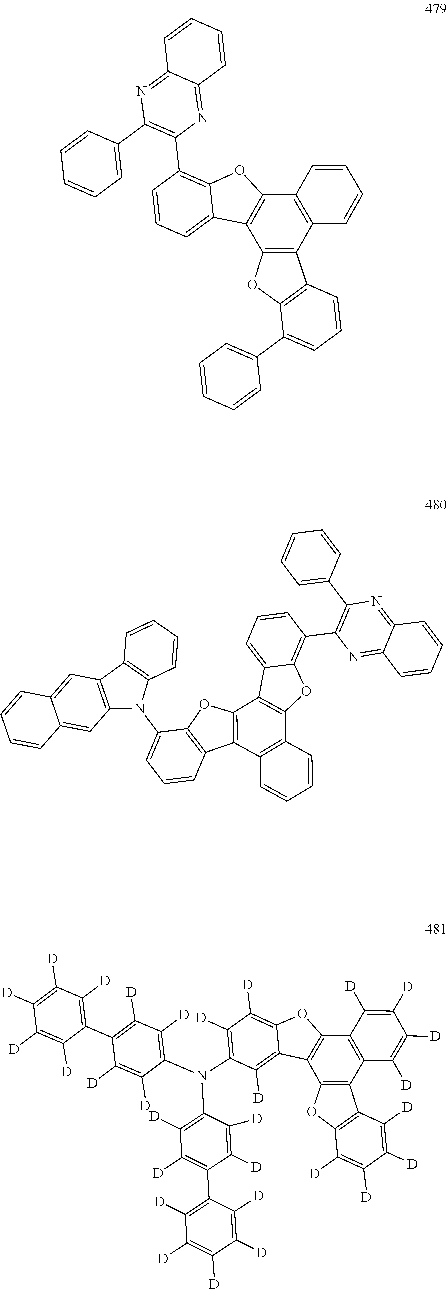 [SMILES: c1ccc(-c2nc3ccccc3nc2-c2cccc3c2oc2c4ccccc4c4c5cccc(-c6ccccc6)c5oc4c32)cc1 ; c1ccc(-c2nc3ccccc3nc2-c2cccc3c2oc2c4ccccc4c4c5cccc(-n6c7ccccc7c7cc8ccccc8cc76)c5oc4c32)cc1 ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4oc5c6c([2H])c([2H])c([2H])c([2H])c6c6c(oc7c([2H])c([2H])c([2H])c([2H])c76)c5c4c3[2H])c([2H])c2[2H])c([2H])c1[2H]]

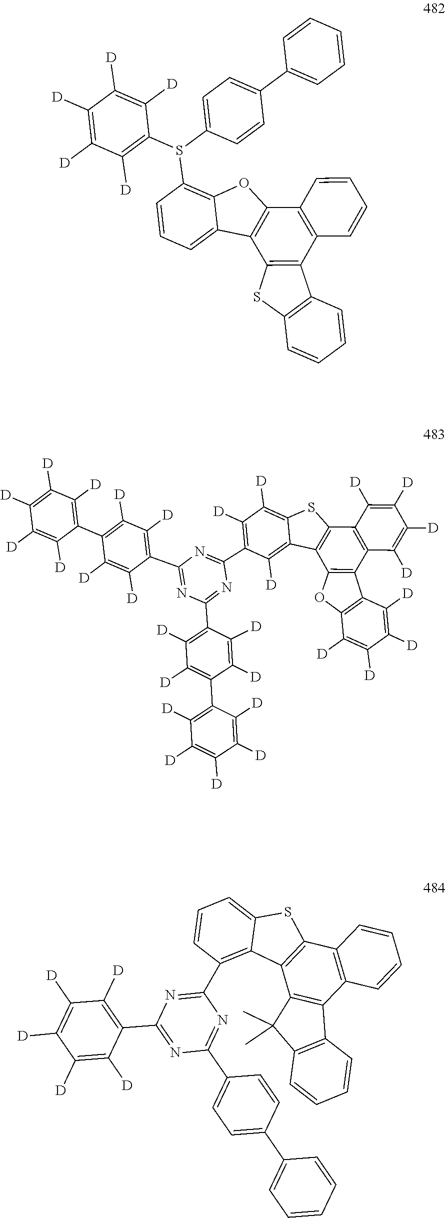

In addition, by introducing various substituents to the structure of Chemical Formula 1, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

One embodiment of the present specification provides an organic light emitting device including a first electrode; a second electrode; and one or more organic material layers provided between the first electrode and the second electrode, wherein one or more layers of the organic material layers include one or more types of the condensed polycyclic compound of Chemical Formula 1.

In one embodiment of the present specification, the first electrode may be an anode, and the second electrode may be a cathode.

In another embodiment of the present specification, the first electrode may be a cathode, and the second electrode may be an anode.

In one embodiment of the present specification, the organic light emitting device may be a blue organic light emitting device, and the condensed polycyclic compound of Chemical Formula 1 may be used as a material of the blue organic light emitting device. For example, the condensed polycyclic compound of Chemical Formula 1 may be included in a light emitting layer of the blue organic light emitting device.

In one embodiment of the present specification, the organic light emitting device may be a green organic light emitting device, and the condensed polycyclic compound of Chemical Formula 1 may be used as a material of the green organic light emitting device. For example, the condensed polycyclic compound of Chemical Formula 1 may be included in a light emitting layer of the green organic light emitting device.

In one embodiment of the present specification, the organic light emitting device may be a red organic light emitting device, and the condensed polycyclic compound of Chemical Formula 1 may be used as a material of the red organic light emitting device. For example, the condensed polycyclic compound of Chemical Formula 1 may be included in a light emitting layer of the red organic light emitting device.

The organic light emitting device of the present specification may be manufactured using common organic light emitting device manufacturing methods and materials except that one or more organic material layers are formed using the condensed polycyclic compound described above.

The condensed polycyclic compound may be formed into an organic material layer using a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present specification may be formed in a single layer structure, but may be formed in a multilayer structure in which two or more organic material layers are laminated. For example, the organic light emitting device of the present disclosure may have a structure including a hole injection layer, a hole transfer layer, a light emitting layer, an electron transfer layer, an electron injection layer and the like as the organic material layer. However, the structure of the organic light emitting device is not limited thereto, and may include a smaller number of organic material layers.

In the organic light emitting device of the present specification, the organic material layer includes a light emitting layer, and the light emitting layer may include one or more types of the condensed polycyclic compound of Chemical Formula 1.

In the organic light emitting device of the present specification, the organic material layer includes a light emitting layer, the light emitting layer includes a host, and the host may include one or more types of the condensed polycyclic compound of Chemical Formula 1.

In the organic light emitting device of the present specification, the organic material layer includes a light emitting layer, the light emitting layer includes a red host, and the red host may include one or more types of the condensed polycyclic compound of Chemical Formula 1.

In the organic light emitting device of the present specification, the organic material layer may include one or two types of the condensed polycyclic compound of Chemical Formula 1.

In the organic light emitting device of the present specification, the organic material layer includes a light emitting layer, and the light emitting layer may include one or two types of the condensed polycyclic compound of Chemical Formula 1.

In the organic light emitting device of the present specification, the organic material layer includes a light emitting layer, the light emitting layer includes a host, and the host may include one or two types of the condensed polycyclic compound of Chemical Formula 1.

In the organic light emitting device of the present specification, the organic material layer includes a light emitting layer, the light emitting layer includes a red host, and the red host may include one or two types of the condensed polycyclic compound of Chemical Formula 1.

In one embodiment of the present specification, the organic material layer may include the two types of condensed polycyclic compound in a weight ratio of 1:10 to 10:1.

In one embodiment of the present specification, the organic material layer may include the two types of condensed polycyclic compound in a weight ratio of 1:5 to 5:1.

In one embodiment of the present specification, the organic material layer may include the two types of condensed polycyclic compound in a weight ratio of 1:2 to 2:1.

In one embodiment of the present specification, the compound including an amine derivative among the condensed polycyclic compounds may be used as a P-type host.

In one embodiment of the present specification, the compound including an azine derivative among the condensed polycyclic compounds may be used as an N-type host.

In the present specification, azine is a heterocyclic compound including a 6-membered aromatic ring, and means a benzene ring analog in which one or more carbon atoms of the benzene ring are replaced by a nitrogen atom. For example, the azine compound in which one carbon atom of the benzene ring is replaced by a nitrogen atom is pyridine, the azine compound in which two carbon atoms of the benzene ring are replaced by a nitrogen atom is pyridazine, pyrimidine or pyrazine, and the azine compound in which three carbon atoms of the benzene ring are replaced by a nitrogen atom is triazine.

The organic light emitting device of the present disclosure may further include one, two or more layers selected from the group consisting of a light emitting layer, a hole injection layer, a hole transfer layer, an electron injection layer, an electron transfer layer, an electron blocking layer and a hole blocking layer.

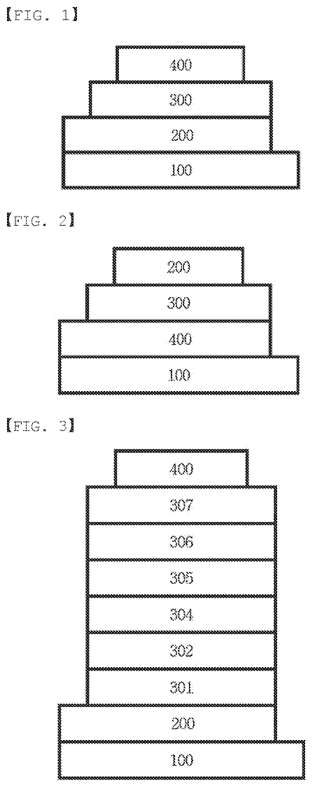

FIG. 1 to FIG. 4 illustrate a lamination order of electrodes and organic material layers of the organic light emitting device according to one embodiment of the present specification. However, the scope of the present application is not limited by these diagrams, and structures of organic light emitting devices known in the art may also be used in the present application.

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

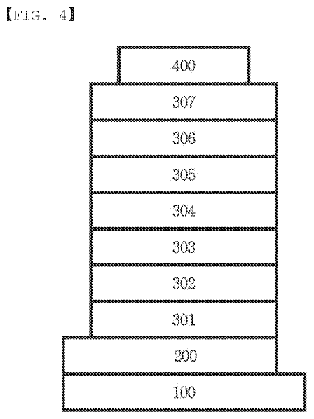

FIG. 3 and FIG. 4 illustrate cases of the organic material layer being a multilayer. The organic light emitting device according to FIG. 4 includes a hole injection layer (301), a hole transfer layer (302), an electron blocking layer (303), a light emitting layer (304), a hole blocking layer (305), an electron transfer layer (306) and an electron injection layer (307). However, the scope of the present application is not limited to such a lamination structure, and as necessary, the layers other than the light emitting layer may not be included, and other necessary functional layers may be further added.

The organic material layer including the condensed polycyclic compound of Chemical Formula 1 may further include other materials as necessary.

In the organic light emitting device according to one embodiment of the present specification, materials other than the compound of Chemical Formula 1 are illustrated below, however, these are for illustrative purposes only and not for limiting the scope of the present application, and these materials may be replaced by materials known in the art.

As the anode material, materials having relatively large work function may be used, and transparent conductive oxides, metals, conductive polymers or the like may be used. Specific examples of the anode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the cathode material, materials having relatively small work function may be used, and metals, metal oxides, conductive polymers or the like may be used. Specific examples of the cathode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB) or 4,4′,4″-tris[2-naphthyl(phenyl)amino]triphenylamine (2-TNATA) described in the literature [Advanced Material, 6, p. 677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrenesulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used. For example, N,N′-di(1-naphthyl)-N,N′-diphenyl-(1,1′-biphenyl)-4,4′-diamine (NPB) may be used.

As the hole blocking material, BCP (bathocuproine) may be used, however, the hole blocking material is not limited thereto.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials. For example, tris(8-hydroxyquinolinato)aluminum (Alq₃) may be used.

As examples of the electron injection material, LiF is typically used in the art, however, the present application is not limited thereto.

As the light emitting material, red, green or blue light emitting materials may be used, and as necessary, two or more light emitting materials may be mixed and used. Herein, the two or more light emitting materials may be deposited with individual sources of supply or premixed and deposited with one source of supply when used. In addition, fluorescent materials may also be used as the light emitting material, however, phosphorescent materials may also be used. As the light emitting material, materials emitting light by bonding holes and electrons injected from an anode and a cathode, respectively, may be used alone, however, materials having a host material and a dopant material involving together in light emission may also be used.

In one embodiment of the present specification, a red phosphorescent dopant may be used as the dopant material.

In one embodiment of the present specification, (piq)₂(Ir)_{(acac)} may be used as the dopant material, however, the dopant material is not limited thereto.

When mixing light emitting material hosts, same series hosts may be mixed, or different series hosts may be mixed. For example, any two or more types of materials among N-type host materials or P-type host materials may be selected and used as a host material of a light emitting layer.

The organic light emitting device according to one embodiment of the present specification may be a top-emission type, a bottom-emission type or a dual-emission type depending on the materials used.

The compound according to one embodiment of the present specification may also be used in an organic electronic device including an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

### Preparation Example 1

### Preparation of Compound 3

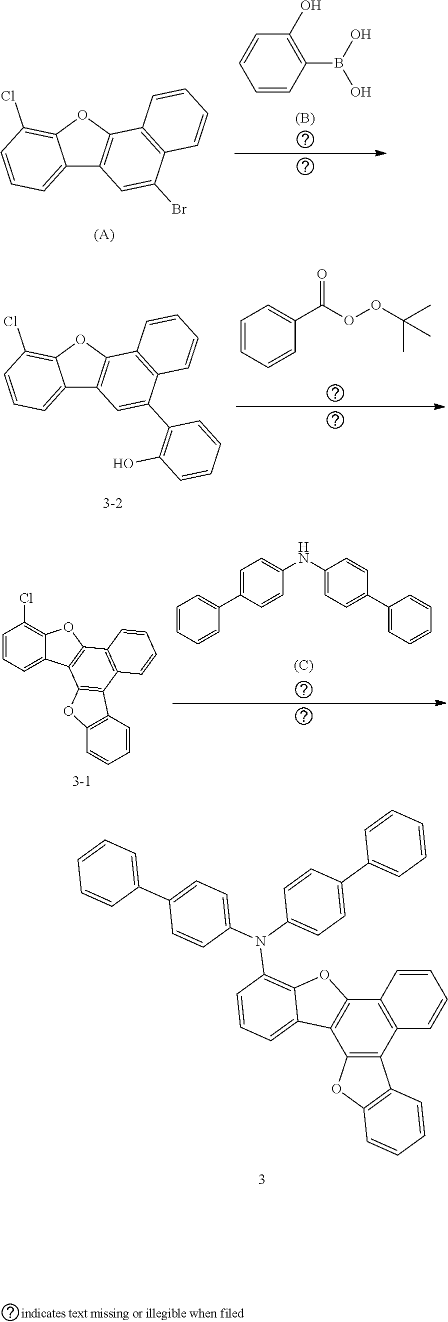

### 1) Preparation of Intermediate 3-2

1,4-Dioxane (200 ml) and H₂O (40 ml) were introduced to 5-bromo-10-chloronaphtho[1,2-b]benzofuran (A) (20 g, 0.060 mol, 1 eq.), (2-hydroxyphenyl)boronic acid (B) (10 g, 0.072 mol, 1.2 eq.), K₂CO₃ (20.8 g, 0.151 mol, 2.5 eq.) and Pd(PPh₃)₄ (tetrakis(triphenylphosphine)palladium(0)) (3.5 g, 0.003 mol, 0.05 eq.) in a reaction flask, and the mixture was stirred for 6 hours at 90° C. After terminating the reaction by introducing water thereto, the result was extracted using methylene chloride (MC) and water. After that, water was removed with MgSO₄. The result was separated by a silica gel column to obtain Intermediate 3-2 (12 g) in a 58% yield.

### 2) Preparation of Intermediate 3-1

C₆F₆ (60 ml) and 1,3-dimethyl-2-imidazolidinone (60 ml) were introduced to Intermediate 3-2 (12 g, 0.029 mol, 1 eq.), tert-butyl peroxybenzoate (13.5 g, 0.069 mol, 2 eq.), Pd(OAc)₂ (palladium(II) acetate) (0.65 g, 0.003 mol, 0.1 eq.) and 3-nitropyridine (0.43 g, 0.006 mol, 0.1 eq.) in a reaction flask, and the mixture was stirred for 6 hours at 90° C. After terminating the reaction by introducing water thereto, the result was extracted using MC and water. After that, water was removed with MgSO₄. The result was separated by a silica gel column to obtain Intermediate 3-1 (6 g) in a 505 yield.

### 3) Preparation of Compound 3

To a reaction flask, Intermediate 3-1 (6 g, 0.017 mol, 1 eq.), di([1,1′-biphenyl]-4-yl)amine (C) (6.2 g, 0.019 mol, 1.1 eq.), NaOt-Bu (sodium tert-butoxide) (2.5 g, 0.026 mol, 1.5 eq.), Pd₂(dba)₃ (tris(dibenzylideneacetone)dipalladium(0)) (0.8 g, 0.0008 mol, 0.05 eq.) and XPhos (2-dicyclohexylphosphino-2′,4′,6′-triisopropylbiphenyl) (0.8 g, 0.0017 mol, 0.1 eq.) were introduced, and after introducing toluene (90 ml) thereto, the mixture was stirred for 6 hours at 100° C. After terminating the reaction by introducing water thereto, the result was extracted using MC and water. After that, water was removed with MgSO₄. The result was separated by a silica gel column to obtain Compound 3 (8 g) in a 73% yield.

### Preparation Example 2

### Preparation of Compound 93

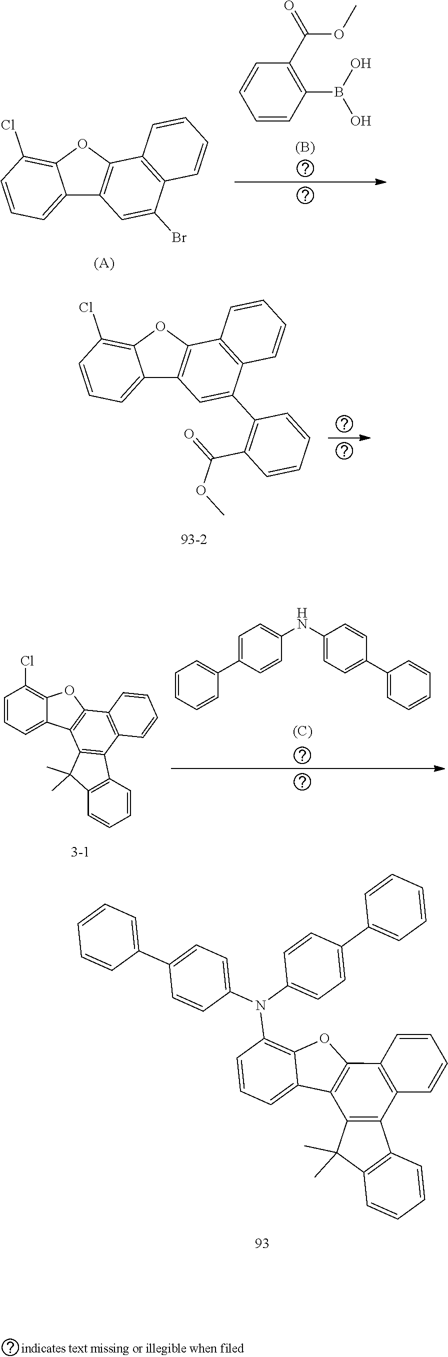

### 1) Preparation of Intermediate 93-2

To a reaction flask, 5-bromo-10-chloronaphtho[1,2-b]benzofuran (A) (20 g, 0.060 mol, 1 eq.), (2-(methoxycarbonyl)phenyl)boronic acid (B′) (11.9 g, 0.066 mol, 1.1 eq.), K₂CO₃ (20.8 g, 0.151 mol, 2.5 eq.) and Pd(PPh₃)₄ (3.5 g, 0.003 mol, 0.05 eq.) were introduced, and after introducing 1,4-dioxane (200 ml) and H₂O (40 ml) thereto, the mixture was stirred for 6 hours at 90° C. After terminating the reaction by introducing water thereto, the result was extracted using MC and water. After that, water was removed with MgSO₄. The result was separated by a silica gel column to obtain Intermediate 93-2 (19 g) in a 81% yield.

### 2) Preparation of Intermediate 93-1

To a reaction flask, Intermediate 93-2 (19 g, 0.049 mol, 1 eq.) was introduced, tetrahydrofuran (THF) (190 ml) was introduced thereto to dissolve Intermediate 93-2, then 3 M methylmagnesium bromide (49.1 ml, 0.147 mol, 3 eq.) was slowly introduced thereto at 0° C., and the mixture was stirred for 6 hours at 80° C. After terminating the reaction by introducing water thereto, the result was extracted using MC and water. After that, water was removed with MgSO₄. The result was dissolved again in MC after removing the solvent, then boron trifluoride diethyl etherate (7 g, 0.049 mol, 1 eq.) was slowly introduced thereto at 0° C., and the result was stirred for 1 hour at room temperature (RT). The result was separated by a silica gel column to obtain Intermediate 93-1 (14 g) in a 77% yield.

### 3) Preparation of Compound 93

To a reaction flask, Intermediate 93-1 (10 g, 0.027 mol, 1 eq.), di([1,1′-biphenyl]-4-yl)amine (C) (9.5 g, 0.030 mol, 1.1 eq.), NaOt-Bu (3.9 g, 0.04 mol, 1.5 eq.), Pd₂(dba)₃ (1.2 g, 0.001 mol, 0.05 eq.) and XPhos (1.2 g, 0.003 mol, 0.1 eq.) were introduced, and after introducing toluene (150 ml) thereto, the mixture was stirred for 6 hours at 100° C. After terminating the reaction by introducing water thereto, the result was extracted using MC and water. After that, water was removed with MgSO₄. The result was separated by a silica gel column to obtain Compound 93 (10 g) in a 56% yield.

### Preparation Example 3

### Preparation of Compound 301

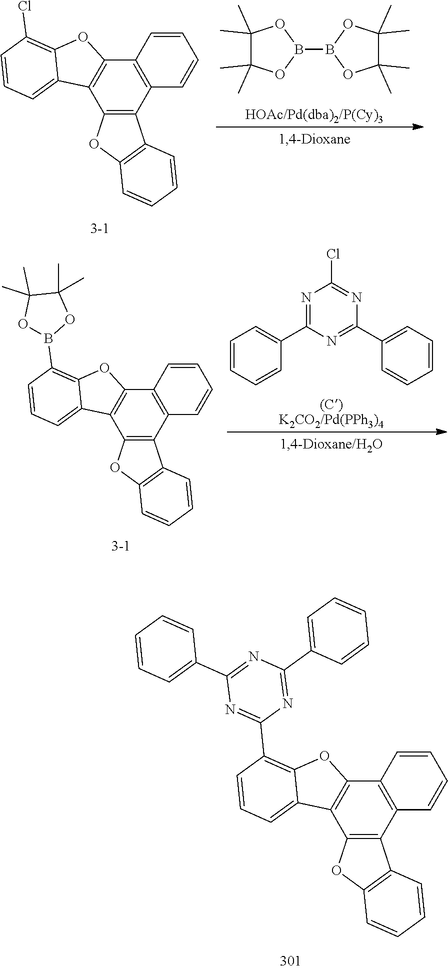

### 1) Preparation of Intermediate 301-1

To a reaction flask, Intermediate 3-1 (20 g, 0.058 mol, 1 eq.) of Preparation Example 1, 4,4,4′,4′,5,5,5′,5′-octamethyl-2,2′-bi(1,3,2-dioxaborolane) (22.2 g, 0.087 mol, 1.5 eq.), KOAc (potassium acetate) (17.2 g, 0.175 mol, 3 eq.), Pd(dba)₂ (bis(dibenzylideneacetone)palladium(0)) (1.7 g, 0.003 mol, 0.05 eq.) and P(Cy)₃ (tricyclohexyl phosphine) (1.6 g, 0.006 mol, 0.1 eq.) were introduced, and after introducing 1,4-dioxane (200 ml) thereto, the mixture was stirred for 8 hours at 100° C. After terminating the reaction by introducing water thereto, the result was extracted using MC and water. After that, water was removed with MgSO₄. The result was separated by a silica gel column to obtain Intermediate 301-1 (18 g) in a 71% yield.

### 2) Preparation of Compound 301

To a reaction flask, Intermediate 301-1 (9 g, 0.02 mol, 1 eq.), 2-chloro-4,6-diphenyl-1,3,5-triazine (C′) (5.8 g, 0.022 mol, 1.05 eq.), K₂CO₃ (7.2 g, 0.052 mol, 2.5 eq.) and Pd(PPh₃)₄ (1.2 g, 0.001 mol, 0.05 eq.) were introduced, and after introducing 1,4-dioxane (200 ml) thereto, the mixture was stirred for 8 hours at 100° C. After terminating the reaction by introducing water thereto, the result was extracted using MC and water. After that, water was removed with MgSO₄. The result was separated by a silica gel column to obtain Compound 301 (8 g) in a 72% yield.

Compounds were synthesized in the same manner as in the preparation examples except that Compound A of the following Table 1 was used instead of Compound (A), Compound B of the following Table 1 was used instead of Compound (B) or (B′), and Compound C of the following Table 1 was used instead of Compound (C) or (C′).

Relevant preparation examples are indicated in the following Table 1, and for compounds with no indication of preparation example, the same method as Preparation Example 1 was used.

TABLE 1

| Com- | | | |
|---|---|---|---|
| pound | | | |
| No. | Compound A | Compound B | Compound C |
| 23 | 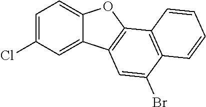 [SMILES: Clc1ccc2oc3c4ccccc4c(Br)cc3c2c1] | 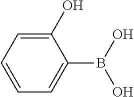 | 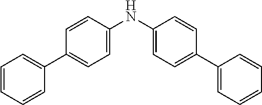 [SMILES: c1ccc(-c2ccc(Nc3ccc(-c4ccccc4)cc3)cc2)cc1] |
| 44 | 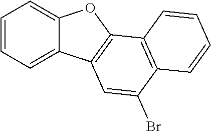 [SMILES: Brc1cc2c3ccccc3oc2c2ccccc12] | 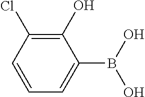 [SMILES: OB(O)c1cccc(Cl)c1O] | 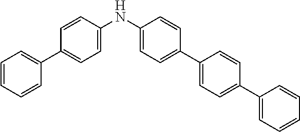 [SMILES: c1ccc(-c2ccc(Nc3ccc(-c4ccc(-c5ccccc5)cc4)cc3)cc2)cc1] |
| 66 | 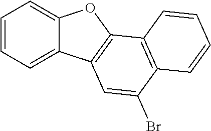 [SMILES: Brc1cc2c3ccccc3oc2c2ccccc12] | 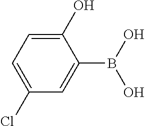 [SMILES: OB(O)c1cc(Cl)ccc1O] | 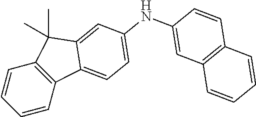 [SMILES: CC1(C)c2ccccc2-c2ccc(Nc3ccc4ccccc4c3)cc21] |
| 103 | 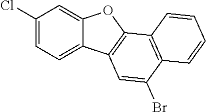 [SMILES: Clc1ccc2c(c1)oc1c3ccccc3c(Br)cc21] | 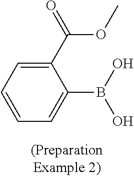 | 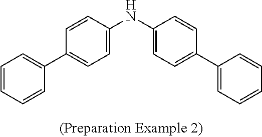 [SMILES: c1ccc(-c2ccc(Nc3ccc(-c4ccccc4)cc3)cc2)cc1] |
| 134 | 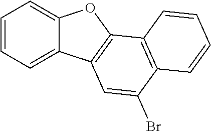 [SMILES: Brc1cc2c3ccccc3oc2c2ccccc12] | 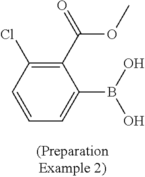 | 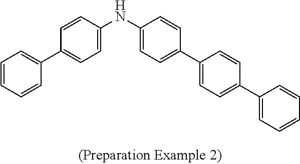 [SMILES: c1ccc(-c2ccc(Nc3ccc(-c4ccc(-c5ccccc5)cc4)cc3)cc2)cc1] |
| 160 | 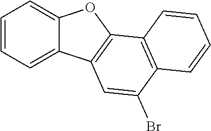 [SMILES: Brc1cc2c3ccccc3oc2c2ccccc12] | 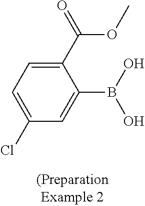 | 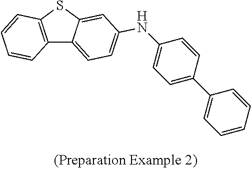 [SMILES: c1ccc(-c2ccc(Nc3ccc4c(c3)sc3ccccc34)cc2)cc1] |
| 170 | 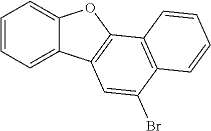 [SMILES: Brc1cc2c3ccccc3oc2c2ccccc12] | 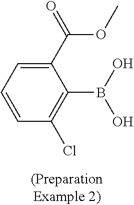 | 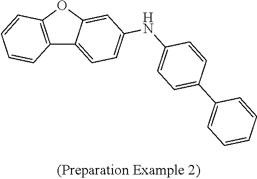 [SMILES: c1ccc(-c2ccc(Nc3ccc4c(c3)oc3ccccc34)cc2)cc1] |
| 194 | 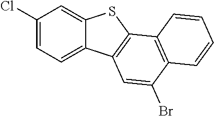 [SMILES: Clc1ccc2c(c1)sc1c3ccccc3c(Br)cc21] | 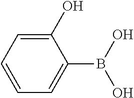 | 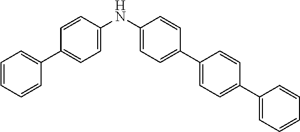 [SMILES: c1ccc(-c2ccc(Nc3ccc(-c4ccc(-c5ccccc5)cc4)cc3)cc2)cc1] |
| 207 | 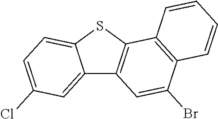 [SMILES: Clc1ccc2sc3c4ccccc4c(Br)cc3c2c1] | 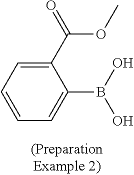 | 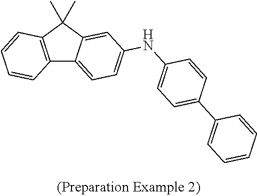 [SMILES: CC1(C)c2ccccc2-c2ccc(Nc3ccc(-c4ccccc4)cc3)cc21] |
| 235 | 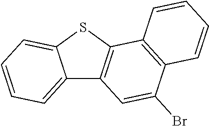 [SMILES: Brc1cc2c3ccccc3sc2c2ccccc12] | 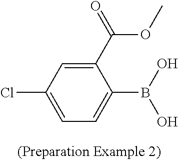 | 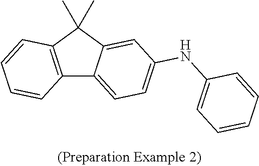 [SMILES: CC1(C)c2ccccc2-c2ccc(Nc3ccccc3)cc21] |
| 258 | 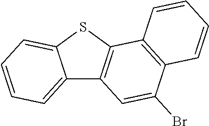 [SMILES: Brc1cc2c3ccccc3sc2c2ccccc12] | 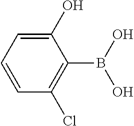 [SMILES: OB(O)c1c(O)cccc1Cl] | 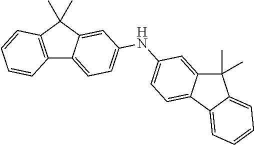 [SMILES: CC1(C)c2ccccc2-c2ccc(Nc3ccc4c(c3)C(C)(C)c3ccccc3-4)cc21] |
| 275 | 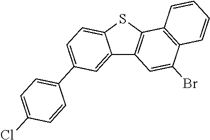 [SMILES: Clc1ccc(-c2ccc3sc4c5ccccc5c(Br)cc4c3c2)cc1] | 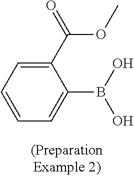 | 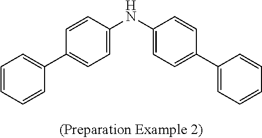 [SMILES: c1ccc(-c2ccc(Nc3ccc(-c4ccccc4)cc3)cc2)cc1] |
| 281 | 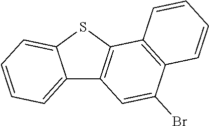 [SMILES: Brc1cc2c3ccccc3sc2c2ccccc12] | 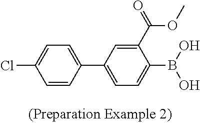 [SMILES: COC(=O)c1cc(-c2ccc(Cl)cc2)ccc1B(O)O] | 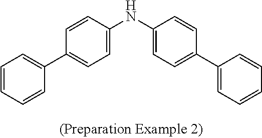 [SMILES: c1ccc(-c2ccc(Nc3ccc(-c4ccccc4)cc3)cc2)cc1] |
| 329 | 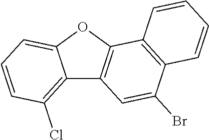 [SMILES: Clc1cccc2oc3c4ccccc4c(Br)cc3c12] | 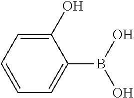 | 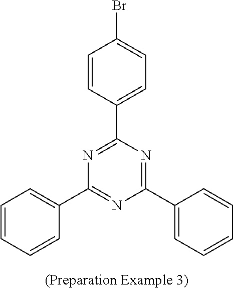 [SMILES: Brc1ccc(-c2nc(-c3ccccc3)nc(-c3ccccc3)n2)cc1] |
| 360 | 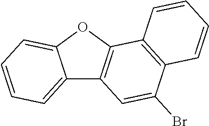 [SMILES: Brc1cc2c3ccccc3oc2c2ccccc12] | 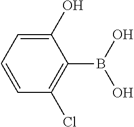 [SMILES: OB(O)c1c(O)cccc1Cl] | 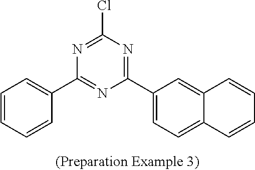 [SMILES: Clc1nc(-c2ccccc2)nc(-c2ccc3ccccc3c2)n1] |
| 378 | 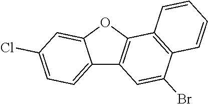 [SMILES: Clc1ccc2c(c1)oc1c3ccccc3c(Br)cc21] | 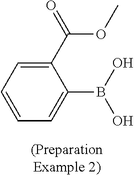 | 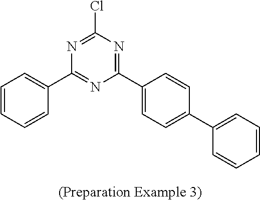 [SMILES: Clc1nc(-c2ccccc2)nc(-c2ccc(-c3ccccc3)cc2)n1] |
| 381 | 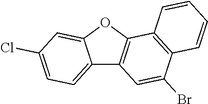 [SMILES: Clc1ccc2c(c1)oc1c3ccccc3c(Br)cc21] | 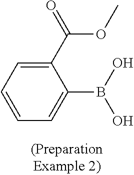 | 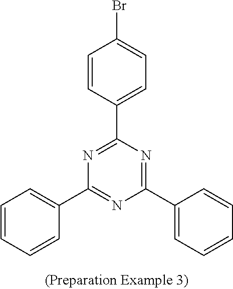 [SMILES: Brc1ccc(-c2nc(-c3ccccc3)nc(-c3ccccc3)n2)cc1] |
| 419 | 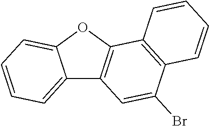 [SMILES: Brc1cc2c3ccccc3oc2c2ccccc12] | 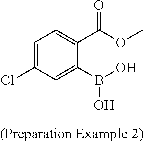 | 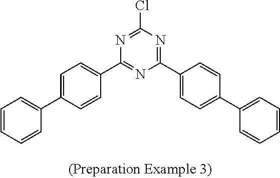 [SMILES: Clc1nc(-c2ccc(-c3ccccc3)cc2)nc(-c2ccc(-c3ccccc3)cc2)n1] |
| 430 | 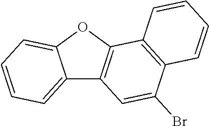 [SMILES: Brc1cc2c3ccccc3oc2c2ccccc12] | 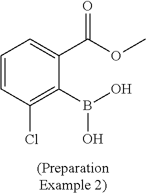 | 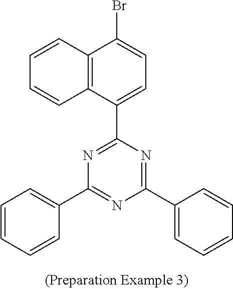 [SMILES: Brc1ccc(-c2nc(-c3ccccc3)nc(-c3ccccc3)n2)c2ccccc12] |
| 443 | 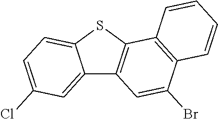 [SMILES: Clc1ccc2sc3c4ccccc4c(Br)cc3c2c1] | 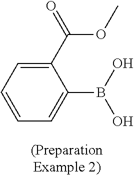 | 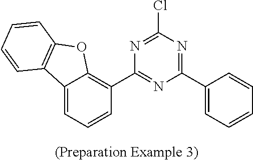 [SMILES: Clc1nc(-c2ccccc2)nc(-c2cccc3c2oc2ccccc23)n1] |

### Preparation Example 4

### Preparation of Compound 467

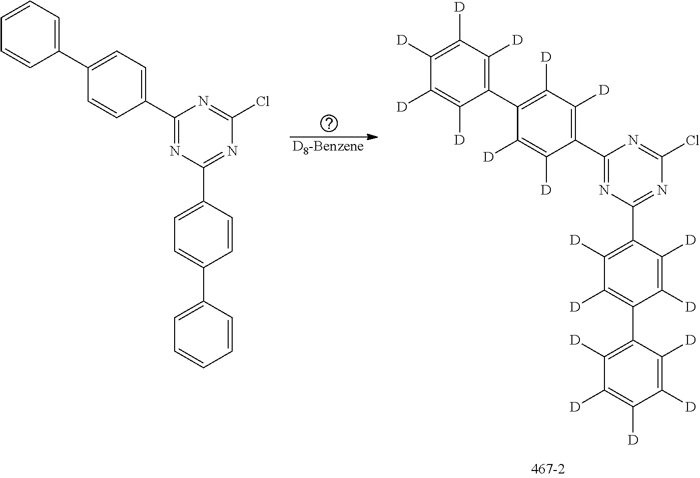 [SMILES: Clc1nc(-c2ccc(-c3ccccc3)cc2)nc(-c2ccc(-c3ccccc3)cc2)n1 ; "D8-Benzene" ; [2H]c1c([2H])c([2H])c(-c2c([2H])c([2H])c(-c3nc(Cl)nc(-c4c([2H])c([2H])c(-c5c([2H])c([2H])c([2H])c([2H])c5[2H])c([2H])c4[2H])n3)c([2H])c2[2H])c([2H])c1[2H]]

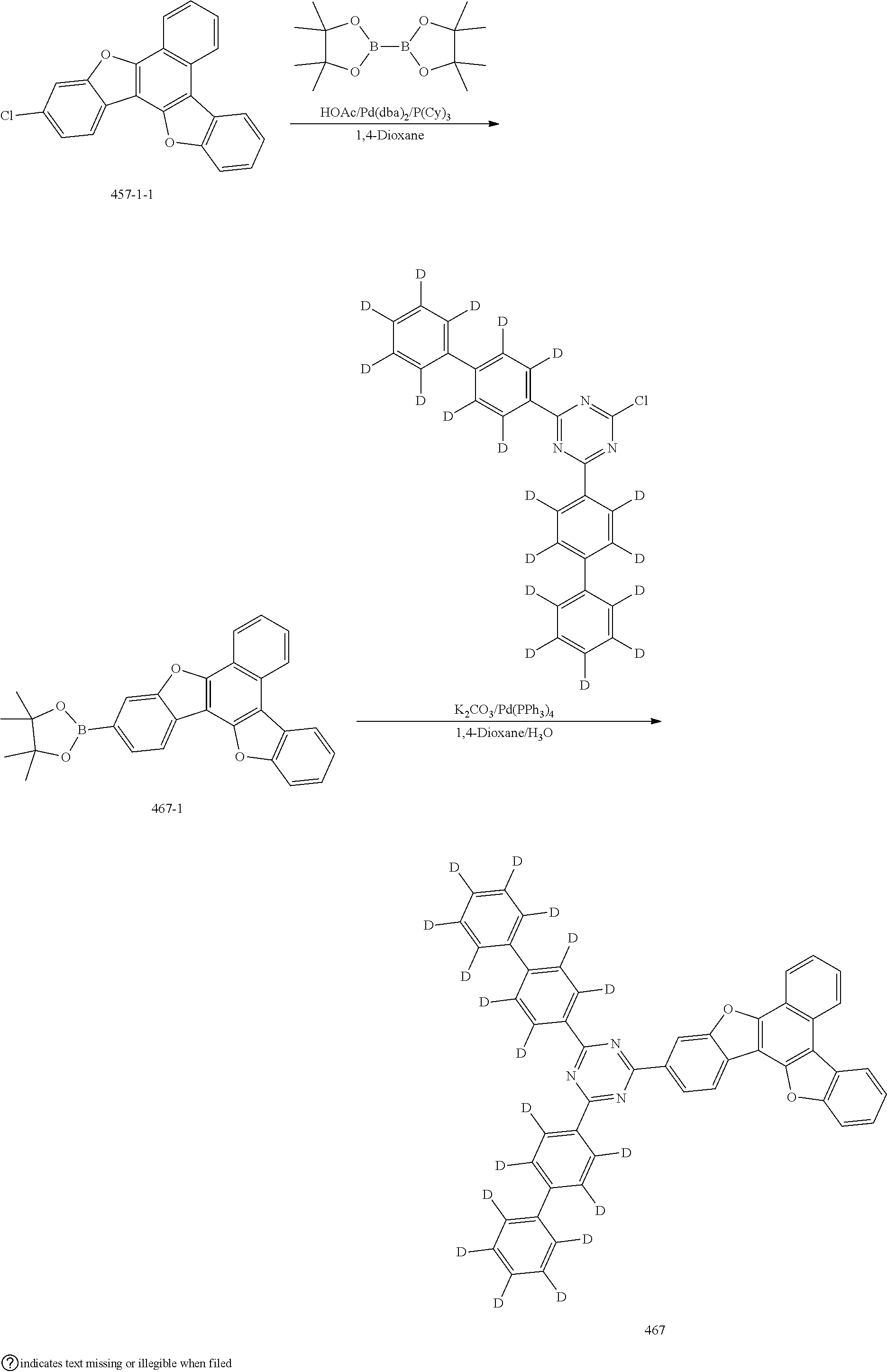

### 1) Preparation of Intermediate 467-2

To a reaction flask, 2,4-di([1,1′-biphenyl]-4-yl)-6-chloro-1,3,5-triazine (15 g, 0.036 mol, 1 eq.), TfOH (8 g, 0.054 mol, 1.5 eq.) and D₆-benzene (150 ml) were introduced, and the mixture was stirred for 8 hours at 100° C. After terminating the reaction by introducing water thereto, the result was extracted using MC and water. After that, water was removed with MgSO₄. The result was separated by a silica gel column to obtain Intermediate 467-2 (10 g) in a 64% yield.

### 2) Preparation of Intermediate 467-1-1

Intermediate 467-1-1 was prepared in the same manner as in 1) and 2) of Preparation Example 1 except that, in 1) of Preparation Example 1, 5-bromo-9-chloronaphtho[1,2-b]benzofuran was used instead of Compound (A).

### 3) Preparation of Intermediate 467-1

Intermediate 467-1 was prepared in the same manner as in 1) of Preparation Example 3 except that Intermediate 467-1-1 was used instead of Intermediate 3-1.

### 4) Preparation of Compound 467

To a reaction flask, Intermediate 467-1 (10.4 g, 0.024 mol, 10.5 eq.), Intermediate 467-2 (10 g, 0.023 mol, 1 eq.), K₂CO₃ (7.9 g, 0.057 mol, 2.5 eq.) and Pd(PPh₃)₄ (1.3 g, 0.001 mol, 0.05 eq.) were introduced, and after introducing 1,4-dioxane (200 ml) and H₂0 (50 ml) thereto, the mixture was stirred for 8 hours at 100° C. After lowering the temperature, the result was extracted using MC and water. After that, water was removed with MgSO₄. The result was separated by a silica gel column to obtain Compound 467 (9 g) in a 56% yield.

### Preparation Example 5

### Preparation of Compound 481

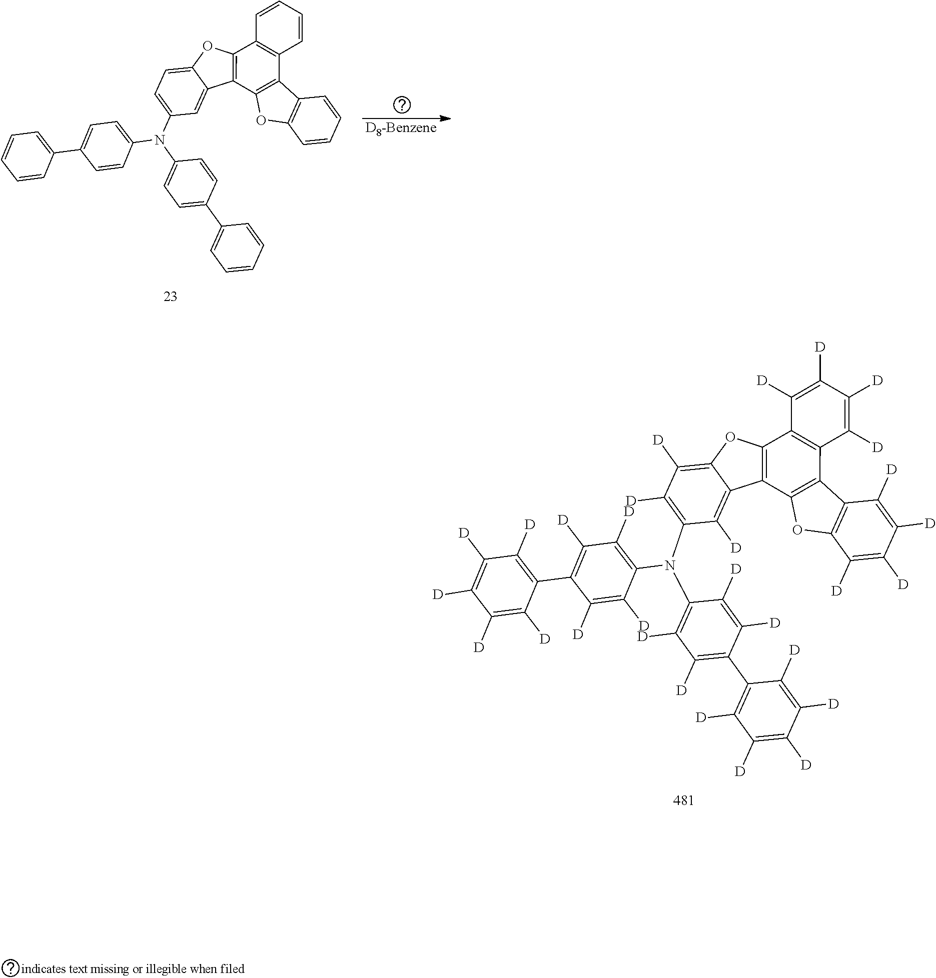

To a reaction flask, Compound 23 (10 g, 0.016 mol, 1 eq.) of Table 1, TfOH (3.6 g, 0.024 mol, 1.5 eq.) and D₆-benzene (100 ml) were introduced, and the mixture was stirred for 8 hours at 100° C. After terminating the reaction by introducing water thereto, the result was extracted using MC and water. After that, water was removed with MgSO₄. The result was separated by a silica gel column to obtain Compound 481 (7 g) in a 67% yield.

Compounds were prepared in the same manner as in the preparation examples, and the synthesis identification results are shown in Table 2 and Table 3. Table 2 shows measurement values of ¹H NMR (CDCl₃, 200 MHz), and Table 3 shows measurement values of FD-mass spectrometry (FD-MS: field desorption mass spectrometry).

TABLE 2

| Compound | ¹H NMR (CDCl₃, 200 Mz) |
|---|---|
| 2 | δ = 8.54(1H, d), 8.16(1H, d), 7.89(1H, d), 7.66(3H, m), |
| | 7.51~7.20(12H, m), 7.07(1H, t), 6.81(1H, t), 6.63(4H, |
| | m), 6.39(1H, d) |
| 23 | δ = 8.54(1H, d), 8.16(1H, d), 7.89(1H, d), 7.67(4H, m), |
| | 7.51~7.32(17H, m), 6.69(4H, m), 6.39(1H, d) |
| 44 | δ = 8.54(1H, d), 8.16(1H, d), 7.89(1H, d), 7.67(3H, m), |
| | 7.51~7.25(21H, m), 7.07(1H, t), 6.69(4H, m), 6.39(1H, |
| | d) |
| 66 | δ = 8.54(1H, d), 8.16(1H, d), 7.89~7.50(14H, m), 7.38(6H, |
| | m), 6.75(1H, s), 6.58(1H, d), 6.39(1H, d), 1.72(6H, s) |
| 93 | δ = 8.54(1H, d), 8.18(1H, d), 8.09(1H, d), 7.61~7.41(18H, |
| | m), 7.25(2H, m), 7.07(1H, t), 6.69(4H, m), 6.39(1H, d), |
| | 1.78(6H, s) |
| 103 | δ = 8.55(1H, d), 8.18(1H, d), 8.09(1H, d), 7.64~7.41(13H, |
| | m), 7.20(3H, m), 6.81(1H, t), 6.69(4H, m), 6.33(1H, d), |
| | 1.78(6H, s) |
| 134 | δ = 8.55(1H, d), 8.18(1H, d), 8.09(1H, d), 7.66(1H, d), |
| | 7.55~7.19(24H, m), 6.69(4H, m), 6.44(1H, d), 1.78(6H, |
| | s) |
| 160 | δ = 8.55(1H, d), 8.45(1H, d), 8.18(1H, d), 7.98(1H, d), |
| | 7.89(1H, d), 7.80(1H, d), 7.66(1H, d), 7.55~7.26(15H, |
| | m), 7.06(1H, s), 6.88(1H, d), 6.69(2H, m), 6.44(1H, d), |
| | 1.78(6H, s) |
| 170 | δ = 8.55(1H, d), 8.18(1H, d), 7.89(2H, m), 7.64(3H, m), |
| | 7.54~7.32(14H, m), 6.99(2H, m), 6.69(3H, m), 6.33(1H, |
| | d), 1.78(6H, s) |
| 194 | δ = 8.55(1H, d), 8.16(1H, d), 7.89(1H, d), 7.80(1H, d), |
| | 7.67(3H, m), 7.54~7.25(20H, m), 7.06(1H, s), 6.88(1H, |
| | d), 6.69(4H, m) |
| 207 | δ = 8.55(1H, d), 8.09(2H, m), 7.87(1H, d), 7.73(1H, d), |
| | 7.62~7.38(15H, m), 7.24(2H, m), 6.86(1H, d), 6.75(1H, |
| | s), 6.69(2H, m), 6.58(1H, d), 1.78(6H, s), 1.72(6H. s) |
| 235 | δ = 8.55(1H, d), 8.45(1H, d), 8.08(1H, d), 7.98(1H, d), |
| | 7.84(2H, m), 7.55(6H, m), 7.38(1H, t), 7.20(3H, m), |
| | 6.81(3H, m), 6.63(4H, m), 1.78(6H, s), 1.72(6H. s) |
| 258 | δ = 8.54(1H, d), 8.45(1H, d), 8.16(1H, d), 7.98(1H, d), |
| | 7.87(2H, m), 7.62~7.50(8H, m), 7.38(2H, m), 7.28(2H, |
| | m), 7.13(1H, d), 7.02(1H, d), 6.75(2H, m), 6.58(2H, m), |
| | 6.33(1H, d), 1.72(12H. s) |
| 275 | δ = 8.55(1H, d), 8.09(2H, d), 8.00(2H, d), 7.86(1H, d), |
| | 7.61~7.41(20H, m), 7.24(1H, t), 6.69(6H, m), 1.78(6H. |
| | s) |
| 281 | δ = 8.55(1H, d), 8.45(1H, d), 8.15(1H, d), 8.08(1H, d), |
| | 7.98(1H, d), 7.83(1H, s), 7.69(1H, d), 7.55(20H, m), |
| | 6.69(6H, m), 1.78(6H. s) |
| 301 | δ = 8.54(1H, d), 8.28(4H, m), 8.16(1H, d), 7.85(3H, m), |
| | 7.67(3H, m), 7.51~7.32(9H. m) |
| 329 | δ = 8.54(1H, d), 8.28(4H, m), 8.16(1H, d), 7.89(3H, m), |
| | 7.62(5H, m), 7.51~7.25(11H. m) |
| 360 | δ = 9.09(1H, s), 8.54(2H, m), 8.28(2H, m), 8.16(1H, d), |
| | 7.89(4H, m), 7.62~7.32(13H. m) |
| 378 | δ = 8.55(1H, d), 8.28(2H, m), 8.18(1H, d), 8.09(1H, d), |
| | 7.95(1H, d), 7.85(2H, m), 7.75(1H, d), 7.64~7.44(13H. |
| | m), 7.25(3H, m), 1.78(6H, s) |
| 381 | δ = 8.55(1H, d), 8.28(4H, m), 8.18(1H, d), 8.09(1H, d), |
| | 7.95(1H, d), 7.85(2H, m), 7.75(1H, d), 7.64~7.44(11H. |
| | m), 7.24(3H, m), 1.78(6H, s) |
| 419 | δ = 8.55(1H, d), 8.28(1H, s), 8.18(1H, d), 7.85(5H, m), |
| | 7.67(2H, m), 7.55~7.25(19H. m), 1.78(6H, s) |
| 430 | δ = 8.55(3H, m), 8.28(4H, m), 8.18(1H, d), 8.01(2H, m), |
| | 7.89(1H, d), 7.69(2H, m), 7.55~7.30(14H. m), 1.78(6H, |
| | s) |
| 443 | δ = 8.55(1H, d), 8.28(2H, m), 8.09(2H, m), 8.00(1H, s), |
| | 7.85(5H, m), 7.66~7.24(12H. m), 1.78(6H, s) |
| 467 | δ = 8.54(1H, d), 8.16(1H, d), 7.95(1H, d), 7.98(1H, d), |
| | 7.64(5H, m), 7.32(2H. m) |

TABLE 3

| Compound | FD-MS | Compound | FD-MS |
|---|---|---|---|
| 2 | m/z = 551.19 | 275 | m/z = 745.28 |
| 23 | m/z = 627.22 | 281 | m/z = 745.28 |
| 44 | m/z = 703.25 | 301 | m/z = 539.16 |
| 66 | m/z = 641.24 | 329 | m/z = 615.19 |
| 93 | m/z = 653.27 | 360 | m/z = 589.18 |
| 103 | m/z = 577.24 | 378 | m/z = 641.25 |
| 134 | m/z = 729.30 | 381 | m/z = 641.25 |
| 160 | m/z = 683.23 | 419 | m/z = 717.28 |
| 170 | m/z = 667.25 | 430 | m/z = 691.26 |
| 194 | m/z = 719.23 | 443 | m/z = 671.20 |
| 207 | m/z = 709.28 | 467 | m/z = 709.34 |
| 235 | m/z = 633.25 | 481 | m/z = 656.40 |
| 258 | m/z = 723.26 | | |

### Experimental Example 1

### 1) Manufacture of Organic Light Emitting Device

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO (ultraviolet ozone) treatment was conducted for 5 minutes using UV in a UV (ultraviolet) cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino]triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. The light emitting layer was deposited to 400 Å by depositing a compound described in the following Table 4 as a red host, and doping (piq)₂(Ir)_{(acac)} to the host by 3 wt % as a red phosphorescent dopant. After that, Bphen (bathophenanthroline) was deposited to 30 Å as a hole blocking layer, and Alq₃ was deposited to 250 Å thereon as an electron transfer layer. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic electroluminescent device was manufactured.

Meanwhile, all the organic compounds required to manufacture the OLED were vacuum sublimation purified under 10⁻⁸ torr to 10⁻⁶ torr for each material to be used in the OLED manufacture.

### 2) Performance Evaluation on Organic Light Emitting Device

For each of the organic electroluminescent devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Results of measuring driving voltage, light emission efficiency, color coordinate (CIE) and lifetime of the organic light emitting devices manufactured according to the present disclosure are as shown in Table 4.

### Comparative Example Compound

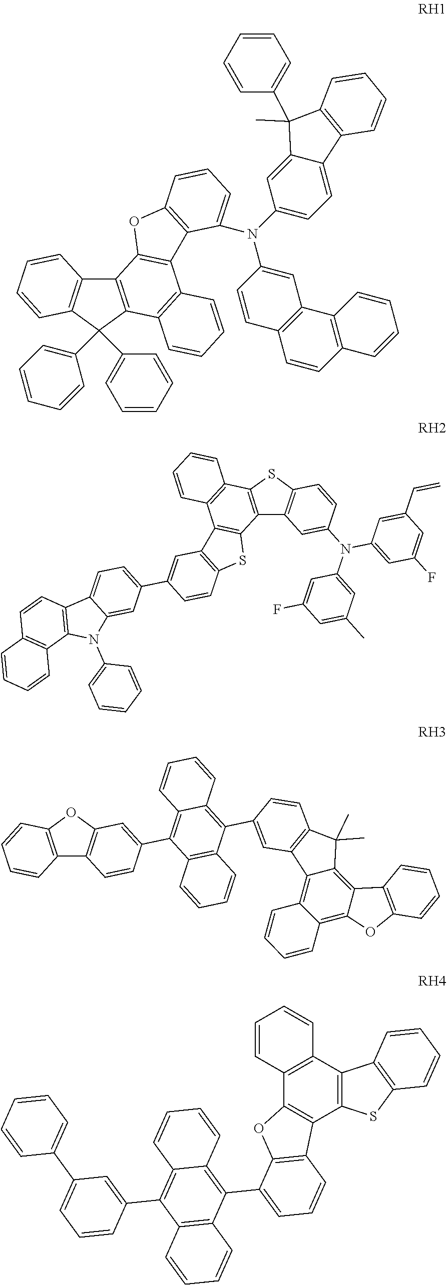 [SMILES: CC1(c2ccccc2)c2ccccc2-c2ccc(N(c3ccc4ccc5ccccc5c4c3)c3cccc4oc5c6c(c7ccccc7c5c34)C(c3ccccc3)(c3ccccc3)c3ccccc3-6)cc21 ; C=Cc1cc(F)cc(N(c2cc(C)cc(F)c2)c2ccc3sc4c5ccccc5c5c6cc(-c7ccc8c9ccc%10ccccc%10c9n(-c9ccccc9)c8c7)ccc6sc5c4c3c2)c1 ; CC1(C)c2ccc(-c3c4ccccc4c(-c4ccc5c(c4)oc4ccccc45)c4ccccc34)cc2-c2c1c1c3ccccc3oc1c1ccccc21 ; c1ccc(-c2cccc(-c3c4ccccc4c(-c4cccc5c4oc4c6ccccc6c6c7ccccc7sc6c54)c4ccccc34)c2)cc1]

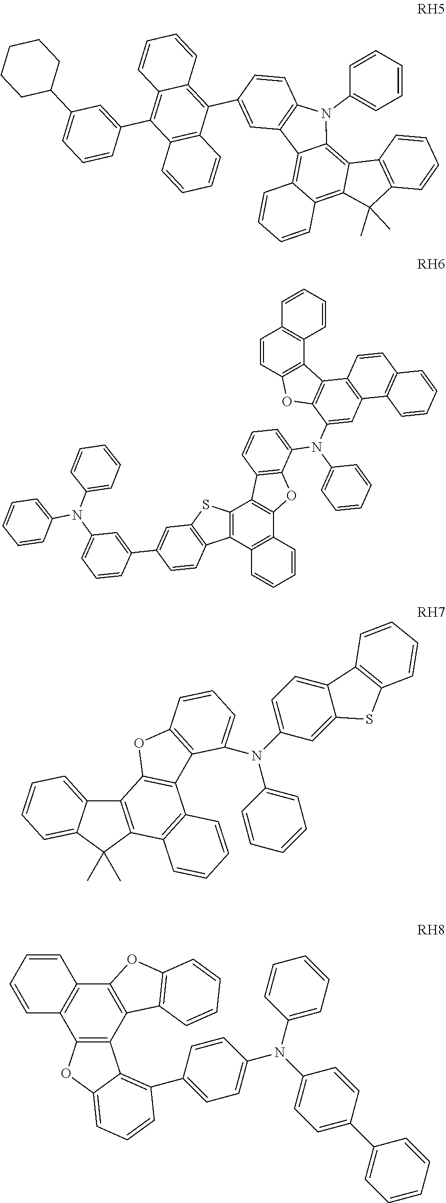 [SMILES: CC1(C)c2ccccc2-c2c1c1ccccc1c1c3cc(-c4c5ccccc5c(-c5cccc(C6CCCCC6)c5)c5ccccc45)ccc3n(-c3ccccc3)c21 ; c1ccc(N(c2ccccc2)c2cccc(-c3ccc4c(c3)sc3c4c4ccccc4c4oc5c(N(c6ccccc6)c6cc7c8ccccc8ccc7c7c6oc6ccc8ccccc8c67)cccc5c43)c2)cc1 ; CC1(C)c2ccccc2-c2c1c1ccccc1c1c2oc2cccc(N(c3ccccc3)c3ccc4c(c3)sc3ccccc34)c21 ; c1ccc(-c2ccc(N(c3ccccc3)c3ccc(-c4cccc5oc6c7ccccc7c7oc8ccccc8c7c6c45)cc3)cc2)cc1]

TABLE 4

| | | | Light | | |
|---|---|---|---|---|---|
| | | Driving | Emission | | |
| | Com- | Voltage | Efficiency | | Lifetime |
| | pound | (V) | (cd/A) | CIE (x, y) | (T₉₀) |
| Example 1 | 2 | 5.38 | 45.7 | (0.681, 0.320) | 99 |
| Example 2 | 23 | 5.30 | 46.8 | (0.678, 0.318) | 103 |
| Example 3 | 44 | 5.43 | 47.2 | (0.677, 0.324) | 99 |
| Example 4 | 66 | 5.38 | 45.6 | (0.682, 0.321) | 91 |
| Example 5 | 93 | 5.44 | 45.4 | (0.679, 0.320) | 94 |
| Example 6 | 103 | 5.38 | 47.5 | (0.683, 0.323) | 97 |
| Example 7 | 134 | 5.40 | 45.3 | (0.676, 0.322) | 103 |
| Example 8 | 160 | 5.41 | 46.9 | (0.681, 0.321) | 98 |
| Example 9 | 170 | 5.38 | 45.6 | (0.680, 0.323) | 95 |
| Example 10 | 194 | 5.39 | 45.4 | (0.682, 0.320) | 98 |
| Example 11 | 207 | 5.40 | 46.3 | (0.684, 0.321) | 100 |
| Example 12 | 235 | 5.45 | 48.7 | (0.678, 0.323) | 96 |
| Example 13 | 258 | 5.37 | 46.7 | (0.683, 0.324) | 97 |
| Example 14 | 275 | 5.36 | 47.4 | (0.676, 0.320) | 101 |
| Example 15 | 281 | 5.31 | 46.0 | (0.681, 0.319) | 98 |
| Example 16 | 302 | 5.40 | 47.1 | (0.682, 0.325) | 95 |
| Example 17 | 329 | 4.97 | 51.8 | (0.683, 0.323) | 127 |
| Example 18 | 360 | 4.90 | 50.9 | (0.676, 0.320) | 123 |
| Example 19 | 378 | 4.91 | 51.5 | (0.681, 0.321) | 128 |
| Example 20 | 381 | 4.93 | 50.4 | (0.683, 0.325) | 129 |
| Example 21 | 419 | 4.97 | 51.6 | (0.682, 0.324) | 125 |
| Example 22 | 430 | 4.91 | 50.1 | (0.677, 0.322) | 122 |
| Example 23 | 443 | 4.96 | 50.8 | (0.681, 0.320) | 127 |
| Example 24 | 467 | 4.98 | 51.2 | (0.680, 0.323) | 124 |
| Example 25 | 481 | 5.37 | 46.9 | (0.677, 0.320) | 105 |
| Comparative | RH1 | 5.83 | 42.9 | (0.674, 0.329) | 73 |
| Example 1 | | | | | |
| Comparative | RH2 | 5.94 | 41.3 | (0.675, 0.328) | 68 |
| Example 2 | | | | | |
| Comparative | RH3 | 5.85 | 41.2 | (0.678, 0.326) | 53 |
| Example 3 | | | | | |
| Comparative | RH4 | 5.92 | 40.5 | (0.681, 0.320) | 55 |
| Example 4 | | | | | |
| Comparative | RH5 | 5.89 | 40.9 | (0.679, 0.324) | 70 |
| Example 5 | | | | | |
| Comparative | RH6 | 5.97 | 41.7 | (0.681, 0.322) | 74 |
| Example 6 | | | | | |
| Comparative | RH7 | 5.80 | 42.8 | (0.680, 0,321) | 78 |
| Example 7 | | | | | |
| Comparative | RH8 | 5.81 | 42.0 | (0.681, 0.322) | 76 |
| Example 8 | | | | | |

As seen from the results of Table 4, it was identified that the organic light emitting device using the host material of the present disclosure had lower driving voltage, and significantly improved light emission efficiency and lifetime compared to the comparative examples. The condensed polycyclic compound according to the present application has, while having high thermal stability, proper molecular weight and band gap to be used in a light emitting layer of an organic light emitting device. The proper molecular weight facilitates formation of a light emitting layer of an organic light emitting device, and the proper band gap prevents loss of electrons and holes in the light emitting layer helping with effective formation of a recombination zone. In addition, the condensed polycyclic compound having electron transfer properties substituted at a proper position resolves a hole blocking phenomenon occurring in the dopant compared to the compounds substituted at other positions, and, as seen from the evaluation results on the device, it is considered that the compound of the present disclosure brings superiority in terms of driving voltage, efficiency and lifetime compared to the comparative examples.

### Experimental Example 2

### 1) Manufacture of Organic Light Emitting Device

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO (ultraviolet ozone) treatment was conducted for 5 minutes using UV in a UV (ultraviolet) cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino]triphenylamine), a hole transfer layer NPB (N,N′-di(1-naphthyl)-N,N′-diphenyl-(1,1′-biphenyl)-4,4′-diamine) and an electron blocking layer TAPC (cyclohexylidenebis[N,N-bis(4-methylphenyl)benzenamine]), which are common layers, were formed.

A light emitting layer was thermal vacuum deposited thereon as follows. The light emitting layer was deposited to 400 Å by depositing two types of compounds described in the following Table 5 in one source of supply in a weight ratio described in the following Table 5 as a red host, and doping (piq)₂(Ir)_{(acac)} to the host by 3 wt % as a red phosphorescent dopant. After that, Bphen was deposited to 30 Å as a hole blocking layer, and TPBI (2,2′,2″-(1,3,5-benzinetriyl)-tris(1-phenyl-1-H-benzimidazole) was deposited to 250 Å thereon as an electron transfer layer. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic electroluminescent device was manufactured.

Meanwhile, all the organic compounds required to manufacture the OLED were vacuum sublimation purified under 10⁻⁸ torr to 10⁻⁶ torr for each material to be used in the OLED manufacture.

### 2) Performance Evaluation on Organic Light Emitting Device

For each of the organic electroluminescent devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Results of measuring driving voltage, light emission efficiency, color coordinate (CIE) and lifetime of the organic light emitting devices manufactured according to the present disclosure are as shown in Table 5.

TABLE 5

| | | | | Light | | |
|---|---|---|---|---|---|---|
| | | | Driving | Emission | | |
| | Compound | Ratio | Voltage | Efficiency | | Lifetime |
| | (P:N) | (P:N) | (V) | (cd/A) | CIE (x, y) | (T₉₀) |
| Example 26 | 2:329 | 1:3 | 4.79 | 54.77 | (0.680, 0.323) | 141 |
| Example 27 | 2:329 | 1:2 | 4.72 | 55.12 | (0.681, 0.321) | 141 |
| Example 28 | 2:329 | 1:1 | 4.69 | 55.74 | (0.680, 0.320) | 155 |
| Example 29 | 2:329 | 2:1 | 4.73 | 54.89 | (0.679, 0.321) | 147 |
| Example 30 | 2:329 | 3:1 | 4.80 | 54.73 | (0.678, 0.320) | 145 |
| Example 31 | 23:329 | 1:1 | 4.78 | 55.67 | (0.679, 0.322) | 141 |
| Example 32 | 23:360 | 1:1 | 4.76 | 56.34 | (0.677, 0.324) | 140 |
| Example 33 | 44:360 | 1:1 | 4.80 | 55.46 | (0.675, 0.325) | 145 |
| Example 34 | 44:378 | 1:1 | 4.71 | 56.19 | (0.680, 0.320) | 139 |
| Example 35 | 66:378 | 1:1 | 4.72 | 54.20 | (0.681, 0.324) | 138 |
| Example 36 | 66:381 | 1:1 | 4.79 | 55.13 | (0.680, 0.323) | 136 |
| Example 37 | 93:381 | 1:1 | 4.82 | 55.67 | (0.681, 0.322) | 143 |
| Example 38 | 93:419 | 1:1 | 4.69 | 54.56 | (0.680, 0.324) | 141 |
| Example 39 | 103:419 | 1:1 | 4.73 | 53.96 | (0.679, 0.319) | 144 |
| Example 40 | 103:430 | 1:1 | 4.71 | 56.01 | (0.683, 0.321) | 132 |
| Example 41 | 134:430 | 1:1 | 4.81 | 55.13 | (0.678, 0.318) | 142 |
| Example 42 | 134:443 | 1:1 | 4.69 | 54.47 | (0.680, 0.321) | 140 |
| Example 43 | 160:443 | 1:1 | 4.73 | 54.65 | (0.679, 0.320) | 134 |
| Example 44 | 160:467 | 1:1 | 4.78 | 56.51 | (0.678, 0.321) | 138 |
| Example 45 | 170:467 | 1:1 | 4.77 | 55.30 | (0.681, 0.321) | 140 |
| Example 46 | 170:329 | 1:1 | 4.69 | 53.70 | (0.680, 0.324) | 138 |
| Example 47 | 194:329 | 1:1 | 4.79 | 54.88 | (0.683, 0.323) | 136 |
| Example 48 | 194:360 | 1:1 | 4.80 | 56.72 | (0.681, 0.322) | 140 |
| Example 49 | 207:360 | 1:1 | 4.76 | 53.93 | (0.678, 0.319) | 141 |
| Example 50 | 207:378 | 1:1 | 4.71 | 56.11 | (0.680, 0.321) | 131 |
| Example 51 | 235:378 | 1:1 | 4.70 | 54.35 | (0.682, 0.320) | 135 |
| Example 52 | 235:381 | 1:1 | 4.70 | 56.56 | (0.681, 0.323) | 142 |
| Example 53 | 258:381 | 1:1 | 4.78 | 55.57 | (0.678, 0.322) | 133 |
| Example 54 | 258:419 | 1:1 | 4.81 | 56.33 | (0.680, 0.320) | 139 |
| Example 55 | 275:419 | 1:1 | 4.80 | 54.46 | (0.679, 0.320) | 141 |
| Example 56 | 275:430 | 1:1 | 4.68 | 53.71 | (0.681, 0.321) | 150 |
| Example 57 | 281:430 | 1:1 | 4.75 | 55.96 | (0.678, 0.322) | 141 |
| Example 58 | 281:443 | 1:1 | 4.76 | 53.52 | (0.680, 0.320) | 139 |
| Example 59 | 481:443 | 1:1 | 4.80 | 54.33 | (0.679, 0.323) | 144 |
| Example 60 | 481:467 | 1:1 | 4.73 | 56.48 | (0.681, 0.321) | 136 |
| Example 61 | 2:467 | 1:1 | 4.68 | 56.98 | (0.680, 0.321) | 151 |
| Comparative | RH1:329 | 1:1 | 5.41 | 45.77 | (0.680, 0.321) | 84 |
| Example 9 | | | | | | |
| Comparative | RH1:360 | 1:1 | 5.32 | 44.23 | (0.683, 0.320) | 85 |
| Example 10 | | | | | | |
| Comparative | RH2:360 | 1:1 | 5.30 | 45.65 | (0.678, 0.324) | 90 |
| Example 11 | | | | | | |
| Comparative | RH2:378 | 1:1 | 5.47 | 44.80 | (0.680, 0.319) | 85 |
| Example 12 | | | | | | |
| Comparative | RH3:378 | 1:1 | 5.33 | 43.51 | (0.676, 0.320) | 88 |
| Example 13 | | | | | | |
| Comparative | RH3:381 | 1:1 | 5.41 | 44.33 | (0.681, 0.321) | 81 |
| Example 14 | | | | | | |
| Comparative | RH4:381 | 1:1 | 5.38 | 45.87 | (0.679, 0.322) | 83 |
| Example 15 | | | | | | |
| Comparative | RH4:419 | 1:1 | 5.44 | 45.42 | (0.681, 0.322) | 87 |
| Example 16 | | | | | | |
| Comparative | RH5:419 | 1:1 | 5.40 | 44.99 | (0.682, 0.321) | 83 |
| Example 17 | | | | | | |
| Comparative | RH5:430 | 1:1 | 5.36 | 45.90 | (0.677, 0.319) | 89 |
| Example 18 | | | | | | |
| Comparative | RH6:430 | 1:1 | 5.39 | 46.53 | (0.679, 0.325) | 82 |
| Example 19 | | | | | | |
| Comparative | RH6:443 | 1:1 | 5.41 | 45.83 | (0.680, 0.324) | 90 |
| Example 20 | | | | | | |
| Comparative | RH7:443 | 1:1 | 5.37 | 43.70 | (0.678, 0.320) | 89 |
| Example 21 | | | | | | |
| Comparative | RH7:467 | 1:1 | 5.30 | 45.22 | (0.682, 0.323) | 81 |
| Example 22 | | | | | | |
| Comparative | RH8:467 | 1:1 | 5.32 | 46.64 | (0.681, 0.322) | 79 |
| Example 23 | | | | | | |
| Comparative | RH8:329 | 1:1 | 5.29 | 45.84 | (0.681, 0.321) | 85 |
| Example 24 | | | | | | |

As seen from the results of Table 5, it was identified that the organic light emitting device had improved driving voltage, efficiency and lifetime when the condensed polycyclic compounds of the present disclosure are each used and deposited as an N-type host and a P-type host.

Specifically, when a donor (P-type host) having a favorable hole transfer ability and an acceptor (N-type host) having a favorable electron transfer ability are used together as a host of a light emitting layer, holes are injected to the P-host and electrons are injected to the N-host due to an exciplex phenomenon of the N+P compound, which balances a charge balance in a device. Accordingly, it was seen that driving voltage, efficiency and lifetime were enhanced when combining the N-type host compound having suitable electron transfer properties and the P-type host compound having suitable hole transfer properties in a proper ratio.

1. A condensed polycyclic compound of the following Chemical Formula 1: 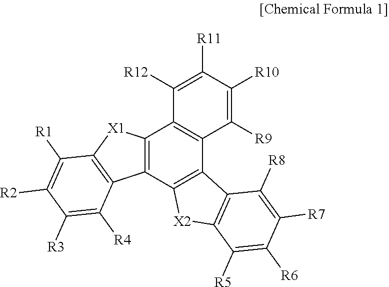
in Chemical Formula 1,
X1 and X2 are the same as or different from each other, and each independently CRR′; O; or S,
R and R′ are the same as or different from each other, and each independently hydrogen; deuterium; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; or a substituted or unsubstituted heteroaryl group having 2 to 60 carbon atoms,
R1 to R12 are the same as or different from each other, and each independently hydrogen; deuterium; a halogen group; a cyano group; —N(R21)m(R22)n; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; or a heteroaryl group having 2 to 60 carbon atoms substituted or unsubstituted and including a double bond of carbon and nitrogen,
m and n are each an integer of 1 to 5,
when m and n are each 2 or greater, substituents in the parentheses are the same as or different from each other,
at least one of R1 to R12 is -(L)q-(Z)r,
L is a direct bond; a substituted or unsubstituted arylene group having 6 to 60 carbon atoms; or a substituted or unsubstituted heteroarylene group having 2 to 60 carbon atoms,
Z is —N(R31)o(R32)p; or a heteroaryl group having 2 to 60 carbon atoms substituted or unsubstituted and including a double bond of carbon and nitrogen,
o, p, q and r are each an integer of 1 to 5,
when o, p, q and r are each 2 or greater, substituents in the parentheses are the same as or different from each other,
when two or more of R1 to R12 are -(L)q-(Z)r, Ls and Zs are each the same as or different from each other,
R21, R22, R31 and R32 are the same as or different from each other, and each independently hydrogen; deuterium; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; or a heteroaryl group having 2 to 60 carbon atoms substituted or unsubstituted and including O or S, or R21 and R22, and R31 and R32 each bond to form a substituted or unsubstituted aliphatic or aromatic hydrocarbon ring having 6 to 60 carbon atoms, or a substituted or unsubstituted aliphatic or aromatic heteroring having 2 to 60 carbon atoms, and
when any one of R21 and R22 or any one of R31 and R32 is a heteroaryl group having 2 to 60 carbon atoms substituted or unsubstituted and including O or S, the other one of R21 and R22 or the other one of R31 and R32 is a substituted or unsubstituted aryl group having 10 to 60 carbon atoms.

2. The condensed polycyclic compound of claim 1, wherein Z is —N(R31)o(R32)p; a substituted or unsubstituted pyrimidine group; a substituted or unsubstituted triazine group; a substituted or unsubstituted quinazoline group; a substituted or unsubstituted quinoxaline group; or a substituted or unsubstituted benzofuropyrimidine group.

3. The condensed polycyclic compound of claim 1, wherein, when Z is —N(R31)o(R32)p, the condensed polycyclic compound satisfies any one of
i) R31 and R32 are the same as or different from each other, and each independently a substituted or unsubstituted aryl group having 6 to 30 carbon atoms,
ii) any one of R31 and R32 is a heteroaryl group having 2 to 30 carbon atoms substituted or unsubstituted and including O or S, and the other one of R31 and R32 is a substituted or unsubstituted aryl group having 10 to 30 carbon atoms, and
iii) R31 and R32 bond to form an aliphatic or aromatic heteroring having 2 to 60 carbon atoms substituted or unsubstituted and including N.

4. The condensed polycyclic compound of claim 1, wherein one or two of R1 to R12 are -(L)q-(Z)r.

5. The condensed polycyclic compound of claim 1, wherein at least one of R1 to R12 is -(L)q-(Z)r, and the rest are hydrogen; deuterium; or a substituted or unsubstituted aryl group having 6 to 30 carbon atoms.

6. The condensed polycyclic compound of claim 1, wherein, when Chemical Formula 1 includes deuterium, a content of the deuterium is from 10% to 100%.

7. The condensed polycyclic compound of claim 1, wherein Chemical Formula 1 is represented by any one of the following compounds:

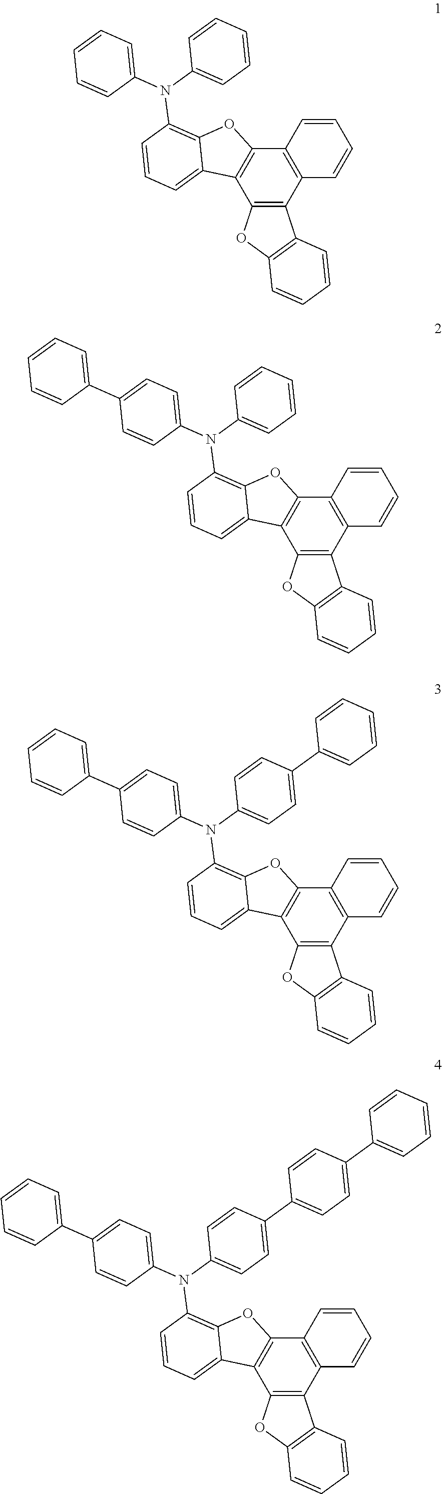

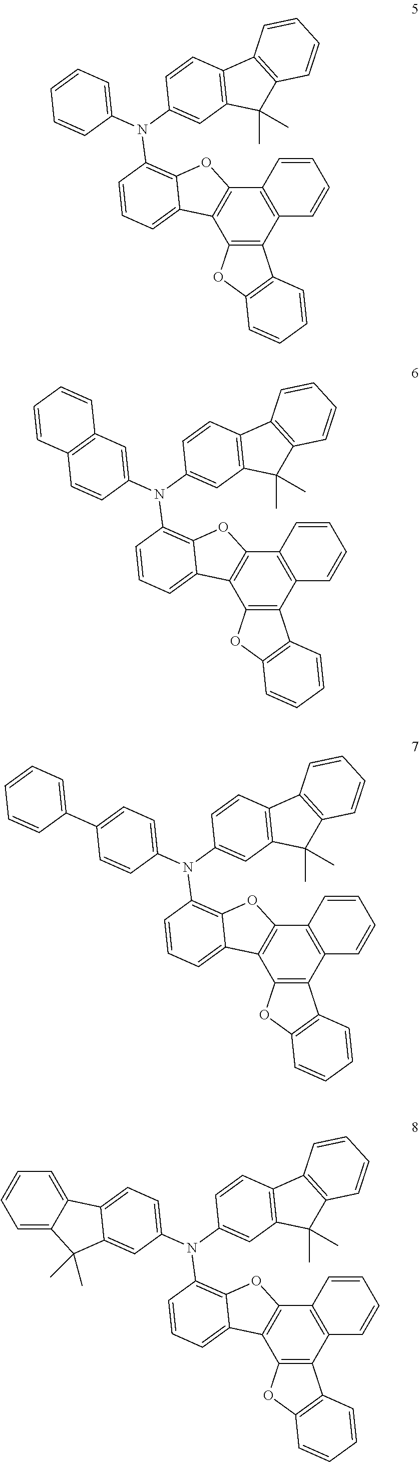

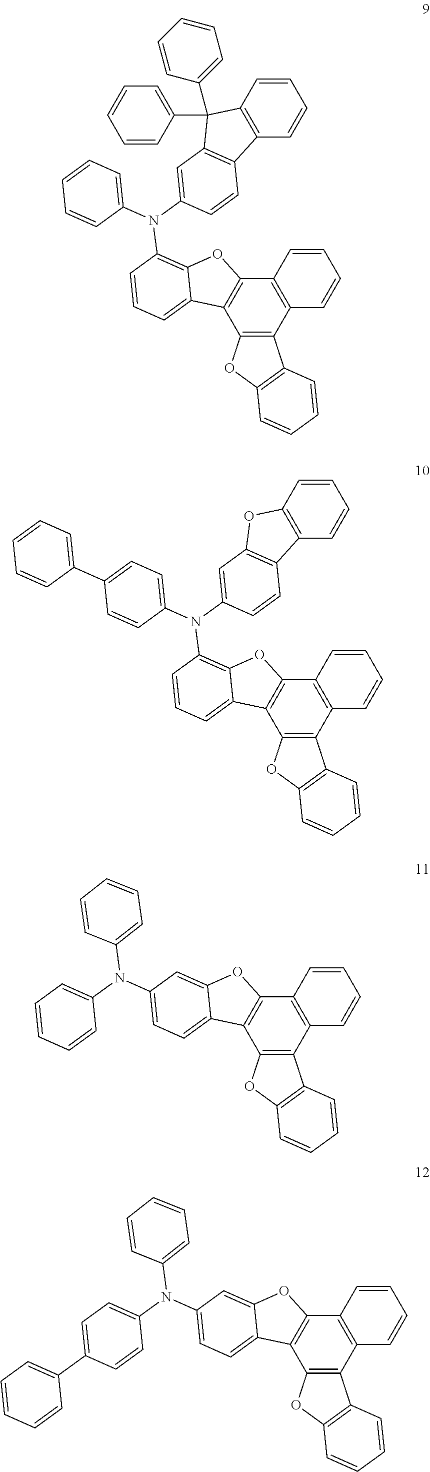

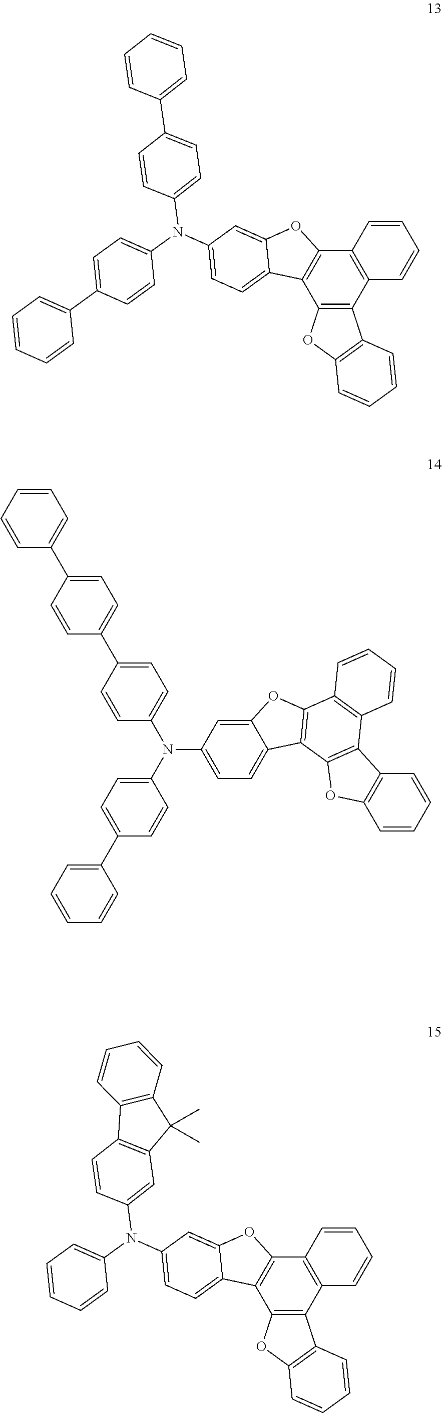 [SMILES: c1ccc(-c2ccc(N(c3ccc(-c4ccccc4)cc3)c3ccc4c(c3)oc3c5ccccc5c5c6ccccc6oc5c43)cc2)cc1 ; c1ccc(-c2ccc(-c3ccc(N(c4ccc(-c5ccccc5)cc4)c4ccc5c(c4)oc4c6ccccc6c6c7ccccc7oc6c54)cc3)cc2)cc1 ; CC1(C)c2ccccc2-c2ccc(N(c3ccccc3)c3ccc4c(c3)oc3c5ccccc5c5c6ccccc6oc5c43)cc21]

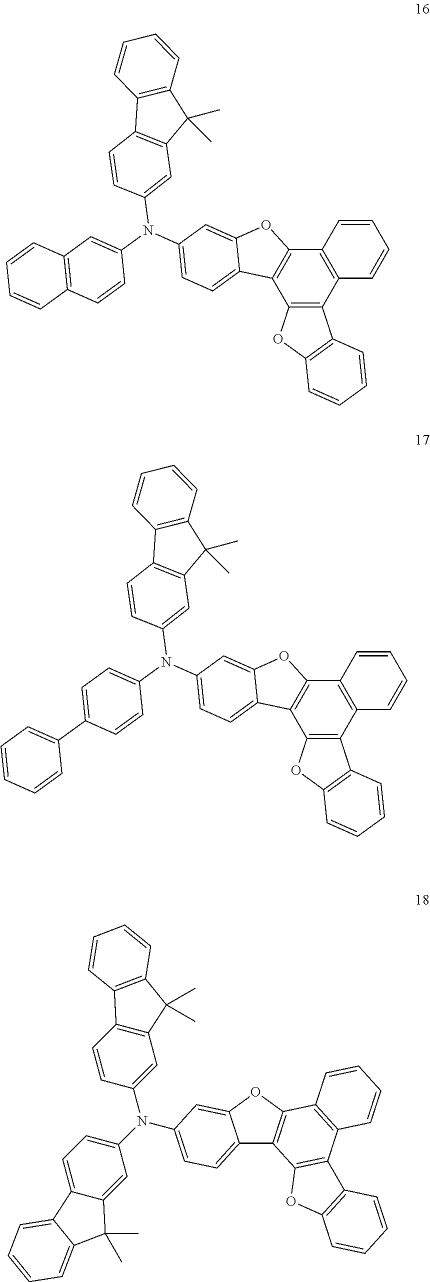 [SMILES: CC1(C)c2ccccc2-c2ccc(N(c3ccc4ccccc4c3)c3ccc4c(c3)oc3c5ccccc5c5c6ccccc6oc5c43)cc21 ; CC1(C)c2ccccc2-c2ccc(N(c3ccc(-c4ccccc4)cc3)c3ccc4c(c3)oc3c5ccccc5c5c6ccccc6oc5c43)cc21 ; CC1(C)c2ccccc2-c2ccc(N(c3ccc4c(c3)C(C)(C)c3ccccc3-4)c3ccc4c(c3)oc3c5ccccc5c5c6ccccc6oc5c43)cc21]

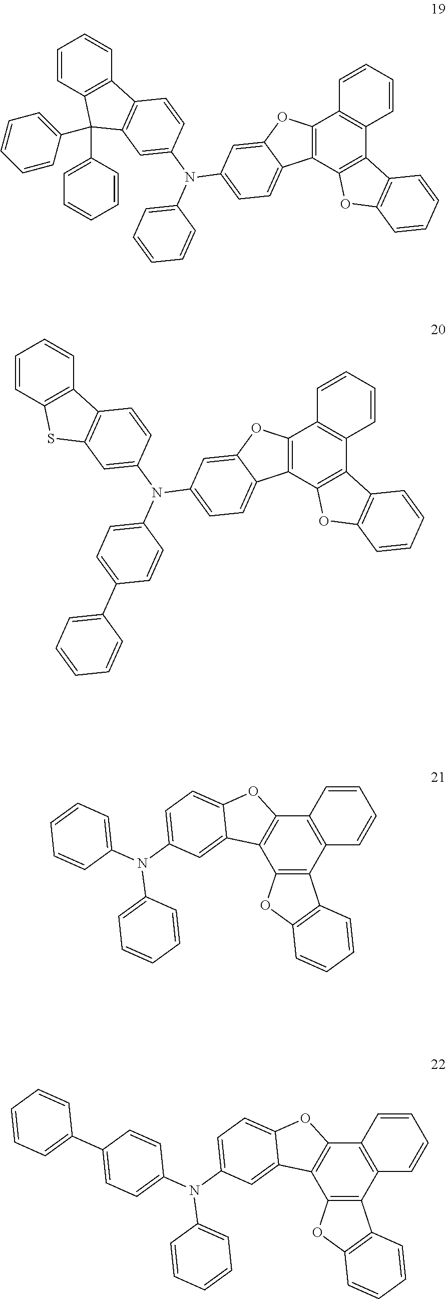 [SMILES: c1ccc(N(c2ccc3c(c2)C(c2ccccc2)(c2ccccc2)c2ccccc2-3)c2ccc3c(c2)oc2c4ccccc4c4c5ccccc5oc4c32)cc1 ; c1ccc(-c2ccc(N(c3ccc4c(c3)oc3c5ccccc5c5c6ccccc6oc5c43)c3ccc4c(c3)sc3ccccc34)cc2)cc1 ; c1ccc(N(c2ccccc2)c2ccc3oc4c5ccccc5c5c6ccccc6oc5c4c3c2)cc1 ; c1ccc(-c2ccc(N(c3ccccc3)c3ccc4oc5c6ccccc6c6c7ccccc7oc6c5c4c3)cc2)cc1]

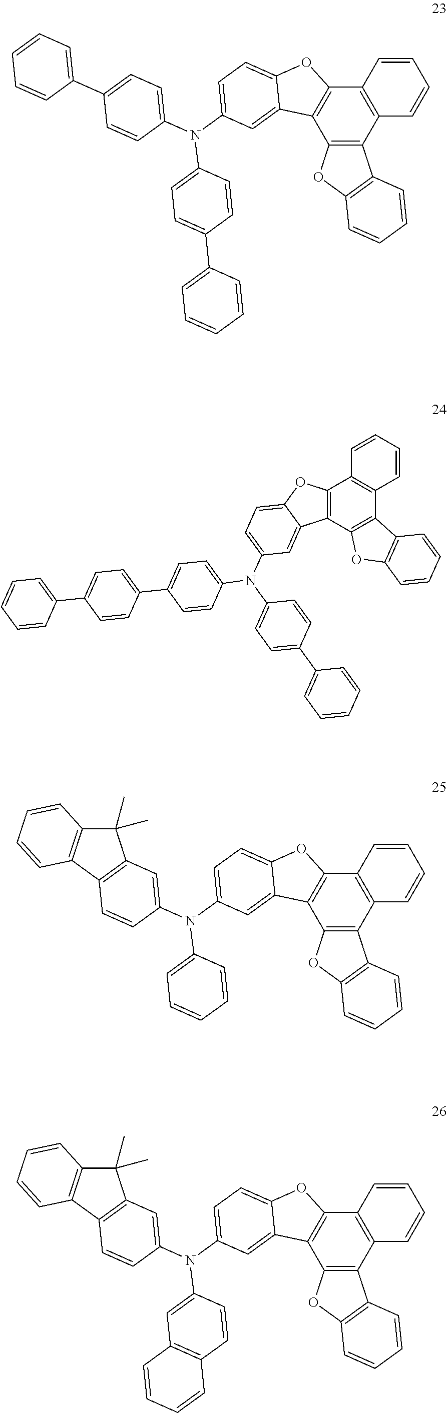 [SMILES: c1ccc(-c2ccc(N(c3ccc(-c4ccccc4)cc3)c3ccc4oc5c6ccccc6c6c7ccccc7oc6c5c4c3)cc2)cc1 ; c1ccc(-c2ccc(-c3ccc(N(c4ccc(-c5ccccc5)cc4)c4ccc5oc6c7ccccc7c7c8ccccc8oc7c6c5c4)cc3)cc2)cc1 ; CC1(C)c2ccccc2-c2ccc(N(c3ccccc3)c3ccc4oc5c6ccccc6c6c7ccccc7oc6c5c4c3)cc21 ; CC1(C)c2ccccc2-c2ccc(N(c3ccc4ccccc4c3)c3ccc4oc5c6ccccc6c6c7ccccc7oc6c5c4c3)cc21]

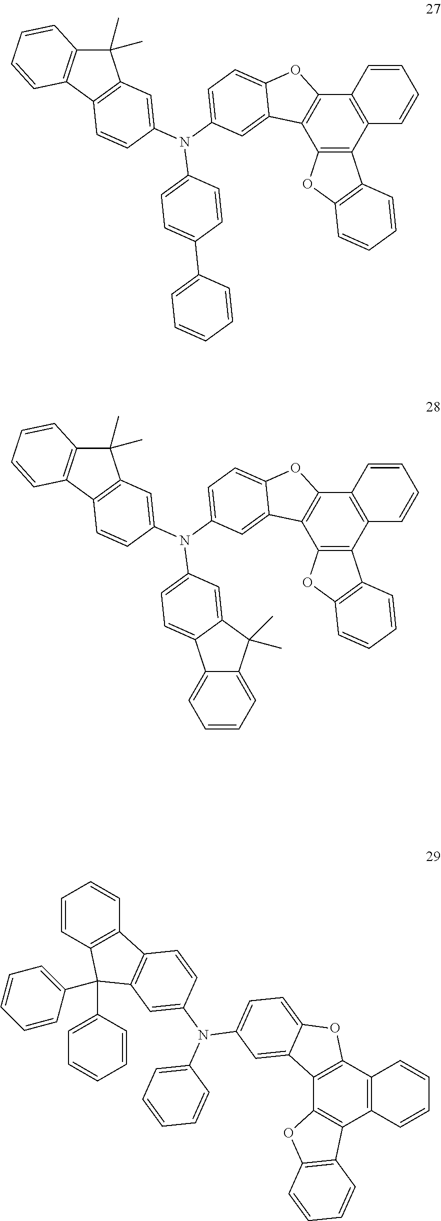 [SMILES: CC1(C)c2ccccc2-c2ccc(N(c3ccc(-c4ccccc4)cc3)c3ccc4oc5c6ccccc6c6c7ccccc7oc6c5c4c3)cc21 ; CC1(C)c2ccccc2-c2ccc(N(c3ccc4c(c3)C(C)(C)c3ccccc3-4)c3ccc4oc5c6ccccc6c6c7ccccc7oc6c5c4c3)cc21 ; c1ccc(N(c2ccc3c(c2)C(c2ccccc2)(c2ccccc2)c2ccccc2-3)c2ccc3oc4c5ccccc5c5c6ccccc6oc5c4c3c2)cc1]

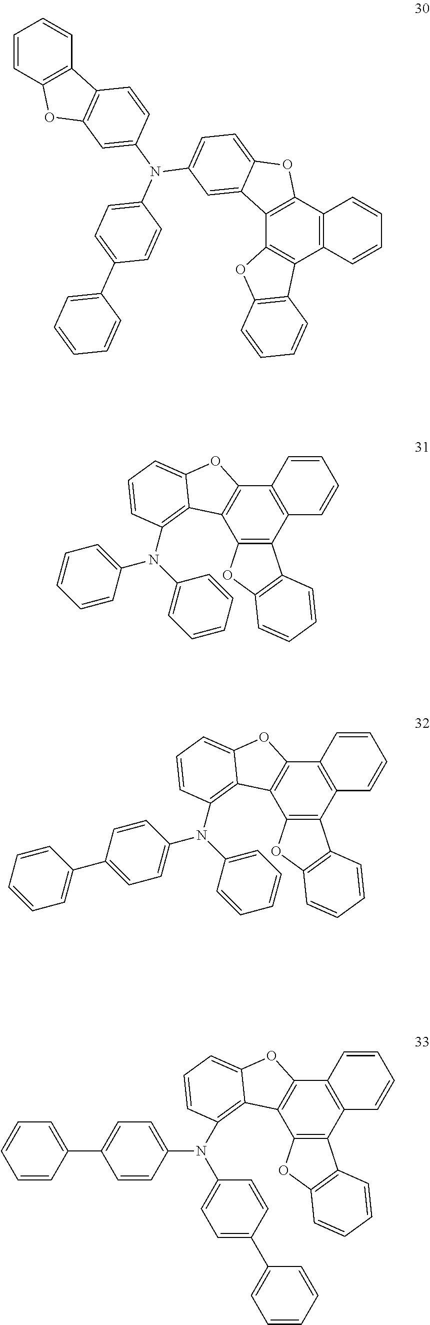 [SMILES: c1ccc(-c2ccc(N(c3ccc4c(c3)oc3ccccc34)c3ccc4oc5c6ccccc6c6c7ccccc7oc6c5c4c3)cc2)cc1 ; c1ccc(N(c2ccccc2)c2cccc3oc4c5ccccc5c5c6ccccc6oc5c4c23)cc1 ; c1ccc(-c2ccc(N(c3ccccc3)c3cccc4oc5c6ccccc6c6c7ccccc7oc6c5c34)cc2)cc1 ; c1ccc(-c2ccc(N(c3ccc(-c4ccccc4)cc3)c3cccc4oc5c6ccccc6c6c7ccccc7oc6c5c34)cc2)cc1]

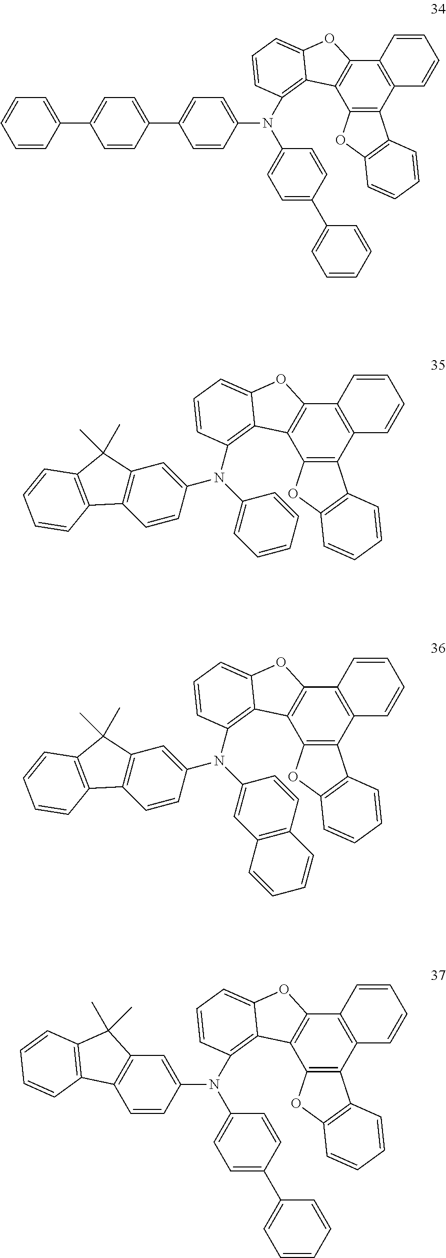 [SMILES: c1ccc(-c2ccc(-c3ccc(N(c4ccc(-c5ccccc5)cc4)c4cccc5oc6c7ccccc7c7c8ccccc8oc7c6c45)cc3)cc2)cc1 ; CC1(C)c2ccccc2-c2ccc(N(c3ccccc3)c3cccc4oc5c6ccccc6c6c7ccccc7oc6c5c34)cc21 ; CC1(C)c2ccccc2-c2ccc(N(c3ccc4ccccc4c3)c3cccc4oc5c6ccccc6c6c7ccccc7oc6c5c34)cc21 ; CC1(C)c2ccccc2-c2ccc(N(c3ccc(-c4ccccc4)cc3)c3cccc4oc5c6ccccc6c6c7ccccc7oc6c5c34)cc21]

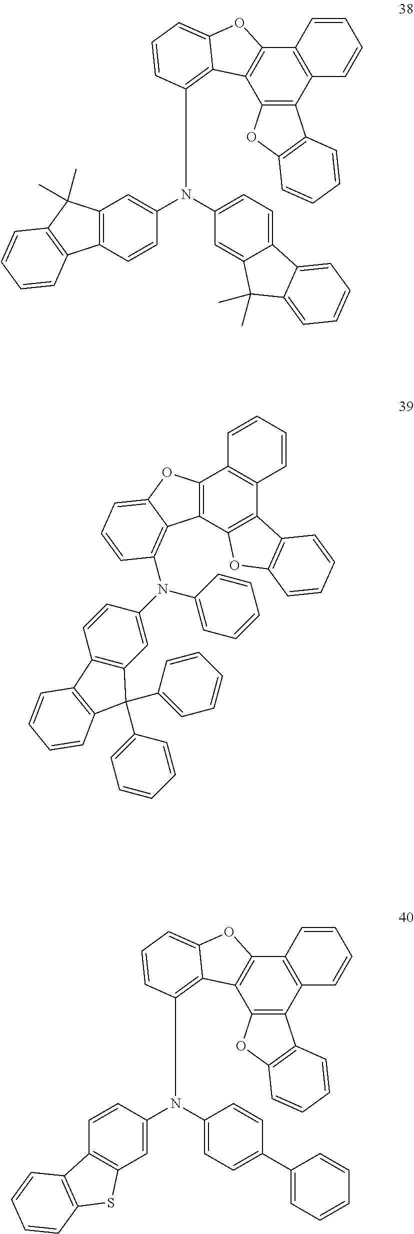 [SMILES: CC1(C)c2ccccc2-c2ccc(N(c3ccc4c(c3)C(C)(C)c3ccccc3-4)c3cccc4oc5c6ccccc6c6c7ccccc7oc6c5c34)cc21 ; c1ccc(N(c2ccc3c(c2)C(c2ccccc2)(c2ccccc2)c2ccccc2-3)c2cccc3oc4c5ccccc5c5c6ccccc6oc5c4c23)cc1 ; c1ccc(-c2ccc(N(c3ccc4c(c3)sc3ccccc34)c3cccc4oc5c6ccccc6c6c7ccccc7oc6c5c34)cc2)cc1]

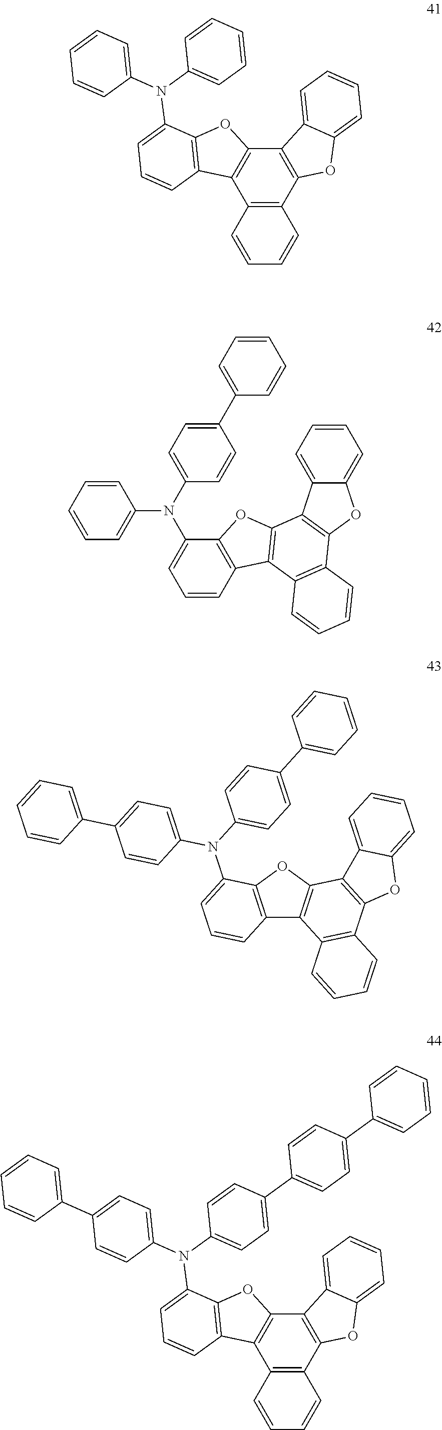 [SMILES: c1ccc(N(c2ccccc2)c2cccc3c2oc2c3c3ccccc3c3oc4ccccc4c32)cc1 ; c1ccc(-c2ccc(N(c3ccccc3)c3cccc4c3oc3c4c4ccccc4c4oc5ccccc5c43)cc2)cc1 ; c1ccc(-c2ccc(N(c3ccc(-c4ccccc4)cc3)c3cccc4c3oc3c4c4ccccc4c4oc5ccccc5c43)cc2)cc1 ; c1ccc(-c2ccc(-c3ccc(N(c4ccc(-c5ccccc5)cc4)c4cccc5c4oc4c5c5ccccc5c5oc6ccccc6c54)cc3)cc2)cc1]

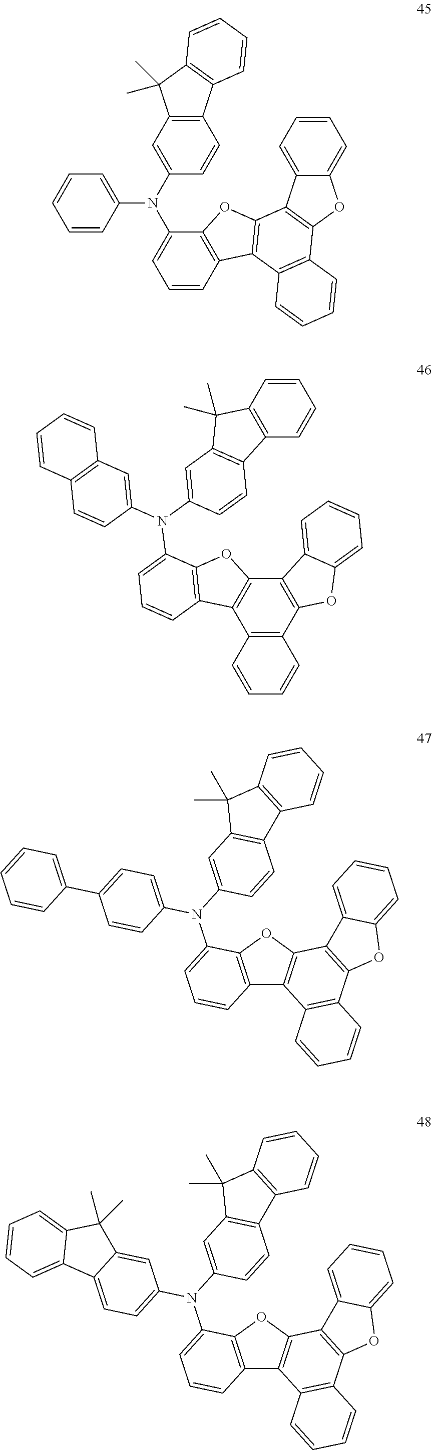 [SMILES: CC1(C)c2ccccc2-c2ccc(N(c3ccccc3)c3cccc4c3oc3c4c4ccccc4c4oc5ccccc5c43)cc21 ; CC1(C)c2ccccc2-c2ccc(N(c3ccc4ccccc4c3)c3cccc4c3oc3c4c4ccccc4c4oc5ccccc5c43)cc21 ; CC1(C)c2ccccc2-c2ccc(N(c3ccc(-c4ccccc4)cc3)c3cccc4c3oc3c4c4ccccc4c4oc5ccccc5c43)cc21 ; CC1(C)c2ccccc2-c2ccc(N(c3ccc4c(c3)C(C)(C)c3ccccc3-4)c3cccc4c3oc3c4c4ccccc4c4oc5ccccc5c43)cc21]

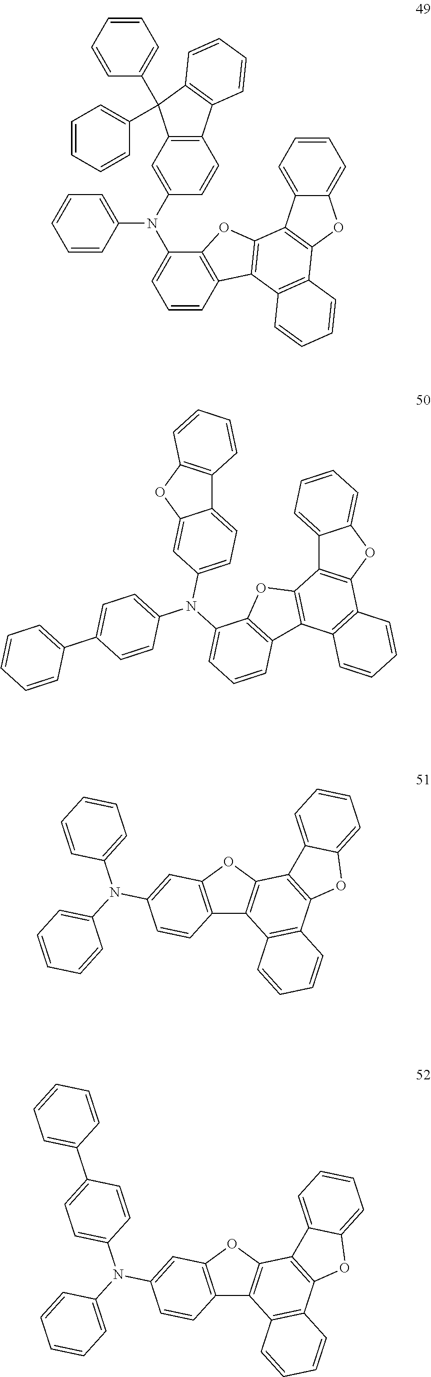 [SMILES: c1ccc(N(c2ccc3c(c2)C(c2ccccc2)(c2ccccc2)c2ccccc2-3)c2cccc3c2oc2c3c3ccccc3c3oc4ccccc4c32)cc1 ; c1ccc(-c2ccc(N(c3ccc4c(c3)oc3ccccc34)c3cccc4c3oc3c4c4ccccc4c4oc5ccccc5c43)cc2)cc1 ; c1ccc(N(c2ccccc2)c2ccc3c(c2)oc2c3c3ccccc3c3oc4ccccc4c32)cc1 ; c1ccc(-c2ccc(N(c3ccccc3)c3ccc4c(c3)oc3c4c4ccccc4c4oc5ccccc5c43)cc2)cc1]

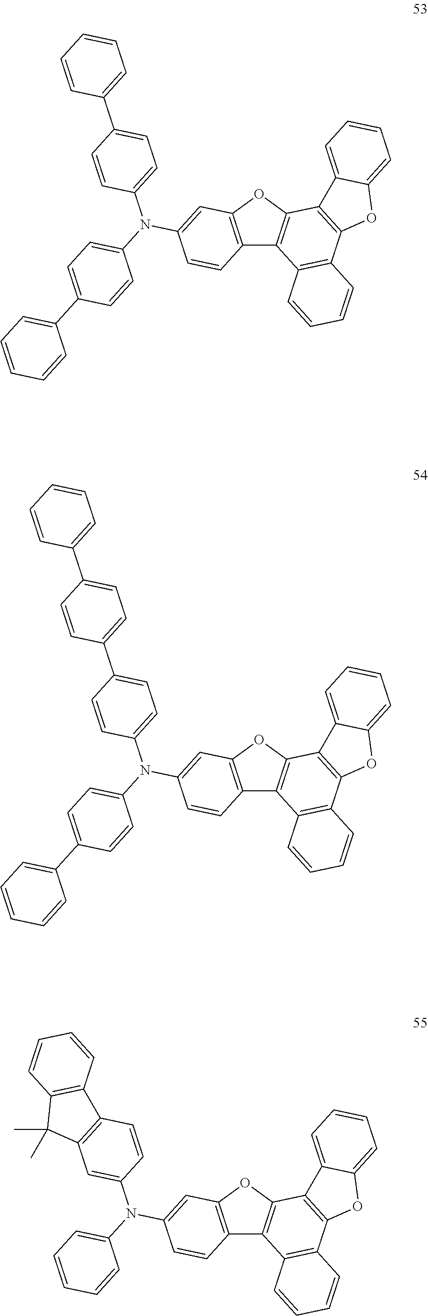 [SMILES: c1ccc(-c2ccc(N(c3ccc(-c4ccccc4)cc3)c3ccc4c(c3)oc3c4c4ccccc4c4oc5ccccc5c43)cc2)cc1 ; c1ccc(-c2ccc(-c3ccc(N(c4ccc(-c5ccccc5)cc4)c4ccc5c(c4)oc4c5c5ccccc5c5oc6ccccc6c54)cc3)cc2)cc1 ; CC1(C)c2ccccc2-c2ccc(N(c3ccccc3)c3ccc4c(c3)oc3c4c4ccccc4c4oc5ccccc5c43)cc21]

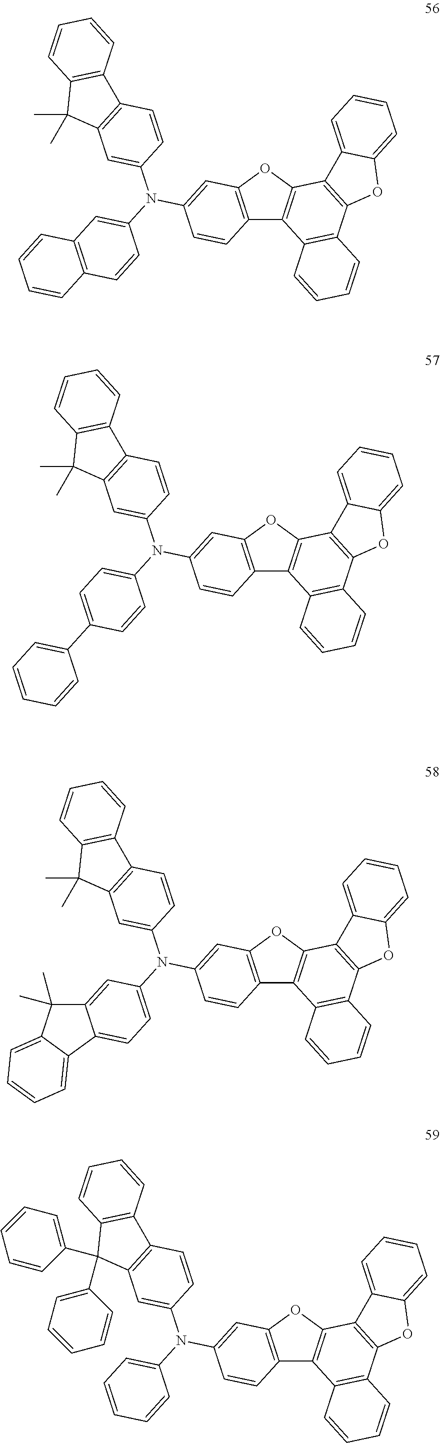 [SMILES: CC1(C)c2ccccc2-c2ccc(N(c3ccc4ccccc4c3)c3ccc4c(c3)oc3c4c4ccccc4c4oc5ccccc5c43)cc21 ; CC1(C)c2ccccc2-c2ccc(N(c3ccc(-c4ccccc4)cc3)c3ccc4c(c3)oc3c4c4ccccc4c4oc5ccccc5c43)cc21 ; CC1(C)c2ccccc2-c2ccc(N(c3ccc4c(c3)C(C)(C)c3ccccc3-4)c3ccc4c(c3)oc3c4c4ccccc4c4oc5ccccc5c43)cc21 ; c1ccc(N(c2ccc3c(c2)C(c2ccccc2)(c2ccccc2)c2ccccc2-3)c2ccc3c(c2)oc2c3c3ccccc3c3oc4ccccc4c32)cc1]

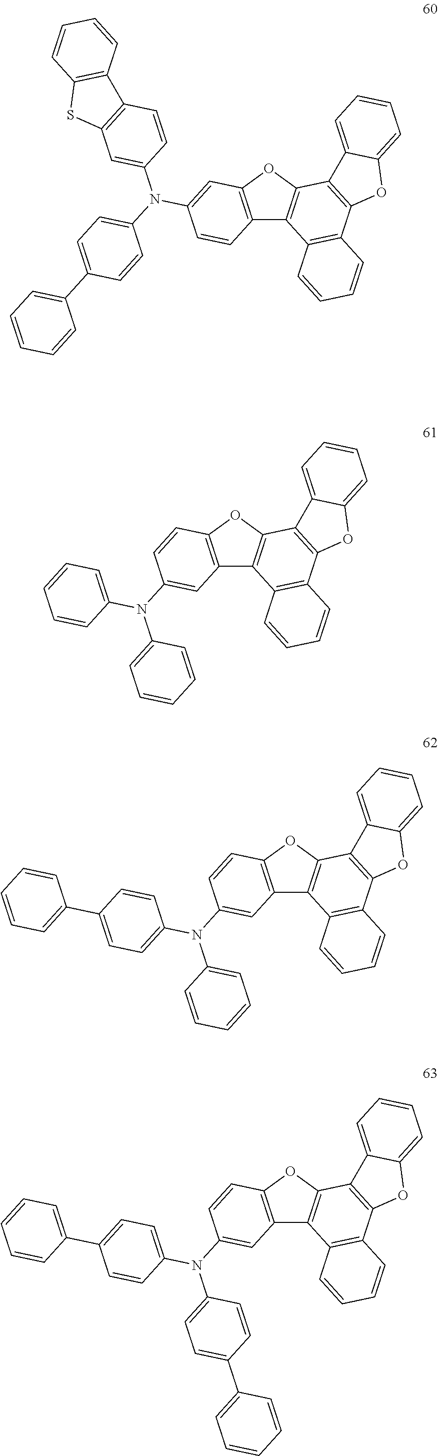 [SMILES: c1ccc(-c2ccc(N(c3ccc4c(c3)oc3c4c4ccccc4c4oc5ccccc5c43)c3ccc4c(c3)sc3ccccc34)cc2)cc1 ; c1ccc(N(c2ccccc2)c2ccc3oc4c(c3c2)c2ccccc2c2oc3ccccc3c24)cc1 ; c1ccc(-c2ccc(N(c3ccccc3)c3ccc4oc5c(c4c3)c3ccccc3c3oc4ccccc4c35)cc2)cc1 ; c1ccc(-c2ccc(N(c3ccc(-c4ccccc4)cc3)c3ccc4oc5c(c4c3)c3ccccc3c3oc4ccccc4c35)cc2)cc1]

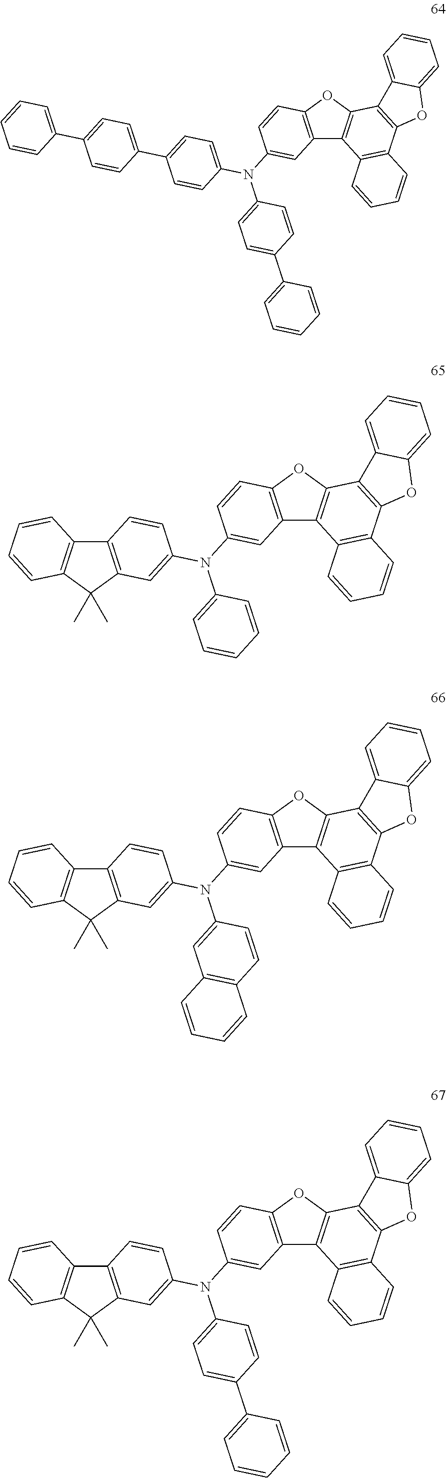 [SMILES: c1ccc(-c2ccc(-c3ccc(N(c4ccc(-c5ccccc5)cc4)c4ccc5oc6c(c5c4)c4ccccc4c4oc5ccccc5c46)cc3)cc2)cc1 ; CC1(C)c2ccccc2-c2ccc(N(c3ccccc3)c3ccc4oc5c(c4c3)c3ccccc3c3oc4ccccc4c35)cc21 ; CC1(C)c2ccccc2-c2ccc(N(c3ccc4ccccc4c3)c3ccc4oc5c(c4c3)c3ccccc3c3oc4ccccc4c35)cc21 ; CC1(C)c2ccccc2-c2ccc(N(c3ccc(-c4ccccc4)cc3)c3ccc4oc5c(c4c3)c3ccccc3c3oc4ccccc4c35)cc21]

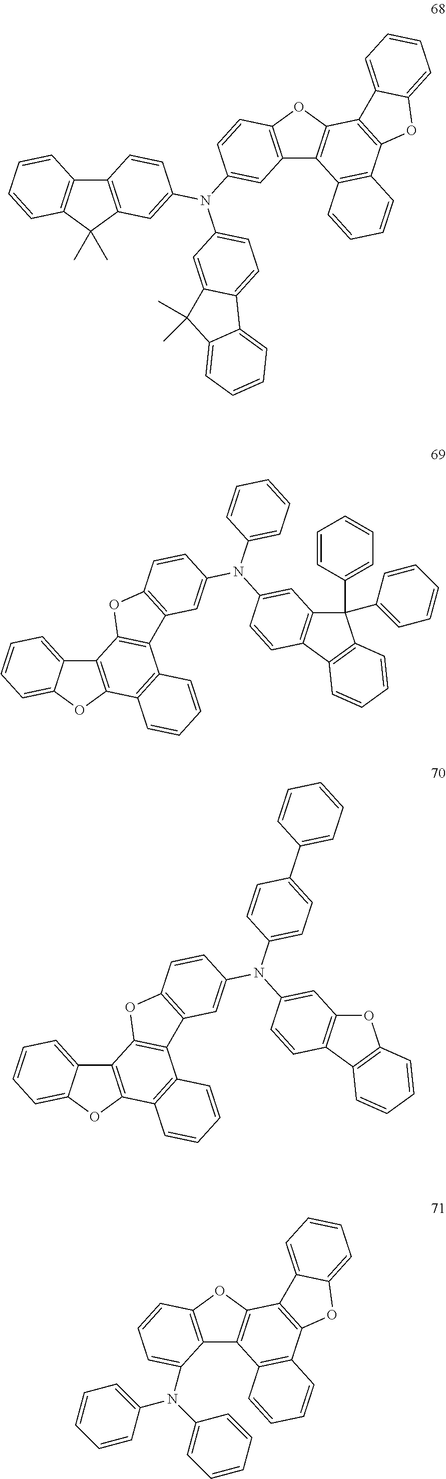 [SMILES: CC1(C)c2ccccc2-c2ccc(N(c3ccc4c(c3)C(C)(C)c3ccccc3-4)c3ccc4oc5c(c4c3)c3ccccc3c3oc4ccccc4c35)cc21 ; c1ccc(N(c2ccc3c(c2)C(c2ccccc2)(c2ccccc2)c2ccccc2-3)c2ccc3oc4c(c3c2)c2ccccc2c2oc3ccccc3c24)cc1 ; c1ccc(-c2ccc(N(c3ccc4c(c3)oc3ccccc34)c3ccc4oc5c(c4c3)c3ccccc3c3oc4ccccc4c35)cc2)cc1 ; c1ccc(N(c2ccccc2)c2cccc3oc4c5c6ccccc6oc5c5ccccc5c4c23)cc1]

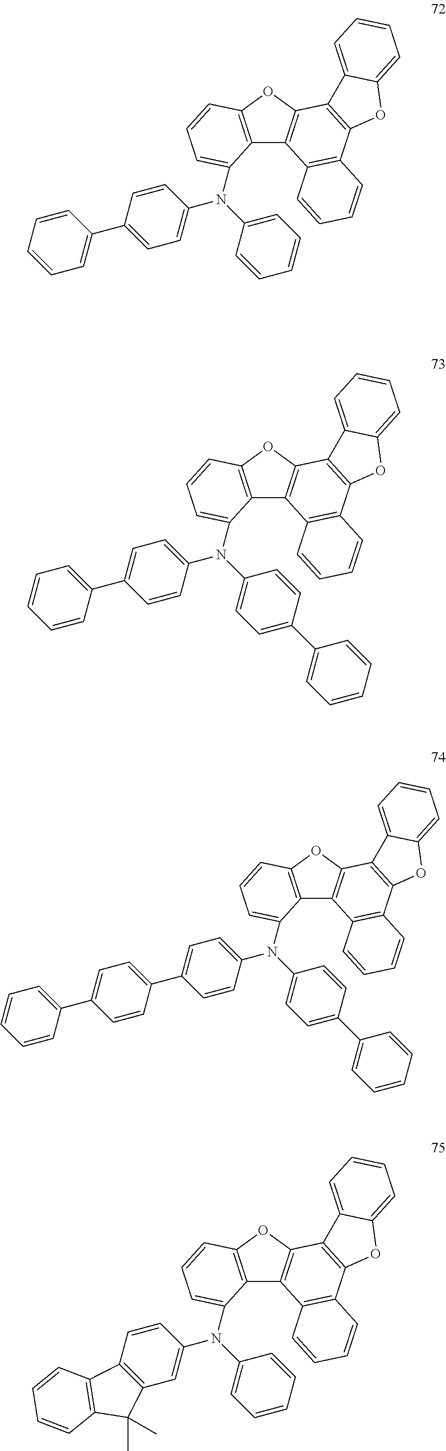 [SMILES: c1ccc(-c2ccc(N(c3ccccc3)c3cccc4oc5c6c7ccccc7oc6c6ccccc6c5c34)cc2)cc1 ; c1ccc(-c2ccc(N(c3ccc(-c4ccccc4)cc3)c3cccc4oc5c6c7ccccc7oc6c6ccccc6c5c34)cc2)cc1 ; c1ccc(-c2ccc(-c3ccc(N(c4ccc(-c5ccccc5)cc4)c4cccc5oc6c7c8ccccc8oc7c7ccccc7c6c45)cc3)cc2)cc1 ; CC1(C)c2ccccc2-c2ccc(N(c3ccccc3)c3cccc4oc5c6c7ccccc7oc6c6ccccc6c5c34)cc21]

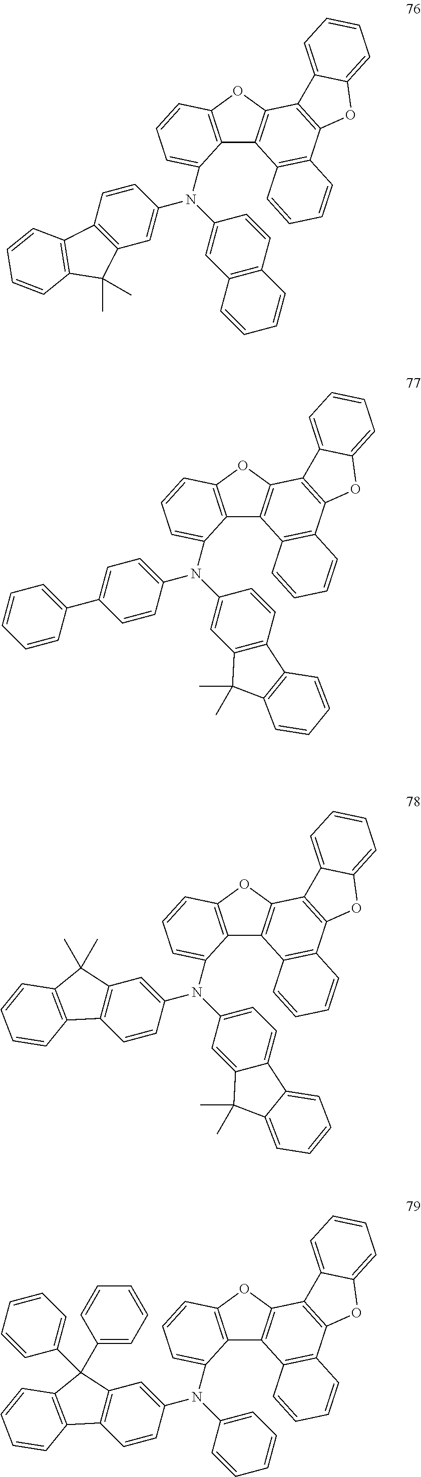 [SMILES: CC1(C)c2ccccc2-c2ccc(N(c3ccc4ccccc4c3)c3cccc4oc5c6c7ccccc7oc6c6ccccc6c5c34)cc21 ; CC1(C)c2ccccc2-c2ccc(N(c3ccc(-c4ccccc4)cc3)c3cccc4oc5c6c7ccccc7oc6c6ccccc6c5c34)cc21 ; CC1(C)c2ccccc2-c2ccc(N(c3ccc4c(c3)C(C)(C)c3ccccc3-4)c3cccc4oc5c6c7ccccc7oc6c6ccccc6c5c34)cc21 ; c1ccc(N(c2ccc3c(c2)C(c2ccccc2)(c2ccccc2)c2ccccc2-3)c2cccc3oc4c5c6ccccc6oc5c5ccccc5c4c23)cc1]

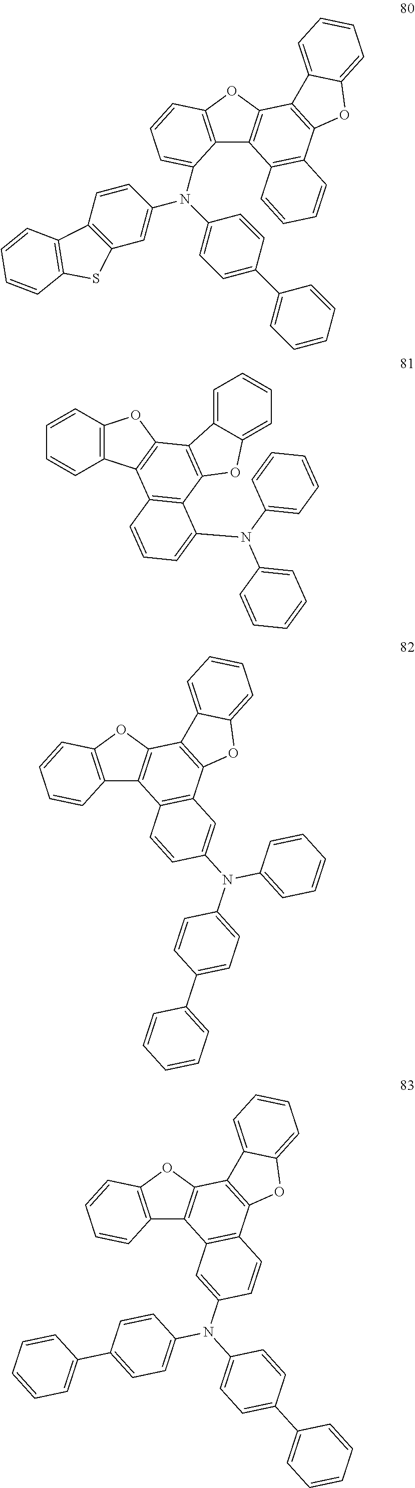

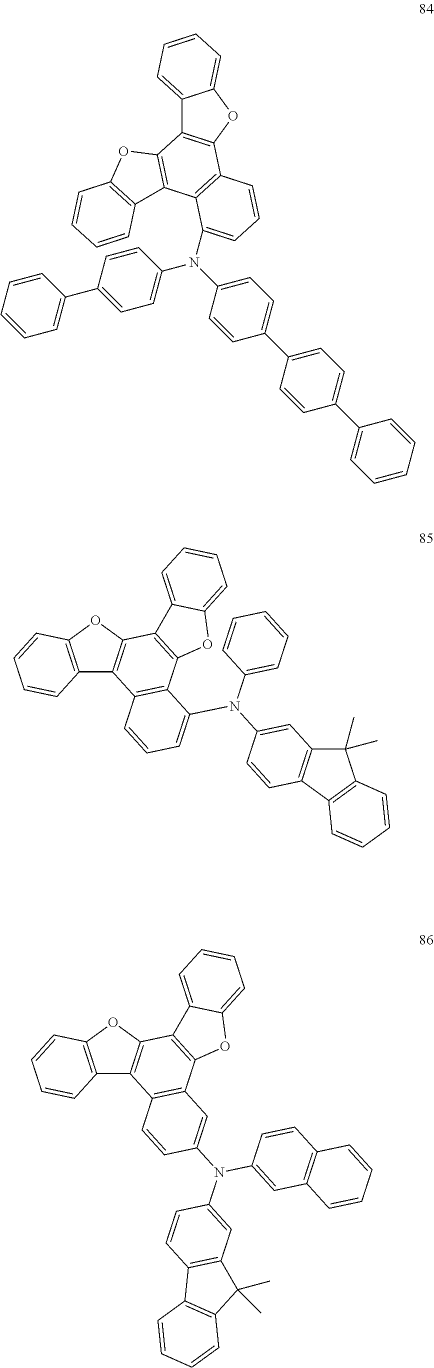 [SMILES: c1ccc(-c2ccc(-c3ccc(N(c4ccc(-c5ccccc5)cc4)c4cccc5c6oc7ccccc7c6c6oc7ccccc7c6c45)cc3)cc2)cc1 ; CC1(C)c2ccccc2-c2ccc(N(c3ccccc3)c3cccc4c3c3oc5ccccc5c3c3oc5ccccc5c43)cc21 ; CC1(C)c2ccccc2-c2ccc(N(c3ccc4ccccc4c3)c3ccc4c(c3)c3oc5ccccc5c3c3oc5ccccc5c43)cc21]

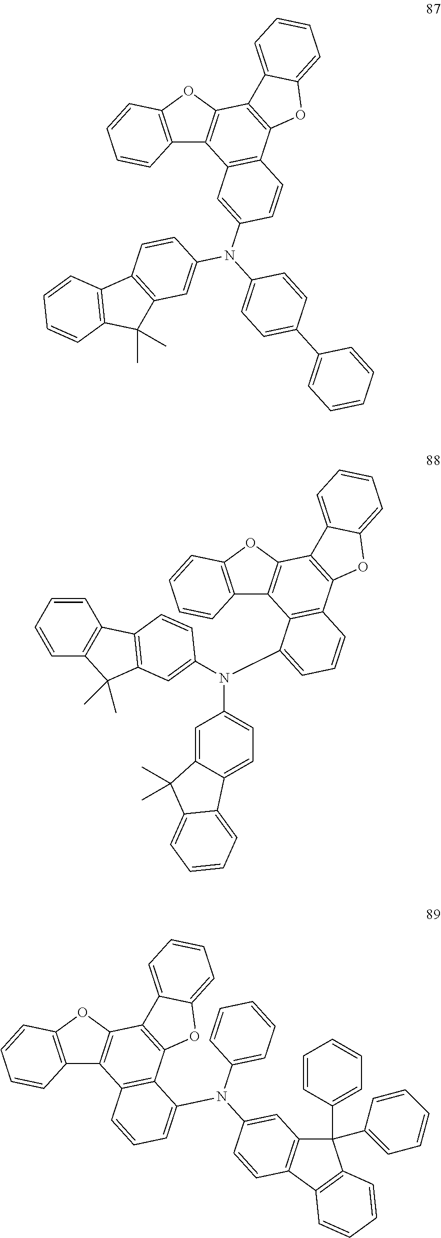 [SMILES: CC1(C)c2ccccc2-c2ccc(N(c3ccc(-c4ccccc4)cc3)c3ccc4c(c3)c3c5ccccc5oc3c3c5ccccc5oc43)cc21 ; CC1(C)c2ccccc2-c2ccc(N(c3ccc4c(c3)C(C)(C)c3ccccc3-4)c3cccc4c5oc6ccccc6c5c5oc6ccccc6c5c34)cc21 ; c1ccc(N(c2ccc3c(c2)C(c2ccccc2)(c2ccccc2)c2ccccc2-3)c2cccc3c2c2oc4ccccc4c2c2oc4ccccc4c32)cc1]

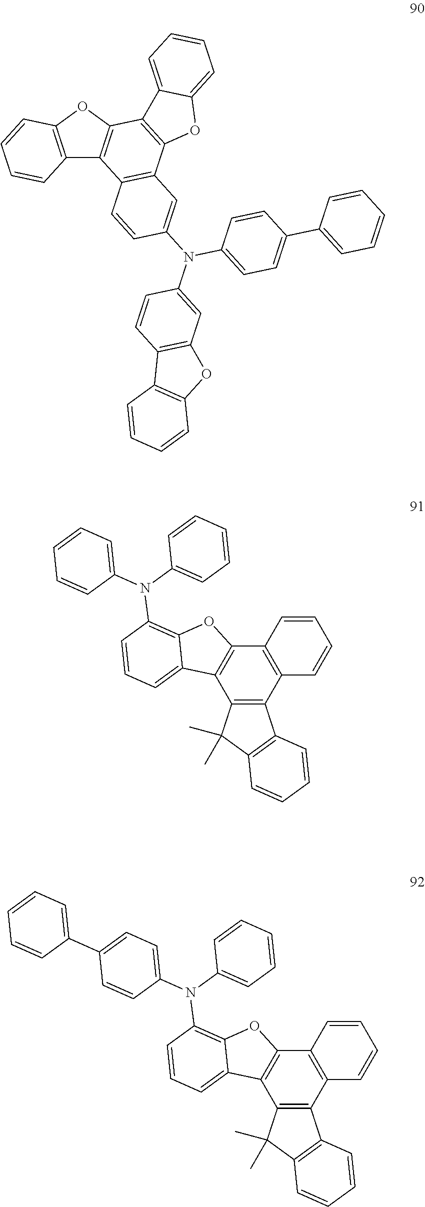 [SMILES: c1ccc(-c2ccc(N(c3ccc4c(c3)oc3ccccc34)c3ccc4c(c3)c3oc5ccccc5c3c3oc5ccccc5c43)cc2)cc1 ; CC1(C)c2ccccc2-c2c1c1c3cccc(N(c4ccccc4)c4ccccc4)c3oc1c1ccccc21 ; CC1(C)c2ccccc2-c2c1c1c3cccc(N(c4ccccc4)c4ccc(-c5ccccc5)cc4)c3oc1c1ccccc21]

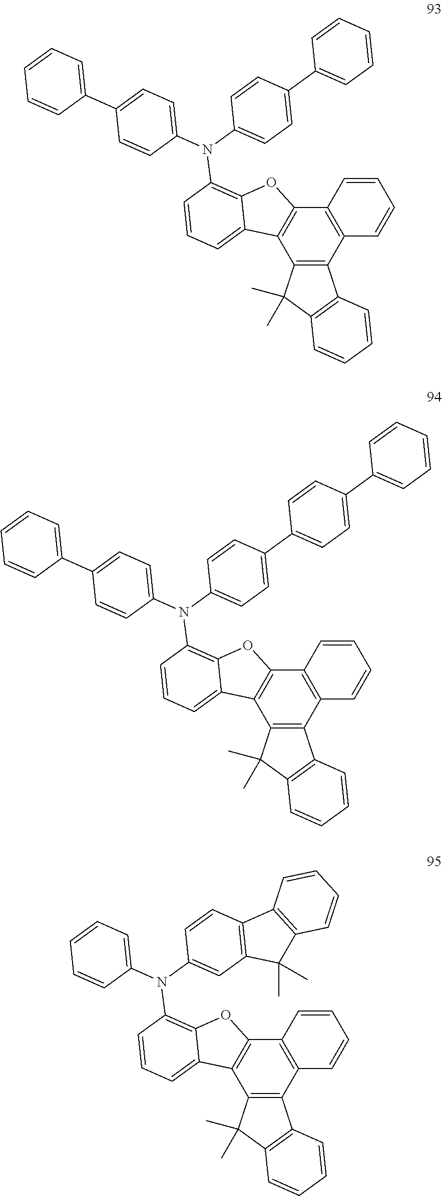

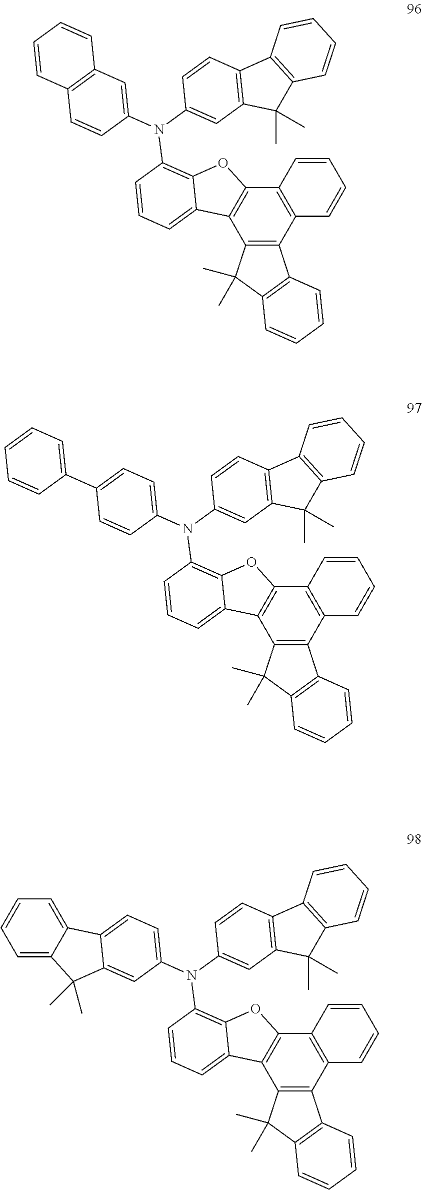 [SMILES: CC1(C)c2ccccc2-c2ccc(N(c3ccc4ccccc4c3)c3cccc4c3oc3c5ccccc5c5c(c43)C(C)(C)c3ccccc3-5)cc21 ; CC1(C)c2ccccc2-c2ccc(N(c3ccc(-c4ccccc4)cc3)c3cccc4c3oc3c5ccccc5c5c(c43)C(C)(C)c3ccccc3-5)cc21 ; CC1(C)c2ccccc2-c2ccc(N(c3ccc4c(c3)C(C)(C)c3ccccc3-4)c3cccc4c3oc3c5ccccc5c5c(c43)C(C)(C)c3ccccc3-5)cc21]

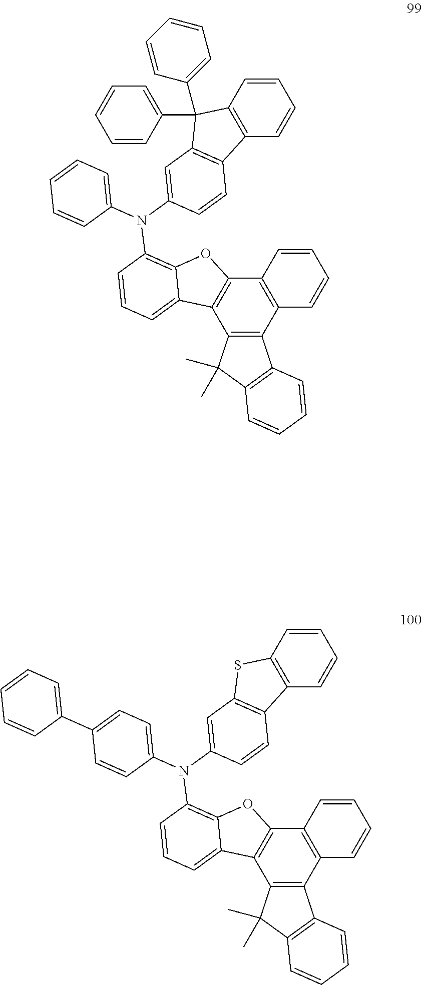 [SMILES: CC1(C)c2ccccc2-c2c1c1c3cccc(N(c4ccccc4)c4ccc5c(c4)C(c4ccccc4)(c4ccccc4)c4ccccc4-5)c3oc1c1ccccc21 ; CC1(C)c2ccccc2-c2c1c1c3cccc(N(c4ccc(-c5ccccc5)cc4)c4ccc5c(c4)sc4ccccc45)c3oc1c1ccccc21]

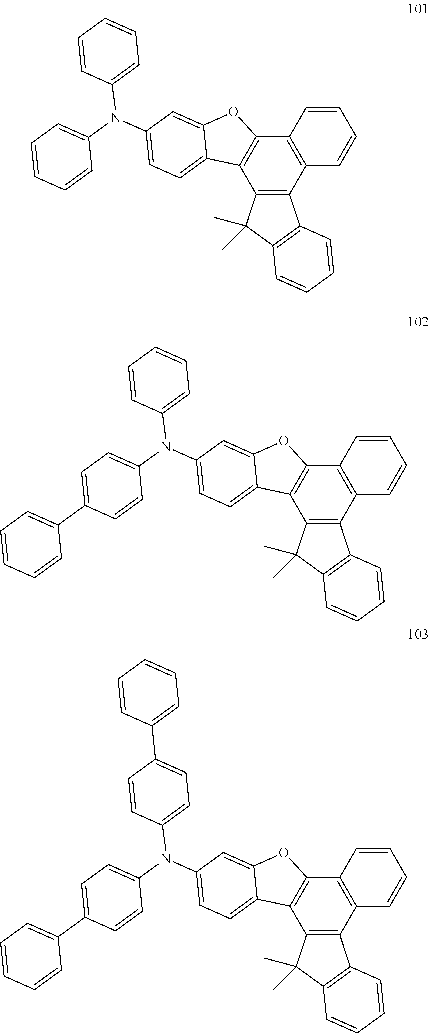 [SMILES: CC1(C)c2ccccc2-c2c1c1c3ccc(N(c4ccccc4)c4ccccc4)cc3oc1c1ccccc21 ; CC1(C)c2ccccc2-c2c1c1c3ccc(N(c4ccccc4)c4ccc(-c5ccccc5)cc4)cc3oc1c1ccccc21 ; CC1(C)c2ccccc2-c2c1c1c3ccc(N(c4ccc(-c5ccccc5)cc4)c4ccc(-c5ccccc5)cc4)cc3oc1c1ccccc21]

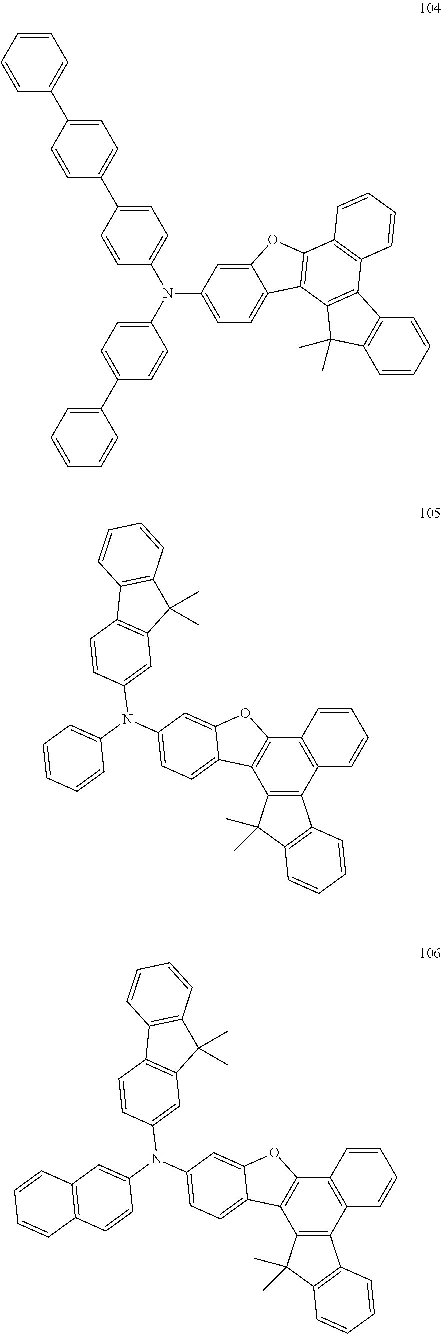 [SMILES: CC1(C)c2ccccc2-c2c1c1c3ccc(N(c4ccc(-c5ccccc5)cc4)c4ccc(-c5ccc(-c6ccccc6)cc5)cc4)cc3oc1c1ccccc21 ; CC1(C)c2ccccc2-c2ccc(N(c3ccccc3)c3ccc4c(c3)oc3c5ccccc5c5c(c43)C(C)(C)c3ccccc3-5)cc21 ; CC1(C)c2ccccc2-c2ccc(N(c3ccc4ccccc4c3)c3ccc4c(c3)oc3c5ccccc5c5c(c43)C(C)(C)c3ccccc3-5)cc21]

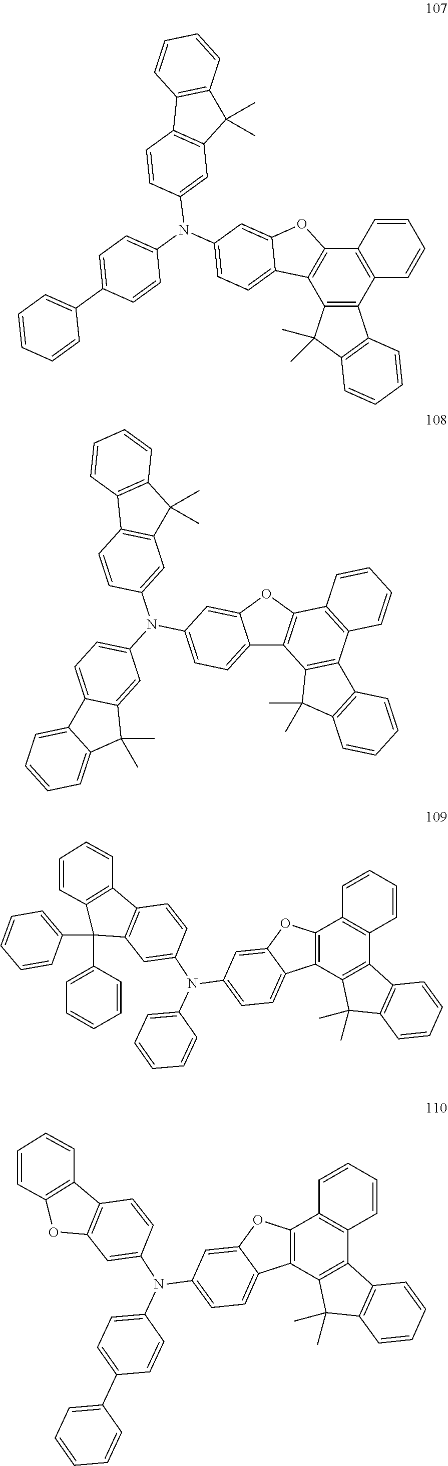 [SMILES: CC1(C)c2ccccc2-c2ccc(N(c3ccc(-c4ccccc4)cc3)c3ccc4c(c3)oc3c5ccccc5c5c(c43)C(C)(C)c3ccccc3-5)cc21 ; CC1(C)c2ccccc2-c2ccc(N(c3ccc4c(c3)C(C)(C)c3ccccc3-4)c3ccc4c(c3)oc3c5ccccc5c5c(c43)C(C)(C)c3ccccc3-5)cc21 ; CC1(C)c2ccccc2-c2c1c1c3ccc(N(c4ccccc4)c4ccc5c(c4)C(c4ccccc4)(c4ccccc4)c4ccccc4-5)cc3oc1c1ccccc21 ; CC1(C)c2ccccc2-c2c1c1c3ccc(N(c4ccc(-c5ccccc5)cc4)c4ccc5c(c4)oc4ccccc45)cc3oc1c1ccccc21]

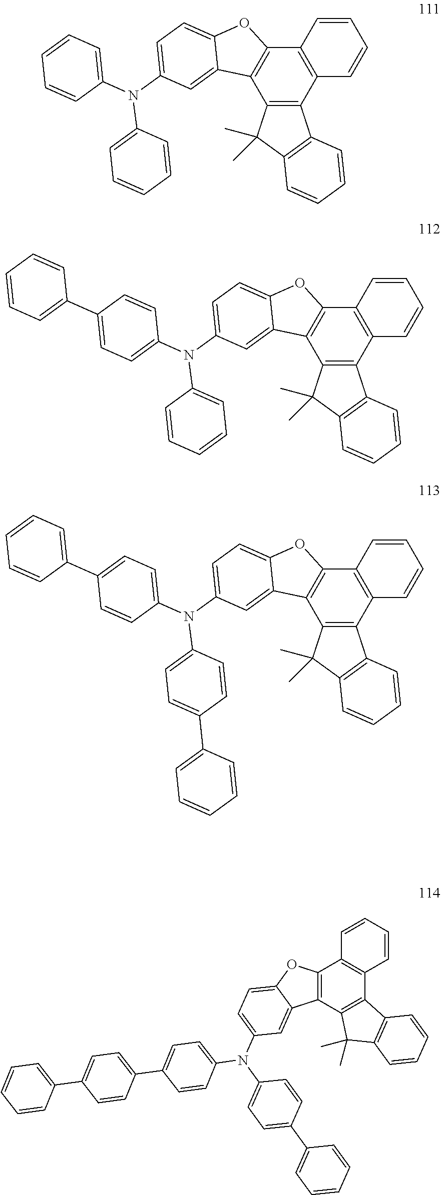 [SMILES: CC1(C)c2ccccc2-c2c1c1c3cc(N(c4ccccc4)c4ccccc4)ccc3oc1c1ccccc21 ; CC1(C)c2ccccc2-c2c1c1c3cc(N(c4ccccc4)c4ccc(-c5ccccc5)cc4)ccc3oc1c1ccccc21 ; CC1(C)c2ccccc2-c2c1c1c3cc(N(c4ccc(-c5ccccc5)cc4)c4ccc(-c5ccccc5)cc4)ccc3oc1c1ccccc21 ; CC1(C)c2ccccc2-c2c1c1c3cc(N(c4ccc(-c5ccccc5)cc4)c4ccc(-c5ccc(-c6ccccc6)cc5)cc4)ccc3oc1c1ccccc21]

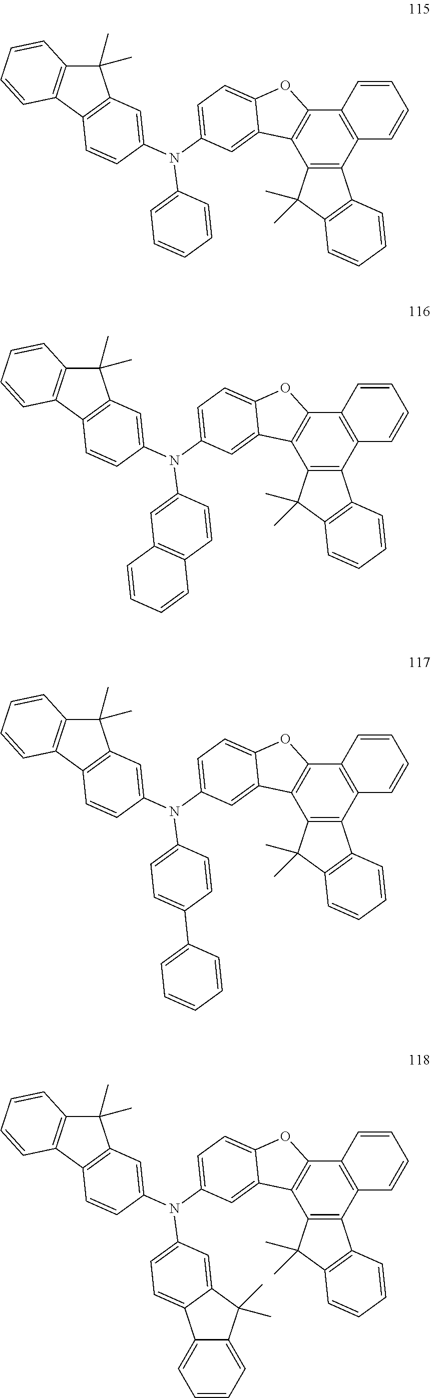 [SMILES: CC1(C)c2ccccc2-c2ccc(N(c3ccccc3)c3ccc4oc5c6ccccc6c6c(c5c4c3)C(C)(C)c3ccccc3-6)cc21 ; CC1(C)c2ccccc2-c2ccc(N(c3ccc4ccccc4c3)c3ccc4oc5c6ccccc6c6c(c5c4c3)C(C)(C)c3ccccc3-6)cc21 ; CC1(C)c2ccccc2-c2ccc(N(c3ccc(-c4ccccc4)cc3)c3ccc4oc5c6ccccc6c6c(c5c4c3)C(C)(C)c3ccccc3-6)cc21 ; CC1(C)c2ccccc2-c2ccc(N(c3ccc4c(c3)C(C)(C)c3ccccc3-4)c3ccc4oc5c6ccccc6c6c(c5c4c3)C(C)(C)c3ccccc3-6)cc21]

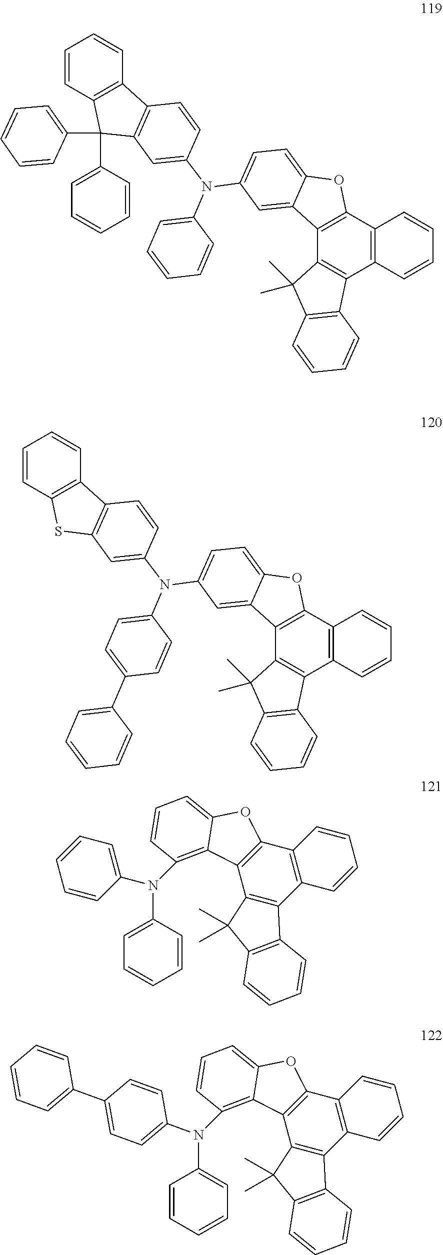 [SMILES: CC1(C)c2ccccc2-c2c1c1c3cc(N(c4ccccc4)c4ccc5c(c4)C(c4ccccc4)(c4ccccc4)c4ccccc4-5)ccc3oc1c1ccccc21 ; CC1(C)c2ccccc2-c2c1c1c3cc(N(c4ccc(-c5ccccc5)cc4)c4ccc5c(c4)sc4ccccc45)ccc3oc1c1ccccc21 ; CC1(C)c2ccccc2-c2c1c1c(oc3cccc(N(c4ccccc4)c4ccccc4)c31)c1ccccc21 ; CC1(C)c2ccccc2-c2c1c1c(oc3cccc(N(c4ccccc4)c4ccc(-c5ccccc5)cc4)c31)c1ccccc21]

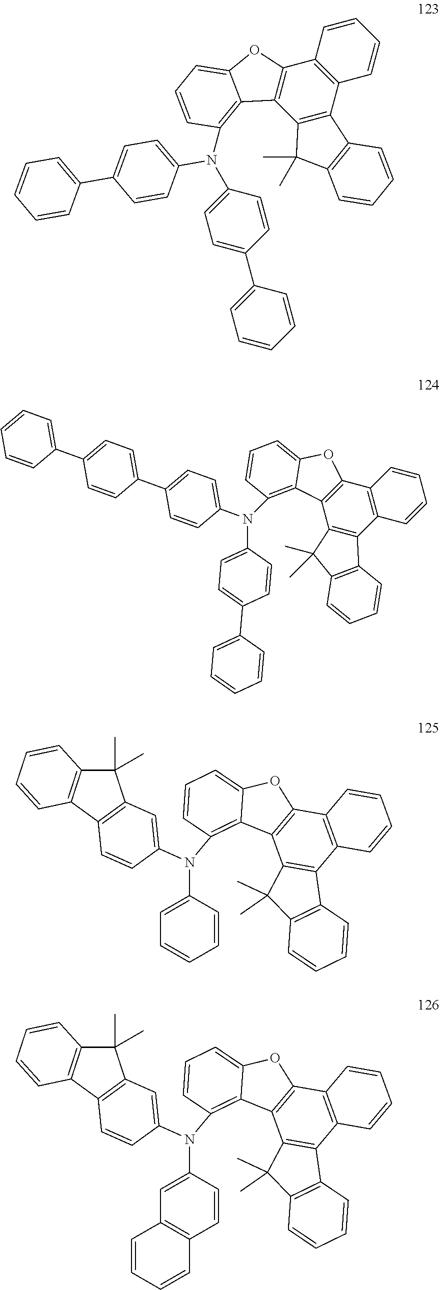 [SMILES: CC1(C)c2ccccc2-c2c1c1c(oc3cccc(N(c4ccc(-c5ccccc5)cc4)c4ccc(-c5ccccc5)cc4)c31)c1ccccc21 ; CC1(C)c2ccccc2-c2c1c1c(oc3cccc(N(c4ccc(-c5ccccc5)cc4)c4ccc(-c5ccc(-c6ccccc6)cc5)cc4)c31)c1ccccc21 ; CC1(C)c2ccccc2-c2ccc(N(c3ccccc3)c3cccc4oc5c6ccccc6c6c(c5c34)C(C)(C)c3ccccc3-6)cc21 ; CC1(C)c2ccccc2-c2ccc(N(c3ccc4ccccc4c3)c3cccc4oc5c6ccccc6c6c(c5c34)C(C)(C)c3ccccc3-6)cc21]

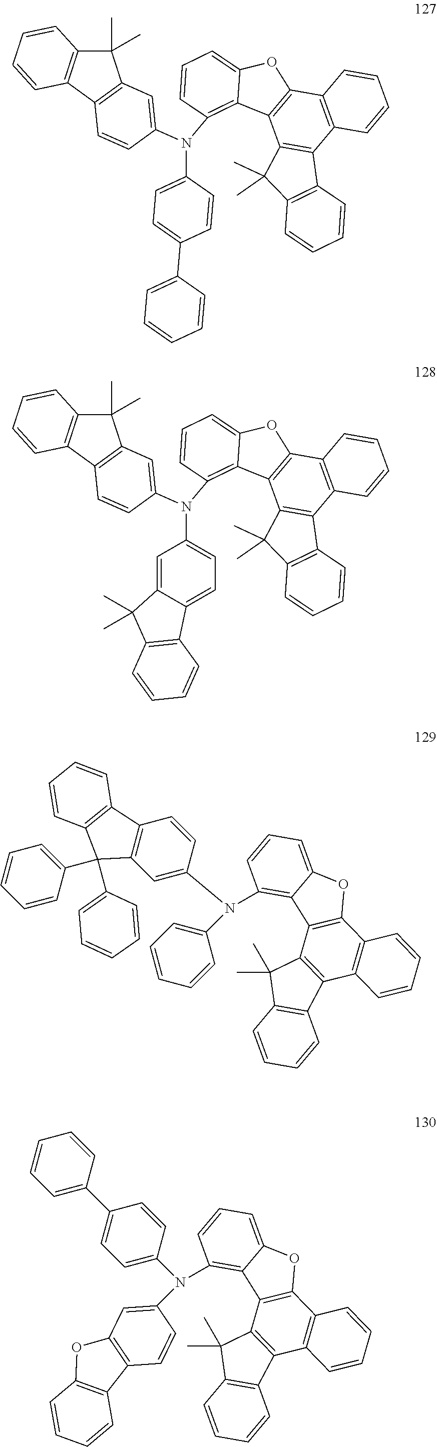 [SMILES: CC1(C)c2ccccc2-c2ccc(N(c3ccc(-c4ccccc4)cc3)c3cccc4oc5c6ccccc6c6c(c5c34)C(C)(C)c3ccccc3-6)cc21 ; CC1(C)c2ccccc2-c2ccc(N(c3ccc4c(c3)C(C)(C)c3ccccc3-4)c3cccc4oc5c6ccccc6c6c(c5c34)C(C)(C)c3ccccc3-6)cc21 ; CC1(C)c2ccccc2-c2c1c1c(oc3cccc(N(c4ccccc4)c4ccc5c(c4)C(c4ccccc4)(c4ccccc4)c4ccccc4-5)c31)c1ccccc21 ; CC1(C)c2ccccc2-c2c1c1c(oc3cccc(N(c4ccc(-c5ccccc5)cc4)c4ccc5c(c4)oc4ccccc45)c31)c1ccccc21]

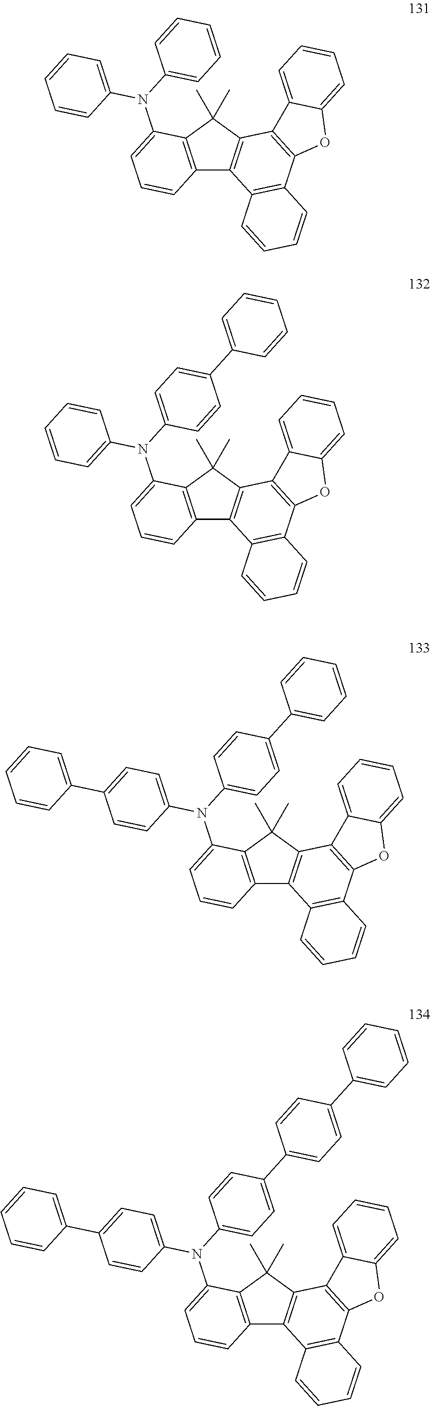 [SMILES: CC1(C)c2c(cccc2N(c2ccccc2)c2ccccc2)-c2c1c1c3ccccc3oc1c1ccccc21 ; CC1(C)c2c(cccc2N(c2ccccc2)c2ccc(-c3ccccc3)cc2)-c2c1c1c3ccccc3oc1c1ccccc21 ; CC1(C)c2c(cccc2N(c2ccc(-c3ccccc3)cc2)c2ccc(-c3ccccc3)cc2)-c2c1c1c3ccccc3oc1c1ccccc21 ; CC1(C)c2c(cccc2N(c2ccc(-c3ccccc3)cc2)c2ccc(-c3ccc(-c4ccccc4)cc3)cc2)-c2c1c1c3ccccc3oc1c1ccccc21]

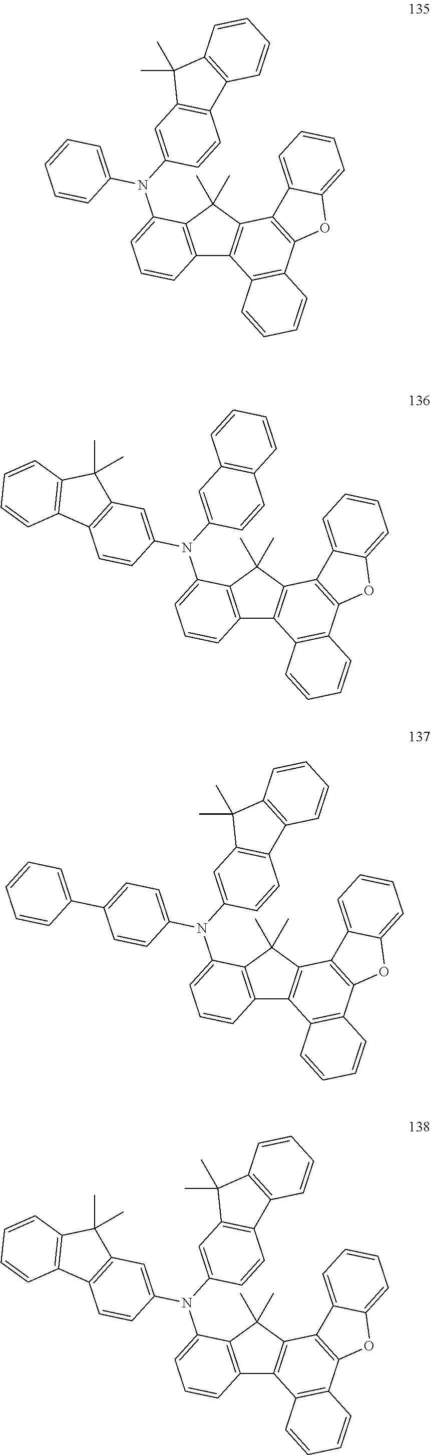 [SMILES: CC1(C)c2ccccc2-c2ccc(N(c3ccccc3)c3cccc4c3C(C)(C)c3c-4c4ccccc4c4oc5ccccc5c34)cc21 ; CC1(C)c2ccccc2-c2ccc(N(c3ccc4ccccc4c3)c3cccc4c3C(C)(C)c3c-4c4ccccc4c4oc5ccccc5c34)cc21 ; CC1(C)c2ccccc2-c2ccc(N(c3ccc(-c4ccccc4)cc3)c3cccc4c3C(C)(C)c3c-4c4ccccc4c4oc5ccccc5c34)cc21 ; CC1(C)c2ccccc2-c2ccc(N(c3ccc4c(c3)C(C)(C)c3ccccc3-4)c3cccc4c3C(C)(C)c3c-4c4ccccc4c4oc5ccccc5c34)cc21]

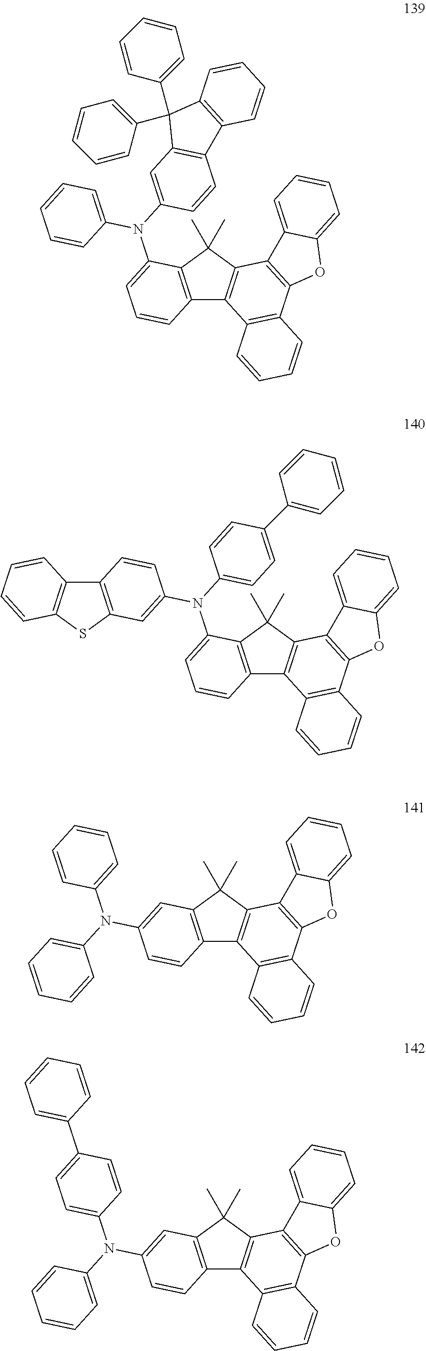 [SMILES: CC1(C)c2c(cccc2N(c2ccccc2)c2ccc3c(c2)C(c2ccccc2)(c2ccccc2)c2ccccc2-3)-c2c1c1c3ccccc3oc1c1ccccc21 ; CC1(C)c2c(cccc2N(c2ccc(-c3ccccc3)cc2)c2ccc3c(c2)sc2ccccc23)-c2c1c1c3ccccc3oc1c1ccccc21 ; CC1(C)c2cc(N(c3ccccc3)c3ccccc3)ccc2-c2c1c1c3ccccc3oc1c1ccccc21 ; CC1(C)c2cc(N(c3ccccc3)c3ccc(-c4ccccc4)cc3)ccc2-c2c1c1c3ccccc3oc1c1ccccc21]

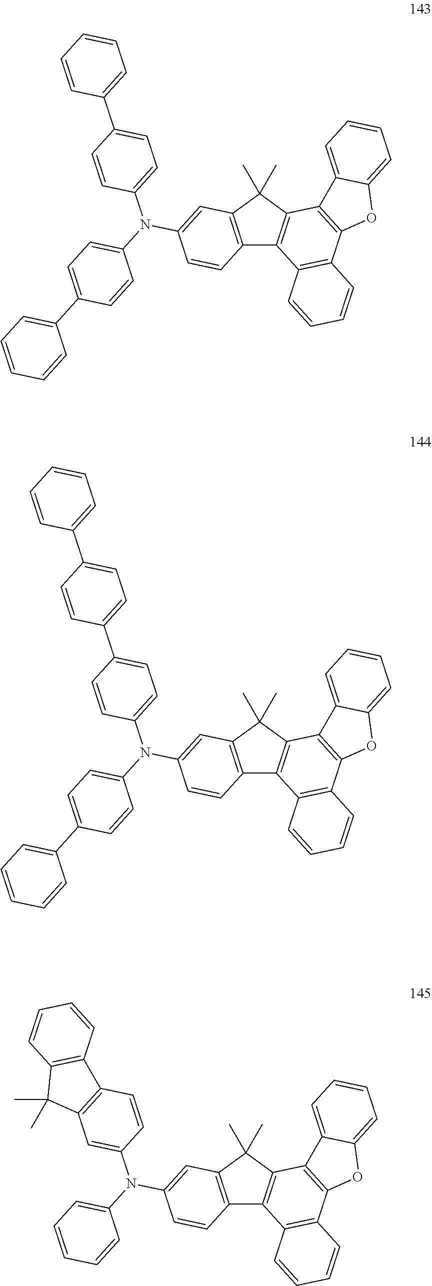 [SMILES: CC1(C)c2cc(N(c3ccc(-c4ccccc4)cc3)c3ccc(-c4ccccc4)cc3)ccc2-c2c1c1c3ccccc3oc1c1ccccc21 ; CC1(C)c2cc(N(c3ccc(-c4ccccc4)cc3)c3ccc(-c4ccc(-c5ccccc5)cc4)cc3)ccc2-c2c1c1c3ccccc3oc1c1ccccc21 ; CC1(C)c2ccccc2-c2ccc(N(c3ccccc3)c3ccc4c(c3)C(C)(C)c3c-4c4ccccc4c4oc5ccccc5c34)cc21]

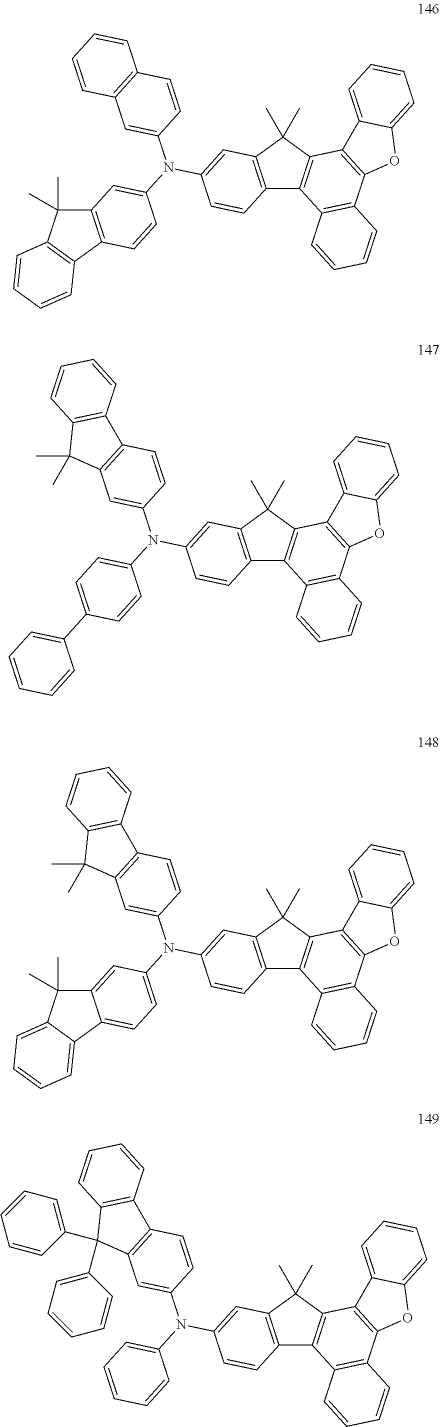 [SMILES: CC1(C)c2ccccc2-c2ccc(N(c3ccc4c(c3)C(C)(C)c3c-4c4ccccc4c4oc5ccccc5c34)c3ccc4ccccc4c3)cc21 ; CC1(C)c2ccccc2-c2ccc(N(c3ccc(-c4ccccc4)cc3)c3ccc4c(c3)C(C)(C)c3c-4c4ccccc4c4oc5ccccc5c34)cc21 ; CC1(C)c2ccccc2-c2ccc(N(c3ccc4c(c3)C(C)(C)c3ccccc3-4)c3ccc4c(c3)C(C)(C)c3c-4c4ccccc4c4oc5ccccc5c34)cc21 ; CC1(C)c2cc(N(c3ccccc3)c3ccc4c(c3)C(c3ccccc3)(c3ccccc3)c3ccccc3-4)ccc2-c2c1c1c3ccccc3oc1c1ccccc21]

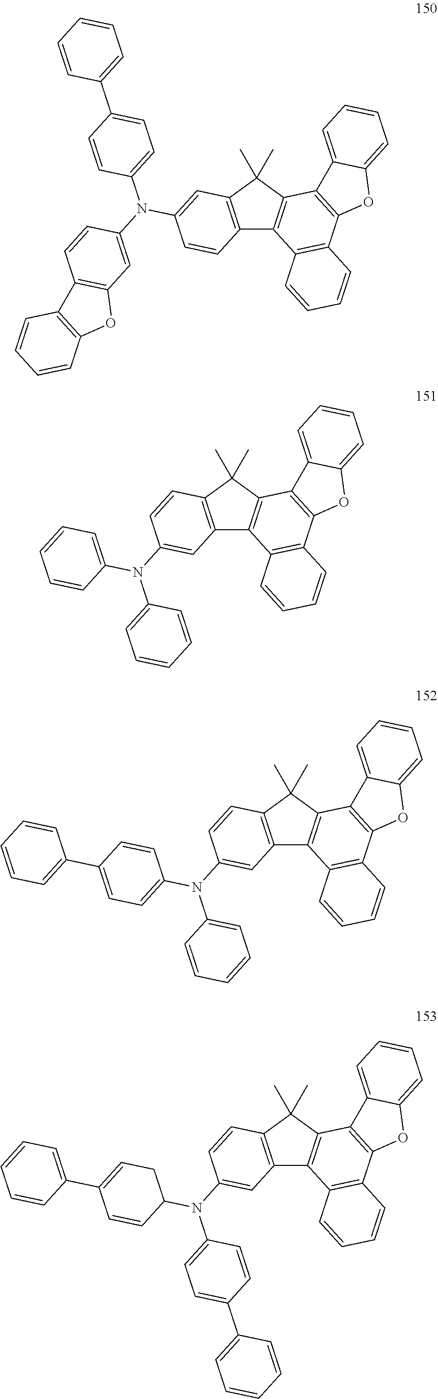 [SMILES: CC1(C)c2cc(N(c3ccc(-c4ccccc4)cc3)c3ccc4c(c3)oc3ccccc34)ccc2-c2c1c1c3ccccc3oc1c1ccccc21 ; CC1(C)c2ccc(N(c3ccccc3)c3ccccc3)cc2-c2c1c1c3ccccc3oc1c1ccccc21 ; CC1(C)c2ccc(N(c3ccccc3)c3ccc(-c4ccccc4)cc3)cc2-c2c1c1c3ccccc3oc1c1ccccc21 ; CC1(C)c2ccc(N(c3ccc(-c4ccccc4)cc3)C3C=CC(c4ccccc4)=CC3)cc2-c2c1c1c3ccccc3oc1c1ccccc21]

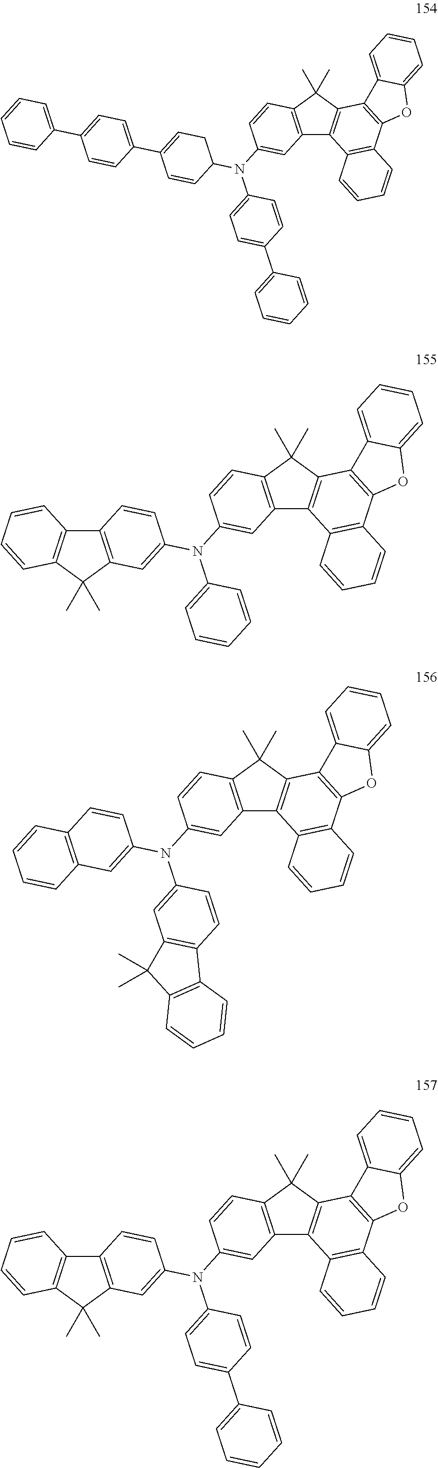 [SMILES: CC1(C)c2ccc(N(c3ccc(-c4ccccc4)cc3)C3C=CC(c4ccc(-c5ccccc5)cc4)=CC3)cc2-c2c1c1c3ccccc3oc1c1ccccc21 ; CC1(C)c2ccccc2-c2ccc(N(c3ccccc3)c3ccc4c(c3)-c3c(c5c6ccccc6oc5c5ccccc35)C4(C)C)cc21 ; CC1(C)c2ccccc2-c2ccc(N(c3ccc4c(c3)-c3c(c5c6ccccc6oc5c5ccccc35)C4(C)C)c3ccc4ccccc4c3)cc21 ; CC1(C)c2ccccc2-c2ccc(N(c3ccc(-c4ccccc4)cc3)c3ccc4c(c3)-c3c(c5c6ccccc6oc5c5ccccc35)C4(C)C)cc21]

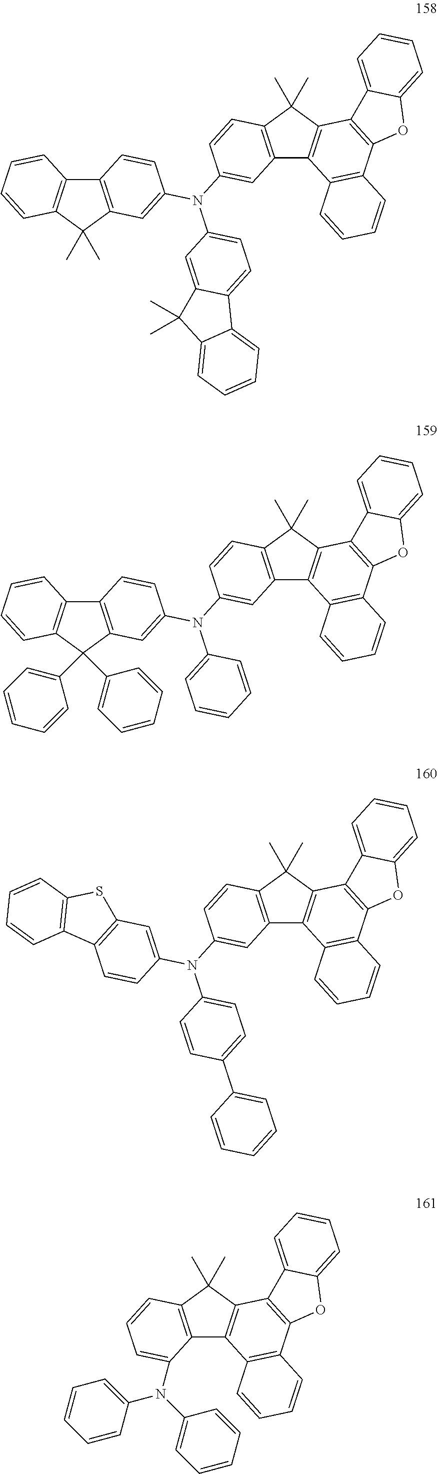 [SMILES: CC1(C)c2ccccc2-c2ccc(N(c3ccc4c(c3)-c3c(c5c6ccccc6oc5c5ccccc35)C4(C)C)c3ccc4c(c3)C(C)(C)c3ccccc3-4)cc21 ; CC1(C)c2ccc(N(c3ccccc3)c3ccc4c(c3)C(c3ccccc3)(c3ccccc3)c3ccccc3-4)cc2-c2c1c1c3ccccc3oc1c1ccccc21 ; CC1(C)c2ccc(N(c3ccc(-c4ccccc4)cc3)c3ccc4c(c3)sc3ccccc34)cc2-c2c1c1c3ccccc3oc1c1ccccc21 ; CC1(C)c2cccc(N(c3ccccc3)c3ccccc3)c2-c2c1c1c3ccccc3oc1c1ccccc21]

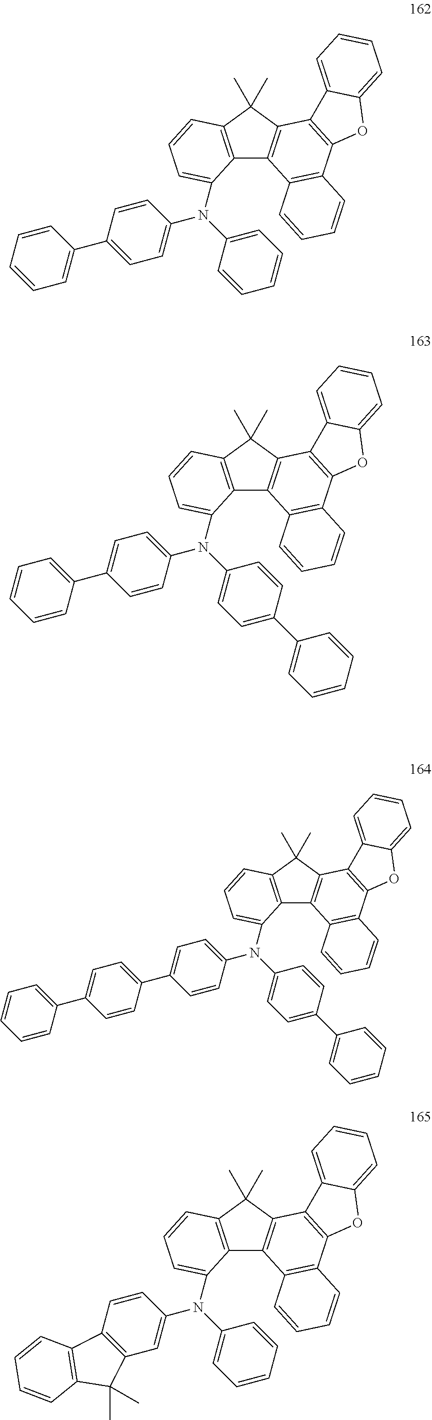

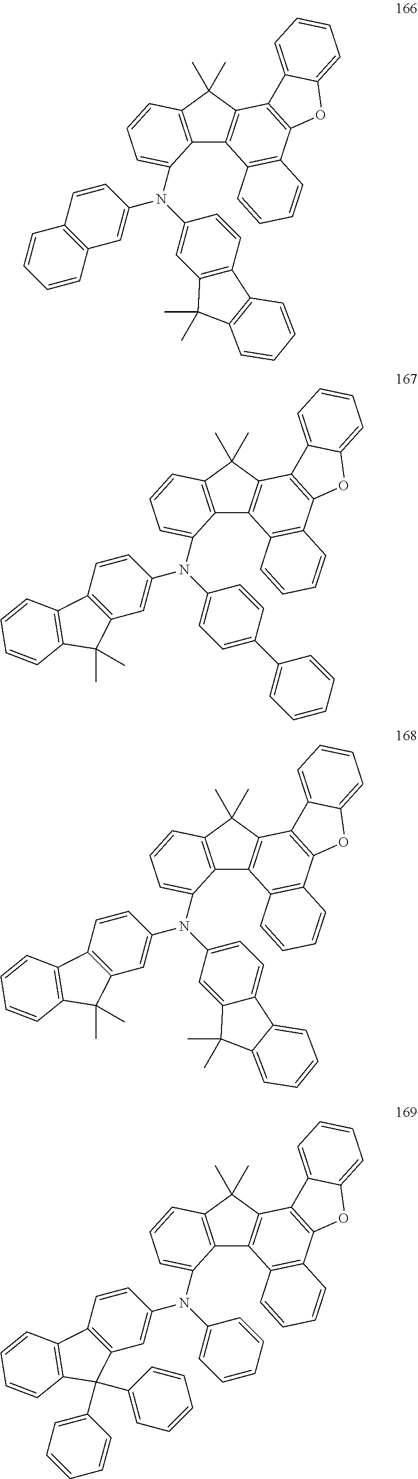

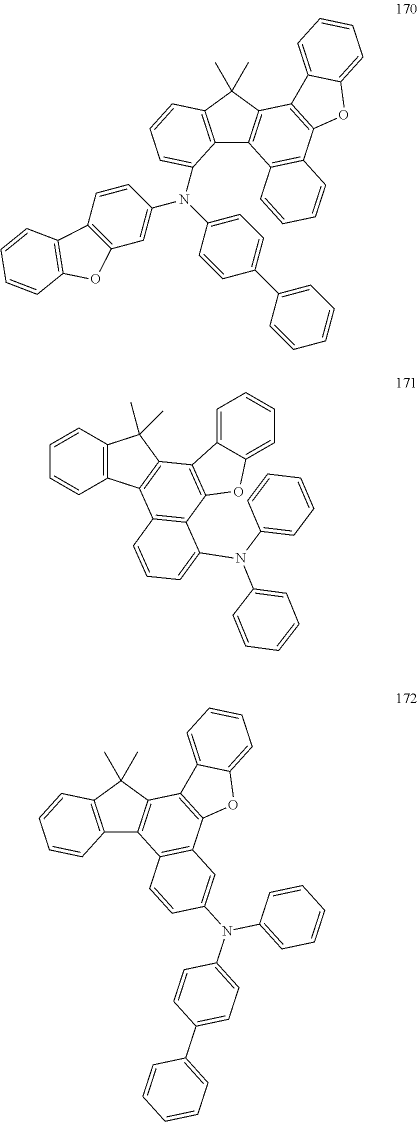 [SMILES: CC1(C)c2cccc(N(c3ccc(-c4ccccc4)cc3)c3ccc4c(c3)oc3ccccc34)c2-c2c1c1c3ccccc3oc1c1ccccc21 ; CC1(C)c2ccccc2-c2c1c1c3ccccc3oc1c1c(N(c3ccccc3)c3ccccc3)cccc21 ; CC1(C)c2ccccc2-c2c1c1c3ccccc3oc1c1cc(N(c3ccccc3)c3ccc(-c4ccccc4)cc3)ccc21]

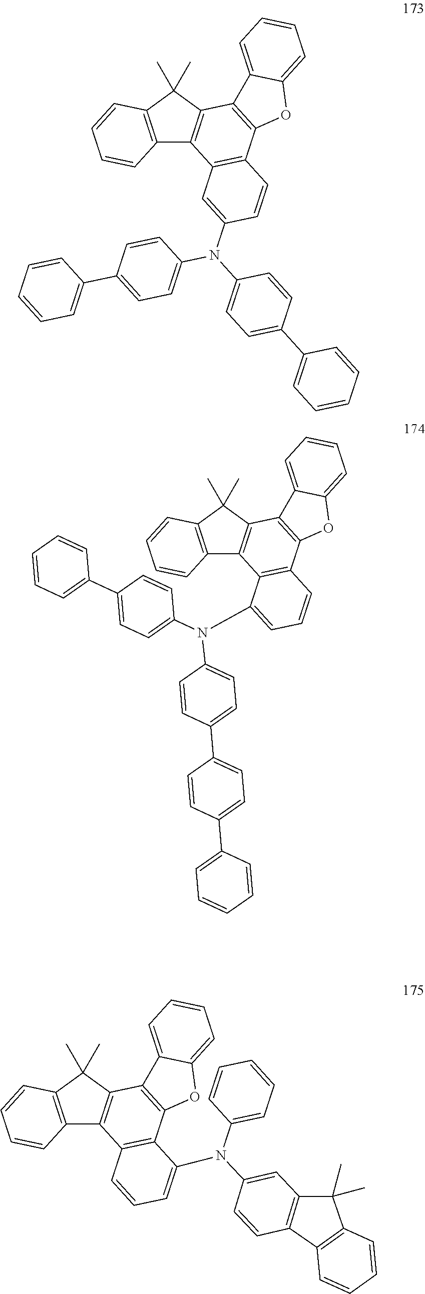

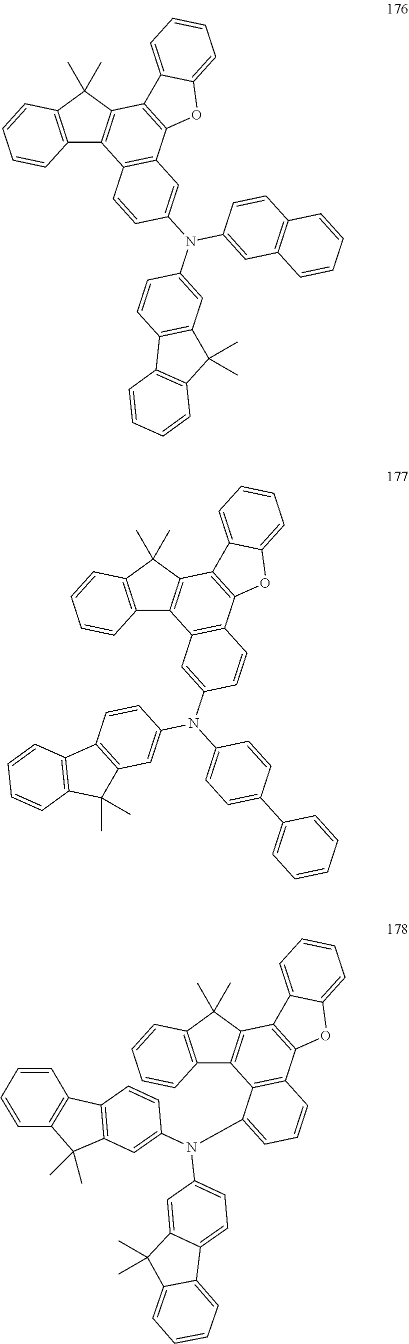 [SMILES: CC1(C)c2ccccc2-c2ccc(N(c3ccc4ccccc4c3)c3ccc4c5c(c6c7ccccc7oc6c4c3)C(C)(C)c3ccccc3-5)cc21 ; CC1(C)c2ccccc2-c2ccc(N(c3ccc(-c4ccccc4)cc3)c3ccc4c(c3)c3c(c5c6ccccc6oc45)C(C)(C)c4ccccc4-3)cc21 ; CC1(C)c2ccccc2-c2ccc(N(c3ccc4c(c3)C(C)(C)c3ccccc3-4)c3cccc4c3c3c(c5c6ccccc6oc45)C(C)(C)c4ccccc4-3)cc21]

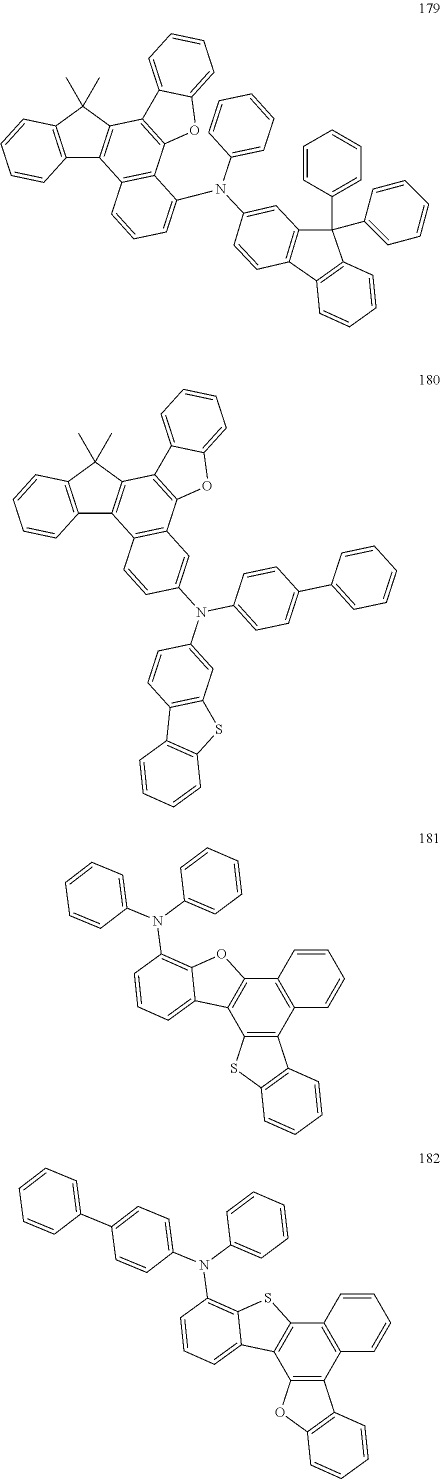 [SMILES: CC1(C)c2ccccc2-c2c1c1c3ccccc3oc1c1c(N(c3ccccc3)c3ccc4c(c3)C(c3ccccc3)(c3ccccc3)c3ccccc3-4)cccc21 ; CC1(C)c2ccccc2-c2c1c1c3ccccc3oc1c1cc(N(c3ccc(-c4ccccc4)cc3)c3ccc4c(c3)sc3ccccc34)ccc21 ; c1ccc(N(c2ccccc2)c2cccc3c2oc2c4ccccc4c4c5ccccc5sc4c32)cc1 ; c1ccc(-c2ccc(N(c3ccccc3)c3cccc4c3sc3c5ccccc5c5c6ccccc6oc5c43)cc2)cc1]

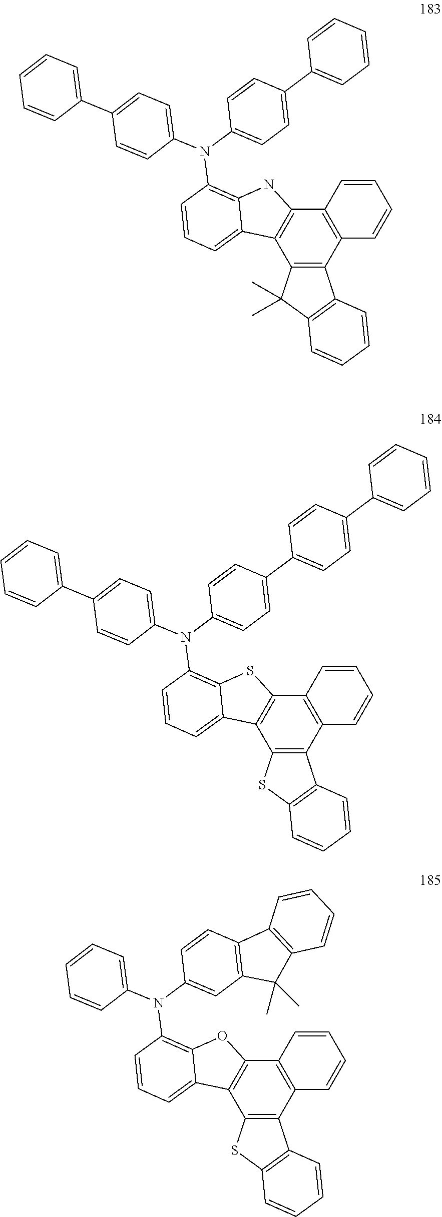

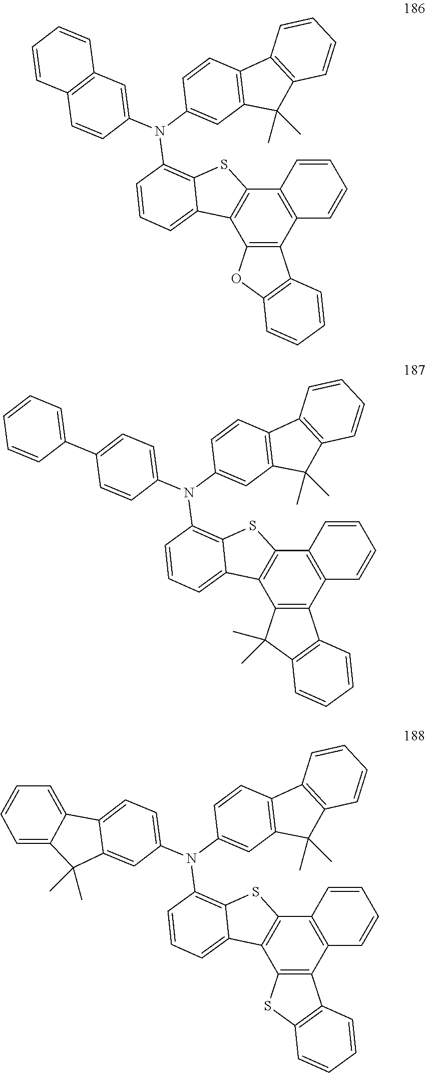

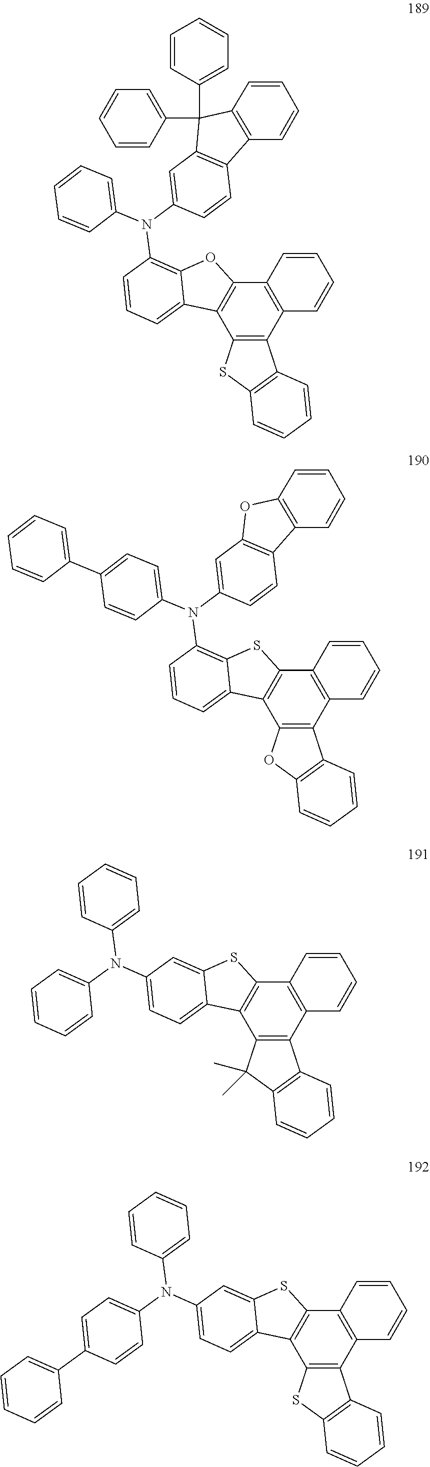

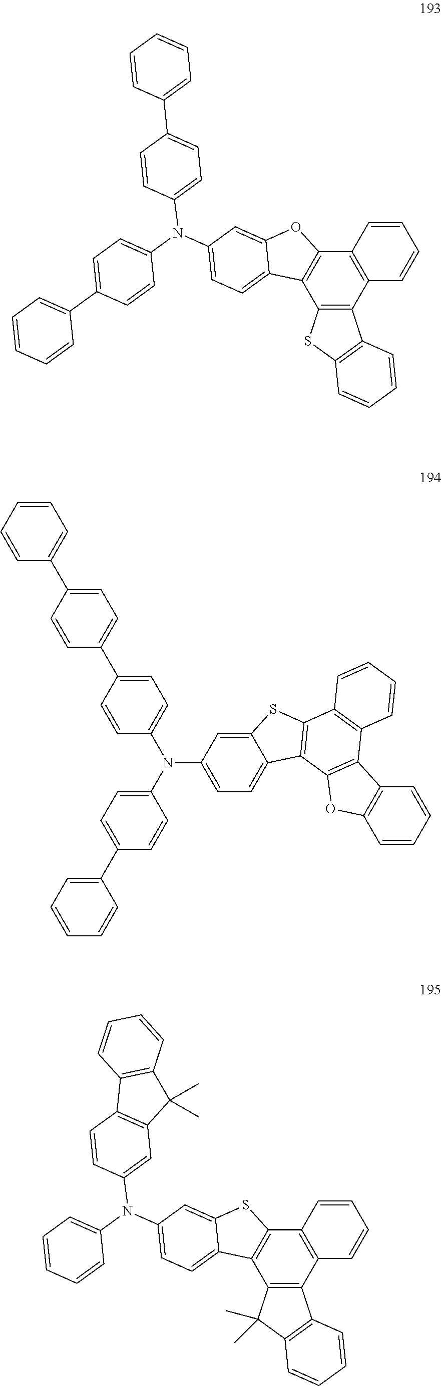 [SMILES: c1ccc(-c2ccc(N(c3ccc(-c4ccccc4)cc3)c3ccc4c(c3)oc3c5ccccc5c5c6ccccc6sc5c43)cc2)cc1 ; c1ccc(-c2ccc(-c3ccc(N(c4ccc(-c5ccccc5)cc4)c4ccc5c(c4)sc4c6ccccc6c6c7ccccc7oc6c54)cc3)cc2)cc1 ; CC1(C)c2ccccc2-c2ccc(N(c3ccccc3)c3ccc4c(c3)sc3c5ccccc5c5c(c43)C(C)(C)c3ccccc3-5)cc21]

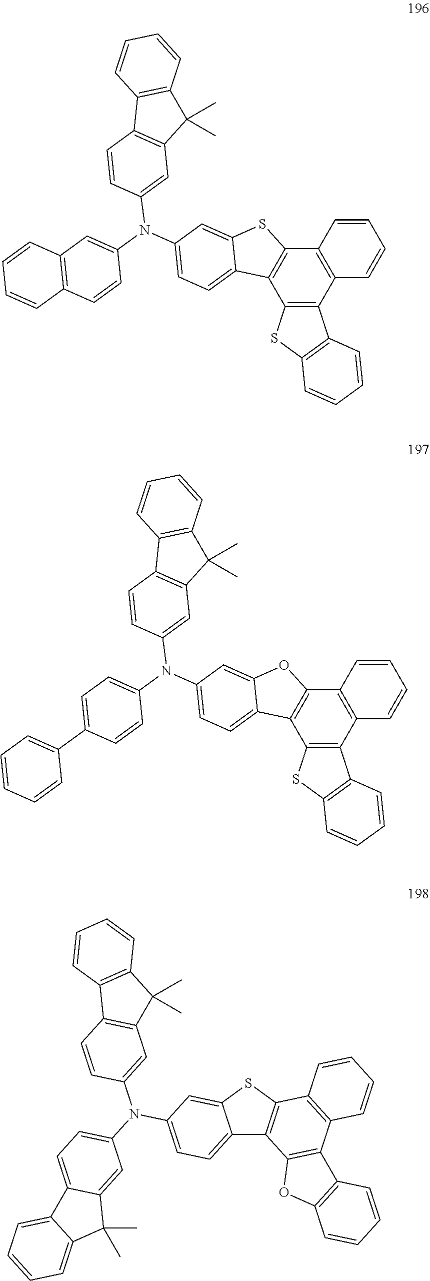 [SMILES: CC1(C)c2ccccc2-c2ccc(N(c3ccc4ccccc4c3)c3ccc4c(c3)sc3c5ccccc5c5c6ccccc6sc5c43)cc21 ; CC1(C)c2ccccc2-c2ccc(N(c3ccc(-c4ccccc4)cc3)c3ccc4c(c3)oc3c5ccccc5c5c6ccccc6sc5c43)cc21 ; CC1(C)c2ccccc2-c2ccc(N(c3ccc4c(c3)C(C)(C)c3ccccc3-4)c3ccc4c(c3)sc3c5ccccc5c5c6ccccc6oc5c43)cc21]

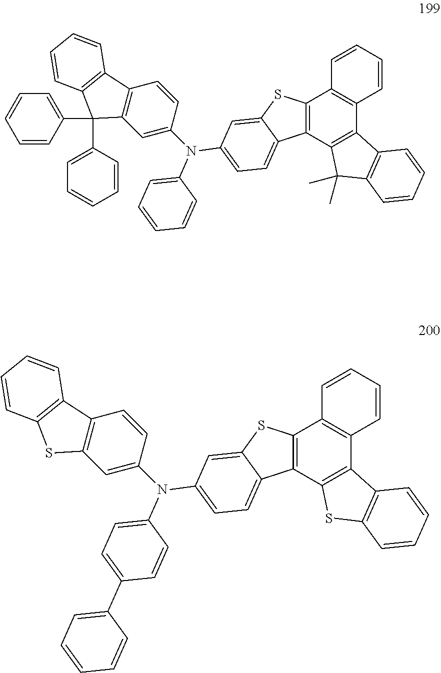 [SMILES: CC1(C)c2ccccc2-c2c1c1c3ccc(N(c4ccccc4)c4ccc5c(c4)C(c4ccccc4)(c4ccccc4)c4ccccc4-5)cc3sc1c1ccccc21 ; c1ccc(-c2ccc(N(c3ccc4c(c3)sc3ccccc34)c3ccc4c(c3)sc3c5ccccc5c5c6ccccc6sc5c43)cc2)cc1]

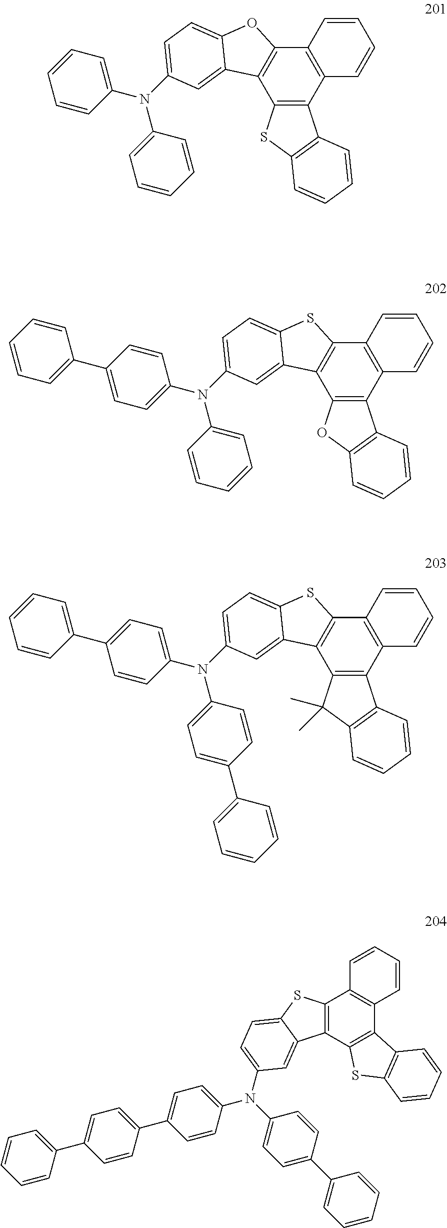 [SMILES: c1ccc(N(c2ccccc2)c2ccc3oc4c5ccccc5c5c6ccccc6sc5c4c3c2)cc1 ; c1ccc(-c2ccc(N(c3ccccc3)c3ccc4sc5c6ccccc6c6c7ccccc7oc6c5c4c3)cc2)cc1 ; CC1(C)c2ccccc2-c2c1c1c3cc(N(c4ccc(-c5ccccc5)cc4)c4ccc(-c5ccccc5)cc4)ccc3sc1c1ccccc21 ; c1ccc(-c2ccc(-c3ccc(N(c4ccc(-c5ccccc5)cc4)c4ccc5sc6c7ccccc7c7c8ccccc8sc7c6c5c4)cc3)cc2)cc1]

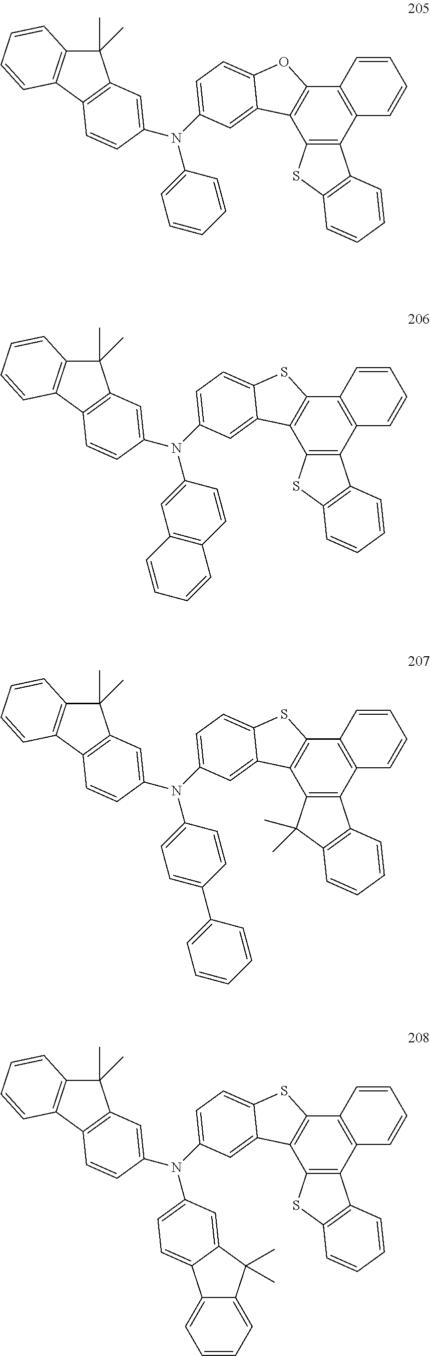 [SMILES: CC1(C)c2ccccc2-c2ccc(N(c3ccccc3)c3ccc4oc5c6ccccc6c6c7ccccc7sc6c5c4c3)cc21 ; CC1(C)c2ccccc2-c2ccc(N(c3ccc4ccccc4c3)c3ccc4sc5c6ccccc6c6c7ccccc7sc6c5c4c3)cc21 ; CC1(C)c2ccccc2-c2ccc(N(c3ccc(-c4ccccc4)cc3)c3ccc4sc5c6ccccc6c6c(c5c4c3)C(C)(C)c3ccccc3-6)cc21 ; CC1(C)c2ccccc2-c2ccc(N(c3ccc4c(c3)C(C)(C)c3ccccc3-4)c3ccc4sc5c6ccccc6c6c7ccccc7sc6c5c4c3)cc21]

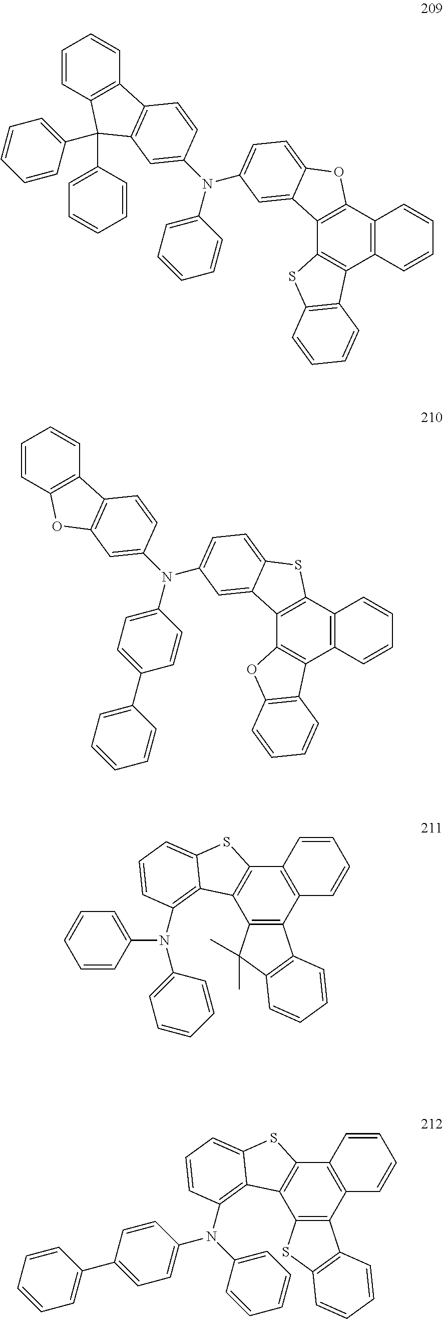 [SMILES: c1ccc(N(c2ccc3c(c2)C(c2ccccc2)(c2ccccc2)c2ccccc2-3)c2ccc3oc4c5ccccc5c5c6ccccc6sc5c4c3c2)cc1 ; c1ccc(-c2ccc(N(c3ccc4c(c3)oc3ccccc34)c3ccc4sc5c6ccccc6c6c7ccccc7oc6c5c4c3)cc2)cc1 ; CC1(C)c2ccccc2-c2c1c1c(sc3cccc(N(c4ccccc4)c4ccccc4)c31)c1ccccc21 ; c1ccc(-c2ccc(N(c3ccccc3)c3cccc4sc5c6ccccc6c6c7ccccc7sc6c5c34)cc2)cc1]

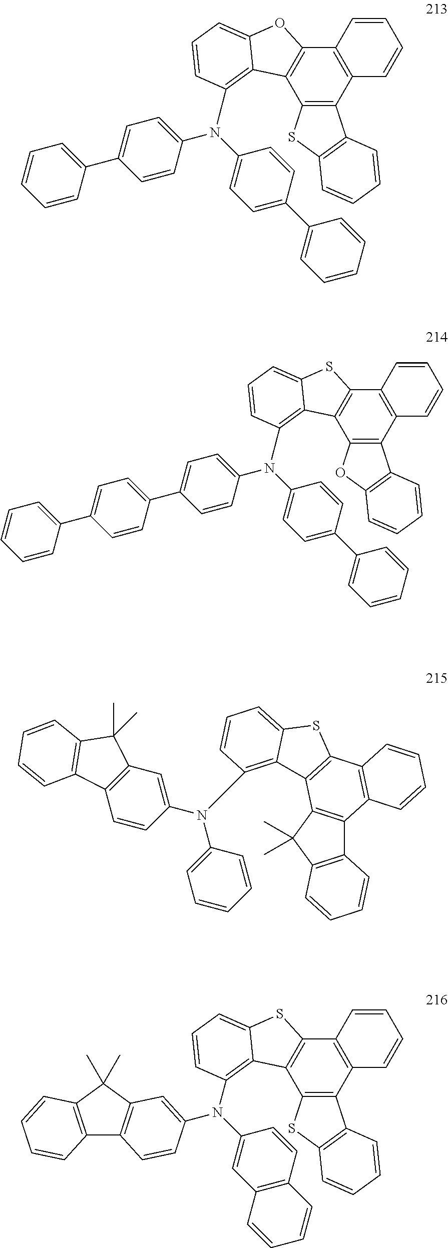 [SMILES: c1ccc(-c2ccc(N(c3ccc(-c4ccccc4)cc3)c3cccc4oc5c6ccccc6c6c7ccccc7sc6c5c34)cc2)cc1 ; c1ccc(-c2ccc(-c3ccc(N(c4ccc(-c5ccccc5)cc4)c4cccc5sc6c7ccccc7c7c8ccccc8oc7c6c45)cc3)cc2)cc1 ; CC1(C)c2ccccc2-c2ccc(N(c3ccccc3)c3cccc4sc5c6ccccc6c6c(c5c34)C(C)(C)c3ccccc3-6)cc21 ; CC1(C)c2ccccc2-c2ccc(N(c3ccc4ccccc4c3)c3cccc4sc5c6ccccc6c6c7ccccc7sc6c5c34)cc21]

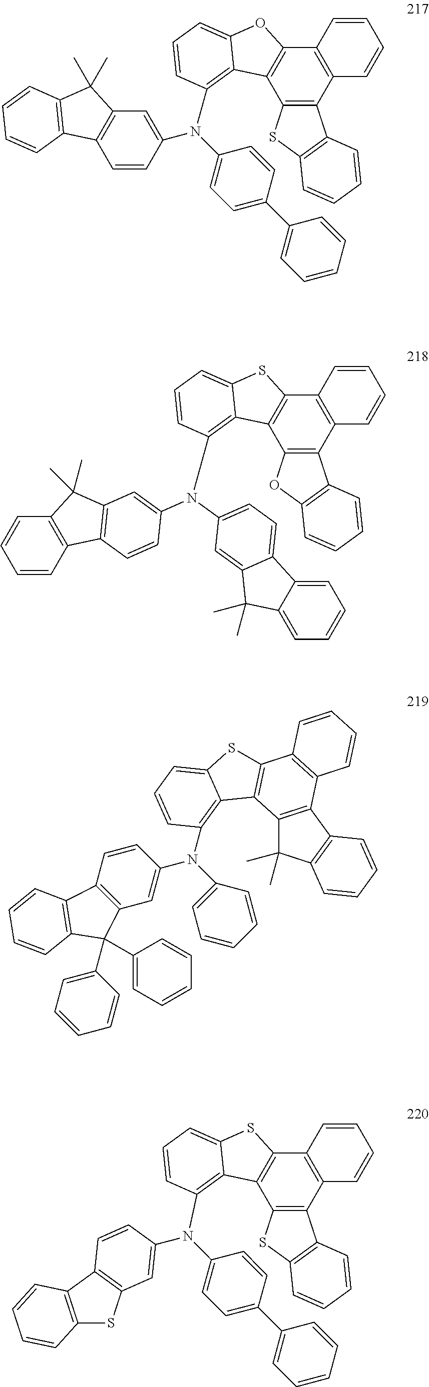 [SMILES: CC1(C)c2ccccc2-c2ccc(N(c3ccc(-c4ccccc4)cc3)c3cccc4oc5c6ccccc6c6c7ccccc7sc6c5c34)cc21 ; CC1(C)c2ccccc2-c2ccc(N(c3ccc4c(c3)C(C)(C)c3ccccc3-4)c3cccc4sc5c6ccccc6c6c7ccccc7oc6c5c34)cc21 ; CC1(C)c2ccccc2-c2c1c1c(sc3cccc(N(c4ccccc4)c4ccc5c(c4)C(c4ccccc4)(c4ccccc4)c4ccccc4-5)c31)c1ccccc21 ; c1ccc(-c2ccc(N(c3ccc4c(c3)sc3ccccc34)c3cccc4sc5c6ccccc6c6c7ccccc7sc6c5c34)cc2)cc1]

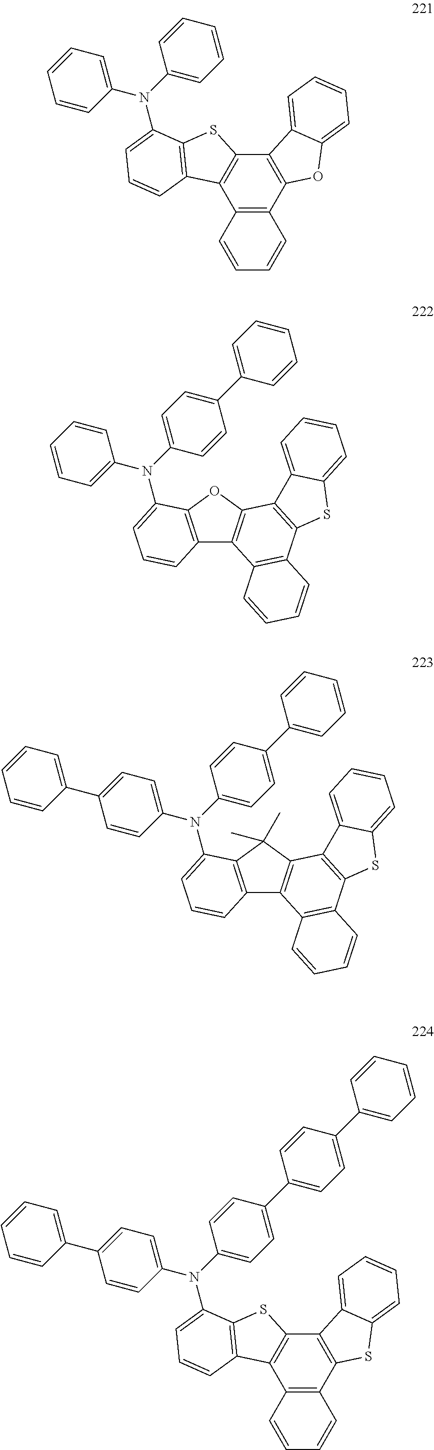 [SMILES: c1ccc(N(c2ccccc2)c2cccc3c2sc2c3c3ccccc3c3oc4ccccc4c32)cc1 ; c1ccc(-c2ccc(N(c3ccccc3)c3cccc4c3oc3c4c4ccccc4c4sc5ccccc5c34)cc2)cc1 ; CC1(C)c2c(cccc2N(c2ccc(-c3ccccc3)cc2)c2ccc(-c3ccccc3)cc2)-c2c1c1c3ccccc3sc1c1ccccc21 ; c1ccc(-c2ccc(-c3ccc(N(c4ccc(-c5ccccc5)cc4)c4cccc5c4sc4c5c5ccccc5c5sc6ccccc6c54)cc3)cc2)cc1]

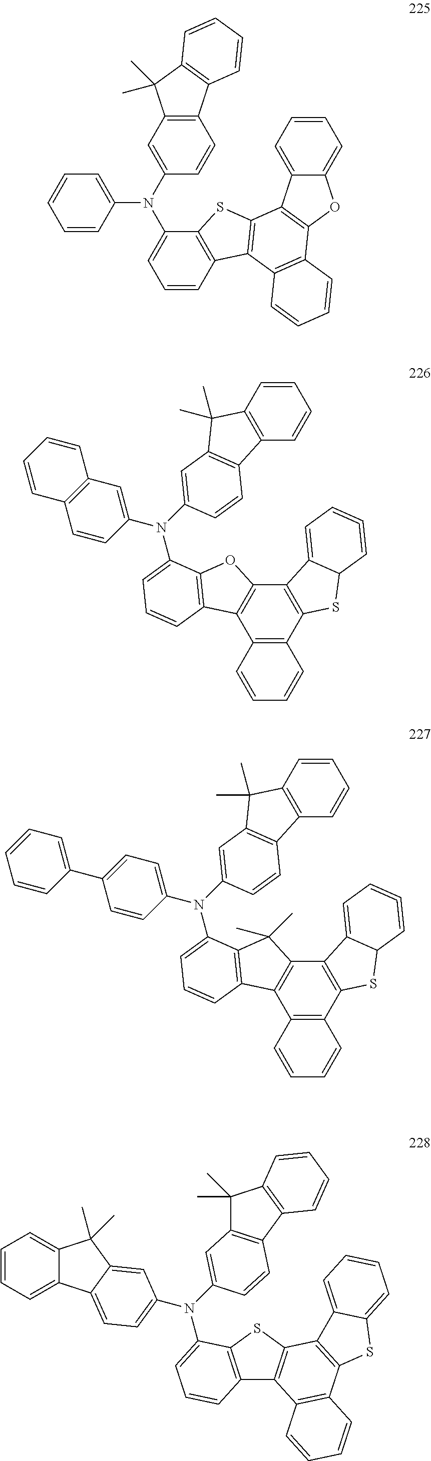 [SMILES: CC1(C)c2ccccc2-c2ccc(N(c3ccccc3)c3cccc4c3sc3c4c4ccccc4c4oc5ccccc5c43)cc21 ; CC1(C)c2ccccc2-c2ccc(N(c3ccc4ccccc4c3)c3cccc4c3oc3c5c(c6ccccc6c34)SC3C=C=CC=C53)cc21 ; CC1(C)c2ccccc2-c2ccc(N(c3ccc(-c4ccccc4)cc3)c3cccc4c3C(C)(C)c3c5c(c6ccccc6c3-4)SC3C=C=CC=C53)cc21 ; CC1(C)c2ccccc2-c2ccc(N(c3ccc4c(c3)C(C)(C)c3ccccc3-4)c3cccc4c3sc3c4c4ccccc4c4sc5ccccc5c43)cc21]

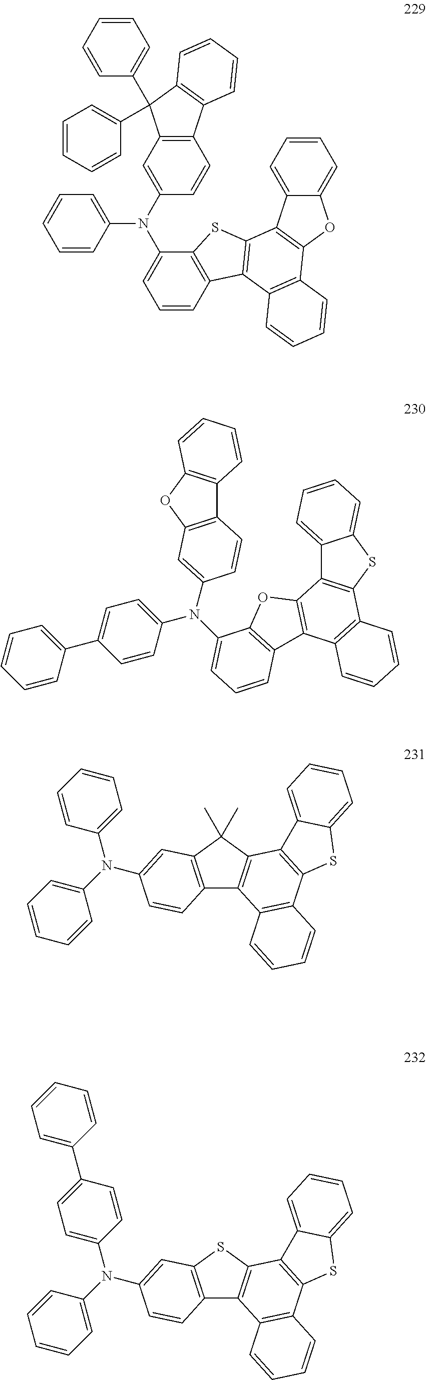 [SMILES: c1ccc(N(c2ccc3c(c2)C(c2ccccc2)(c2ccccc2)c2ccccc2-3)c2cccc3c2sc2c3c3ccccc3c3oc4ccccc4c32)cc1 ; c1ccc(-c2ccc(N(c3ccc4c(c3)oc3ccccc34)c3cccc4c3oc3c4c4ccccc4c4sc5ccccc5c34)cc2)cc1 ; CC1(C)c2cc(N(c3ccccc3)c3ccccc3)ccc2-c2c1c1c3ccccc3sc1c1ccccc21 ; c1ccc(-c2ccc(N(c3ccccc3)c3ccc4c(c3)sc3c4c4ccccc4c4sc5ccccc5c43)cc2)cc1]

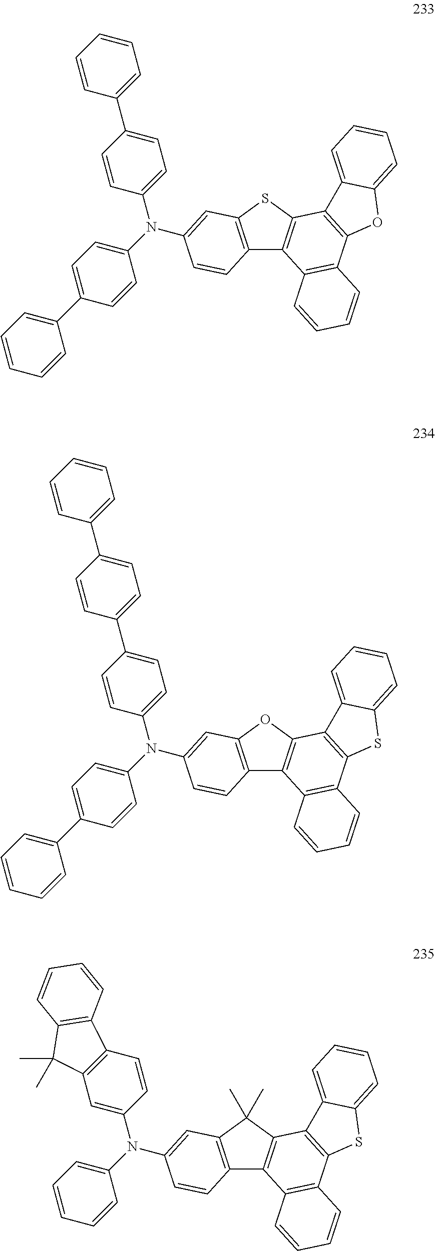 [SMILES: c1ccc(-c2ccc(N(c3ccc(-c4ccccc4)cc3)c3ccc4c(c3)sc3c4c4ccccc4c4oc5ccccc5c43)cc2)cc1 ; c1ccc(-c2ccc(-c3ccc(N(c4ccc(-c5ccccc5)cc4)c4ccc5c(c4)oc4c5c5ccccc5c5sc6ccccc6c45)cc3)cc2)cc1 ; CC1(C)c2ccccc2-c2ccc(N(c3ccccc3)c3ccc4c(c3)C(C)(C)c3c-4c4ccccc4c4sc5ccccc5c34)cc21]

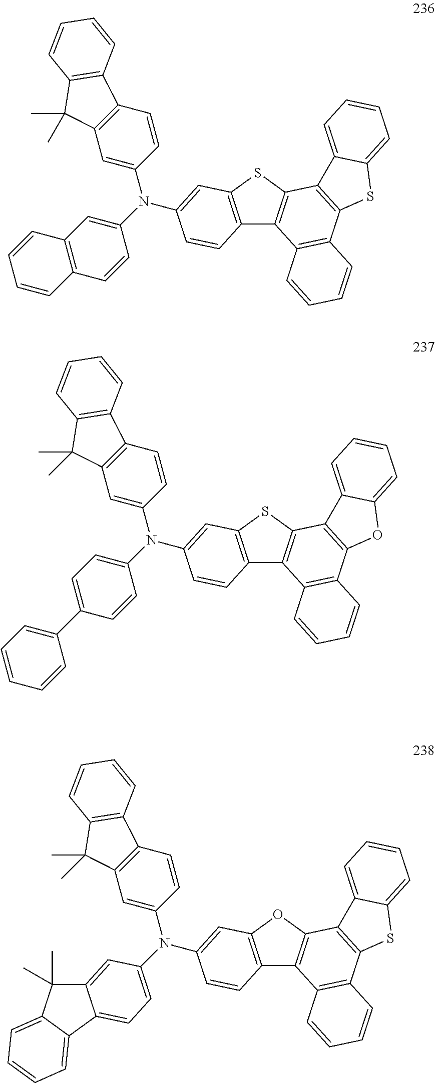 [SMILES: CC1(C)c2ccccc2-c2ccc(N(c3ccc4ccccc4c3)c3ccc4c(c3)sc3c4c4ccccc4c4sc5ccccc5c43)cc21 ; CC1(C)c2ccccc2-c2ccc(N(c3ccc(-c4ccccc4)cc3)c3ccc4c(c3)sc3c4c4ccccc4c4oc5ccccc5c43)cc21 ; CC1(C)c2ccccc2-c2ccc(N(c3ccc4c(c3)C(C)(C)c3ccccc3-4)c3ccc4c(c3)oc3c4c4ccccc4c4sc5ccccc5c34)cc21]

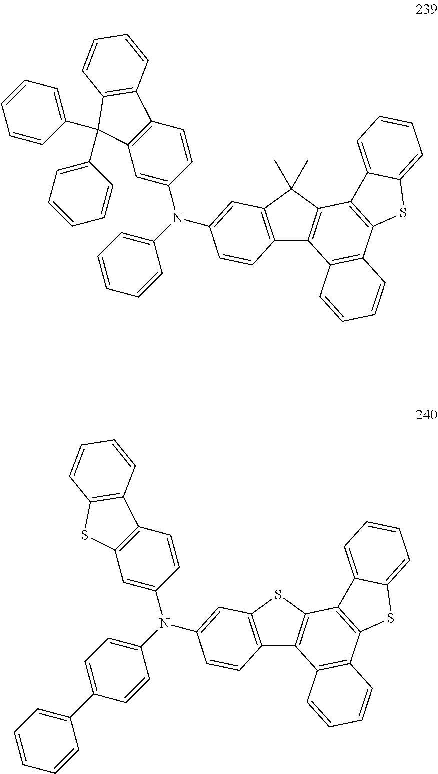 [SMILES: CC1(C)c2cc(N(c3ccccc3)c3ccc4c(c3)C(c3ccccc3)(c3ccccc3)c3ccccc3-4)ccc2-c2c1c1c3ccccc3sc1c1ccccc21 ; c1ccc(-c2ccc(N(c3ccc4c(c3)sc3ccccc34)c3ccc4c(c3)sc3c4c4ccccc4c4sc5ccccc5c43)cc2)cc1]

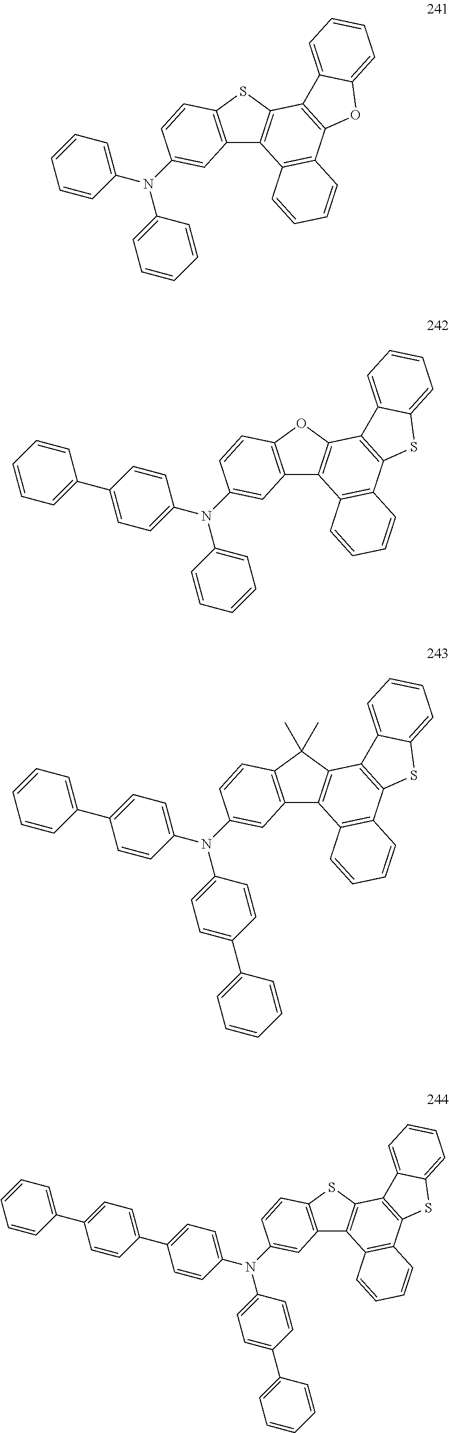 [SMILES: c1ccc(N(c2ccccc2)c2ccc3sc4c(c3c2)c2ccccc2c2oc3ccccc3c24)cc1 ; c1ccc(-c2ccc(N(c3ccccc3)c3ccc4oc5c(c4c3)c3ccccc3c3sc4ccccc4c53)cc2)cc1 ; CC1(C)c2ccc(N(c3ccc(-c4ccccc4)cc3)c3ccc(-c4ccccc4)cc3)cc2-c2c1c1c3ccccc3sc1c1ccccc21 ; c1ccc(-c2ccc(-c3ccc(N(c4ccc(-c5ccccc5)cc4)c4ccc5sc6c(c5c4)c4ccccc4c4sc5ccccc5c46)cc3)cc2)cc1]

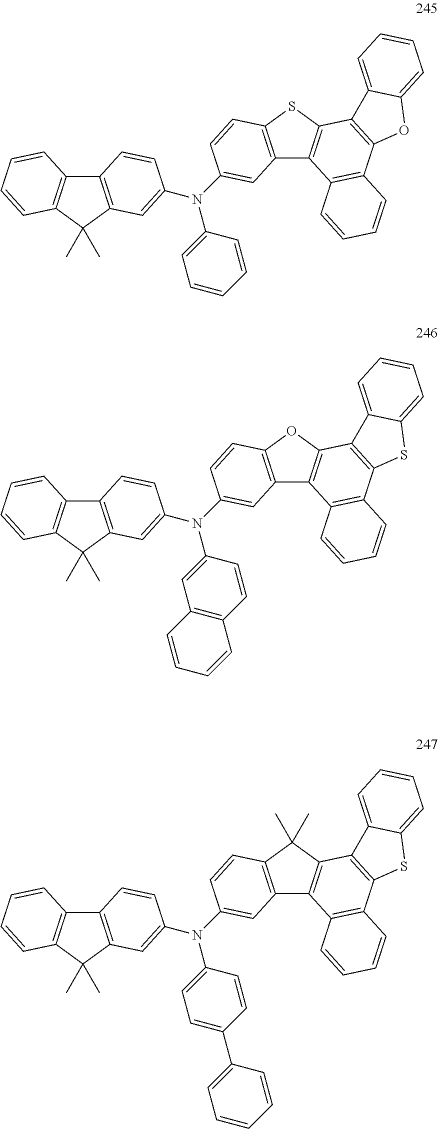 [SMILES: CC1(C)c2ccccc2-c2ccc(N(c3ccccc3)c3ccc4sc5c(c4c3)c3ccccc3c3oc4ccccc4c35)cc21 ; CC1(C)c2ccccc2-c2ccc(N(c3ccc4ccccc4c3)c3ccc4oc5c(c4c3)c3ccccc3c3sc4ccccc4c53)cc21 ; CC1(C)c2ccccc2-c2ccc(N(c3ccc(-c4ccccc4)cc3)c3ccc4c(c3)-c3c(c5c6ccccc6sc5c5ccccc35)C4(C)C)cc21]

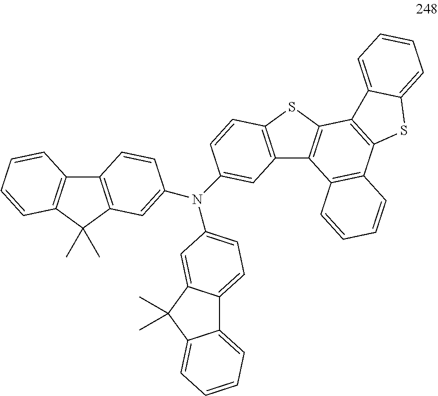 [SMILES: CC1(C)c2ccccc2-c2ccc(N(c3ccc4c(c3)C(C)(C)c3ccccc3-4)c3ccc4sc5c(c4c3)c3ccccc3c3sc4ccccc4c35)cc21]

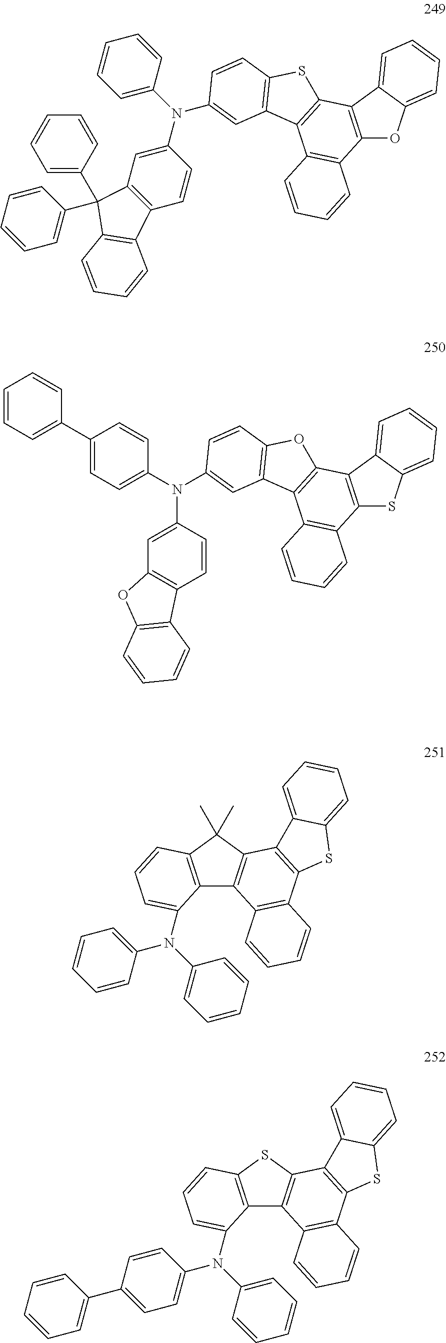 [SMILES: c1ccc(N(c2ccc3c(c2)C(c2ccccc2)(c2ccccc2)c2ccccc2-3)c2ccc3sc4c(c3c2)c2ccccc2c2oc3ccccc3c24)cc1 ; c1ccc(-c2ccc(N(c3ccc4c(c3)oc3ccccc34)c3ccc4oc5c(c4c3)c3ccccc3c3sc4ccccc4c53)cc2)cc1 ; CC1(C)c2cccc(N(c3ccccc3)c3ccccc3)c2-c2c1c1c3ccccc3sc1c1ccccc21 ; c1ccc(-c2ccc(N(c3ccccc3)c3cccc4sc5c6c7ccccc7sc6c6ccccc6c5c34)cc2)cc1]

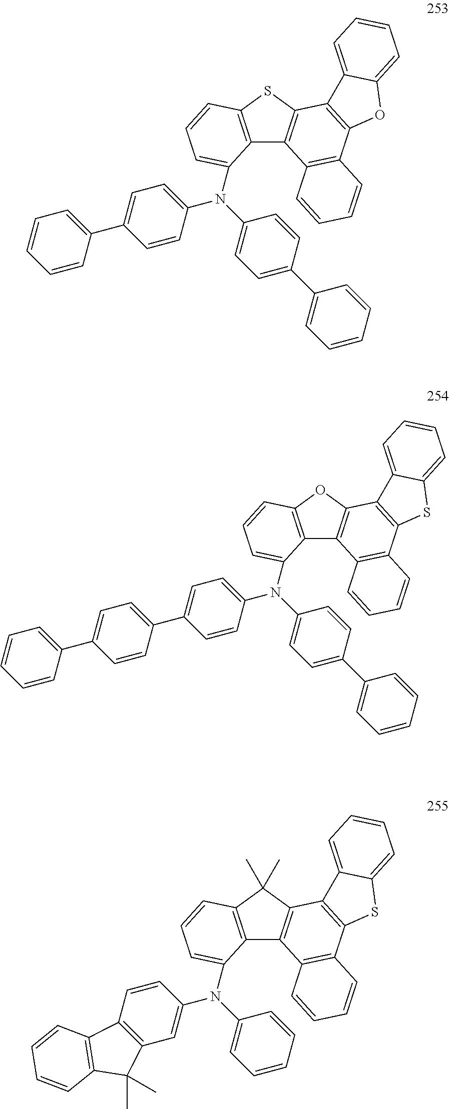 [SMILES: c1ccc(-c2ccc(N(c3ccc(-c4ccccc4)cc3)c3cccc4sc5c6c7ccccc7oc6c6ccccc6c5c34)cc2)cc1 ; c1ccc(-c2ccc(-c3ccc(N(c4ccc(-c5ccccc5)cc4)c4cccc5oc6c7c8ccccc8sc7c7ccccc7c6c45)cc3)cc2)cc1 ; CC1(C)c2ccccc2-c2ccc(N(c3ccccc3)c3cccc4c3-c3c(c5c6ccccc6sc5c5ccccc35)C4(C)C)cc21]

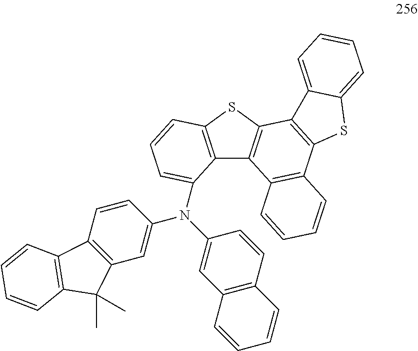 [SMILES: CC1(C)c2ccccc2-c2ccc(N(c3ccc4ccccc4c3)c3cccc4sc5c6c7ccccc7sc6c6ccccc6c5c34)cc21]

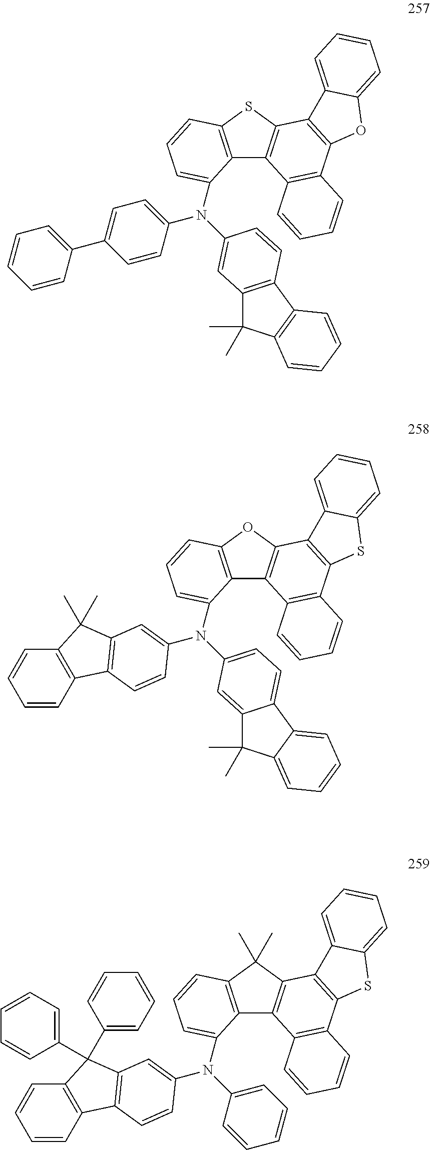 [SMILES: CC1(C)c2ccccc2-c2ccc(N(c3ccc(-c4ccccc4)cc3)c3cccc4sc5c6c7ccccc7oc6c6ccccc6c5c34)cc21 ; CC1(C)c2ccccc2-c2ccc(N(c3ccc4c(c3)C(C)(C)c3ccccc3-4)c3cccc4oc5c6c7ccccc7sc6c6ccccc6c5c34)cc21 ; CC1(C)c2cccc(N(c3ccccc3)c3ccc4c(c3)C(c3ccccc3)(c3ccccc3)c3ccccc3-4)c2-c2c1c1c3ccccc3sc1c1ccccc21]

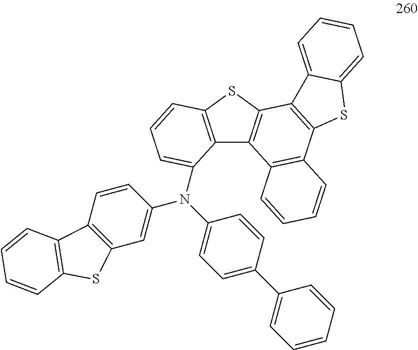 [SMILES: c1ccc(-c2ccc(N(c3ccc4c(c3)sc3ccccc34)c3cccc4sc5c6c7ccccc7sc6c6ccccc6c5c34)cc2)cc1]

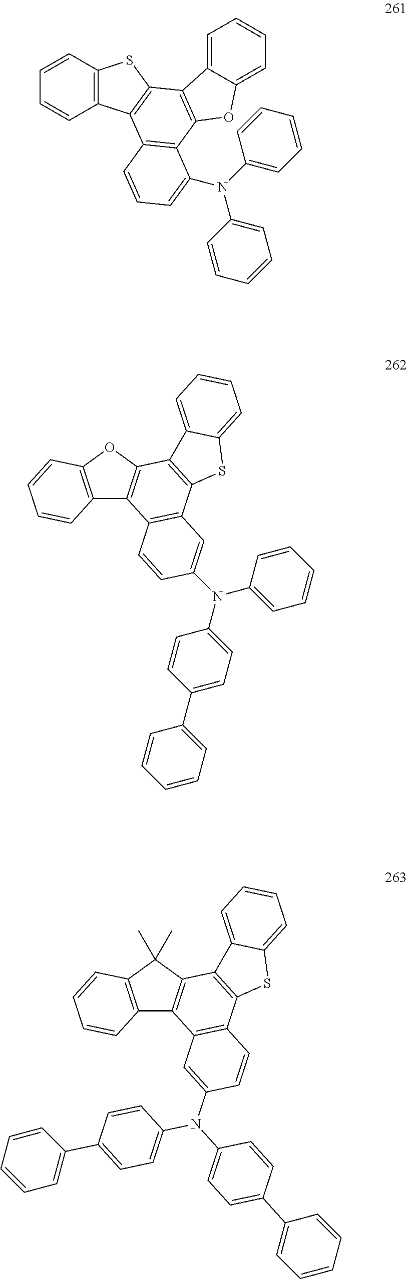 [SMILES: c1ccc(N(c2ccccc2)c2cccc3c2c2oc4ccccc4c2c2sc4ccccc4c32)cc1 ; c1ccc(-c2ccc(N(c3ccccc3)c3ccc4c(c3)c3sc5ccccc5c3c3oc5ccccc5c43)cc2)cc1 ; CC1(C)c2ccccc2-c2c1c1c3ccccc3sc1c1ccc(N(c3ccc(-c4ccccc4)cc3)c3ccc(-c4ccccc4)cc3)cc21]

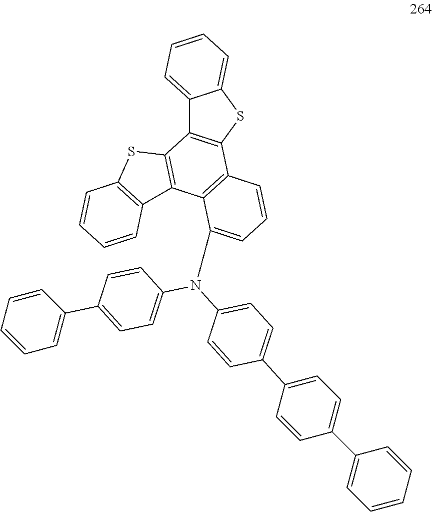 [SMILES: c1ccc(-c2ccc(-c3ccc(N(c4ccc(-c5ccccc5)cc4)c4cccc5c6sc7ccccc7c6c6sc7ccccc7c6c45)cc3)cc2)cc1]

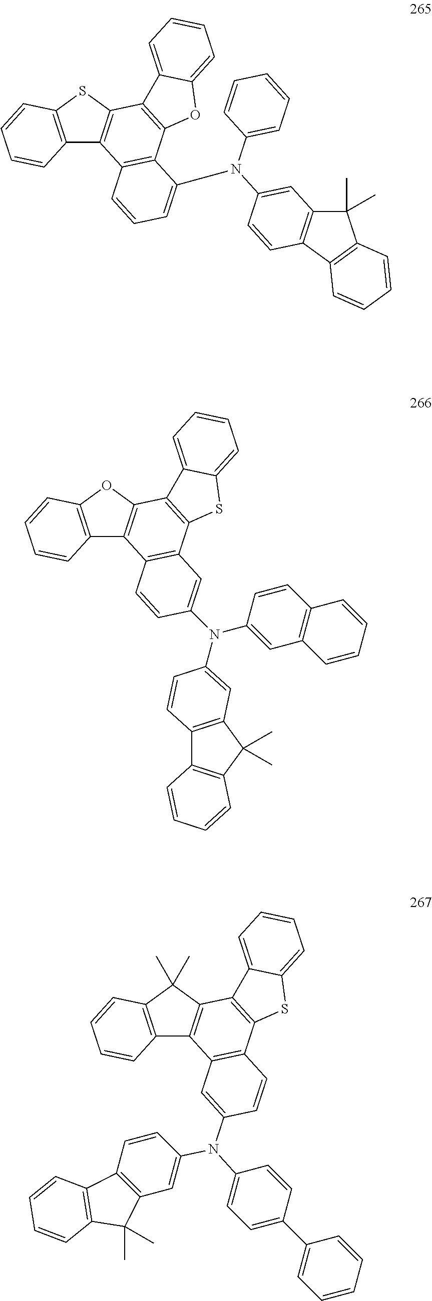 [SMILES: CC1(C)c2ccccc2-c2ccc(N(c3ccccc3)c3cccc4c3c3oc5ccccc5c3c3sc5ccccc5c43)cc21 ; CC1(C)c2ccccc2-c2ccc(N(c3ccc4ccccc4c3)c3ccc4c(c3)c3sc5ccccc5c3c3oc5ccccc5c43)cc21 ; CC1(C)c2ccccc2-c2ccc(N(c3ccc(-c4ccccc4)cc3)c3ccc4c(c3)c3c(c5c6ccccc6sc45)C(C)(C)c4ccccc4-3)cc21]

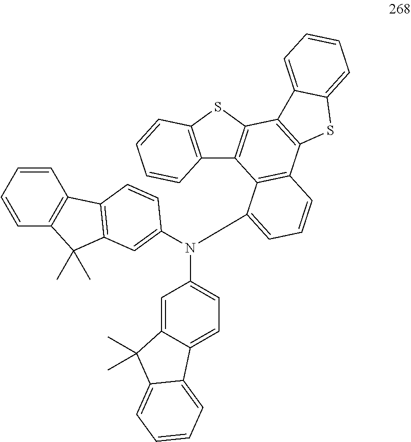 [SMILES: CC1(C)c2ccccc2-c2ccc(N(c3ccc4c(c3)C(C)(C)c3ccccc3-4)c3cccc4c5sc6ccccc6c5c5sc6ccccc6c5c34)cc21]

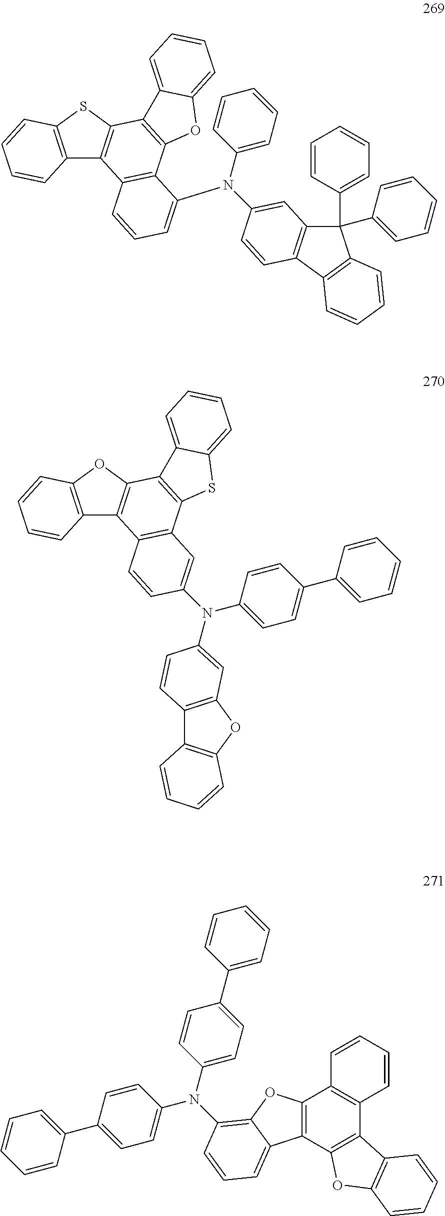 [SMILES: c1ccc(N(c2ccc3c(c2)C(c2ccccc2)(c2ccccc2)c2ccccc2-3)c2cccc3c2c2oc4ccccc4c2c2sc4ccccc4c32)cc1 ; c1ccc(-c2ccc(N(c3ccc4c(c3)oc3ccccc34)c3ccc4c(c3)c3sc5ccccc5c3c3oc5ccccc5c43)cc2)cc1 ; c1ccc(-c2ccc(N(c3ccc(-c4ccccc4)cc3)c3cccc4c3oc3c5ccccc5c5c6ccccc6oc5c43)cc2)cc1]

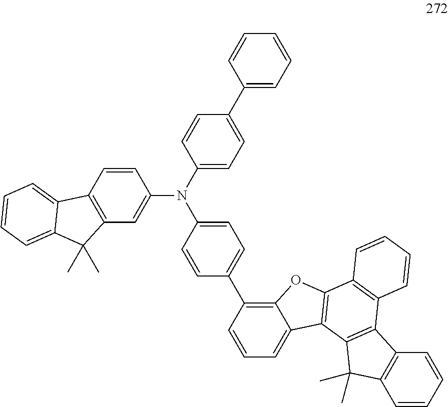 [SMILES: CC1(C)c2ccccc2-c2ccc(N(c3ccc(-c4ccccc4)cc3)c3ccc(-c4cccc5c4oc4c6ccccc6c6c(c54)C(C)(C)c4ccccc4-6)cc3)cc21]

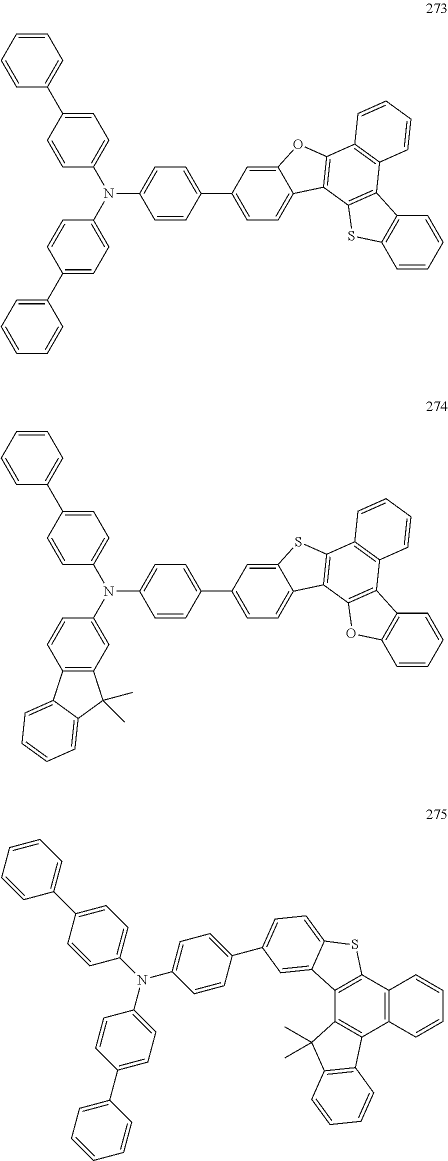 [SMILES: c1ccc(-c2ccc(N(c3ccc(-c4ccccc4)cc3)c3ccc(-c4ccc5c(c4)oc4c6ccccc6c6c7ccccc7sc6c54)cc3)cc2)cc1 ; CC1(C)c2ccccc2-c2ccc(N(c3ccc(-c4ccccc4)cc3)c3ccc(-c4ccc5c(c4)sc4c6ccccc6c6c7ccccc7oc6c54)cc3)cc21 ; CC1(C)c2ccccc2-c2c1c1c3cc(-c4ccc(N(c5ccc(-c6ccccc6)cc5)c5ccc(-c6ccccc6)cc5)cc4)ccc3sc1c1ccccc21]

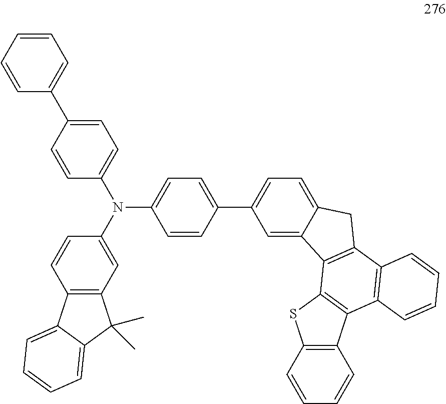 [SMILES: CC1(C)c2ccccc2-c2ccc(N(c3ccc(-c4ccccc4)cc3)c3ccc(-c4ccc5c(c4)-c4c(c6ccccc6c6c4sc4ccccc46)C5)cc3)cc21]

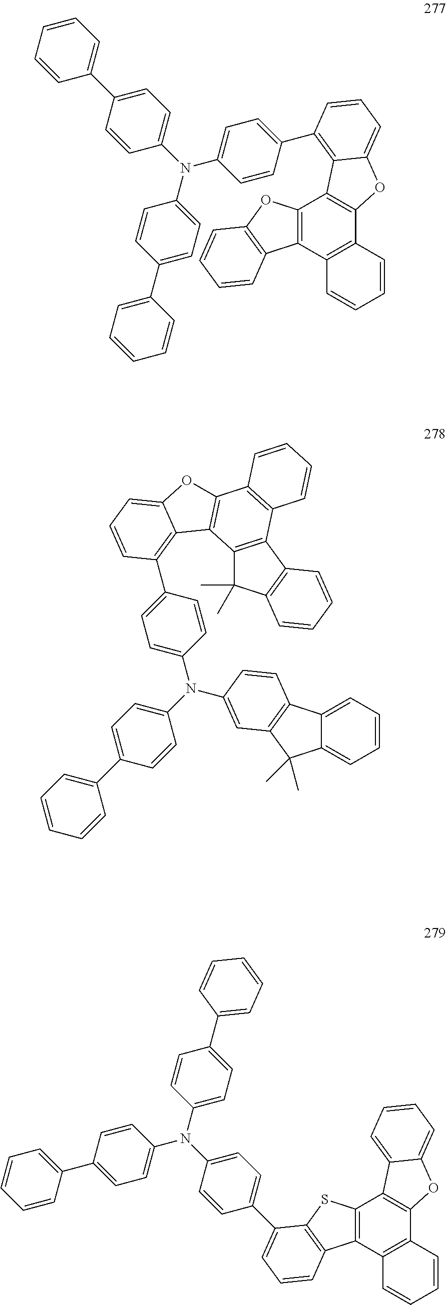 [SMILES: c1ccc(-c2ccc(N(c3ccc(-c4ccccc4)cc3)c3ccc(-c4cccc5oc6c7ccccc7c7c8ccccc8oc7c6c45)cc3)cc2)cc1 ; CC1(C)c2ccccc2-c2ccc(N(c3ccc(-c4ccccc4)cc3)c3ccc(-c4cccc5oc6c7ccccc7c7c(c6c45)C(C)(C)c4ccccc4-7)cc3)cc21 ; c1ccc(-c2ccc(N(c3ccc(-c4ccccc4)cc3)c3ccc(-c4cccc5c4sc4c5c5ccccc5c5oc6ccccc6c54)cc3)cc2)cc1]

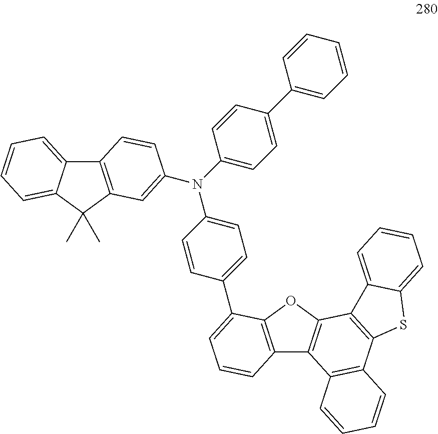 [SMILES: CC1(C)c2ccccc2-c2ccc(N(c3ccc(-c4ccccc4)cc3)c3ccc(-c4cccc5c4oc4c5c5ccccc5c5sc6ccccc6c45)cc3)cc21]

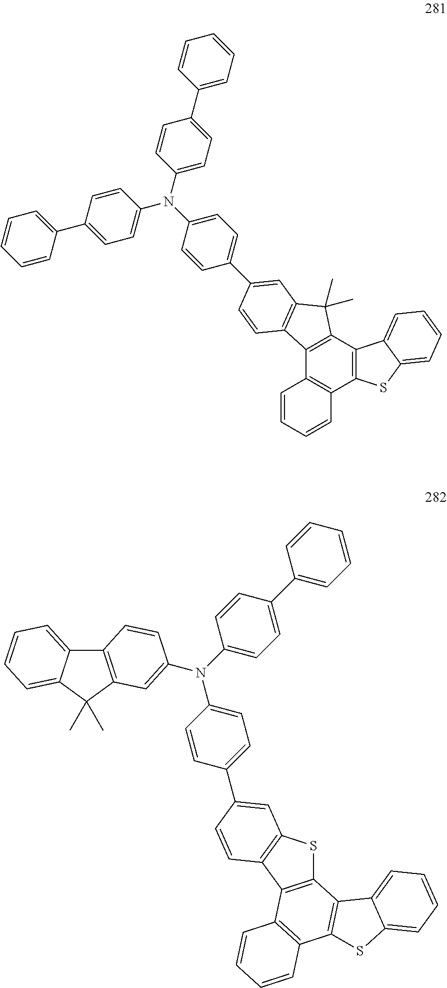 [SMILES: CC1(C)c2cc(-c3ccc(N(c4ccc(-c5ccccc5)cc4)c4ccc(-c5ccccc5)cc4)cc3)ccc2-c2c1c1c3ccccc3sc1c1ccccc21 ; CC1(C)c2ccccc2-c2ccc(N(c3ccc(-c4ccccc4)cc3)c3ccc(-c4ccc5c(c4)sc4c5c5ccccc5c5sc6ccccc6c54)cc3)cc21]

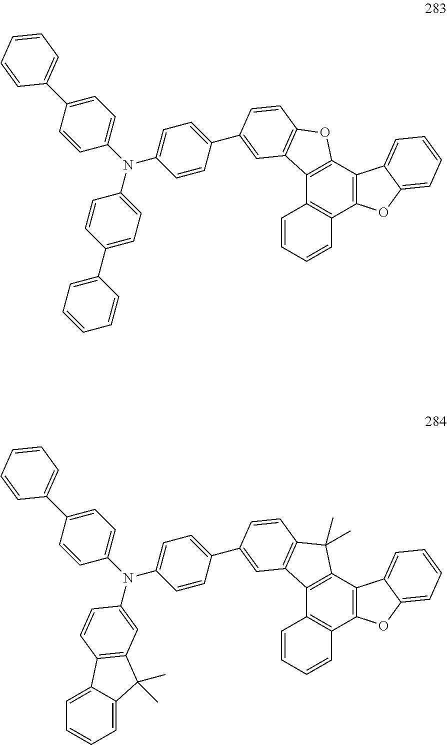 [SMILES: c1ccc(-c2ccc(N(c3ccc(-c4ccccc4)cc3)c3ccc(-c4ccc5oc6c(c5c4)c4ccccc4c4oc5ccccc5c46)cc3)cc2)cc1 ; CC1(C)c2ccccc2-c2ccc(N(c3ccc(-c4ccccc4)cc3)c3ccc(-c4ccc5c(c4)-c4c(c6c7ccccc7oc6c6ccccc46)C5(C)C)cc3)cc21]

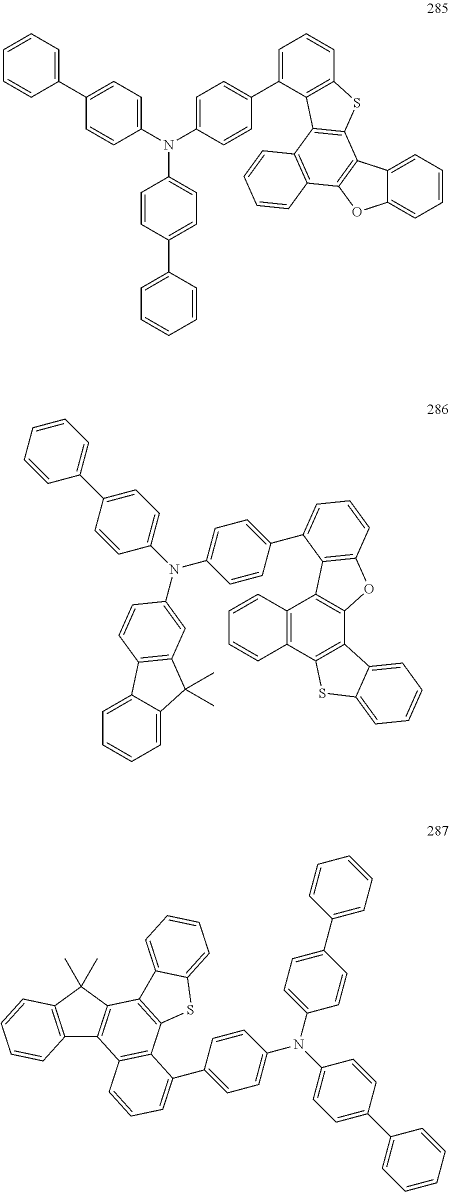 [SMILES: c1ccc(-c2ccc(N(c3ccc(-c4ccccc4)cc3)c3ccc(-c4cccc5sc6c7c8ccccc8oc7c7ccccc7c6c45)cc3)cc2)cc1 ; CC1(C)c2ccccc2-c2ccc(N(c3ccc(-c4ccccc4)cc3)c3ccc(-c4cccc5oc6c7c8ccccc8sc7c7ccccc7c6c45)cc3)cc21 ; CC1(C)c2ccccc2-c2c1c1c3ccccc3sc1c1c(-c3ccc(N(c4ccc(-c5ccccc5)cc4)c4ccc(-c5ccccc5)cc4)cc3)cccc21]

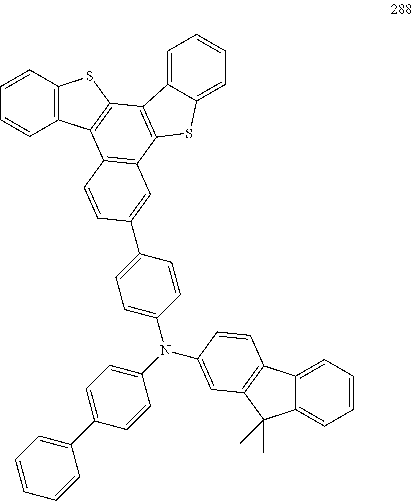 [SMILES: CC1(C)c2ccccc2-c2ccc(N(c3ccc(-c4ccccc4)cc3)c3ccc(-c4ccc5c(c4)c4sc6ccccc6c4c4sc6ccccc6c54)cc3)cc21]

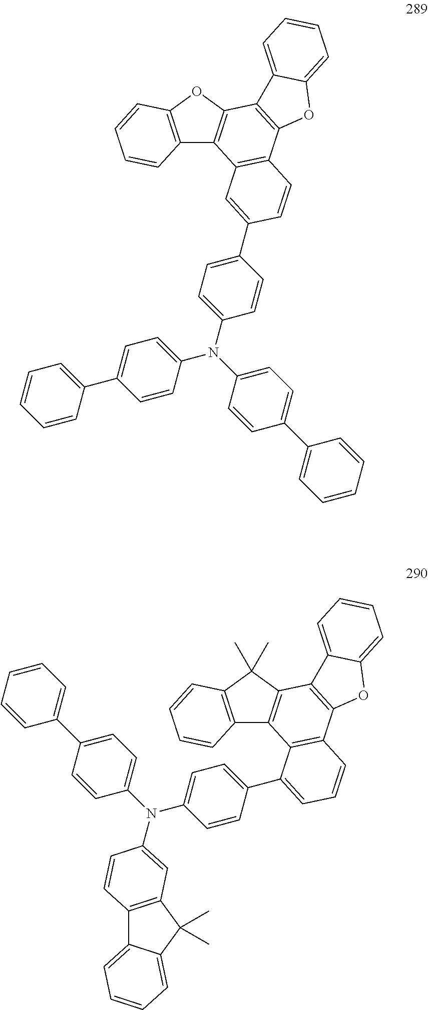 [SMILES: c1ccc(-c2ccc(N(c3ccc(-c4ccccc4)cc3)c3ccc(-c4ccc5c(c4)c4c6ccccc6oc4c4c6ccccc6oc54)cc3)cc2)cc1 ; CC1(C)c2ccccc2-c2ccc(N(c3ccc(-c4ccccc4)cc3)c3ccc(-c4cccc5c4c4c(c6c7ccccc7oc56)C(C)(C)c5ccccc5-4)cc3)cc21]

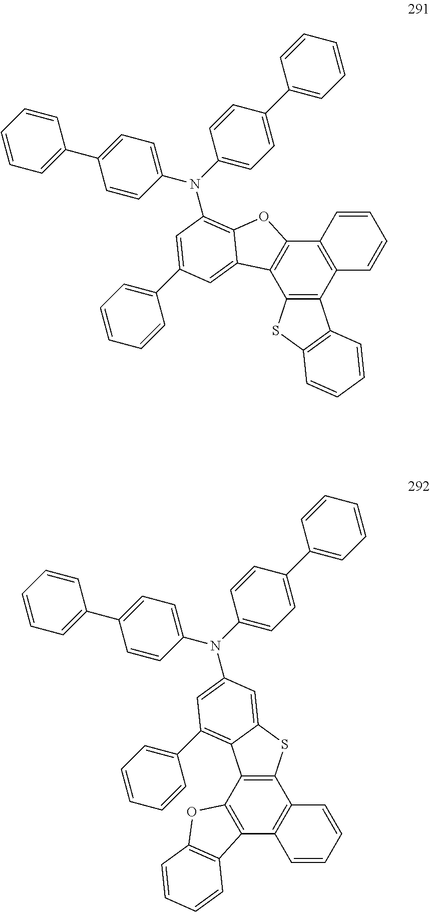 [SMILES: c1ccc(-c2ccc(N(c3ccc(-c4ccccc4)cc3)c3cc(-c4ccccc4)cc4c3oc3c5ccccc5c5c6ccccc6sc5c43)cc2)cc1 ; c1ccc(-c2ccc(N(c3ccc(-c4ccccc4)cc3)c3cc(-c4ccccc4)c4c(c3)sc3c5ccccc5c5c6ccccc6oc5c34)cc2)cc1]

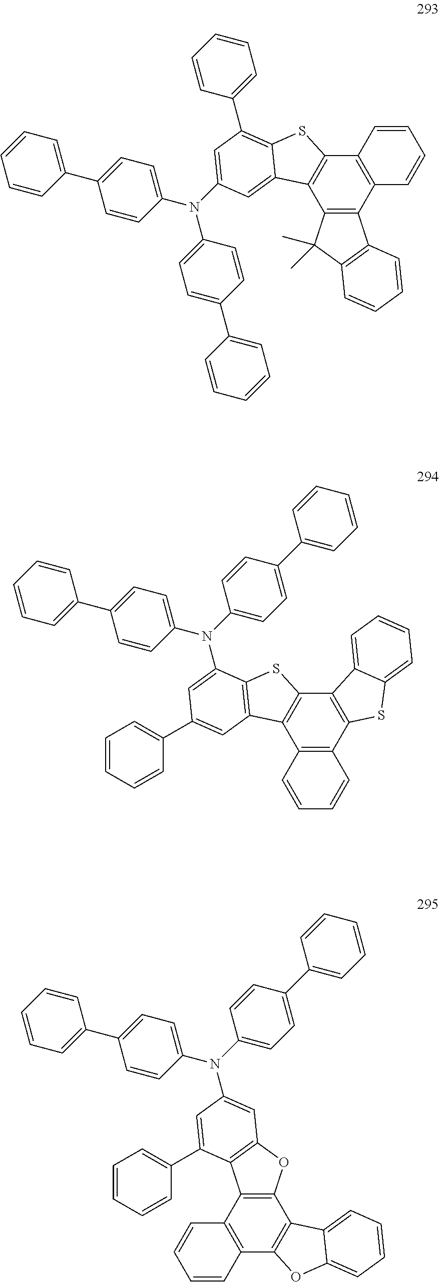 [SMILES: CC1(C)c2ccccc2-c2c1c1c3cc(N(c4ccc(-c5ccccc5)cc4)c4ccc(-c5ccccc5)cc4)cc(-c4ccccc4)c3sc1c1ccccc21 ; c1ccc(-c2ccc(N(c3ccc(-c4ccccc4)cc3)c3cc(-c4ccccc4)cc4c3sc3c4c4ccccc4c4sc5ccccc5c43)cc2)cc1 ; c1ccc(-c2ccc(N(c3ccc(-c4ccccc4)cc3)c3cc(-c4ccccc4)c4c(c3)oc3c5c6ccccc6oc5c5ccccc5c34)cc2)cc1]

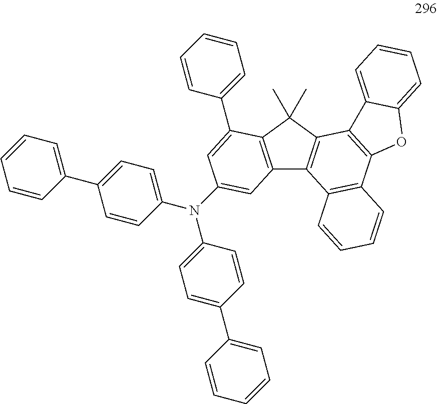 [SMILES: CC1(C)c2c(-c3ccccc3)cc(N(c3ccc(-c4ccccc4)cc3)c3ccc(-c4ccccc4)cc3)cc2-c2c1c1c3ccccc3oc1c1ccccc21]

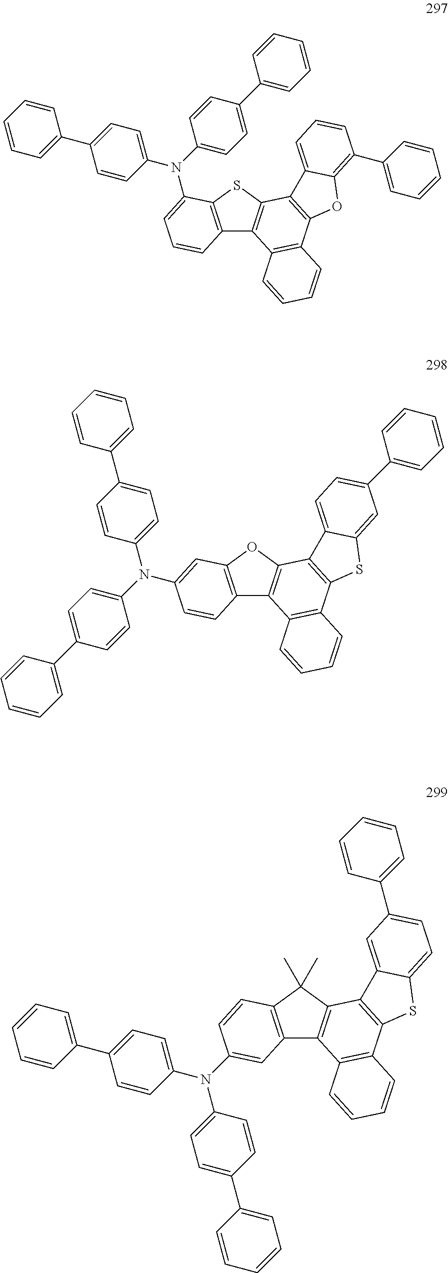 [SMILES: c1ccc(-c2ccc(N(c3ccc(-c4ccccc4)cc3)c3cccc4c3sc3c4c4ccccc4c4oc5c(-c6ccccc6)cccc5c43)cc2)cc1 ; c1ccc(-c2ccc(N(c3ccc(-c4ccccc4)cc3)c3ccc4c(c3)oc3c4c4ccccc4c4sc5cc(-c6ccccc6)ccc5c34)cc2)cc1 ; CC1(C)c2ccc(N(c3ccc(-c4ccccc4)cc3)c3ccc(-c4ccccc4)cc3)cc2-c2c1c1c3cc(-c4ccccc4)ccc3sc1c1ccccc21]

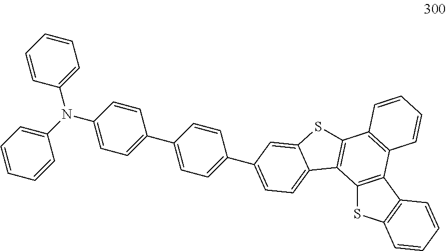 [SMILES: c1ccc(N(c2ccccc2)c2ccc(-c3ccc(-c4ccc5c(c4)sc4c6ccccc6c6c7ccccc7sc6c54)cc3)cc2)cc1]

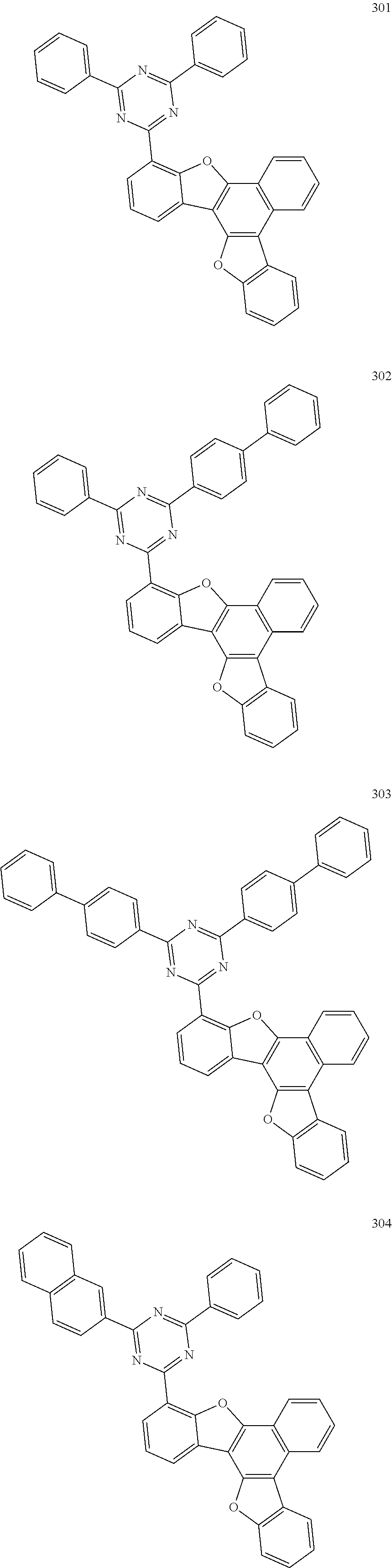 [SMILES: c1ccc(-c2nc(-c3ccccc3)nc(-c3cccc4c3oc3c5ccccc5c5c6ccccc6oc5c43)n2)cc1 ; c1ccc(-c2ccc(-c3nc(-c4ccccc4)nc(-c4cccc5c4oc4c6ccccc6c6c7ccccc7oc6c54)n3)cc2)cc1 ; c1ccc(-c2ccc(-c3nc(-c4ccc(-c5ccccc5)cc4)nc(-c4cccc5c4oc4c6ccccc6c6c7ccccc7oc6c54)n3)cc2)cc1 ; c1ccc(-c2nc(-c3ccc4ccccc4c3)nc(-c3cccc4c3oc3c5ccccc5c5c6ccccc6oc5c43)n2)cc1]

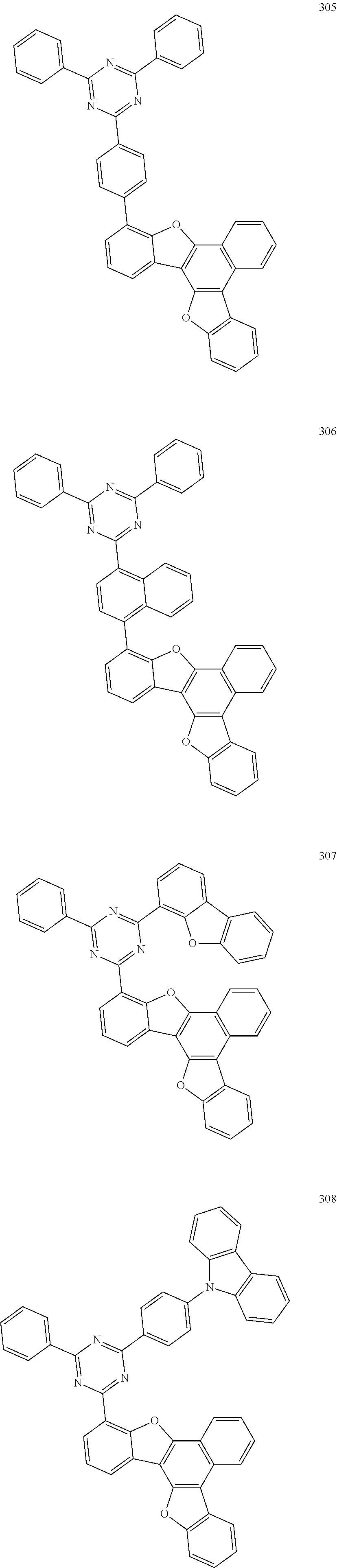 [SMILES: c1ccc(-c2nc(-c3ccccc3)nc(-c3ccc(-c4cccc5c4oc4c6ccccc6c6c7ccccc7oc6c54)cc3)n2)cc1 ; c1ccc(-c2nc(-c3ccccc3)nc(-c3ccc(-c4cccc5c4oc4c6ccccc6c6c7ccccc7oc6c54)c4ccccc34)n2)cc1 ; c1ccc(-c2nc(-c3cccc4c3oc3ccccc34)nc(-c3cccc4c3oc3c5ccccc5c5c6ccccc6oc5c43)n2)cc1 ; c1ccc(-c2nc(-c3ccc(-n4c5ccccc5c5ccccc54)cc3)nc(-c3cccc4c3oc3c5ccccc5c5c6ccccc6oc5c43)n2)cc1]

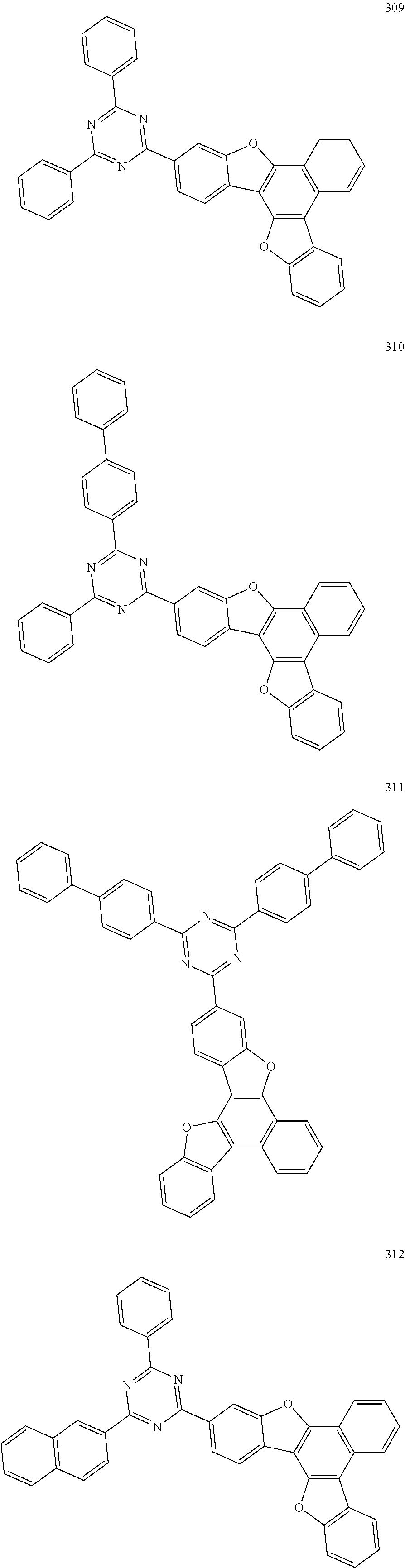 [SMILES: c1ccc(-c2nc(-c3ccccc3)nc(-c3ccc4c(c3)oc3c5ccccc5c5c6ccccc6oc5c43)n2)cc1 ; c1ccc(-c2ccc(-c3nc(-c4ccccc4)nc(-c4ccc5c(c4)oc4c6ccccc6c6c7ccccc7oc6c54)n3)cc2)cc1 ; c1ccc(-c2ccc(-c3nc(-c4ccc(-c5ccccc5)cc4)nc(-c4ccc5c(c4)oc4c6ccccc6c6c7ccccc7oc6c54)n3)cc2)cc1 ; c1ccc(-c2nc(-c3ccc4ccccc4c3)nc(-c3ccc4c(c3)oc3c5ccccc5c5c6ccccc6oc5c43)n2)cc1]

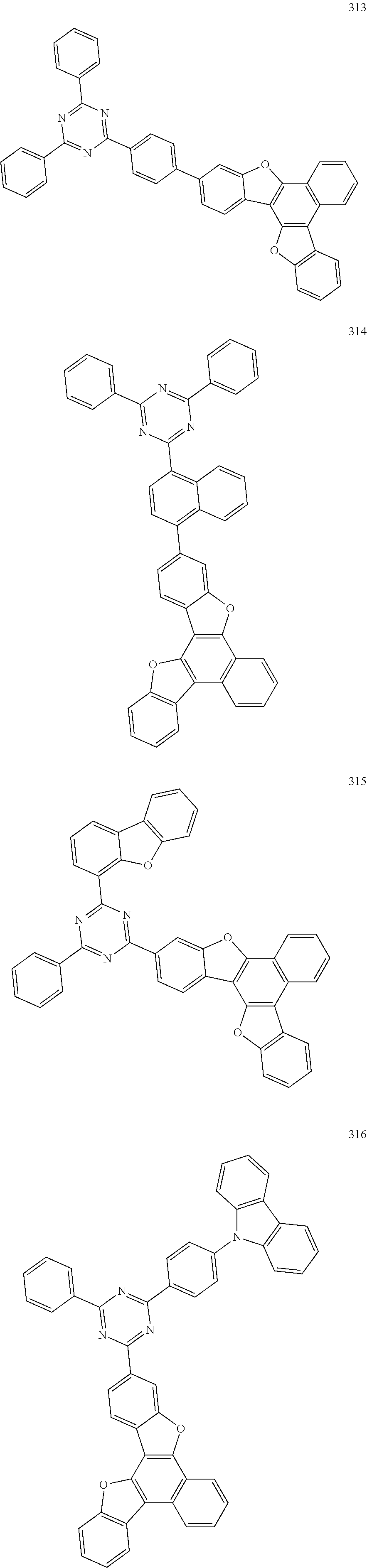 [SMILES: c1ccc(-c2nc(-c3ccccc3)nc(-c3ccc(-c4ccc5c(c4)oc4c6ccccc6c6c7ccccc7oc6c54)cc3)n2)cc1 ; c1ccc(-c2nc(-c3ccccc3)nc(-c3ccc(-c4ccc5c(c4)oc4c6ccccc6c6c7ccccc7oc6c54)c4ccccc34)n2)cc1 ; c1ccc(-c2nc(-c3ccc4c(c3)oc3c5ccccc5c5c6ccccc6oc5c43)nc(-c3cccc4c3oc3ccccc34)n2)cc1 ; c1ccc(-c2nc(-c3ccc(-n4c5ccccc5c5ccccc54)cc3)nc(-c3ccc4c(c3)oc3c5ccccc5c5c6ccccc6oc5c43)n2)cc1]

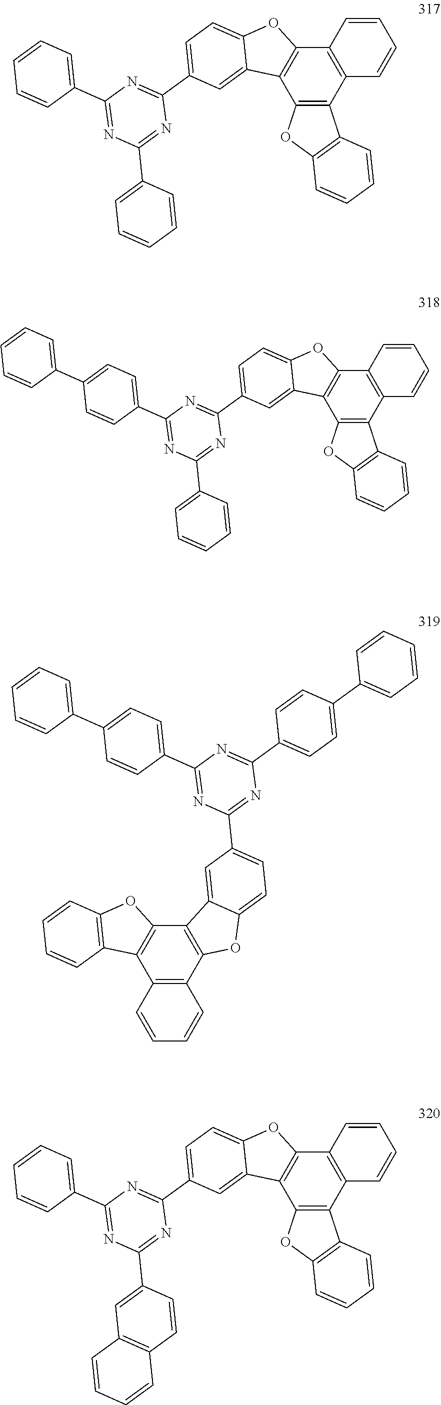 [SMILES: c1ccc(-c2nc(-c3ccccc3)nc(-c3ccc4oc5c6ccccc6c6c7ccccc7oc6c5c4c3)n2)cc1 ; c1ccc(-c2ccc(-c3nc(-c4ccccc4)nc(-c4ccc5oc6c7ccccc7c7c8ccccc8oc7c6c5c4)n3)cc2)cc1 ; c1ccc(-c2ccc(-c3nc(-c4ccc(-c5ccccc5)cc4)nc(-c4ccc5oc6c7ccccc7c7c8ccccc8oc7c6c5c4)n3)cc2)cc1 ; c1ccc(-c2nc(-c3ccc4ccccc4c3)nc(-c3ccc4oc5c6ccccc6c6c7ccccc7oc6c5c4c3)n2)cc1]

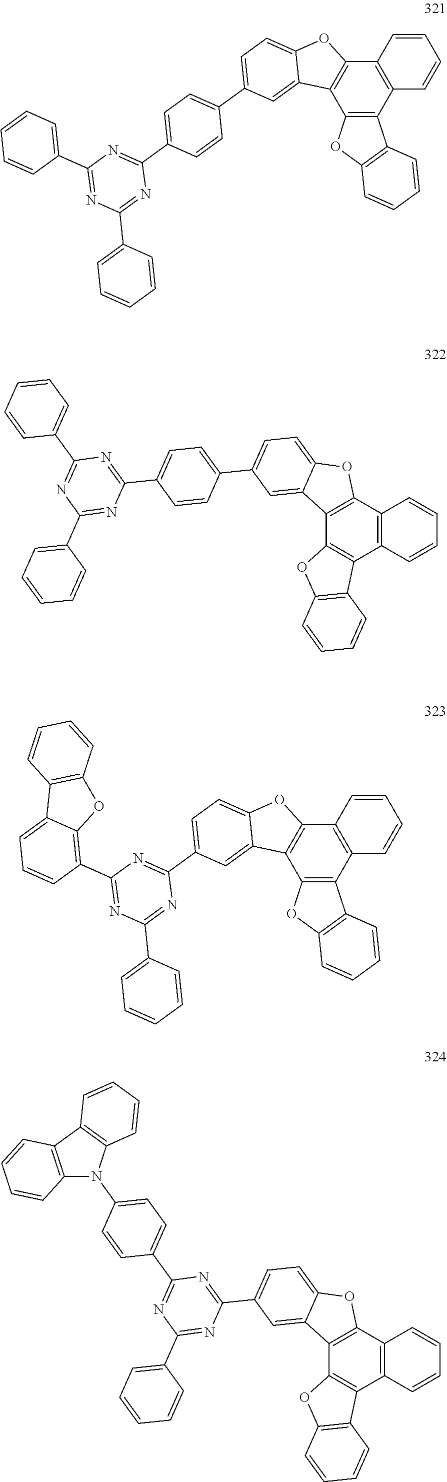 [SMILES: c1ccc(-c2nc(-c3ccccc3)nc(-c3ccc(-c4ccc5oc6c7ccccc7c7c8ccccc8oc7c6c5c4)cc3)n2)cc1 ; c1ccc(-c2nc(-c3ccccc3)nc(-c3ccc(-c4ccc5oc6c7ccccc7c7c8ccccc8oc7c6c5c4)cc3)n2)cc1 ; c1ccc(-c2nc(-c3ccc4oc5c6ccccc6c6c7ccccc7oc6c5c4c3)nc(-c3cccc4c3oc3ccccc34)n2)cc1 ; c1ccc(-c2nc(-c3ccc(-n4c5ccccc5c5ccccc54)cc3)nc(-c3ccc4oc5c6ccccc6c6c7ccccc7oc6c5c4c3)n2)cc1]

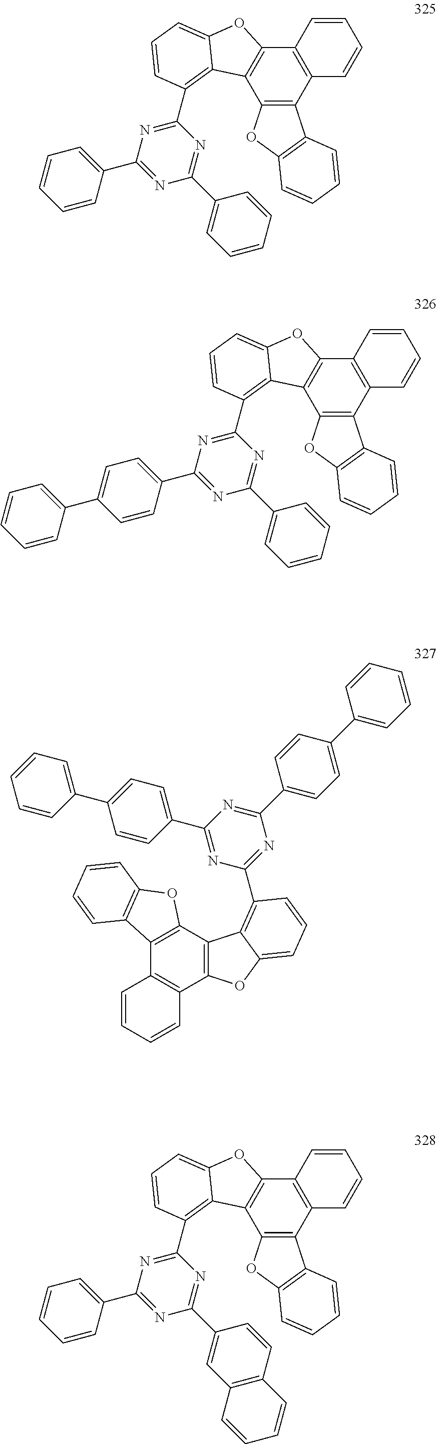 [SMILES: c1ccc(-c2nc(-c3ccccc3)nc(-c3cccc4oc5c6ccccc6c6c7ccccc7oc6c5c34)n2)cc1 ; c1ccc(-c2ccc(-c3nc(-c4ccccc4)nc(-c4cccc5oc6c7ccccc7c7c8ccccc8oc7c6c45)n3)cc2)cc1 ; c1ccc(-c2ccc(-c3nc(-c4ccc(-c5ccccc5)cc4)nc(-c4cccc5oc6c7ccccc7c7c8ccccc8oc7c6c45)n3)cc2)cc1 ; c1ccc(-c2nc(-c3ccc4ccccc4c3)nc(-c3cccc4oc5c6ccccc6c6c7ccccc7oc6c5c34)n2)cc1]

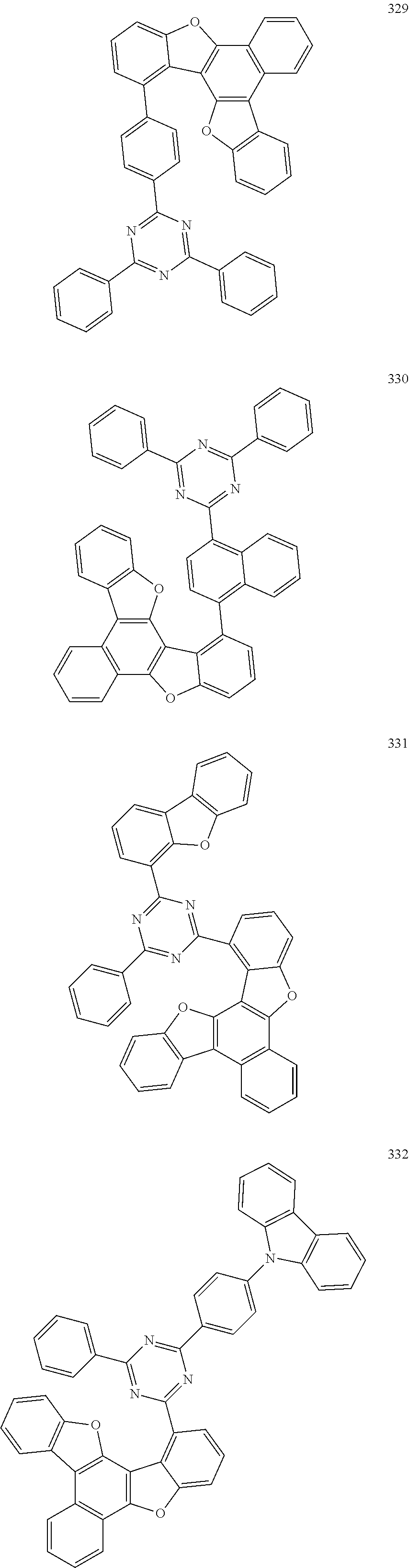 [SMILES: c1ccc(-c2nc(-c3ccccc3)nc(-c3ccc(-c4cccc5oc6c7ccccc7c7c8ccccc8oc7c6c45)cc3)n2)cc1 ; c1ccc(-c2nc(-c3ccccc3)nc(-c3ccc(-c4cccc5oc6c7ccccc7c7c8ccccc8oc7c6c45)c4ccccc34)n2)cc1 ; c1ccc(-c2nc(-c3cccc4c3oc3ccccc34)nc(-c3cccc4oc5c6ccccc6c6c7ccccc7oc6c5c34)n2)cc1 ; c1ccc(-c2nc(-c3ccc(-n4c5ccccc5c5ccccc54)cc3)nc(-c3cccc4oc5c6ccccc6c6c7ccccc7oc6c5c34)n2)cc1]

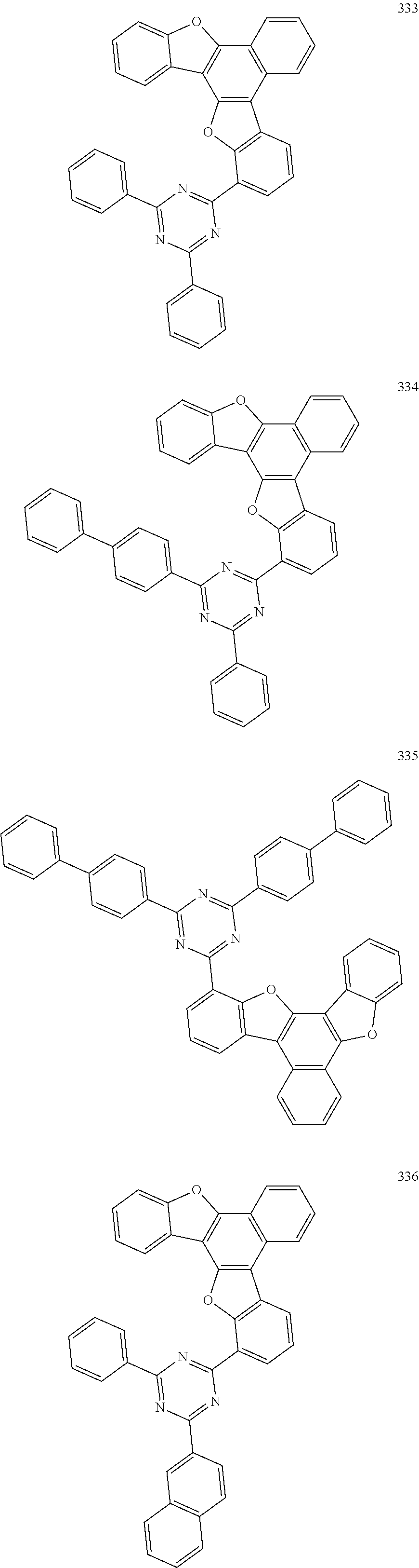 [SMILES: c1ccc(-c2nc(-c3ccccc3)nc(-c3cccc4c3oc3c4c4ccccc4c4oc5ccccc5c43)n2)cc1 ; c1ccc(-c2ccc(-c3nc(-c4ccccc4)nc(-c4cccc5c4oc4c5c5ccccc5c5oc6ccccc6c54)n3)cc2)cc1 ; c1ccc(-c2ccc(-c3nc(-c4ccc(-c5ccccc5)cc4)nc(-c4cccc5c4oc4c5c5ccccc5c5oc6ccccc6c54)n3)cc2)cc1 ; c1ccc(-c2nc(-c3ccc4ccccc4c3)nc(-c3cccc4c3oc3c4c4ccccc4c4oc5ccccc5c43)n2)cc1]

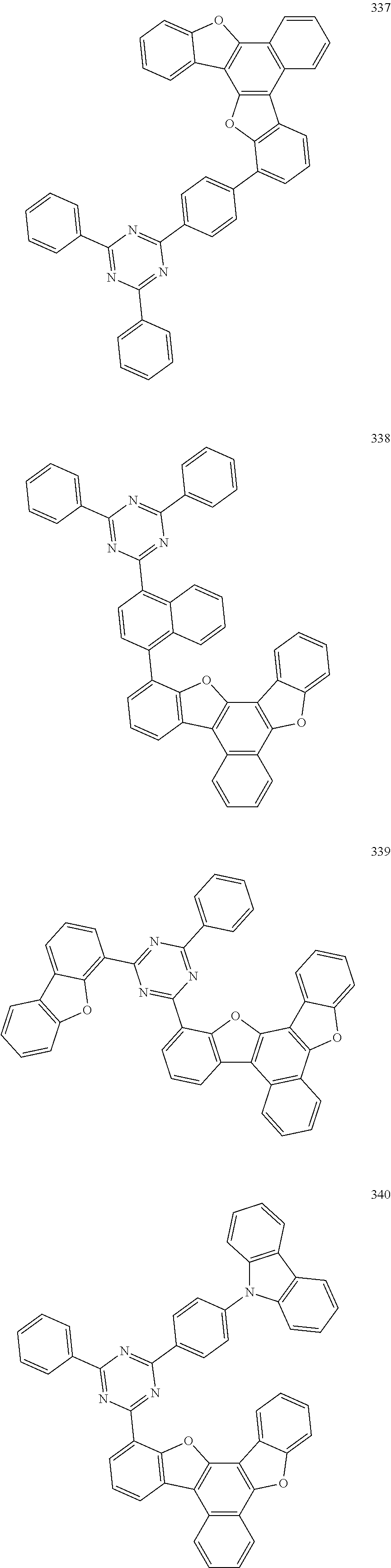 [SMILES: c1ccc(-c2nc(-c3ccccc3)nc(-c3ccc(-c4cccc5c4oc4c5c5ccccc5c5oc6ccccc6c54)cc3)n2)cc1 ; c1ccc(-c2nc(-c3ccccc3)nc(-c3ccc(-c4cccc5c4oc4c5c5ccccc5c5oc6ccccc6c54)c4ccccc34)n2)cc1 ; c1ccc(-c2nc(-c3cccc4c3oc3ccccc34)nc(-c3cccc4c3oc3c4c4ccccc4c4oc5ccccc5c43)n2)cc1 ; c1ccc(-c2nc(-c3ccc(-n4c5ccccc5c5ccccc54)cc3)nc(-c3cccc4c3oc3c4c4ccccc4c4oc5ccccc5c43)n2)cc1]

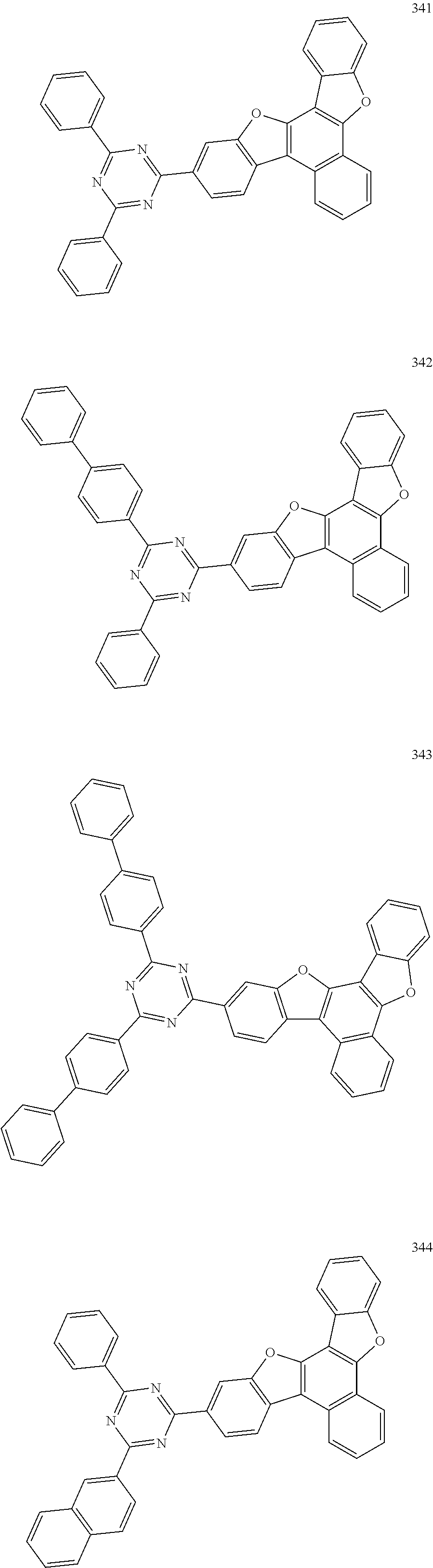 [SMILES: c1ccc(-c2nc(-c3ccccc3)nc(-c3ccc4c(c3)oc3c4c4ccccc4c4oc5ccccc5c43)n2)cc1 ; c1ccc(-c2ccc(-c3nc(-c4ccccc4)nc(-c4ccc5c(c4)oc4c5c5ccccc5c5oc6ccccc6c54)n3)cc2)cc1 ; c1ccc(-c2ccc(-c3nc(-c4ccc(-c5ccccc5)cc4)nc(-c4ccc5c(c4)oc4c5c5ccccc5c5oc6ccccc6c54)n3)cc2)cc1 ; c1ccc(-c2nc(-c3ccc4ccccc4c3)nc(-c3ccc4c(c3)oc3c4c4ccccc4c4oc5ccccc5c43)n2)cc1]

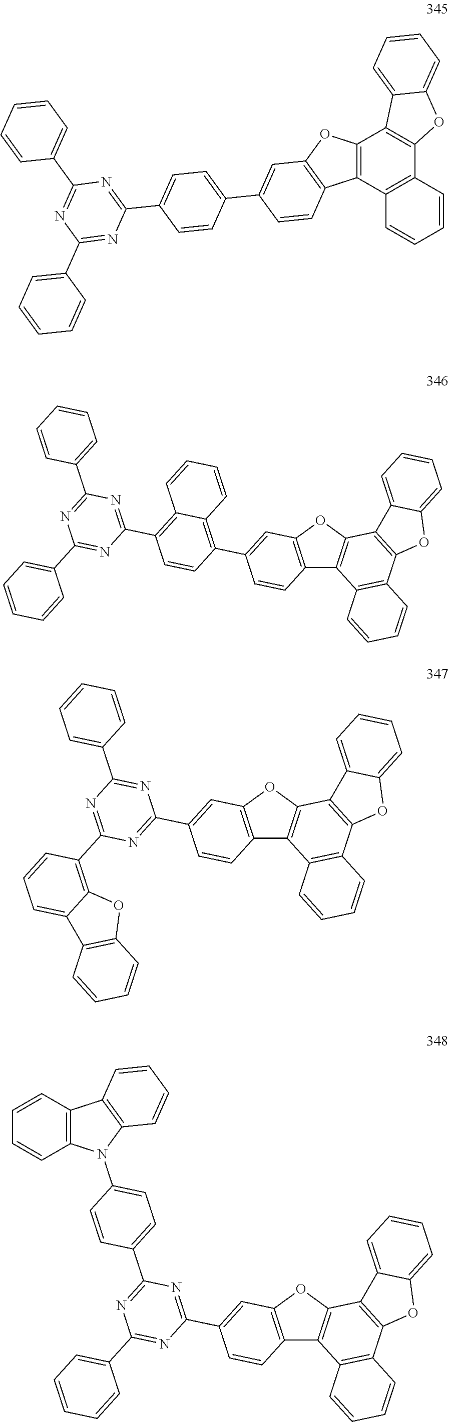 [SMILES: c1ccc(-c2nc(-c3ccccc3)nc(-c3ccc(-c4ccc5c(c4)oc4c5c5ccccc5c5oc6ccccc6c54)cc3)n2)cc1 ; c1ccc(-c2nc(-c3ccccc3)nc(-c3ccc(-c4ccc5c(c4)oc4c5c5ccccc5c5oc6ccccc6c54)c4ccccc34)n2)cc1 ; c1ccc(-c2nc(-c3ccc4c(c3)oc3c4c4ccccc4c4oc5ccccc5c43)nc(-c3cccc4c3oc3ccccc34)n2)cc1 ; c1ccc(-c2nc(-c3ccc(-n4c5ccccc5c5ccccc54)cc3)nc(-c3ccc4c(c3)oc3c4c4ccccc4c4oc5ccccc5c43)n2)cc1]

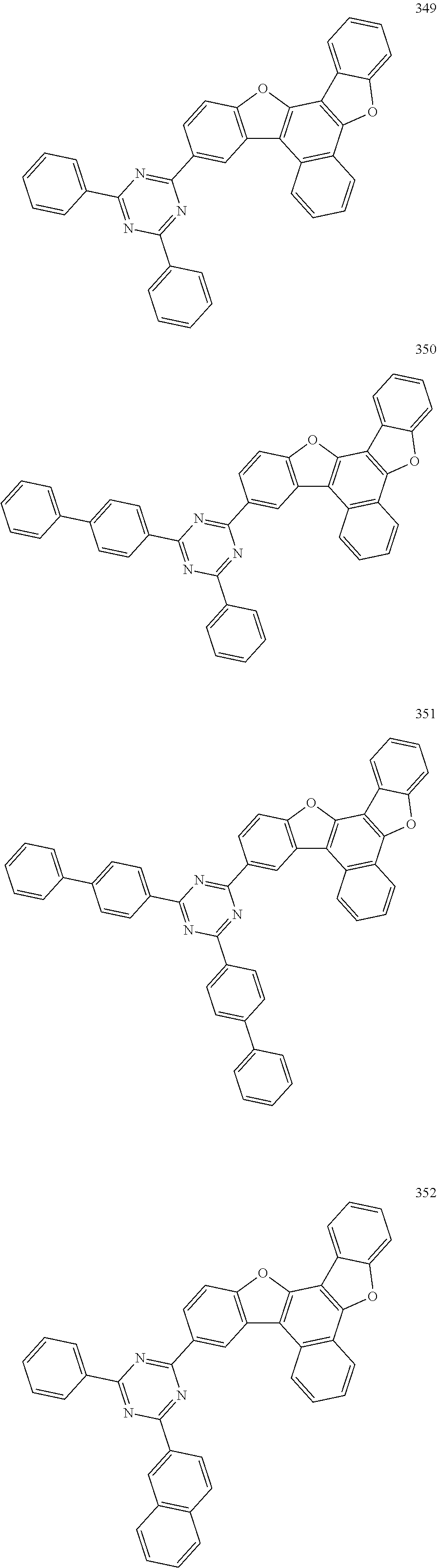 [SMILES: c1ccc(-c2nc(-c3ccccc3)nc(-c3ccc4oc5c(c4c3)c3ccccc3c3oc4ccccc4c35)n2)cc1 ; c1ccc(-c2ccc(-c3nc(-c4ccccc4)nc(-c4ccc5oc6c(c5c4)c4ccccc4c4oc5ccccc5c46)n3)cc2)cc1 ; c1ccc(-c2ccc(-c3nc(-c4ccc(-c5ccccc5)cc4)nc(-c4ccc5oc6c(c5c4)c4ccccc4c4oc5ccccc5c46)n3)cc2)cc1 ; c1ccc(-c2nc(-c3ccc4ccccc4c3)nc(-c3ccc4oc5c(c4c3)c3ccccc3c3oc4ccccc4c35)n2)cc1]

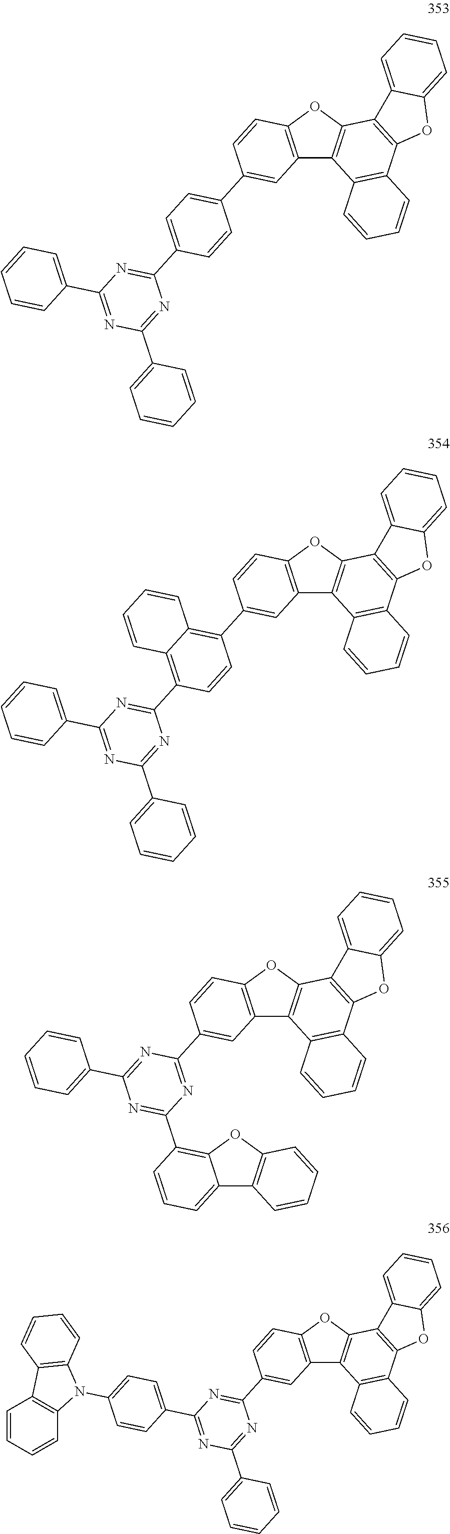 [SMILES: c1ccc(-c2nc(-c3ccccc3)nc(-c3ccc(-c4ccc5oc6c(c5c4)c4ccccc4c4oc5ccccc5c46)cc3)n2)cc1 ; c1ccc(-c2nc(-c3ccccc3)nc(-c3ccc(-c4ccc5oc6c(c5c4)c4ccccc4c4oc5ccccc5c46)c4ccccc34)n2)cc1 ; c1ccc(-c2nc(-c3ccc4oc5c(c4c3)c3ccccc3c3oc4ccccc4c35)nc(-c3cccc4c3oc3ccccc34)n2)cc1 ; c1ccc(-c2nc(-c3ccc(-n4c5ccccc5c5ccccc54)cc3)nc(-c3ccc4oc5c(c4c3)c3ccccc3c3oc4ccccc4c35)n2)cc1]

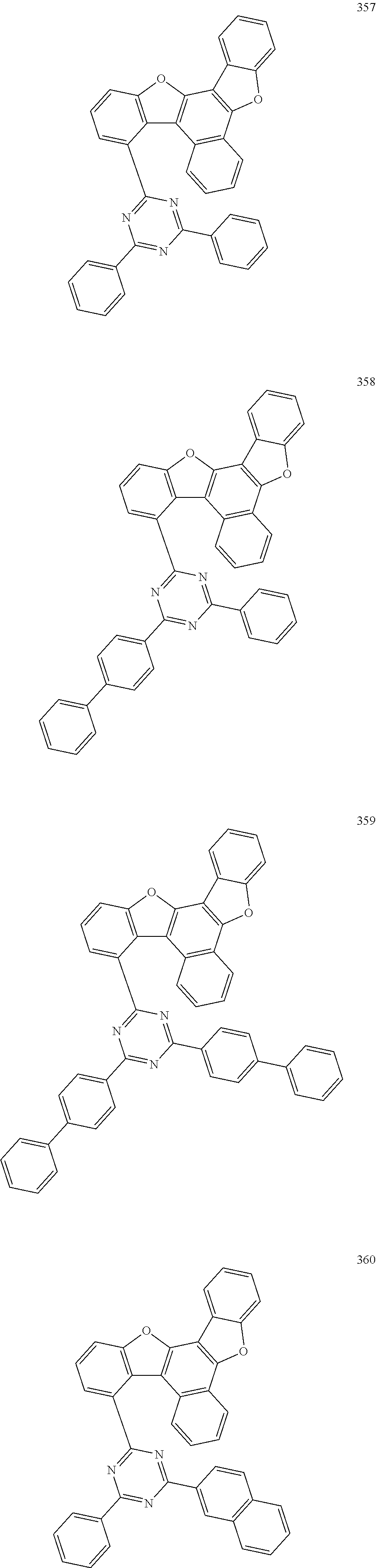 [SMILES: c1ccc(-c2nc(-c3ccccc3)nc(-c3cccc4oc5c6c7ccccc7oc6c6ccccc6c5c34)n2)cc1 ; c1ccc(-c2ccc(-c3nc(-c4ccccc4)nc(-c4cccc5oc6c7c8ccccc8oc7c7ccccc7c6c45)n3)cc2)cc1 ; c1ccc(-c2ccc(-c3nc(-c4ccc(-c5ccccc5)cc4)nc(-c4cccc5oc6c7c8ccccc8oc7c7ccccc7c6c45)n3)cc2)cc1 ; c1ccc(-c2nc(-c3ccc4ccccc4c3)nc(-c3cccc4oc5c6c7ccccc7oc6c6ccccc6c5c34)n2)cc1]

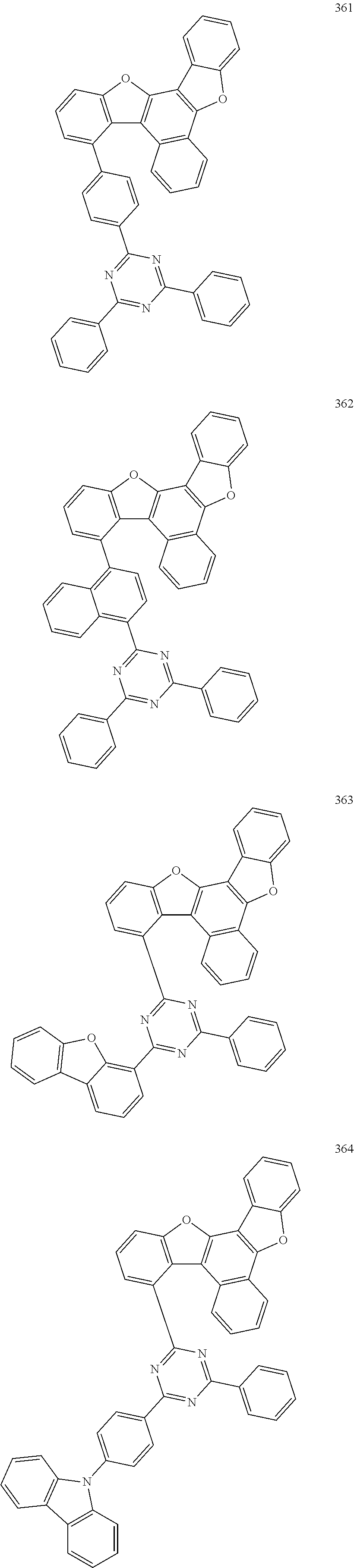 [SMILES: c1ccc(-c2nc(-c3ccccc3)nc(-c3ccc(-c4cccc5oc6c7c8ccccc8oc7c7ccccc7c6c45)cc3)n2)cc1 ; c1ccc(-c2nc(-c3ccccc3)nc(-c3ccc(-c4cccc5oc6c7c8ccccc8oc7c7ccccc7c6c45)c4ccccc34)n2)cc1 ; c1ccc(-c2nc(-c3cccc4c3oc3ccccc34)nc(-c3cccc4oc5c6c7ccccc7oc6c6ccccc6c5c34)n2)cc1 ; c1ccc(-c2nc(-c3ccc(-n4c5ccccc5c5ccccc54)cc3)nc(-c3cccc4oc5c6c7ccccc7oc6c6ccccc6c5c34)n2)cc1]

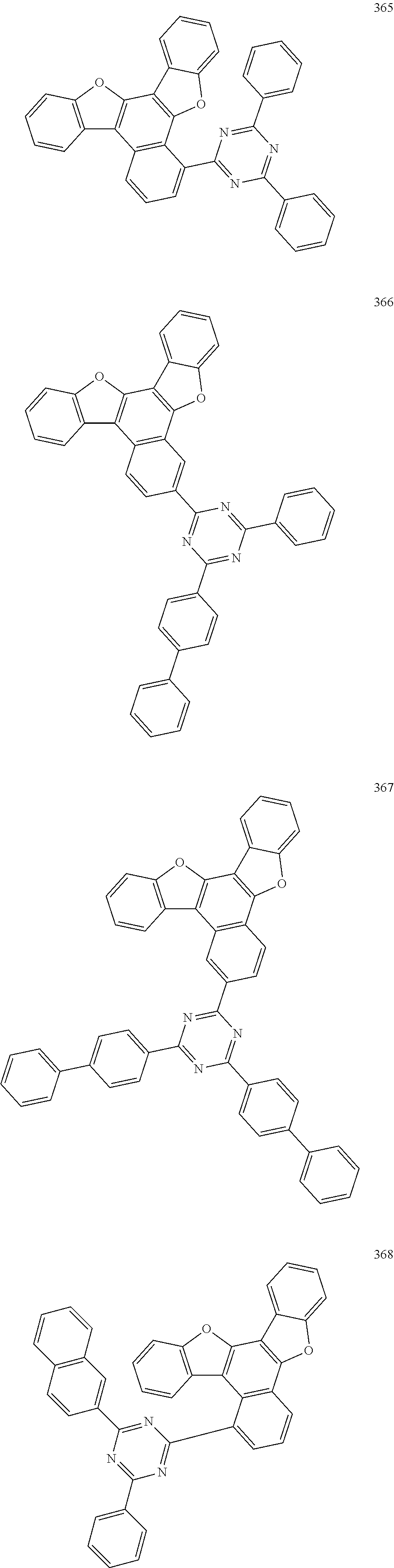 [SMILES: c1ccc(-c2nc(-c3ccccc3)nc(-c3cccc4c3c3oc5ccccc5c3c3oc5ccccc5c43)n2)cc1 ; c1ccc(-c2ccc(-c3nc(-c4ccccc4)nc(-c4ccc5c(c4)c4oc6ccccc6c4c4oc6ccccc6c54)n3)cc2)cc1 ; c1ccc(-c2ccc(-c3nc(-c4ccc(-c5ccccc5)cc4)nc(-c4ccc5c(c4)c4c6ccccc6oc4c4c6ccccc6oc54)n3)cc2)cc1 ; c1ccc(-c2nc(-c3ccc4ccccc4c3)nc(-c3cccc4c5oc6ccccc6c5c5oc6ccccc6c5c34)n2)cc1]

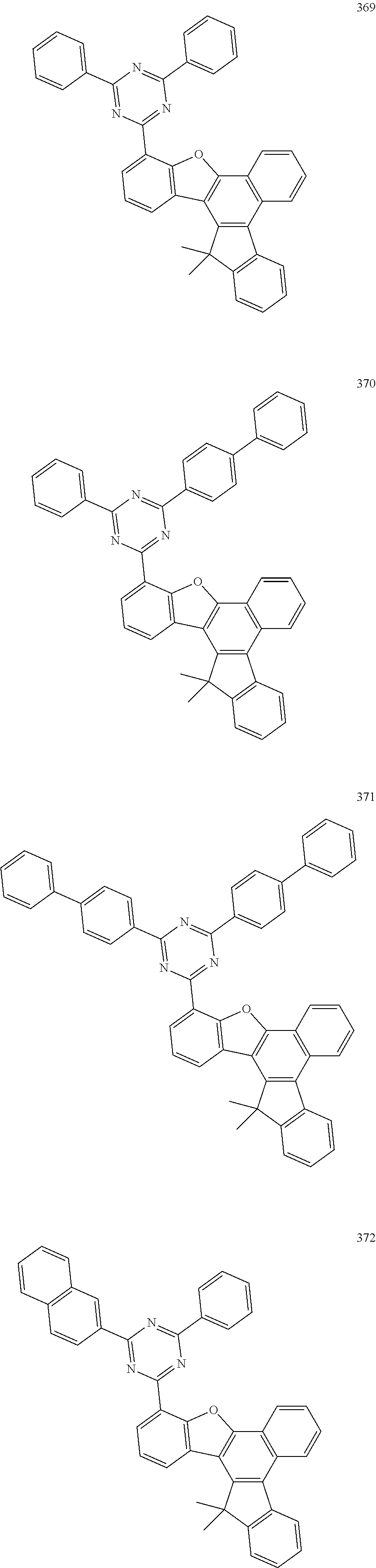 [SMILES: CC1(C)c2ccccc2-c2c1c1c3cccc(-c4nc(-c5ccccc5)nc(-c5ccccc5)n4)c3oc1c1ccccc21 ; CC1(C)c2ccccc2-c2c1c1c3cccc(-c4nc(-c5ccccc5)nc(-c5ccc(-c6ccccc6)cc5)n4)c3oc1c1ccccc21 ; CC1(C)c2ccccc2-c2c1c1c3cccc(-c4nc(-c5ccc(-c6ccccc6)cc5)nc(-c5ccc(-c6ccccc6)cc5)n4)c3oc1c1ccccc21 ; CC1(C)c2ccccc2-c2c1c1c3cccc(-c4nc(-c5ccccc5)nc(-c5ccc6ccccc6c5)n4)c3oc1c1ccccc21]

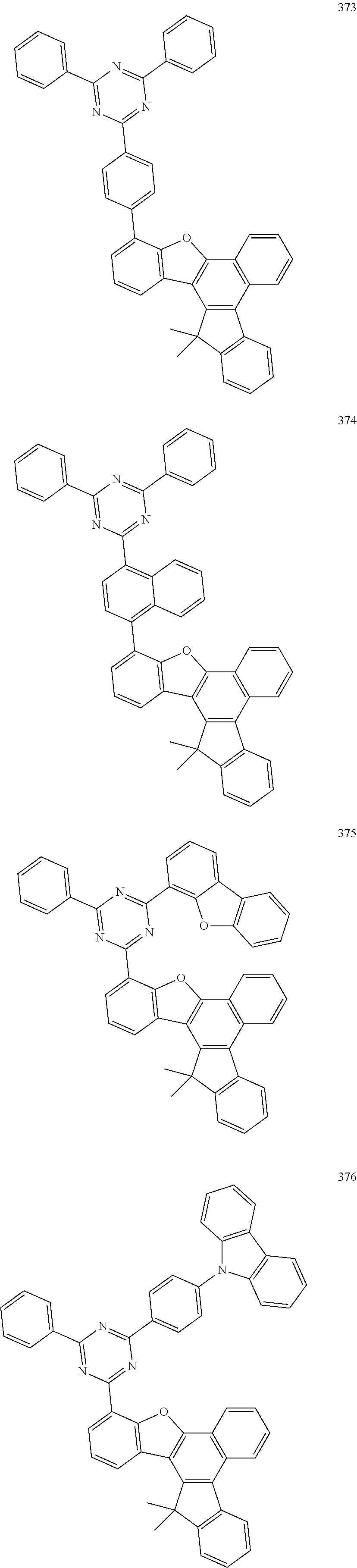 [SMILES: CC1(C)c2ccccc2-c2c1c1c3cccc(-c4ccc(-c5nc(-c6ccccc6)nc(-c6ccccc6)n5)cc4)c3oc1c1ccccc21 ; CC1(C)c2ccccc2-c2c1c1c3cccc(-c4ccc(-c5nc(-c6ccccc6)nc(-c6ccccc6)n5)c5ccccc45)c3oc1c1ccccc21 ; CC1(C)c2ccccc2-c2c1c1c3cccc(-c4nc(-c5ccccc5)nc(-c5cccc6c5oc5ccccc56)n4)c3oc1c1ccccc21 ; CC1(C)c2ccccc2-c2c1c1c3cccc(-c4nc(-c5ccccc5)nc(-c5ccc(-n6c7ccccc7c7ccccc76)cc5)n4)c3oc1c1ccccc21]

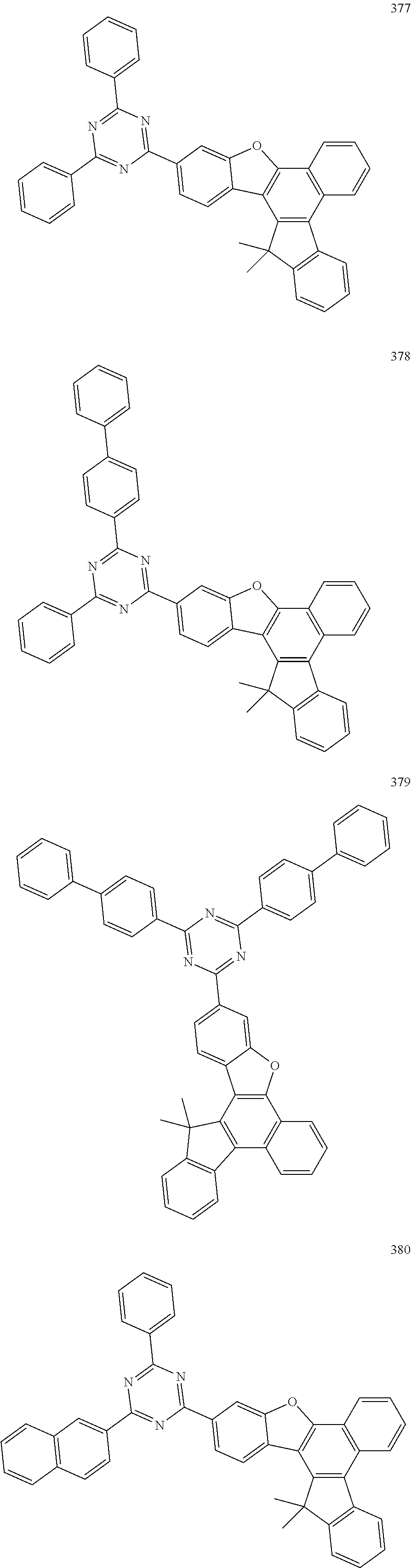 [SMILES: CC1(C)c2ccccc2-c2c1c1c3ccc(-c4nc(-c5ccccc5)nc(-c5ccccc5)n4)cc3oc1c1ccccc21 ; CC1(C)c2ccccc2-c2c1c1c3ccc(-c4nc(-c5ccccc5)nc(-c5ccc(-c6ccccc6)cc5)n4)cc3oc1c1ccccc21 ; CC1(C)c2ccccc2-c2c1c1c3ccc(-c4nc(-c5ccc(-c6ccccc6)cc5)nc(-c5ccc(-c6ccccc6)cc5)n4)cc3oc1c1ccccc21 ; CC1(C)c2ccccc2-c2c1c1c3ccc(-c4nc(-c5ccccc5)nc(-c5ccc6ccccc6c5)n4)cc3oc1c1ccccc21]

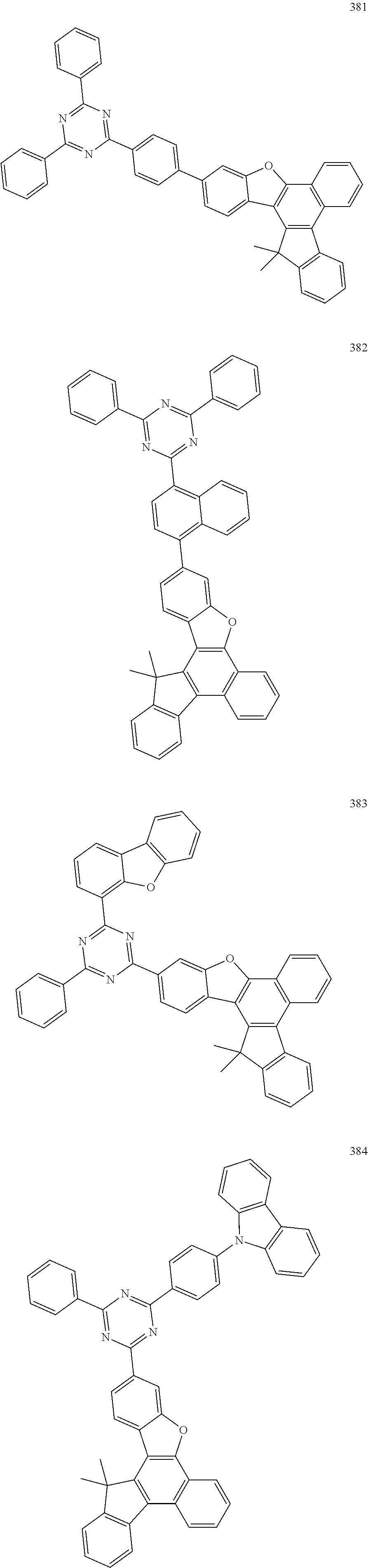 [SMILES: CC1(C)c2ccccc2-c2c1c1c3ccc(-c4ccc(-c5nc(-c6ccccc6)nc(-c6ccccc6)n5)cc4)cc3oc1c1ccccc21 ; CC1(C)c2ccccc2-c2c1c1c3ccc(-c4ccc(-c5nc(-c6ccccc6)nc(-c6ccccc6)n5)c5ccccc45)cc3oc1c1ccccc21 ; CC1(C)c2ccccc2-c2c1c1c3ccc(-c4nc(-c5ccccc5)nc(-c5cccc6c5oc5ccccc56)n4)cc3oc1c1ccccc21 ; CC1(C)c2ccccc2-c2c1c1c3ccc(-c4nc(-c5ccccc5)nc(-c5ccc(-n6c7ccccc7c7ccccc76)cc5)n4)cc3oc1c1ccccc21]

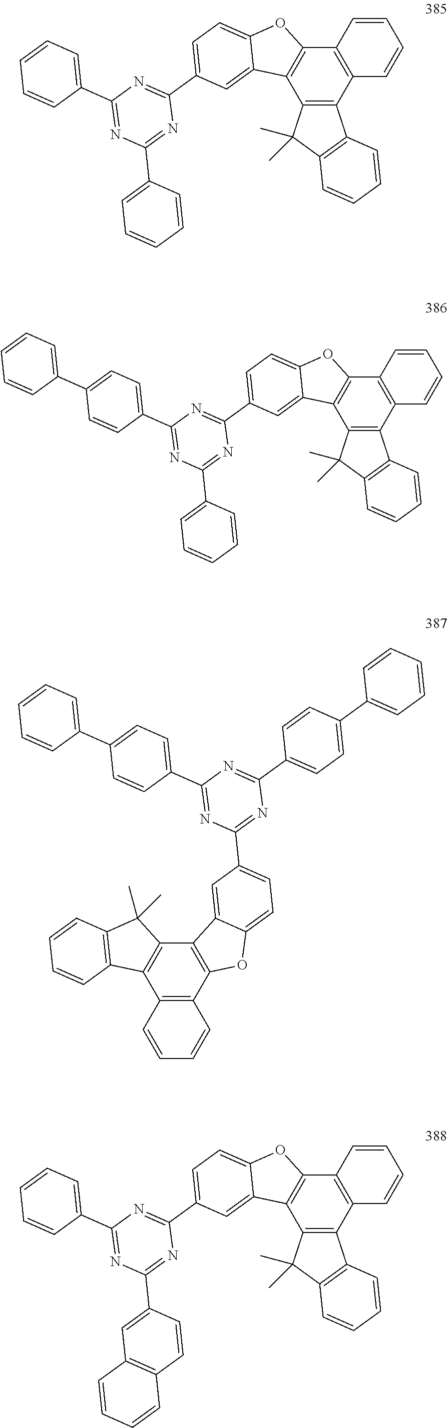 [SMILES: CC1(C)c2ccccc2-c2c1c1c3cc(-c4nc(-c5ccccc5)nc(-c5ccccc5)n4)ccc3oc1c1ccccc21 ; CC1(C)c2ccccc2-c2c1c1c3cc(-c4nc(-c5ccccc5)nc(-c5ccc(-c6ccccc6)cc5)n4)ccc3oc1c1ccccc21 ; CC1(C)c2ccccc2-c2c1c1c3cc(-c4nc(-c5ccc(-c6ccccc6)cc5)nc(-c5ccc(-c6ccccc6)cc5)n4)ccc3oc1c1ccccc21 ; CC1(C)c2ccccc2-c2c1c1c3cc(-c4nc(-c5ccccc5)nc(-c5ccc6ccccc6c5)n4)ccc3oc1c1ccccc21]

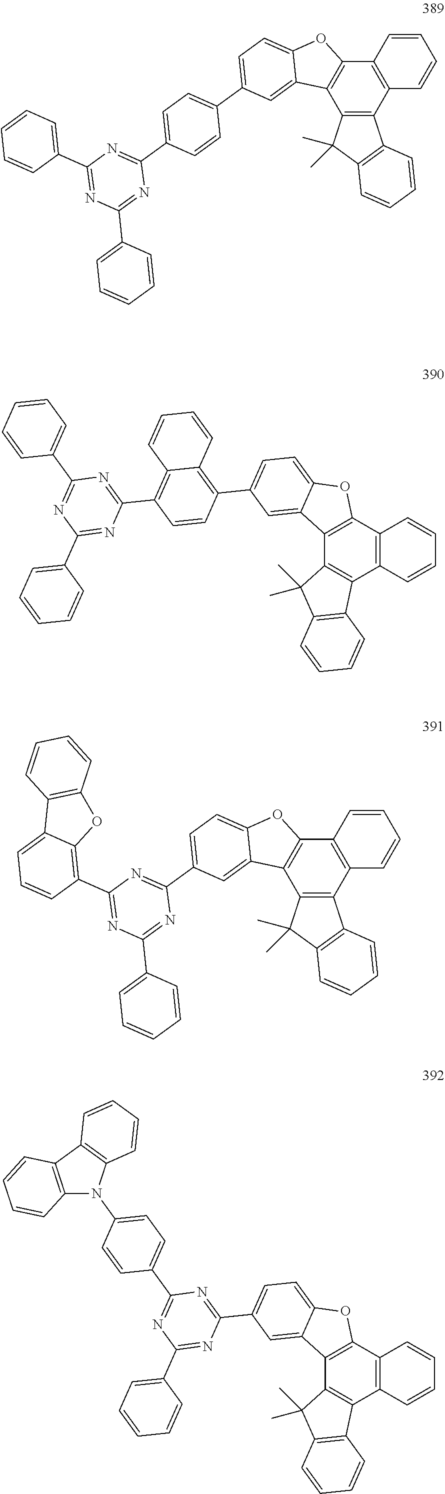 [SMILES: CC1(C)c2ccccc2-c2c1c1c3cc(-c4ccc(-c5nc(-c6ccccc6)nc(-c6ccccc6)n5)cc4)ccc3oc1c1ccccc21 ; CC1(C)c2ccccc2-c2c1c1c3cc(-c4ccc(-c5nc(-c6ccccc6)nc(-c6ccccc6)n5)c5ccccc45)ccc3oc1c1ccccc21 ; CC1(C)c2ccccc2-c2c1c1c3cc(-c4nc(-c5ccccc5)nc(-c5cccc6c5oc5ccccc56)n4)ccc3oc1c1ccccc21 ; CC1(C)c2ccccc2-c2c1c1c3cc(-c4nc(-c5ccccc5)nc(-c5ccc(-n6c7ccccc7c7ccccc76)cc5)n4)ccc3oc1c1ccccc21]

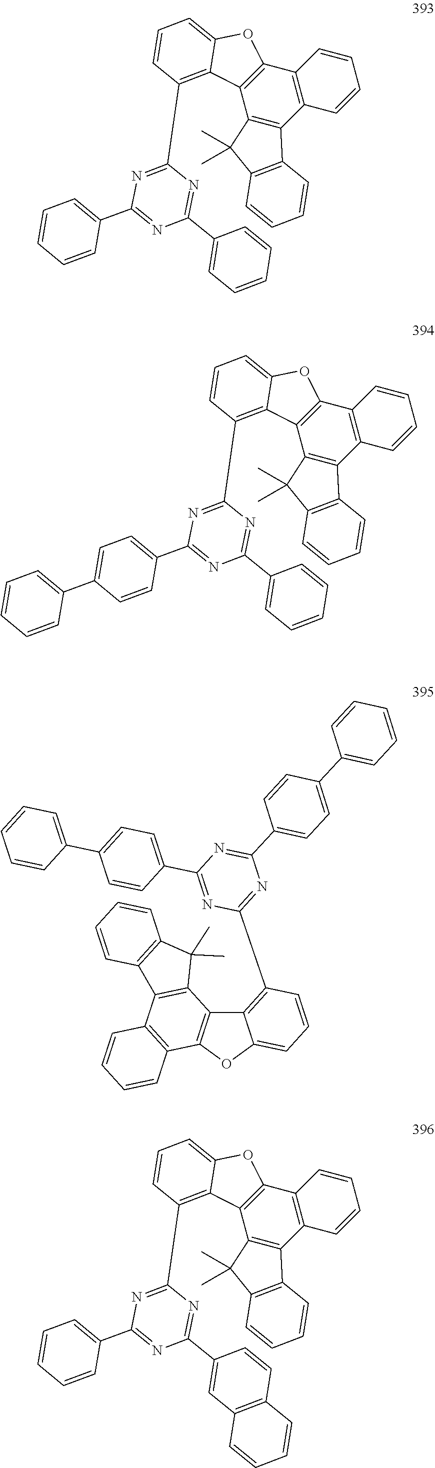 [SMILES: CC1(C)c2ccccc2-c2c1c1c(oc3cccc(-c4nc(-c5ccccc5)nc(-c5ccccc5)n4)c31)c1ccccc21 ; CC1(C)c2ccccc2-c2c1c1c(oc3cccc(-c4nc(-c5ccccc5)nc(-c5ccc(-c6ccccc6)cc5)n4)c31)c1ccccc21 ; CC1(C)c2ccccc2-c2c1c1c(oc3cccc(-c4nc(-c5ccc(-c6ccccc6)cc5)nc(-c5ccc(-c6ccccc6)cc5)n4)c31)c1ccccc21 ; CC1(C)c2ccccc2-c2c1c1c(oc3cccc(-c4nc(-c5ccccc5)nc(-c5ccc6ccccc6c5)n4)c31)c1ccccc21]

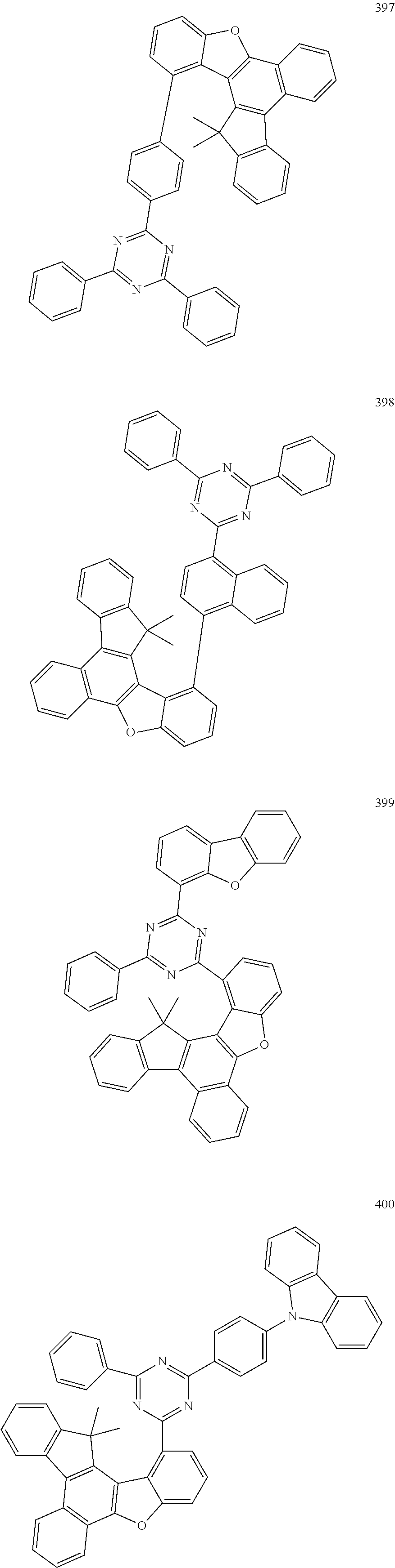 [SMILES: CC1(C)c2ccccc2-c2c1c1c(oc3cccc(-c4ccc(-c5nc(-c6ccccc6)nc(-c6ccccc6)n5)cc4)c31)c1ccccc21 ; CC1(C)c2ccccc2-c2c1c1c(oc3cccc(-c4ccc(-c5nc(-c6ccccc6)nc(-c6ccccc6)n5)c5ccccc45)c31)c1ccccc21 ; CC1(C)c2ccccc2-c2c1c1c(oc3cccc(-c4nc(-c5ccccc5)nc(-c5cccc6c5oc5ccccc56)n4)c31)c1ccccc21 ; CC1(C)c2ccccc2-c2c1c1c(oc3cccc(-c4nc(-c5ccccc5)nc(-c5ccc(-n6c7ccccc7c7ccccc76)cc5)n4)c31)c1ccccc21]

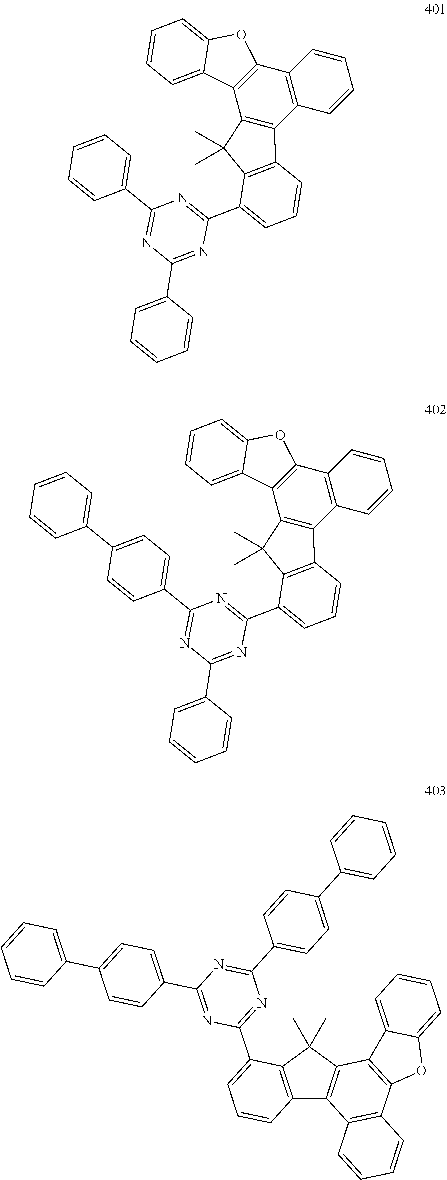 [SMILES: CC1(C)c2c(-c3nc(-c4ccccc4)nc(-c4ccccc4)n3)cccc2-c2c1c1c3ccccc3oc1c1ccccc21 ; CC1(C)c2c(-c3nc(-c4ccccc4)nc(-c4ccc(-c5ccccc5)cc4)n3)cccc2-c2c1c1c3ccccc3oc1c1ccccc21 ; CC1(C)c2c(-c3nc(-c4ccc(-c5ccccc5)cc4)nc(-c4ccc(-c5ccccc5)cc4)n3)cccc2-c2c1c1c3ccccc3oc1c1ccccc21]

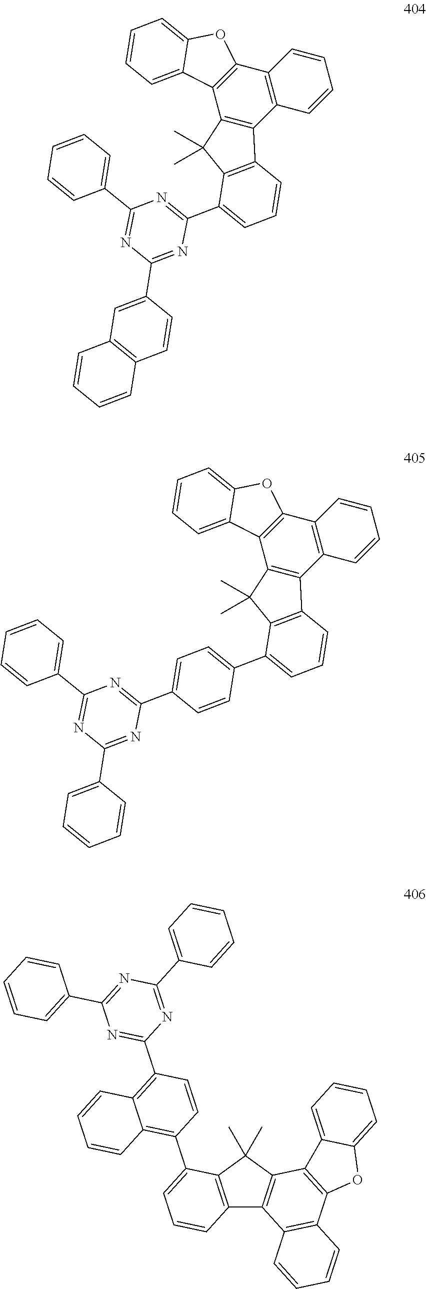 [SMILES: CC1(C)c2c(-c3nc(-c4ccccc4)nc(-c4ccc5ccccc5c4)n3)cccc2-c2c1c1c3ccccc3oc1c1ccccc21 ; CC1(C)c2c(-c3ccc(-c4nc(-c5ccccc5)nc(-c5ccccc5)n4)cc3)cccc2-c2c1c1c3ccccc3oc1c1ccccc21 ; CC1(C)c2c(-c3ccc(-c4nc(-c5ccccc5)nc(-c5ccccc5)n4)c4ccccc34)cccc2-c2c1c1c3ccccc3oc1c1ccccc21]

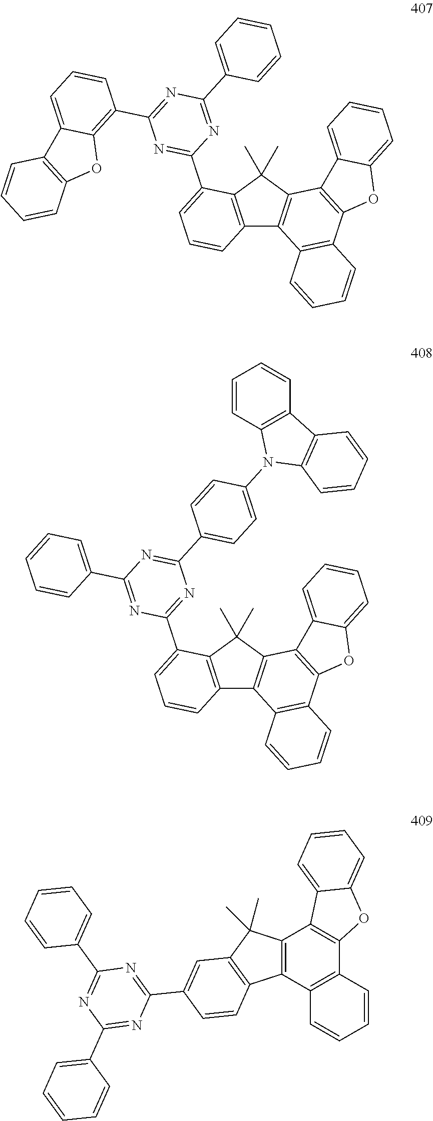 [SMILES: CC1(C)c2c(-c3nc(-c4ccccc4)nc(-c4cccc5c4oc4ccccc45)n3)cccc2-c2c1c1c3ccccc3oc1c1ccccc21 ; CC1(C)c2c(-c3nc(-c4ccccc4)nc(-c4ccc(-n5c6ccccc6c6ccccc65)cc4)n3)cccc2-c2c1c1c3ccccc3oc1c1ccccc21 ; CC1(C)c2cc(-c3nc(-c4ccccc4)nc(-c4ccccc4)n3)ccc2-c2c1c1c3ccccc3oc1c1ccccc21]

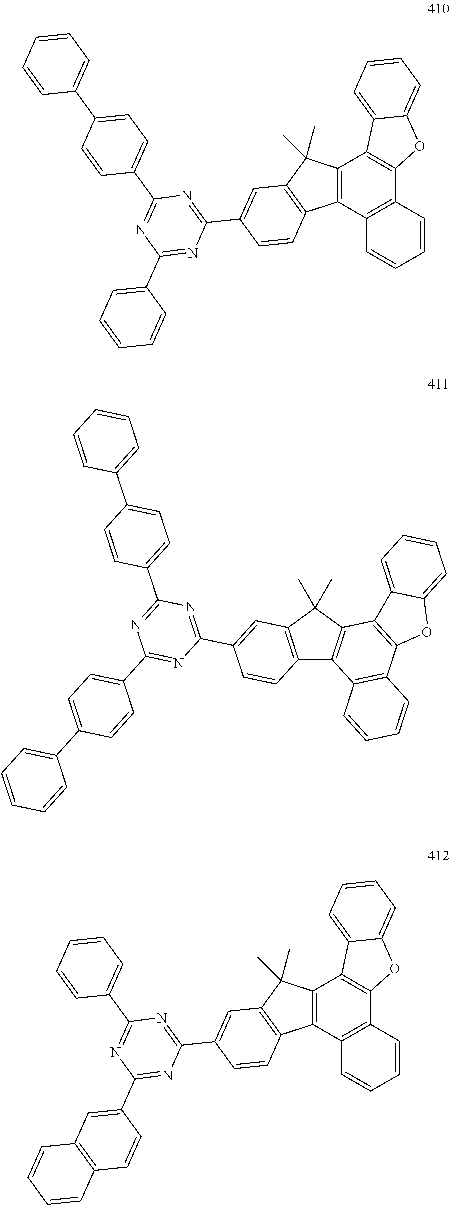 [SMILES: CC1(C)c2cc(-c3nc(-c4ccccc4)nc(-c4ccc(-c5ccccc5)cc4)n3)ccc2-c2c1c1c3ccccc3oc1c1ccccc21 ; CC1(C)c2cc(-c3nc(-c4ccc(-c5ccccc5)cc4)nc(-c4ccc(-c5ccccc5)cc4)n3)ccc2-c2c1c1c3ccccc3oc1c1ccccc21 ; CC1(C)c2cc(-c3nc(-c4ccccc4)nc(-c4ccc5ccccc5c4)n3)ccc2-c2c1c1c3ccccc3oc1c1ccccc21]

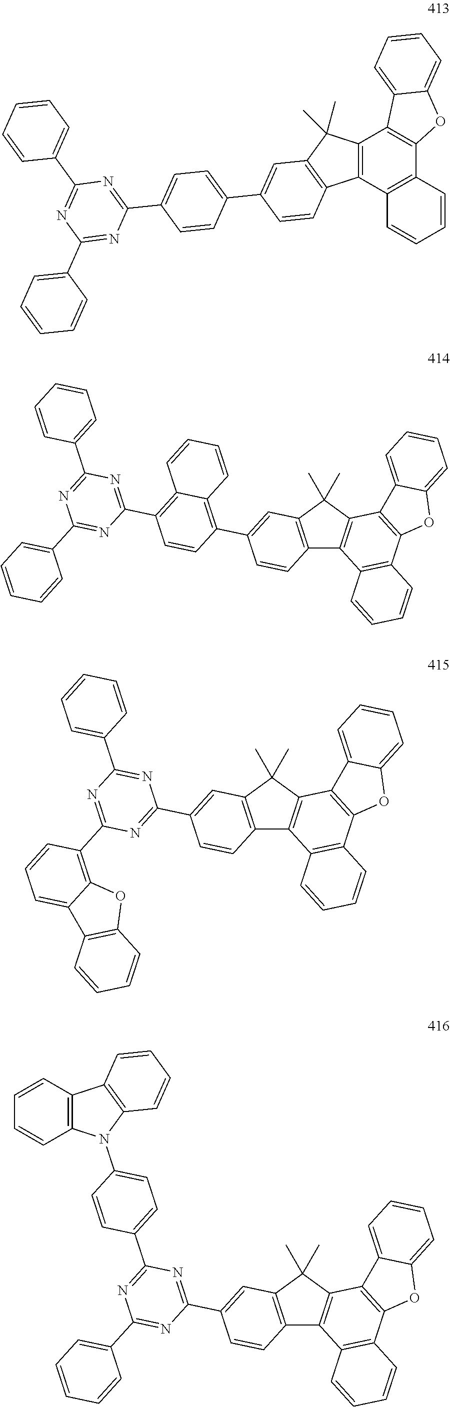 [SMILES: CC1(C)c2cc(-c3ccc(-c4nc(-c5ccccc5)nc(-c5ccccc5)n4)cc3)ccc2-c2c1c1c3ccccc3oc1c1ccccc21 ; CC1(C)c2cc(-c3ccc(-c4nc(-c5ccccc5)nc(-c5ccccc5)n4)c4ccccc34)ccc2-c2c1c1c3ccccc3oc1c1ccccc21 ; CC1(C)c2cc(-c3nc(-c4ccccc4)nc(-c4cccc5c4oc4ccccc45)n3)ccc2-c2c1c1c3ccccc3oc1c1ccccc21 ; CC1(C)c2cc(-c3nc(-c4ccccc4)nc(-c4ccc(-n5c6ccccc6c6ccccc65)cc4)n3)ccc2-c2c1c1c3ccccc3oc1c1ccccc21]

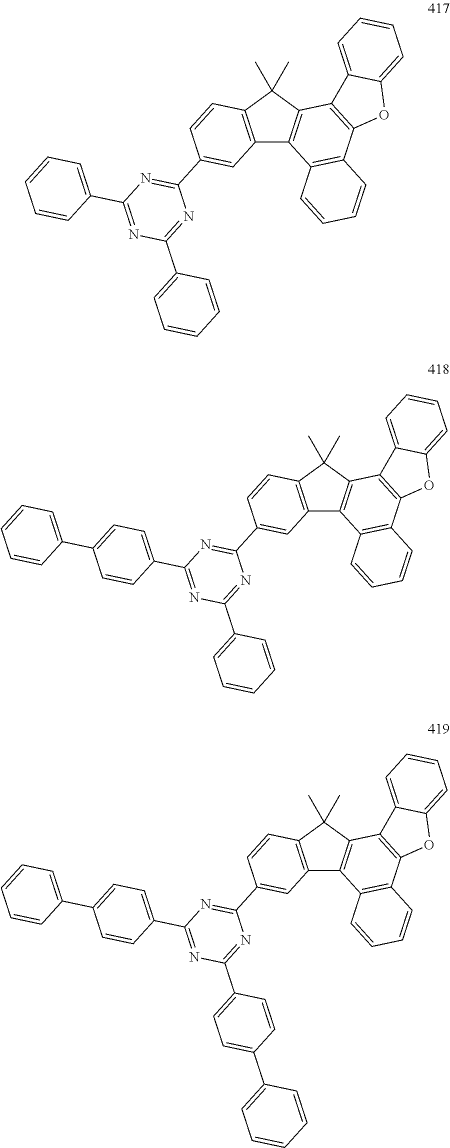 [SMILES: CC1(C)c2ccc(-c3nc(-c4ccccc4)nc(-c4ccccc4)n3)cc2-c2c1c1c3ccccc3oc1c1ccccc21 ; CC1(C)c2ccc(-c3nc(-c4ccccc4)nc(-c4ccc(-c5ccccc5)cc4)n3)cc2-c2c1c1c3ccccc3oc1c1ccccc21 ; CC1(C)c2ccc(-c3nc(-c4ccc(-c5ccccc5)cc4)nc(-c4ccc(-c5ccccc5)cc4)n3)cc2-c2c1c1c3ccccc3oc1c1ccccc21]

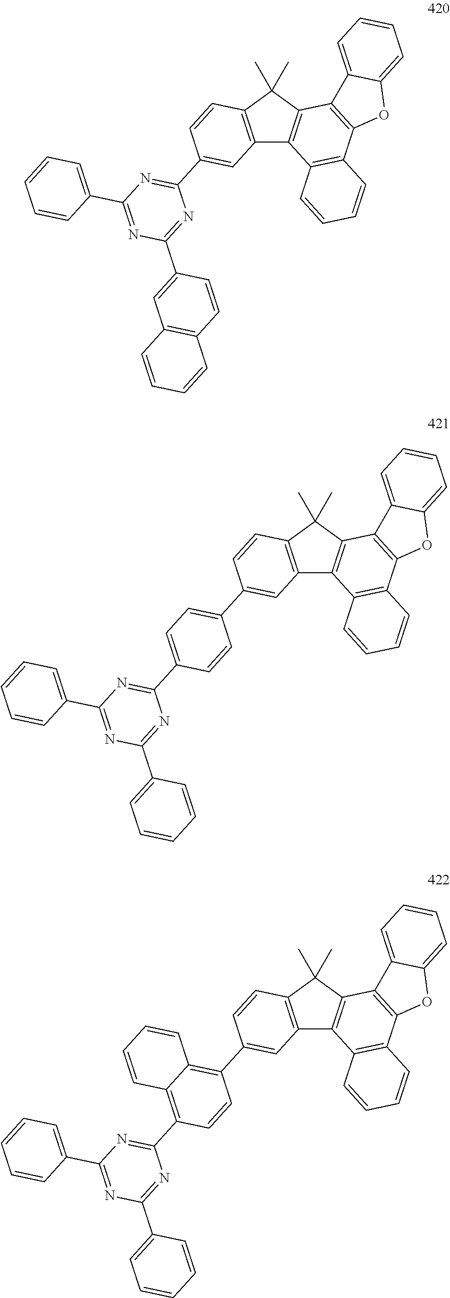 [SMILES: CC1(C)c2ccc(-c3nc(-c4ccccc4)nc(-c4ccc5ccccc5c4)n3)cc2-c2c1c1c3ccccc3oc1c1ccccc21 ; CC1(C)c2ccc(-c3ccc(-c4nc(-c5ccccc5)nc(-c5ccccc5)n4)cc3)cc2-c2c1c1c3ccccc3oc1c1ccccc21 ; CC1(C)c2ccc(-c3ccc(-c4nc(-c5ccccc5)nc(-c5ccccc5)n4)c4ccccc34)cc2-c2c1c1c3ccccc3oc1c1ccccc21]

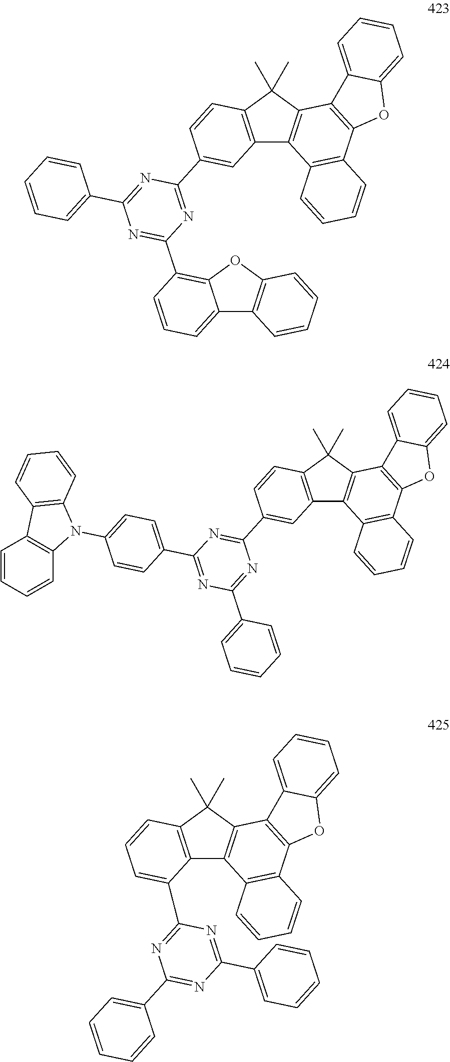 [SMILES: CC1(C)c2ccc(-c3nc(-c4ccccc4)nc(-c4cccc5c4oc4ccccc45)n3)cc2-c2c1c1c3ccccc3oc1c1ccccc21 ; CC1(C)c2ccc(-c3nc(-c4ccccc4)nc(-c4ccc(-n5c6ccccc6c6ccccc65)cc4)n3)cc2-c2c1c1c3ccccc3oc1c1ccccc21 ; CC1(C)c2cccc(-c3nc(-c4ccccc4)nc(-c4ccccc4)n3)c2-c2c1c1c3ccccc3oc1c1ccccc21]

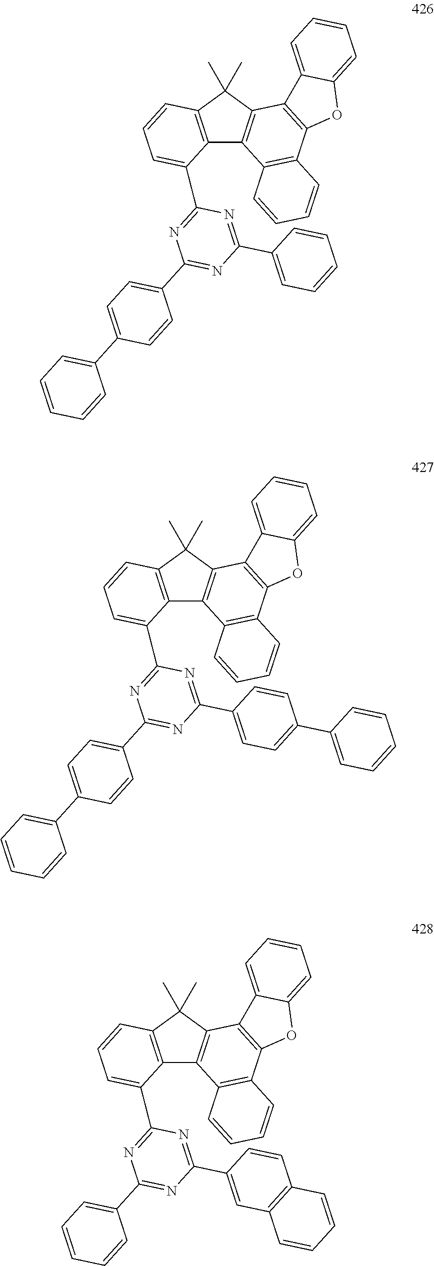 [SMILES: CC1(C)c2cccc(-c3nc(-c4ccccc4)nc(-c4ccc(-c5ccccc5)cc4)n3)c2-c2c1c1c3ccccc3oc1c1ccccc21 ; CC1(C)c2cccc(-c3nc(-c4ccc(-c5ccccc5)cc4)nc(-c4ccc(-c5ccccc5)cc4)n3)c2-c2c1c1c3ccccc3oc1c1ccccc21 ; CC1(C)c2cccc(-c3nc(-c4ccccc4)nc(-c4ccc5ccccc5c4)n3)c2-c2c1c1c3ccccc3oc1c1ccccc21]

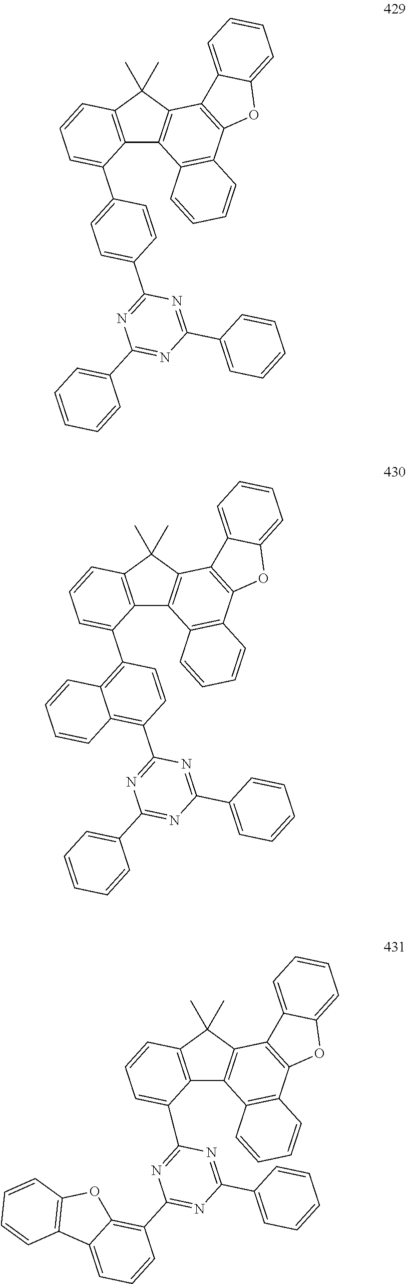 [SMILES: CC1(C)c2cccc(-c3ccc(-c4nc(-c5ccccc5)nc(-c5ccccc5)n4)cc3)c2-c2c1c1c3ccccc3oc1c1ccccc21 ; CC1(C)c2cccc(-c3ccc(-c4nc(-c5ccccc5)nc(-c5ccccc5)n4)c4ccccc34)c2-c2c1c1c3ccccc3oc1c1ccccc21 ; CC1(C)c2cccc(-c3nc(-c4ccccc4)nc(-c4cccc5c4oc4ccccc45)n3)c2-c2c1c1c3ccccc3oc1c1ccccc21]

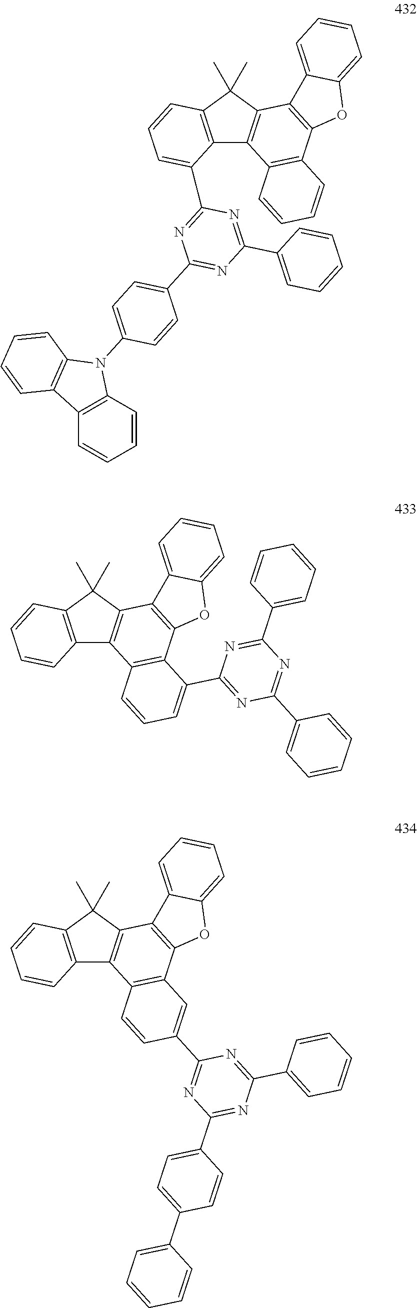 [SMILES: CC1(C)c2cccc(-c3nc(-c4ccccc4)nc(-c4ccc(-n5c6ccccc6c6ccccc65)cc4)n3)c2-c2c1c1c3ccccc3oc1c1ccccc21 ; CC1(C)c2ccccc2-c2c1c1c3ccccc3oc1c1c(-c3nc(-c4ccccc4)nc(-c4ccccc4)n3)cccc21 ; CC1(C)c2ccccc2-c2c1c1c3ccccc3oc1c1cc(-c3nc(-c4ccccc4)nc(-c4ccc(-c5ccccc5)cc4)n3)ccc21]

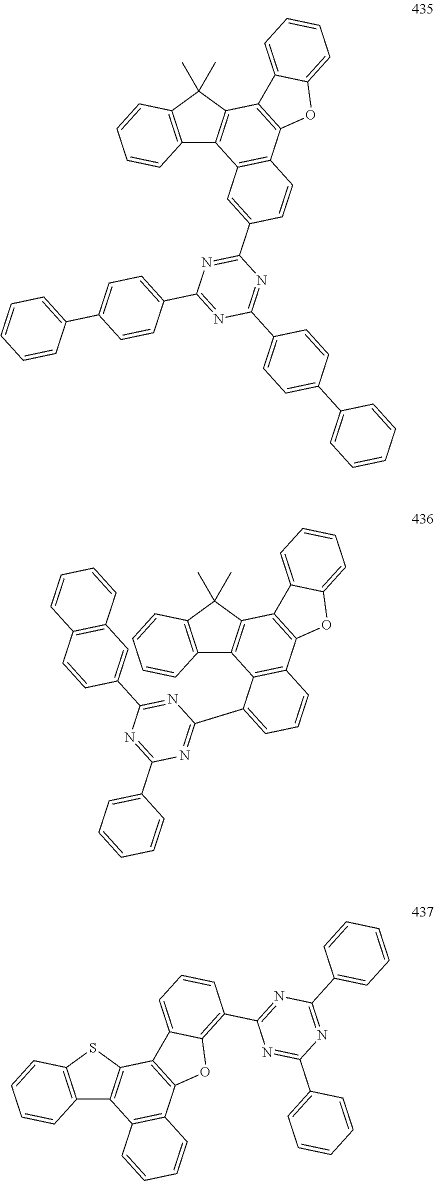

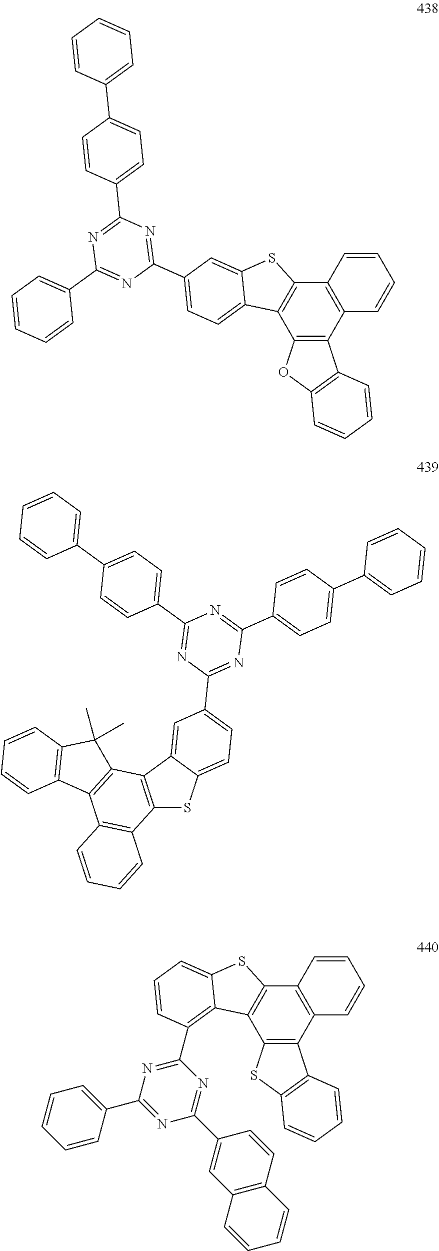 [SMILES: c1ccc(-c2ccc(-c3nc(-c4ccccc4)nc(-c4ccc5c(c4)sc4c6ccccc6c6c7ccccc7oc6c54)n3)cc2)cc1 ; CC1(C)c2ccccc2-c2c1c1c3cc(-c4nc(-c5ccc(-c6ccccc6)cc5)nc(-c5ccc(-c6ccccc6)cc5)n4)ccc3sc1c1ccccc21 ; c1ccc(-c2nc(-c3ccc4ccccc4c3)nc(-c3cccc4sc5c6ccccc6c6c7ccccc7sc6c5c34)n2)cc1]

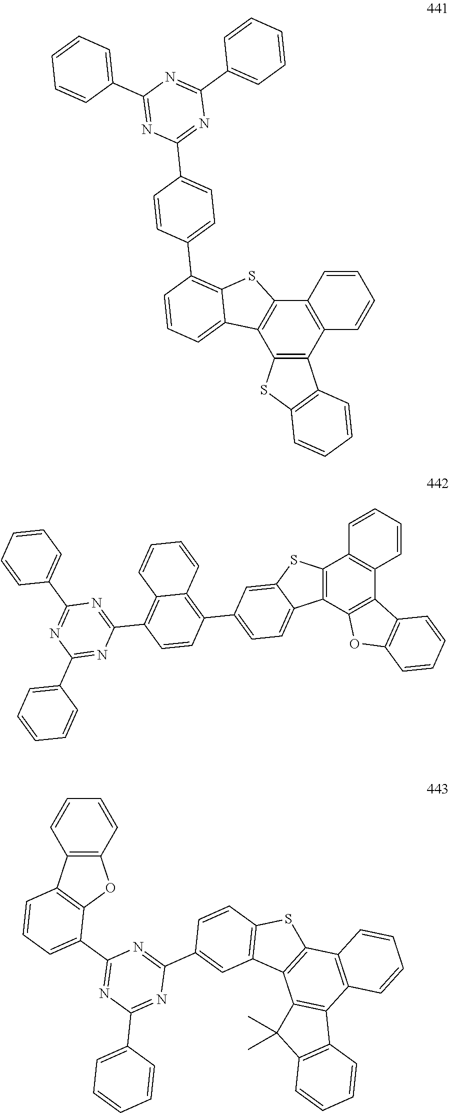 [SMILES: c1ccc(-c2nc(-c3ccccc3)nc(-c3ccc(-c4cccc5c4sc4c6ccccc6c6c7ccccc7sc6c54)cc3)n2)cc1 ; c1ccc(-c2nc(-c3ccccc3)nc(-c3ccc(-c4ccc5c(c4)sc4c6ccccc6c6c7ccccc7oc6c54)c4ccccc34)n2)cc1 ; CC1(C)c2ccccc2-c2c1c1c3cc(-c4nc(-c5ccccc5)nc(-c5cccc6c5oc5ccccc56)n4)ccc3sc1c1ccccc21]

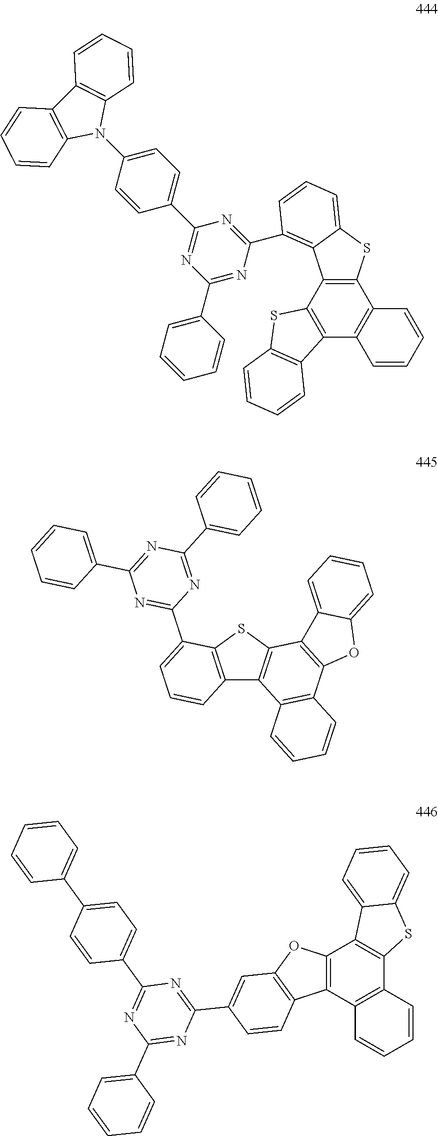 [SMILES: c1ccc(-c2nc(-c3ccc(-n4c5ccccc5c5ccccc54)cc3)nc(-c3cccc4sc5c6ccccc6c6c7ccccc7sc6c5c34)n2)cc1 ; c1ccc(-c2nc(-c3ccccc3)nc(-c3cccc4c3sc3c4c4ccccc4c4oc5ccccc5c43)n2)cc1 ; c1ccc(-c2ccc(-c3nc(-c4ccccc4)nc(-c4ccc5c(c4)oc4c5c5ccccc5c5sc6ccccc6c45)n3)cc2)cc1]

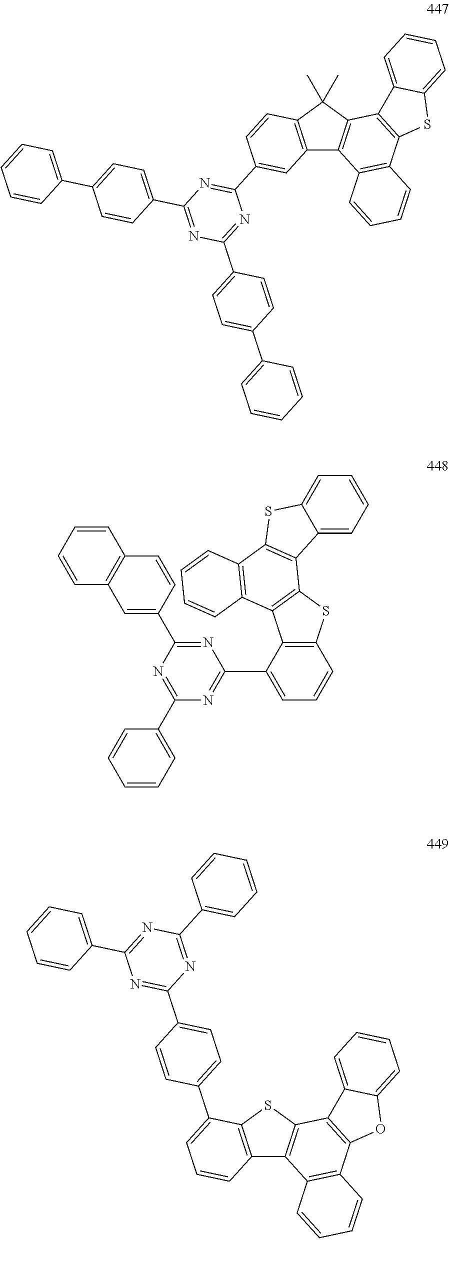

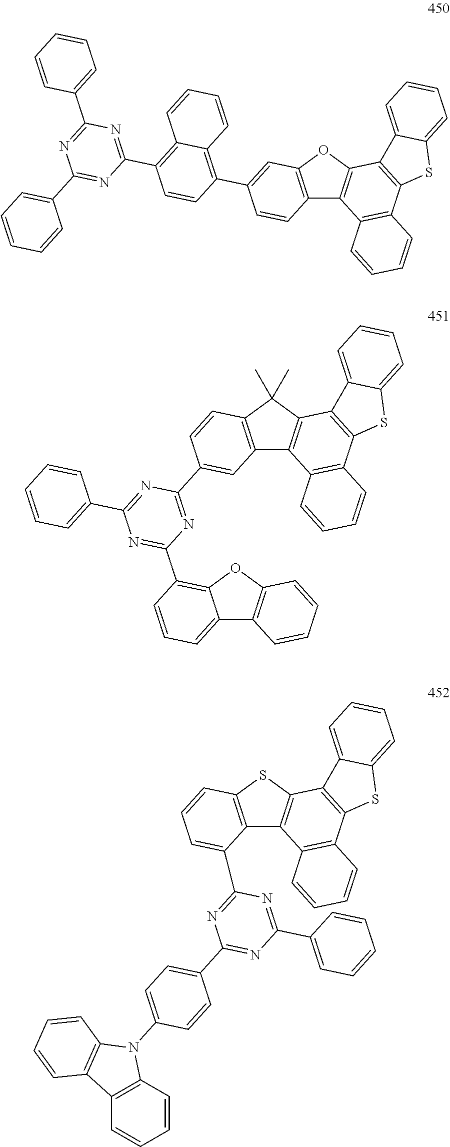 [SMILES: c1ccc(-c2nc(-c3ccccc3)nc(-c3ccc(-c4ccc5c(c4)oc4c5c5ccccc5c5sc6ccccc6c45)c4ccccc34)n2)cc1 ; CC1(C)c2ccc(-c3nc(-c4ccccc4)nc(-c4cccc5c4oc4ccccc45)n3)cc2-c2c1c1c3ccccc3sc1c1ccccc21 ; c1ccc(-c2nc(-c3ccc(-n4c5ccccc5c5ccccc54)cc3)nc(-c3cccc4sc5c6c7ccccc7sc6c6ccccc6c5c34)n2)cc1]

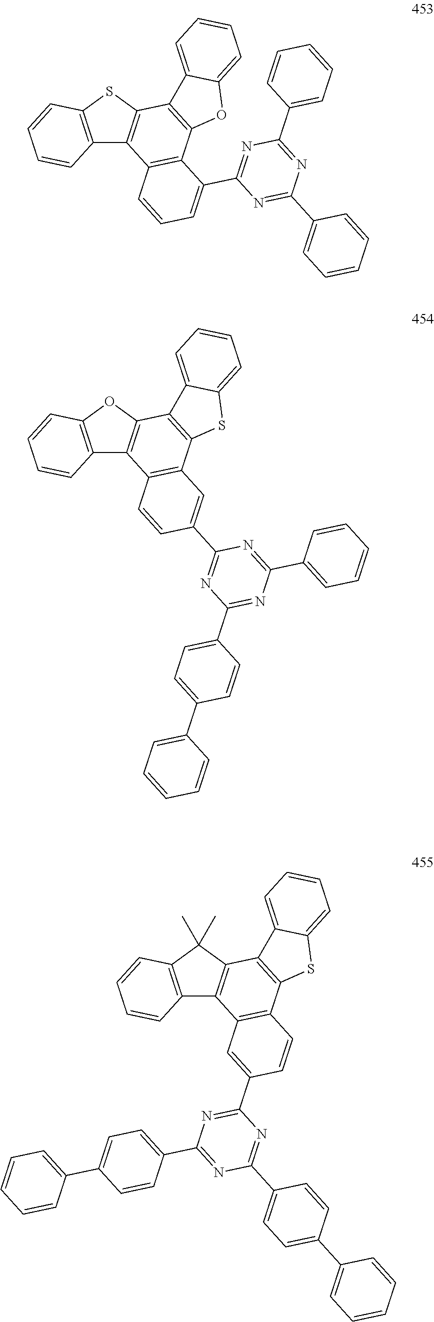 [SMILES: c1ccc(-c2nc(-c3ccccc3)nc(-c3cccc4c3c3oc5ccccc5c3c3sc5ccccc5c43)n2)cc1 ; c1ccc(-c2ccc(-c3nc(-c4ccccc4)nc(-c4ccc5c(c4)c4sc6ccccc6c4c4oc6ccccc6c54)n3)cc2)cc1 ; CC1(C)c2ccccc2-c2c1c1c3ccccc3sc1c1ccc(-c3nc(-c4ccc(-c5ccccc5)cc4)nc(-c4ccc(-c5ccccc5)cc4)n3)cc21]

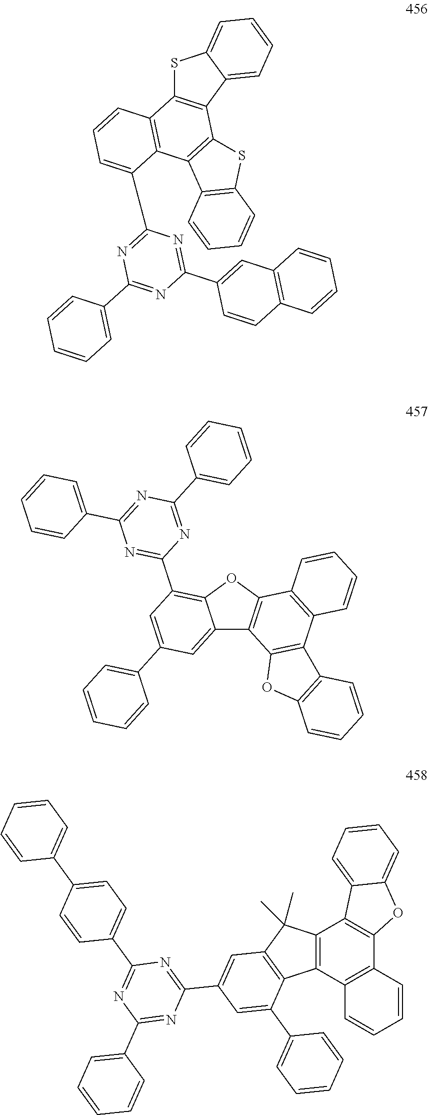 [SMILES: c1ccc(-c2nc(-c3ccc4ccccc4c3)nc(-c3cccc4c5sc6ccccc6c5c5sc6ccccc6c5c34)n2)cc1 ; c1ccc(-c2cc(-c3nc(-c4ccccc4)nc(-c4ccccc4)n3)c3oc4c5ccccc5c5c6ccccc6oc5c4c3c2)cc1 ; CC1(C)c2cc(-c3nc(-c4ccccc4)nc(-c4ccc(-c5ccccc5)cc4)n3)cc(-c3ccccc3)c2-c2c1c1c3ccccc3oc1c1ccccc21]

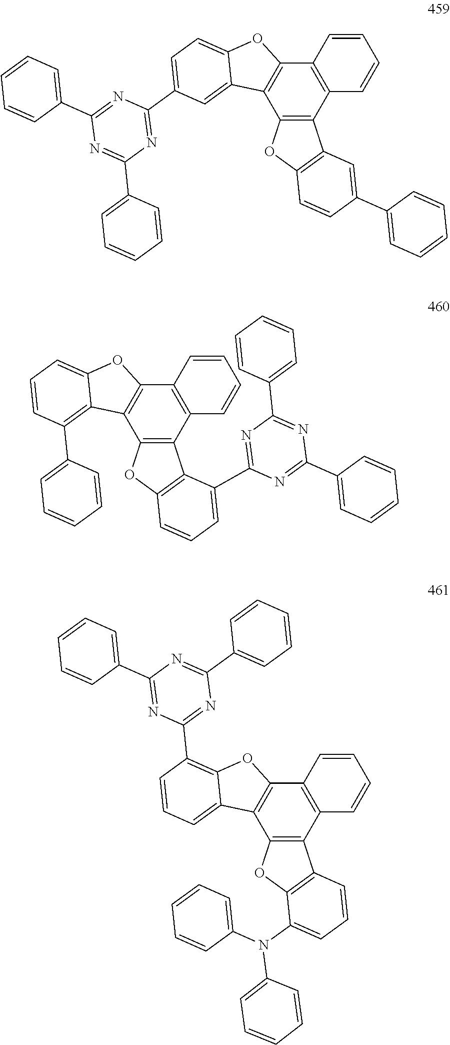 [SMILES: c1ccc(-c2ccc3oc4c(c3c2)c2ccccc2c2oc3ccc(-c5nc(-c6ccccc6)nc(-c6ccccc6)n5)cc3c24)cc1 ; c1ccc(-c2nc(-c3ccccc3)nc(-c3cccc4oc5c(c6ccccc6c6oc7cccc(-c8ccccc8)c7c65)c34)n2)cc1 ; c1ccc(-c2nc(-c3ccccc3)nc(-c3cccc4c3oc3c5ccccc5c5c6cccc(N(c7ccccc7)c7ccccc7)c6oc5c43)n2)cc1]

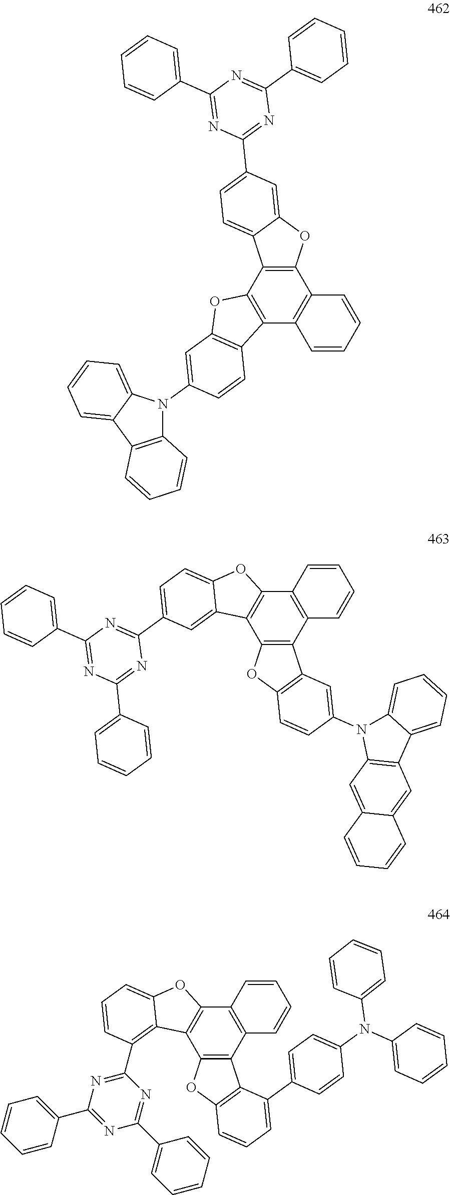 [SMILES: c1ccc(-c2nc(-c3ccccc3)nc(-c3ccc4c(c3)oc3c5ccccc5c5c6ccc(-n7c8ccccc8c8ccccc87)cc6oc5c43)n2)cc1 ; c1ccc(-c2nc(-c3ccccc3)nc(-c3ccc4oc5c6ccccc6c6c7cc(-n8c9ccccc9c9cc%10ccccc%10cc98)ccc7oc6c5c4c3)n2)cc1 ; c1ccc(-c2nc(-c3ccccc3)nc(-c3cccc4oc5c6ccccc6c6c(oc7cccc(-c8ccc(N(c9ccccc9)c9ccccc9)cc8)c76)c5c34)n2)cc1]

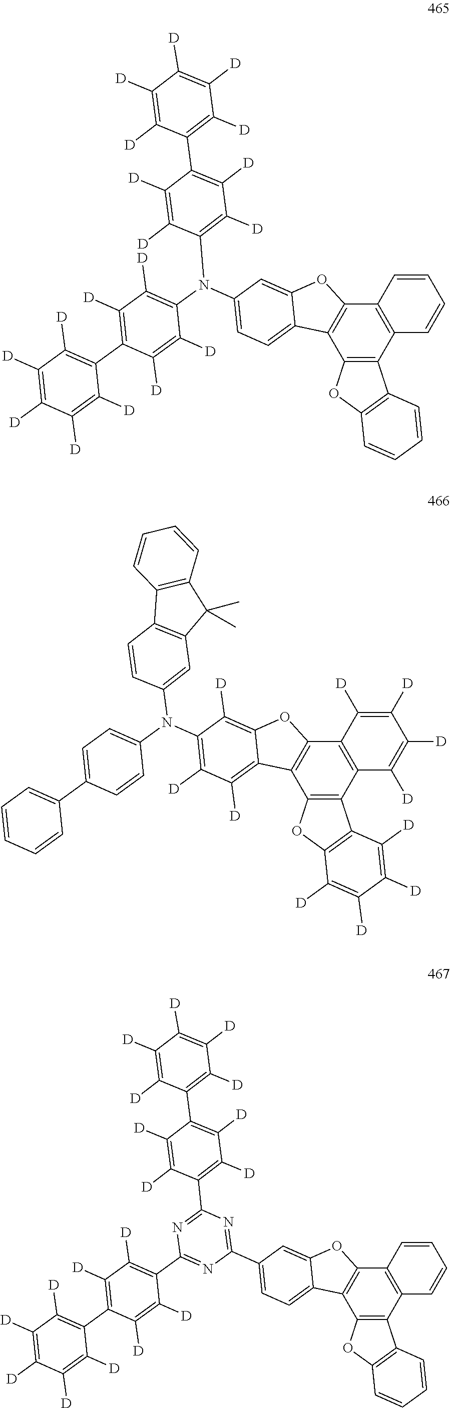

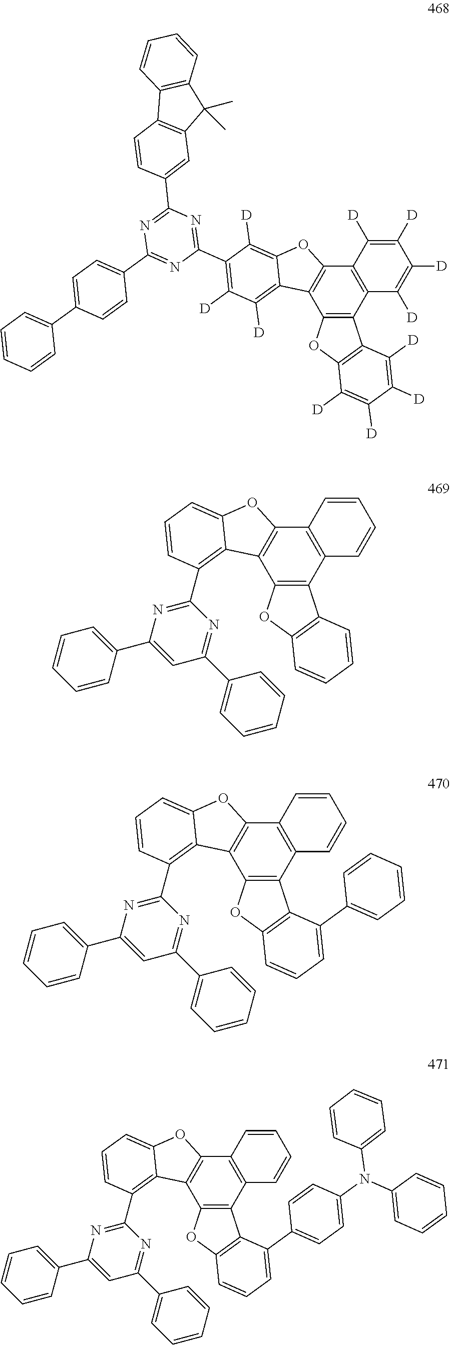 [SMILES: [2H]c1c([2H])c([2H])c2c(oc3c4c(oc5c([2H])c(-c6nc(-c7ccc(-c8ccccc8)cc7)nc(-c7ccc8c(c7)C(C)(C)c7ccccc7-8)n6)c([2H])c([2H])c54)c4c([2H])c([2H])c([2H])c([2H])c4c32)c1[2H] ; c1ccc(-c2cc(-c3ccccc3)nc(-c3cccc4oc5c6ccccc6c6c7ccccc7oc6c5c34)n2)cc1 ; c1ccc(-c2cc(-c3ccccc3)nc(-c3cccc4oc5c6ccccc6c6c(oc7cccc(-c8ccccc8)c76)c5c34)n2)cc1 ; c1ccc(-c2cc(-c3ccccc3)nc(-c3cccc4oc5c6ccccc6c6c(oc7cccc(-c8ccc(N(c9ccccc9)c9ccccc9)cc8)c76)c5c34)n2)cc1]

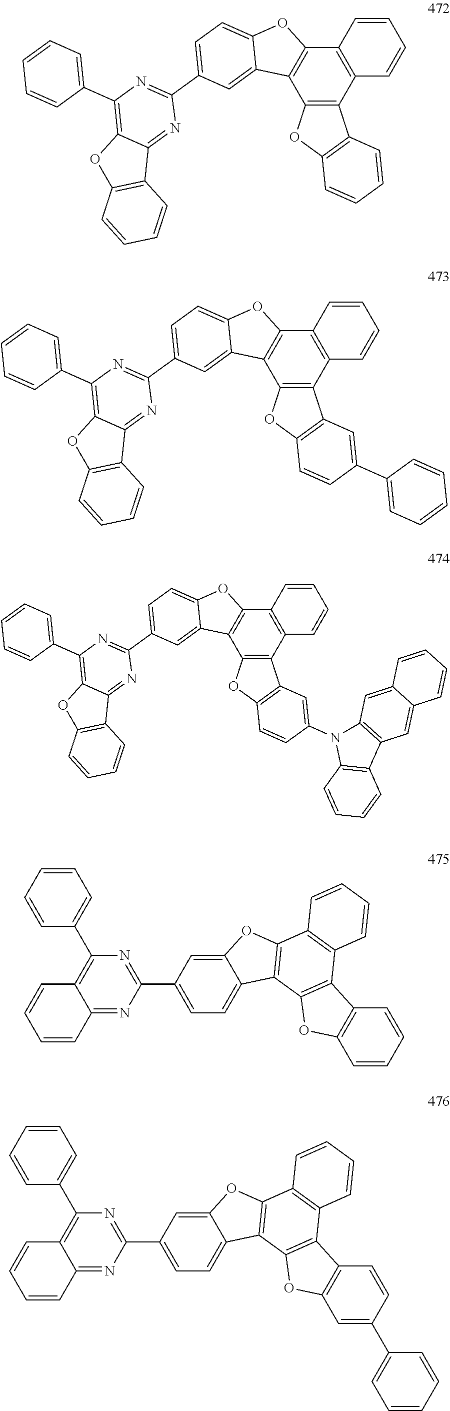 [SMILES: c1ccc(-c2nc(-c3ccc4oc5c6ccccc6c6c7ccccc7oc6c5c4c3)nc3c2oc2ccccc23)cc1 ; c1ccc(-c2ccc3oc4c(c3c2)c2ccccc2c2oc3ccc(-c5nc(-c6ccccc6)c6oc7ccccc7c6n5)cc3c24)cc1 ; c1ccc(-c2nc(-c3ccc4oc5c6ccccc6c6c7cc(-n8c9ccccc9c9cc%10ccccc%10cc98)ccc7oc6c5c4c3)nc3c2oc2ccccc23)cc1 ; c1ccc(-c2nc(-c3ccc4c(c3)oc3c5ccccc5c5c6ccccc6oc5c43)nc3ccccc23)cc1 ; c1ccc(-c2ccc3c(c2)oc2c3c3ccccc3c3oc4cc(-c5nc(-c6ccccc6)c6ccccc6n5)ccc4c32)cc1]

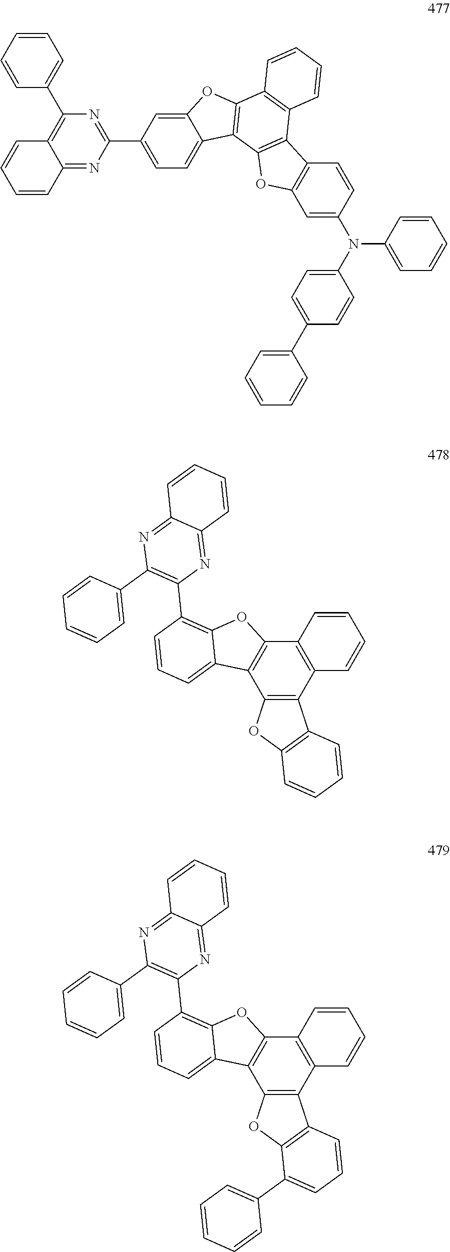 [SMILES: c1ccc(-c2ccc(N(c3ccccc3)c3ccc4c(c3)oc3c4c4ccccc4c4oc5cc(-c6nc(-c7ccccc7)c7ccccc7n6)ccc5c43)cc2)cc1 ; c1ccc(-c2nc3ccccc3nc2-c2cccc3c2oc2c4ccccc4c4c5ccccc5oc4c32)cc1 ; c1ccc(-c2nc3ccccc3nc2-c2cccc3c2oc2c4ccccc4c4c5cccc(-c6ccccc6)c5oc4c32)cc1]

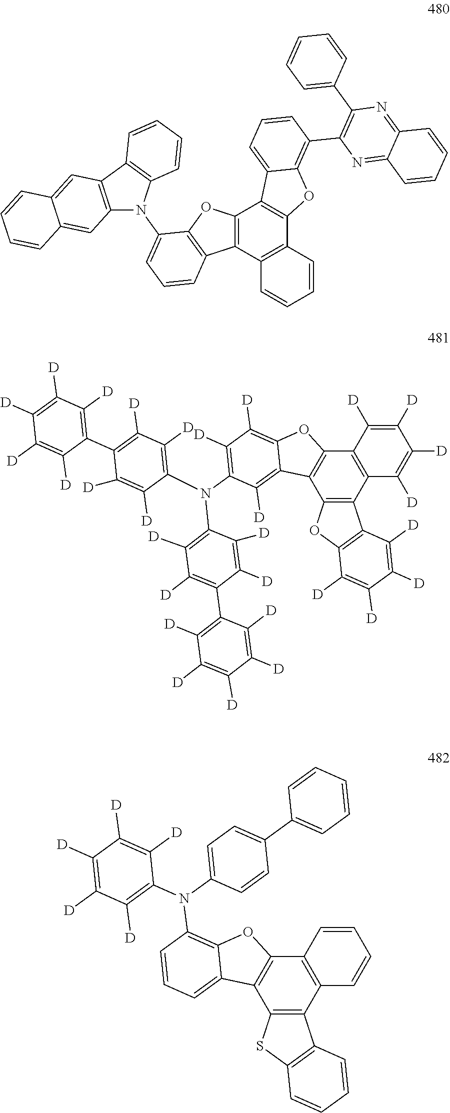 [SMILES: c1ccc(-c2nc3ccccc3nc2-c2cccc3c2oc2c4ccccc4c4c5cccc(-n6c7ccccc7c7cc8ccccc8cc76)c5oc4c32)cc1 ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4oc5c6c([2H])c([2H])c([2H])c([2H])c6c6c(oc7c([2H])c([2H])c([2H])c([2H])c76)c5c4c3[2H])c([2H])c2[2H])c([2H])c1[2H] ; [2H]c1c([2H])c([2H])c(N(c2ccc(-c3ccccc3)cc2)c2cccc3c2oc2c4ccccc4c4c5ccccc5sc4c32)c([2H])c1[2H]]

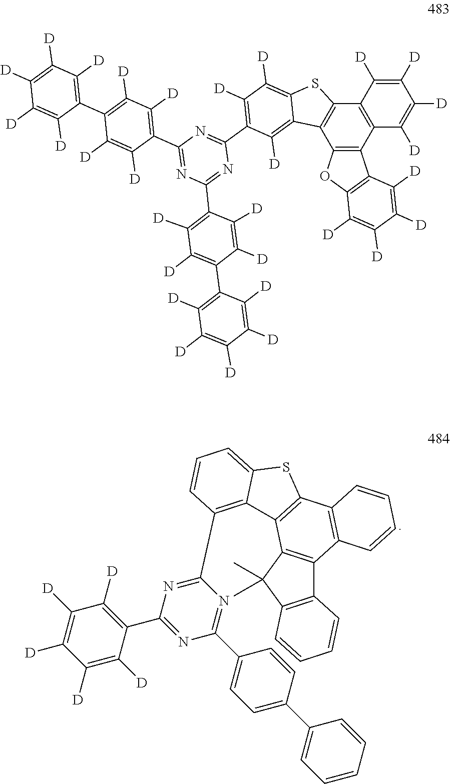 [SMILES: [2H]c1c([2H])c([2H])c(-c2c([2H])c([2H])c(-c3nc(-c4c([2H])c([2H])c(-c5c([2H])c([2H])c([2H])c([2H])c5[2H])c([2H])c4[2H])nc(-c4c([2H])c([2H])c5sc6c7c([2H])c([2H])c([2H])c([2H])c7c7c(oc8c([2H])c([2H])c([2H])c([2H])c87)c6c5c4[2H])n3)c([2H])c2[2H])c([2H])c1[2H] ; [2H]c1c([2H])c([2H])c(-c2nc(-c3ccc(-c4ccccc4)cc3)nc(-c3cccc4sc5c6ccccc6c6c(c5c34)C(C)(C)c3ccccc3-6)n2)c([2H])c1[2H]]

8. An organic light emitting device comprising:
a first electrode;
a second electrode; and
one or more organic material layers provided between the first electrode and the second electrode,
wherein one or more layers of the organic material layers include one or more types of the condensed polycyclic compound of claim 1.

9. The organic light emitting device of claim 8, wherein the organic material layer includes a light emitting layer, and the light emitting layer includes one or more types of the condensed polycyclic compound.

10. The organic light emitting device of claim 8, wherein the organic material layer includes a light emitting layer, the light emitting layer includes a host, and the host includes one or more types of the condensed polycyclic compound.

11. The organic light emitting device of claim 8, further comprising one or more layers selected from the group consisting of a light emitting layer, a hole injection layer, a hole transfer layer, an electron injection layer, an electron transfer layer, an electron blocking layer and a hole blocking layer.
